(19) United States
(12) Patent Application Publication
(10) Pub. No.: US 2022/0102660 A1
(43) Pub. Date: Mar. 31, 2022

(54) DEFECT SUPPRESSED METAL HALIDE PEROVSKITE LIGHT-EMITTING MATERIAL AND LIGHT-EMITTING DIODE COMPRISING THE SAME

(71) Applicant: SEOUL NATIONAL UNIVERSITY R&DB FOUNDATION, Seoul (KR)

(72) Inventors: Tae-Woo LEE, Seoul (KR); Joo Sung KIM, Seoul (KR); Sungjin KIM, Seoul (KR); Jung Min HEO, Seoul (KR); Young-Hoon KIM, Seoul (KR); Jin Woo PARK, Seoul (KR)

(21) Appl. No.: 17/268,462

(22) PCT Filed: Dec. 23, 2019
(86) PCT No.: PCT/KR2019/018289

§ 371 Date: Feb. 13, 2021

(30) Foreign Application Priority Data
Dec. 16, 2019 (KR) 10-2019-0168361

(51) Int. Cl. H01L 51/50; H01L 51/00; C07C 277/00; C01G 21/16

(52) U.S. Cl. CPC H01L 51/502; H01L 51/0003; B82Y 20/00; C01G 21/16; C07C 277/00

(57) ABSTRACT

Disclosed are a metal halide perovskite light-emitting material with controlled defects and wavelength converting body having the same, and light-emitting device. Monvalent organic cation (A₂) contained in the perovskite nanocrystal can stabilize the perovskite nanocrystal and suppress the generation of defects in the crystal due to the entropy effect. Remnant A₂ cations not included in the perovskite nanocrystal form a structure surrounding the perovskite nanocrystal particles, and passivate defects generated on the surface of the perovskite nanocrystal particles. Photoluminescence quantum efficiency, photoluminescence lifetime, and stability are improved through passivation of defects, and the metal halide perovskite light-emitting material can be effectively used in a light-emitting layer or a wavelength conversion layer of a light-emitting device.

### TECHNICAL FIELD

The present disclosure relates to light-emitting material of a defect suppressed metal halide perovskite using the organic cation engineering and light-emitting diode comprising the same.

### BACKGROUND ART

The current megatrend in the display market is moving to a high-efficiency, high-resolution display that aims to achieve high-purity and natural colors in addition to the existing high-efficiency, high-resolution displays. From this point of view, an organic light-emitting-diode (OLED) device based on an organic light emitter has made a leap forward, and an inorganic quantum dot LED with improved color purity is being actively researched and developed as another alternative. However, both the organic and the inorganic quantum dot light emitters have inherent limitations in terms of materials.

Existing organic light emitters have the advantage of high efficiency, but their color purity is poor due to a wide emission spectrum. In addition, inorganic quantum dot emitters have been known to have good color purity, but because they emit light by quantum confinement effect or quantum size effect, the luminous color changes according to the size of the nanocrystal particle which are mainly of diameters (or edge length in the case of a cube, or the thickness in the case of a plate) of 10 nm or less for spheres and wires, but there is a problem in that the color purity decreases because it is difficult to control the quantum dot size to be uniform as it goes toward the blue color. Moreover, since the inorganic quantum dots have a very deep valence band, there is a problem in that hole injection is difficult because the hole injection barrier at the interface with the organic hole injection layer is very large. In addition, the organic light emitters and the inorganic quantum dot light emitters have a disadvantage of being expensive. Accordingly, there is a need for a new type of organic-inorganic hybrid light emitters that compensates for the shortcomings of the organic light emitters and the inorganic quantum dot emitters and still maintains their advantages.

On the other hand, organic-inorganic hybrid materials have the advantages of low manufacturing costs, simple manufacturing and device fabrication processes, and the advantages of organic materials that are easy to control optical and electrical properties, and of inorganic materials having high charge mobility and mechanical and thermal stability. It is in the spotlight academically and industrially because you can have both advantages of organic and inorganic light emitters.

Among them, the metal halide perovskite material has a high color purity, simple color control, and low synthesis cost, so the possibility of development as a light emitter is very high. In addition, since it has a high color purity (Full width at half maximum (FWHM)≈20 nm), it is possible to implement a light-emitting device that emit a color closer to natural color.

A material having a conventional perovskite structure (ABX₃) is an inorganic metal oxide.

These inorganic metal oxides are generally oxides, and metals such as Ti, Sr, Ca, Cs, Ba, Y, Gd, La, Fe, and Mn having different sizes at the A and B sites (alkali metals, alkaline earth metals, transition metals, and lanthanum groups) cations are located, and oxygen anions are located at the X site, and the metal cations at the B site are 6-fold coordination with the oxygen anions at the X site. It is a material that is bound in the form of a corner-sharing octahedron. Examples thereof include SrFeO₃, LaMnO₃, and CaFeO₃.

In contrast, metal halide perovskite has an organic ammonium (RN) cation or metal cation located at the A site in the ABX₃ structure, and a halide anion (Cl⁻, Br⁻, I⁻) at the X site. As a result, a metal halide perovskite material is formed, so the composition is completely different from that of the inorganic metal oxide perovskite material.

In addition, the properties of the material are also changed according to the difference between these constituent materials. Inorganic metal oxide perovskite typically exhibits properties such as superconductivity, ferroelectricity, and colossal magnetoresistance, and therefore, research has been generally applied to sensors, fuel cells, and memory devices. As an example, yttrium barium copper oxide has superconducting or insulating properties depending on the oxygen contents.

On the other hand, metal halide perovskite is similar to lamellar crystal structure because the organic or alkali metal plane and the inorganic plane are stacked alternately, so the excitons can be confined within the inorganic plane of the crystal. Therefore, since the properties of the metal halide perovskite are essentially determined by the crystal structure rather than the size of the material, the metal halide perovskite itself can be an ideal light emitter that emits light of very high color purity.

Even among metal halide perovskite materials, organic-inorganic hybrid perovskite (i.e., organometal halide perovskite), if organic ammonium (A) contains a chromophore (mainly including a conjugated structure) which have a smaller band gap than the central metal-halogen crystal structure (BX₃), light of high color purity cannot be emitted, and the full-width-at-half-maximum (FWHM) of the emission spectrum becomes wider than 50 nm, making it unsuitable as a light emitting layer. Therefore, in this case, it is not very suitable for the high color purity emitters emphasized in this patent. Therefore, in order to make a high-color-purity light emitters, it is important that organic ammonium does not contain a chromophore and that light emission occurs in an inorganic lattice composed of a central metal-halogen element. In other words, this patent focuses on the development of high color purity and high efficiency light emitters that emit light that originates from an inorganic lattice.

For example, Korean Published Patent No. 10-2001-0015084 (2001, Feb. 26) discloses an electroluminescent device using a dye-containing organic-inorganic hybrid material as a light emitting layer by forming their thin film instead of particles. It does not emit light from the perovskite lattice structure.

However, since metal halide perovskite has small exciton binding energy, it is possible to emit light at low temperatures, but at room temperature, excitons do not undergo light emission due to thermal ionization and delocalization of charge carriers: this is a fundamental problem. In addition, when free charges recombine to form excitons, there is a problem in that excitons are quenched by a layer having a high conductivity adjacent to them, and thus light emission cannot occur.

In general, a metal halide perovskite crystal that is a light emitter used as a light absorbing layer of a solar cell have fundamental structural property differences from a metal halide perovskite emitter. For solar cells, a polycrystalline perovskite thin film with a large grain size (>100 nm) is used. In a polycrystalline thin film having a grain size of a certain size (approximately 100 nm) or more, metal halide perovskite intrinsically has a small exciton binding energy, so it can emit light at low temperatures, but at room temperature, excitons thermally ionize into free carriers. There is a fundamental problem in that excitons do not emit light due to thermal ionization and delocalization of the charge carriers but are separated into free charge carriers and then disappeared. In addition, when free charge carriers recombine to form excitons, the excitons are annihilated by the surrounding high conductivity layer so that light emission does not occur. Thus, the metal halide perovskite thin film is suitable for use as a light absorbing layer of a solar cell, but not as a light emitter. To solve this problem, research is being conducted to synthesize a metal halide perovskite into nanocrystal particle instead of a thin film. Regarding the synthesis of metal halide nanocrystal particle, the inventors disclosed in Korean Patent Registration No. 10-1815588 (2017, Dec. 29), which synthesizes nanocrystal particle with improved efficiency and durability-stability.

However, metal halide perovskite nanocrystals used as light emitters have a large surface-to-volume ratio due to the small particle size of several nanometers to tens of nanometers, and thus high defect concentration. Therefore, it is essential to develop a technology capable of simultaneously effectively controlling defects that may be formed on the surface of nanocrystals as well as inside the metal halide perovskite crystals.

Particles are distinguished from grains; one particle acts independently, and most of them are colloidal particles obtained by synthesizing in a solution state, and in this case, there is also a case where there is a ligand surrounding the particles due to chemical interaction. In the case of grains, they do not include ligands that surround the grains in a polycrystalline thin film, form a grain boundary, and are connected to each other, and are mainly formed into a polycrystalline thin film by being crystallized directly from precursors. At this time, since one grain can look exactly like a particle but it is an accurate expression only when it is called a grain, and it is not called a particle because it cannot be defined separately. In the case of particles without ligands, all settle within a few hours and do not form a stable dispersion. As a ligand, small molecules, which act as surfactants, are mainly used. In addition, a form that cannot be considered by separating only the particles individually because they are converted directly from the precursor solution and are irregularly attached to any other substance (e.g. Chemistry Letters, 2012, 41, 397399) is distinguished from colloidal nanocrystal particle solution. In addition, some other materials (polymers, ceramics) and perovskite precursors are mixed and reacted without a ligand to form particles to form a solid state such as a thin film (in this case, it is called an in-situ crystal formation method) is also distinguished from colloidal nanocrystal particle. When the perovskite precursor is mixed with a substance and then converted to perovskite, the bulk perovskite grains become very large, forming a precipitate rather than a particle; in this case, it has the same properties as the bulk nano grains. (e.g. Journal of Materials Chemistry, 2012, 22, 8271-8280).

### DISCLOSURE

### Technical Problem

Accordingly, the present disclosure was conceived in order to solve the above problems, and a first object of the present disclosure is to provide a metal halide perovskite light emitting material whose defects are controlled using a medium-sized monovalent organic cation.

A second objective of the present disclosure is to provide a metal halide perovskite light emitting material whose defects are controlled using four or more (Quadruple) organic cations.

In addition, a third objective of the present disclosure is to provide a wavelength converting body including a metal halide perovskite light emitting material in which the above defects are controlled.

In addition, a fourth objective of the present disclosure is to provide a light emitting device including a metal halide perovskite light emitting material in which the above defects are controlled.

### Technical Solution

To achieve the above-mentioned first objective, this disclosure offers the metal halide perovskite with the structure of ABX₃(3D), A₄BX(0D), AB₂X₅(2D), A₂BX₄(2D), A₂B⁺B³⁺X₆(3D), A₃B₂X₉(2D), or Aₙ₋₁BₙX₃ₙ₊₁ (quasi-2D) where A is monovalent cation, B is metal, X is halogen.

While tolerance factor is defined as

$$t = \frac{{R}_{A} + {R}_{X}}{\sqrt{2} ( {R}_{B} + {R}_{X} )}$$

(R_{A}, R_{B}, R_{X} is the ionic radius of A, B, X respectively), the above mentioned A is the mixture of 1^{st} monovalent cation (A₁) which can provide the tolerance factor smaller than 1.01 when incorporated into perovskite structure, and the 2^{nd} monovalent cation (A₂) which can make the tolerance factor larger than 1.01 and smaller than 3. A₂ monovalent cation is incorporated inside the metal halide perovskite crystal as well as the surface of the nanocrystal simultaneously.

Also preferably, the perovskite light emitting material may be a colloidal nanoparticle dispersed in a solvent or a polycrystalline bulk thin film.

In addition, preferably, the first monovalent cation (A₁) is at least one selected from the group consisting of methylammonium, formamidinium, Cs and Rb or a combination thereof which is capable of making a tolerance factor of 1.01 or less, and the second monovalent cation is ethylammonium, guanidinium, tert-butylammonium, and diethylammonium, dimethylammonium, ethane-1,2,-diammonium, imidazolium, n-propylammonium, iso-propylammonium and pyrrolidinium, or a combination thereof which can make the tolerance factor from 1.01 to less than 3.

In addition, preferably, the ratio of the second monovalent cation (A₂) among the monovalent cations at the A site may be 5% or more and 60% or less relative to the total amount of a mixture of the first monovalent (A₁) cation and the second monovalent (A₂) cation. For example, 5%, 5.5%, 6%, 6.5%, 7%, 7.5%, 8%, 8.3%, 8.5%, 8.7%, 9%, 9.3%, 9.5%, 9.7%, 10%, 10.3%, 10.5%, 10.7%, 11%, 11.3%, 11.5%, 11.7%, 12%, 12.5%, 13%, 13.5%, 14%, 14.5%, 15%, 15.5%, 16%, 16.5%, 17%, 17.5%, 18%, 18.5%, 19%, 19.5%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%. Preferably it may be 7% or more and 30% or less. More preferably, it may be 8% or more and 20% or less. More preferably, it may be 9% or more and 15% or less.

In addition, preferably, when the perovskite light-emitting material is a nanoparticle, the nanoparticles can have a crystal size equal to or greater than an exciton Bohr diameter (about 10 nm based on MAPbBr₃, about 7 nm based on CsPbBr₃) or equal to or less than 30 nm. For example, crystal size can be 7 nm, 7.5 nm, 8 nm, 8.3 nm, 8.5 nm, 8.7 nm, 9 nm, 9.3 nm, 9.5 nm, 9.7 nm, 10 nm, 10.3 nm, 10.5 nm, 10.7 nm, 11 nm, 11.3 nm, 11.5 nm, 11.7 nm, 12 nm, 12.3 nm, 12.5 nm, 12.7 nm, 13 nm, 13.3 nm, 13.5 nm, 13.7 nm, 14 nm, 14.3 nm, 14.5 nm, 14.7 nm, 15 nm, 15.3 nm, 15.5 nm, 15.7 nm, 16 nm, 16.5 nm 17 nm, 17.5 nm, 18 nm, 18.5 nm, 19 nm, 19.5 nm, 20 nm, 21 nm, 22 nm, 23 nm, 24 nm, 25 nm, 26 nm, 27 nm, 28 nm, 29 nm, 30 nm. Preferably, it may be 7 nm or more and 25 nm or less. More preferably, it may be 10 nm or more and 20 nm or less. More preferably, it may be 10 nm or more and 15 nm or less.

Also preferably, the perovskite light-emitting material may emit light in a region of 450 nm to 650 nm. More preferably, light in a region of 430 nm to 780 nm may be emitted. More preferably, light in a region of 450 nm to 650 nm may be emitted. For example, it may be 200 nm, 250 nm, 300 nm, 350 nm, 400 nm, 410 nm, 420 nm, 430 nm, 440 nm, 450 nm, 455 nm, 460 nm, 465 nm, 470 nm, 475 nm, 480 nm, 485 nm, 490 nm, 500 nm, 510 nm, 520 nm, 525 nm, 530 nm, 535 nm, 540 nm, 545 nm, 550 nm, 560 nm, 570 nm, 580 nm, 590 nm, 600 nm, 610 nm, 620 nm, 625 nm, 630 nm, 635 nm, 640 nm, 650 nm.

Also preferably, the first monovalent (A₁) cation is a combination of methylammonium, formamidinium, and cesium, the second monovalent (A₂) cation is guanidinium, and the guanidinium may be contained in an amount of 5% or more and 60% or less relative to the total amount of the mixture of methylammonium, formamidinium, cesium and guanidinium. For example, it may be 5%, 5.5%, 6%, 6.5%, 7%, 7.5%, 8%, 8.3%, 8.5%, 8.7%, 9%, 9.3%, 9.5%, 9.7%, 10%, 10.3%, 10.5%, 10.7%, 11%, 11.3%, 11.5%, 11.7%, 12%, 12.5%, 13%, 13.5%, 14%, 14.5%, 15%, 15.5%, 16%, 16.5%, 17%, 17.5%, 18%, 18.5% 19%, 19.5%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%. Preferably it may be 7% or more and 30% or less. More preferably, it may be 8% or more and 20% or less. More preferably, it may be 9% or more and 15% or less.

In addition, preferably, when the perovskite light-emitting material is a nanoparticle, the perovskite light-emitting material may convert a wavelength of light generated from an excitation light source into a specific wavelength.

In addition, preferably, when the perovskite light emitting material is a nanoparticle, it may further include a plurality of organic ligands surrounding the nanocrystal in the perovskite nanoparticle, and may be dispersed in an organic solvent. Since the presence of ligands improves dispersion stability, it becomes very slow to settle into precipitation after dispersion than when there is no organic ligand. For example, when there is no ligand, precipitation occurs within several hours, but in the case of particles surrounded by a plurality of ligands, it is possible to maintain a stable dispersion state without precipitation for at least 10 hours, preferably for several days or more.

To achieve the above-mentioned second objective, this disclosure offers the metal halide perovskite with the structure of ABX₃(3D), A₄BX(0D), AB₂X₅(2D), A₂BX₄(2D), A₂B⁺B³⁺X₆(3D), A₃B₂X₉(2D), or Aₙ₋₁BₙX₃ₙ₊₁ (quasi-2D) (n is an integer between 2 and 6) where A is monovalent cation, B is metal, X is halogen.

While tolerance factor is defined as

$$t = \frac{{R}_{A} + {R}_{X}}{\sqrt{2} ( {R}_{B} + {R}_{X} )}$$

(R_{A}, R_{B}, R_{X} is the ionic radius of A, B, X respectively), the above-mentioned A is the mixture of at least three different 1^{st} monovalent cations (A₁, A₃, A₄) which provide the tolerance factor smaller than 1.01 when incorporated into perovskite structure, and the 2^{nd} monovalent cation (A₂) which makes tolerance factor larger than 1.01 and smaller than 3, preferably, smaller than 2. The 1^{st} monovalent cations and the 2^{nd} monovalent cation (A₂) are incorporated evenly at the A sites inside the metal halide perovskite crystal.

Also preferably, the perovskite light emitting material may be a colloidal nanoparticle dispersed in a solvent or a polycrystalline bulk thin film.

In addition, preferably, the first monovalent cation (A₁) is at least one selected from the group consisting of methylammonium, formamidinium, Cs and Rb or a combination thereof which is capable of making a tolerance factor of 1.01 or less, and the second monovalent cation is ethylammonium, guanidinium, tert-butylammonium, and diethylammonium, dimethylammonium, ethane-1,2,-diammonium, imidazolium, n-propylammonium, iso-propylammonium and pyrrolidinium, or a combination thereof which can make the tolerance factor from 1.01 to less than 3.

In addition, preferably, the ratio of the second monovalent cation (A₂) among the monovalent cations at the A site may be 5% or more and 60% or less relative to the total amount of a mixture of the first monovalent (A₁) cations and the second monovalent (A₂) cations. For example, 5%, 6%, 7%, 8%, 9%, 10%, 11%, 12%, 13%, 14%, 15%, 15.1%, 15.2%, 15.3%, 15.4%, 15.5%, 15.6%, 15.7%, 15.8%, 15.9%, 16%, 16.1%, 16.2%, 16.3%, 16.4%, 16.5%, 16.6%, 16.7%, 16.8%, 16.9%, 17%, 17.1%, 17.2%, 17.3%, 17.4%, 17.5%, 17.6%, 17.7%, 17.8%, 17.9%, 18%, 18.1%, 18.2%, 18.3%, 18.4%, 18.5%, 18.6%, 18.7%, 18.8%, 18.9%, 19%, 19.1%, 19.1%, 19.1%, 19.1%, 19.1%, 19.1%, 19.1%, 19.1%, 19.1%, 20%, 20.1%, 20.2%, 20.3%, 20.4%, 20.5%, 20.6%, 20.7%, 20.8%, 20.9%, 21%, 21.1%, 21.2%, 21.3%, 21.4%, 21.5%, 21.6%, 21.7%, 21.8%, 21.9%, 22%, 22.1%, 22.2%, 22.3%, 22.4%, 22.5%, 22.6%, 22.7%, 22.8%, 22.9%, 23%, 23.1%, 23.2%, 23.3%, 23.4%, 23.5%, 23.6%, 23.7%, 23.8%, 23.9%, 24%, 24.1%, 24.2%, 24.3%, 24.4%, 24.5%, 24.6%, 24.7%, 24.8%, 24.9%, 25%, 25.5%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%. Preferably it may be 5% or more and 30% or less. More preferably, it may be 15% or more and 25% or less. More preferably, it may be 18% or more and 22% or less.

In addition, preferably, when the perovskite light-emitting material is a nanoparticle, the nanoparticles can have a crystal size equal to or greater than an exciton Bohr diameter (about 10 nm based on MAPbBr₃, about 7 nm based on CsPbBr₃) or equal to or less than 30 nm. For example, crystal size can be 7 nm, 7.5 nm, 8 nm, 8.3 nm, 8.5 nm, 8.7 nm, 9 nm, 9.3 nm, 9.5 nm, 9.7 nm, 10 nm, 10.3 nm, 10.5 nm, 10.7 nm, 11 nm, 11.3 nm, 11.5 nm, 11.7 nm, 12 nm, 12.3 nm, 12.5 nm, 12.7 nm, 13 nm, 13.3 nm, 13.5 nm, 13.7 nm, 14 nm, 14.3 nm, 14.5 nm, 14.7 nm, 15 nm, 15.3 nm, 15.5 nm, 15.7 nm, 16 nm, 16.5 nm 17 nm, 17.5 nm, 18 nm, 18.5 nm, 19 nm, 19.5 nm, 20 nm, 21 nm, 22 nm, 23 nm, 24 nm, 25 nm, 26 nm, 27 nm, 28 nm, 29 nm, 30 nm. Preferably, it may be 7 nm or more and 25 nm or less. More preferably, it may be 10 nm or more and 20 nm or less. More preferably, it may be 10 nm or more and 15 nm or less.

Also preferably, the perovskite light-emitting material may emit light in a region of 300 nm to 1500 nm. More preferably, light in a region of 430 nm to 780 nm may be emitted. More preferably, light in a region of 450 nm to 650 nm may be emitted. For example, it may be 400 nm, 410 nm, 420 nm, 430 nm, 440 nm, 450 nm, 455 nm, 460 nm, 465 nm, 470 nm, 475 nm, 480 nm, 485 nm, 490 nm, 500 nm, 510 nm, 520 nm, 525 nm, 530 nm, 535 nm, 540 nm, 545 nm, 550 nm, 560 nm, 570 nm, 580 nm, 590 nm, 600 nm, 610 nm, 620 nm, 625 nm, 630 nm, 635 nm, 640 nm, 650 nm.

Also preferably, the first monovalent (A₁) cation is a combination of methylammonium, formamidinium, and cesium, the second monovalent (A₂) cation is guanidinium, and the guanidinium may be contained in an amount of 5% or more and 60% or less relative to the total amount of the mixture of methylammonium, formamidinium, cesium and guanidinium. For example, it may be %, 6%, 7%, 8%, 9%, 10%, 11%, 12%, 13%, 14%, 15%, 15.1%, 15.2%, 15.3%, 15.4%, 15.5%, 15.6%, 15.7%, 15.8%, 15.9%, 16%, 16.1%, 16.2%, 16.3%, 16.4%, 16.5%, 16.6%, 16.7%, 16.8%, 16.9%, 17%, 17.1%, 17.2%, 17.3%, 17.4%, 17.5%, 17.6%, 17.7%, 17.8%, 17.9%, 18%, 18.1%, 18.2%, 18.3%, 18.4%, 18.5%, 18.6%, 18.7%, 18.8%, 18.9%, 19%, 19.1%, 19.1%, 19.1%, 19.1%, 19.1%, 19.1%, 19.1%, 19.1%, 19.1%, 20%, 20.1%, 20.2%, 20.3%, 20.4%, 20.5%, 20.6%, 20.7%, 20.8%, 20.9%, 21%, 21.1%, 21.2%, 21.3%, 21.4%, 21.5%, 21.6%, 21.7%, 21.8%, 21.9%, 22%, 22.1%, 22.2%, 22.3%, 22.4%, 22.5%, 22.6%, 22.7%, 22.8%, 22.9%, 23%, 23.1%, 23.2%, 23.3%, 23.4%, 23.5%, 23.6%, 23.7%, 23.8%, 23.9%, 24%, 24.1%, 24.2%, 24.3%, 24.4%, 24.5%, 24.6%, 24.7%, 24.8%, 24.9%, 25%, 25.5%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%. Preferably it may be 5% or more and 30% or less. More preferably, it may be 15% or more and 25% or less. More preferably, it may be 18% or more and 22% or less.

Also preferably, the first monovalent (A₁) cation is a combination of methylammonium, formamidinium, and cesium, the second monovalent (A₂) cation is guanidinium, and the methylammonium may be contained in an amount of 1% or more and 20% or less relative to the total amount of the mixture of 4 cations composed of methylammonium, formamidinium, cesium and guanidinium. For example, it may be 1%, 1.5%, 2%, 2.5%, 3%, 3.5%, 4%, 4.5%, 5%, 5.1%, 5.2%, 5.3%, 5.4%, 5.5%, 5.6%, 5.7%, 5.8%, 5.9%, 6%, 6.1%, 6.2%, 6.3%, 6.4%, 6.5%, 6.6%, 6.7%, 6.8%, 6.9%, 7%, 7.1%, 7.2%, 7.3%, 7.4%, 7.5%, 7.6%, 7.7%, 7.8%, 7.9%, 8%, 8.1%, 8.2%, 8.3%, 8.4%, 8.5%, 8.6%, 8.7%, 8.8%, 8.9%, 9%, 9.1%, 9.2%, 9.3%, 9.4%, 9.5%, 9.6%, 9.7%, 9.8%, 9.9%, 10%, 10.1%, 10.2%, 10.3%, 10.4%, 10.5%, 10.6%, 10.7%, 10.8%, 10.9%, 11%, 11.1%, 11.2%, 11.3%, 11.4%, 11.5%, 11.6%, 11.7%, 11.8%, 11.9%, 12%, 12.1%, 12.2%, 12.3%, 12.4%, 12.5%, 12.6%, 12.7%, 12.8%, 12.9%, 13%, 13.1%, 13.2%, 13.3%, 13.4%, 13.5%, 13.6%, 13.7%, 13.8%, 13.9%, 14%, 14.1%, 14.2%, 14.3%, 14.4%, 14.5%, 14.6%, 14.7%, 14.8%, 14.9%, 15%, 15.1%, 15.2%, 15.3%, 15.4%, 15.5%, 15.6%, 15.7%, 15.8%, 15.9%, 16%, 16.1%, 16.2%, 16.3%, 16.4%, 16.5%, 16.6%, 16.7%, 16.8%, 16.9%, 17%, 17.1%, 17.2%, 17.3%, 17.4%, 17.5%, 17.6%, 17.7%, 17.8%, 17.9%, 18%, 18.1%, 18.2%, 18.3%, 18.4%, 18.5%, 18.6%, 18.7%, 18.8%, 18.9%, 19%, 19.1%, 19.2%, 19.3%, 19.4%, 19.5%, 19.6%, 19.7%, 19.8%, 19.9%, 20%. Preferably it may be 5% or more and 15% or less. More preferably, it may be 7.5% or more and 12.5% or less. More preferably, it may be 9% or more and 11% or less.

In addition, in order to achieve the third objective, the present disclosure includes the perovskite nanoparticles for converting a wavelength of light generated from an excitation light source into a specific wavelength; And it provides a perovskite wavelength converting body comprising a dispersion medium for dispersing the perovskite nanoparticles.

In addition, in order to achieve the third objective, the present disclosure provides a substrate; A first electrode on the substrate; An emission layer positioned on the first electrode; And a second electrode positioned on the emission layer, wherein the emission layer is the perovskite light emitting material.

In addition, the present disclosure is a base structure; At least one excitation light source for emitting light of a predetermined wavelength located on the base structure; And it provides a perovskite light emitting device including the perovskite wavelength converting body located in the optical path of the excitation light source.

### Advantageous Effects

According to the present disclosure, the medium-sized monovalent organic cation (A₂) contained in the perovskite crystal stabilizes the perovskite crystal due to the entropy effect and can suppress the generation of defects in the crystal. Excessive A₂ cations that are not included inside the crystal form a structure that surrounds the perovskite nanocrystal particles and passivate the defects generated on the surface of the perovskite nanocrystal particles, thereby enhance the photoluminescence quantum efficiency, photoluminescence lifetime and stability. It can be usefully used in a light-emitting layer or a wavelength conversion layer of a light-emitting device.

In addition, according to the present disclosure, the medium-sized monovalent organic cation (A₂) capable of stabilizing the perovskite crystal can stabilize the perovskite crystal due to the entropy effect and suppress the generation of defects inside the crystal. In order that the average tolerance factor of the crystal does not increase to more than 1 even if it is mixed at a higher ratio inside the crystal, a mixture of three or more monovalent organic cations (A₁, A₃, A₄, . . . ) with a tolerance factor of less than 1 is used. By maximizing the stabilization of the perovskite crystal and minimizing the formation of defects, photoluminescence quantum efficiency, photoluminescence lifetime, and stability are improved, and thus, it can be usefully used in a light emitting layer or a wavelength conversion layer of a light emitting device.

### DESCRIPTION OF DRAWINGS

FIG. 1 is a schematic diagram showing the difference between a metal halide perovskite bulk thin film and a metal halide perovskite nanocrystal particle according to an embodiment of the present disclosure.

FIG. 2 is a schematic diagram showing a metal halide perovskite nanocrystal particles according to an embodiment of the present disclosure.

FIG. 3 is a schematic diagram showing a method of manufacturing a metal halide perovskite nanocrystal particle according to an embodiment of the present disclosure.

FIG. 4 is a schematic diagram showing a core-shell structured metal halide perovskite nanocrystal particle and an energy band diagram thereof according to an embodiment of the present disclosure.

FIG. 5 is a schematic diagram showing a method of manufacturing a metal halide perovskite nanocrystal particle having a core-shell structure according to an embodiment of the present disclosure.

FIG. 6 is a schematic diagram showing a metal halide perovskite nanocrystal particle having a gradient composition structure according to an embodiment of the present disclosure.

FIG. 7 is a schematic diagram showing a metal halide perovskite nanocrystal particle having a structure having a gradient composition and an energy band diagram thereof according to an embodiment of the present disclosure.

FIG. 8 is a schematic diagram showing a doped metal halide perovskite nanocrystal particle and an energy band diagram thereof according to an embodiment of the present disclosure.

FIG. 9 is a schematic diagram illustrating the Ostwald Ripening phenomenon of a metal halide perovskite nanocrystal particle according to an embodiment of the present disclosure.

FIG. 10 is a schematic diagram showing a method of controlling the size distribution of perovskite nanocrystal particles according to an embodiment of the present disclosure.

FIG. 11 is a graph showing the photoluminescence characteristics of metal halide perovskite nanocrystal particles prepared in air according to a conventional method.

FIG. 12 is a graph showing the photoluminescence characteristics of metal halide perovskite nanocrystal particles prepared in a nitrogen atmosphere according to an embodiment of the present disclosure.

FIG. 13 is a schematic diagram of a process of removing a solvent remaining after a bar coating process through air injection according to an embodiment of the present disclosure.

FIG. 14 is a schematic diagram showing a light emitting device according to an embodiment of the present disclosure.

FIG. 15 is a schematic diagram showing a light emitting device according to another embodiment of the present disclosure.

FIG. 16 is a schematic diagram showing the structure of a metal halide perovskite light emitting transistor according to an embodiment of the present disclosure.

FIG. 17 is a schematic diagram showing an organic nanowire lithography process sequence according to an embodiment of the present disclosure.

FIG. 18 is a schematic diagram of an electric field assisted robotic nozzle printer.

FIG. 19 is a schematic diagram showing the structure of a metal halide perovskite light emitting transistor according to another embodiment of the present disclosure.

FIG. 20 is a schematic diagram illustrating a light emitting transistor in which a semiconductor layer including a metal halide perovskite having a polycrystalline structure is located according to an embodiment of the present disclosure.

FIG. 21 is a schematic diagram illustrating a light emitting transistor in which a semiconductor layer including a metal halide perovskite having a single crystal structure is located according to another embodiment of the present disclosure.

FIG. 22 is a schematic diagram showing a metal halide perovskite light emitting device according to an embodiment of the present disclosure.

FIG. 23 shows transient photoluminescence and normal light emission before and after coating a TBMM thin film as a passivation layer on a metal halide perovskite nanocrystal particle emission layer in a perovskite light emitting device according to an embodiment of the present disclosure. It is a graph showing steady-state photoluminescence.

FIG. 24 is an X-ray photoelectron spectrum (XPS) before and after coating a TBMM thin film as a passivation layer on a metal halide perovskite nanocrystal particle emission layer in a perovskite light emitting device according to an embodiment of the present disclosure. Show.

FIG. 25 is a metal halide perovskite nanocrystal particle emission layer in a single hole device and an electron-only device among perovskite light emitting devices according to an embodiment of the present disclosure. It is a graph showing the hole current density and electron current density before and after coating the TBMM thin film as a passivation layer on the top.

FIG. 26 is a graph showing capacitance-voltage characteristics before and after coating a TBMM thin film as a passivation layer on a metal halide perovskite nanocrystal particle emission layer in a perovskite light emitting device according to an embodiment of the present disclosure to be.

FIG. 27 is a graph showing luminescence efficiency characteristics before and after coating a TBMM thin film as a passivation layer on a metal halide perovskite nanocrystal particle emission layer in a perovskite light emitting device according to an embodiment of the present disclosure.

FIG. 28 is a schematic diagram showing a method of manufacturing a light emitting device including an exciton buffer layer according to an embodiment of the present disclosure.

FIG. 29 is a schematic diagram showing the effect of an exciton buffer layer according to an embodiment of the present disclosure.

FIG. 30 is a graph showing the effect of acidity and work function when a basic additive is added to PEDOT:PSS:PFI, which is a conductive polymer hole injection layer, in a conductive polymer hole injection layer according to an embodiment of the present disclosure.

FIG. 31 illustrates a change in strength according to binding energy when aniline is deposited on the ITO electrode in PEDOT:PSS:PFI in the conductive polymer hole injection layer according to an embodiment of the present disclosure. It is a graph showing.

FIG. 32 is a diagram illustrating an interface between the hole injection layer and the metal halide perovskite light emitting layer when aniline is deposited on the ITO electrode in PEDOT:PSS:PFI in the conductive polymer hole injection layer according to an embodiment of the present disclosure. It is a graph showing the ionic strength of.

FIG. 33 shows the surface roughness of the formed thin film according to the amount of aniline added when aniline is added to PEDOT:PSS in the conductive polymer hole injection layer according to an embodiment of the present disclosure.

FIG. 34 shows the surface roughness of the formed thin film according to the amount of aniline added when aniline is added to PEDOT:PSS:PFI in the conductive polymer hole injection layer according to an embodiment of the present disclosure.

FIG. 35 is a graph showing luminescence intensity and luminescence lifetime of a metal halide perovskite in a polycrystalline metal halide perovskite layer/PEDOT:PSS:PFI:aniline/ITO electrode according to an embodiment of the present disclosure.

FIG. 36 is a graph showing emission intensity and emission lifetime of metal halide perovskite in a metal halide perovskite nanoparticle layer/PEDOT:PSS:PFI:aniline/ITO electrode according to an embodiment of the present disclosure.

FIG. 37 is a graph showing device efficiency of a polycrystalline metal halide perovskite device and a metal halide perovskite nanoparticle device using a PEDOT:PSS:PFI:aniline hole injection layer according to an embodiment of the present disclosure.

FIG. 38 is a schematic diagram showing a method of coating by dropping a low-molecular organic material solution before the solvent of the light-emitting layer evaporates while the metal halide perovskite light-emitting layer according to an embodiment of the present disclosure is coated, that is, an organic material-assisted nanocrystal immobilization process to be.

FIG. 39 is a graph showing a point in time when a low molecular weight organic material solution is dropped while the metal halide perovskite emission layer is coated when the metal halide perovskite emission layer is manufactured according to an embodiment of the present disclosure.

FIG. 40 is a cross-sectional view illustrating a metal halide perovskite-organic low-molecular host mixed light emitting layer according to an embodiment of the present disclosure.

FIG. 41 shows energy levels of perovskite and organic low-molecular hosts used in the perovskite-organic low-molecular host mixed emission layer according to an embodiment of the present disclosure.

FIG. 42 shows energy levels of an organic small molecule host used in a metal halide perovskite-organic small molecule host mixed emission layer according to an embodiment of the present disclosure.

FIG. 43 is a schematic diagram showing the structure of a high vacuum evaporator for manufacturing a perovskite-organic low-molecular host mixed light emitting layer according to an embodiment of the present disclosure.

FIG. 44 shows energy levels of constituent layers in a light-emitting device (structure) including a metal halide perovskite-organic low-molecular host mixed light-emitting layer according to an embodiment of the present disclosure.

FIG. 45 shows energy levels of constituent layers in a light-emitting device (inverse structure) including a metal halide perovskite-organic low-molecular host mixed light-emitting layer according to another embodiment of the present disclosure.

FIG. 46 is an example of a structure of a light emitting diode including a multidimensional metal halide perovskite hybrid light emitting layer according to an embodiment of the present disclosure.

FIG. 47 is a schematic diagram showing examples of various methods of manufacturing a multidimensional perovskite hybrid light emitting layer according to an embodiment of the present disclosure.

FIG. 48 shows a core/shell structure of a perovskite film according to an embodiment of the present disclosure.

FIG. 49 shows a mechanism for forming a core/shell structure of a metal halide perovskite film according to an embodiment of the present disclosure.

FIG. 50 shows a principle of forming each of the core and shell structures of a metal halide perovskite film according to an embodiment of the present disclosure.

FIG. 51 shows a proton nuclear magnetic resonance analysis spectrum of a perovskite film with or without a self-assembled shell according to an embodiment of the present disclosure.

FIG. 52 is a schematic diagram and a scanning electron microscope image of a crystal shape of a perovskite film with or without a self-assembled shell according to an embodiment of the present disclosure.

FIG. 53 is a graph showing photoluminescence characteristics of a perovskite film with or without a self-assembled shell according to an embodiment of the present disclosure.

FIG. 54 is a graph showing charge life characteristics of a perovskite film with or without a self-assembled shell according to an embodiment of the present disclosure.

FIG. 55 is a schematic diagram of a self-assembled polymer-metal halide perovskite light emitting layer according to an embodiment of the present disclosure.

FIG. 56 is a schematic diagram of a self-assembled polymer-perovskite light emitting layer according to another embodiment of the present disclosure.

FIG. 57 is a flowchart illustrating a method of manufacturing a self-assembled polymer-perovskite light emitting layer according to an embodiment of the present disclosure.

FIG. 58 is a schematic diagram showing a process of forming a self-assembled polymer pattern on a substrate according to an embodiment of the present disclosure.

FIG. 59 is a flowchart illustrating a method of manufacturing a self-assembled polymer-perovskite light emitting layer according to another embodiment of the present disclosure.

FIG. 60 illustrates an organic material layer on a self-assembled polymer pattern formed on a substrate according to an embodiment of the present disclosure.

FIG. 61 is a schematic diagram showing a process of disposing perovskite nanocrystal particles in a self-assembled polymer pattern formed on a substrate according to an embodiment of the present disclosure.

FIG. 62 is a flowchart illustrating a method of manufacturing a self-assembled polymer-perovskite light emitting layer according to another embodiment of the present disclosure.

FIG. 63 is a schematic diagram showing a method of manufacturing a quasi-two-dimensional perovskite film in which the structure of a nanocrystal is adjusted according to an embodiment of the present disclosure.

FIG. 64 is an image of a quasi-two-dimensional perovskite crystal in which the structure of a nanocrystal is adjusted according to an embodiment of the present disclosure.

FIG. 65 is a flowchart illustrating a method of manufacturing a metal halide perovskite nanocrystal particle light emitter in which an organic ligand is substituted according to an embodiment of the present disclosure.

FIG. 66 is a flowchart illustrating a method of manufacturing an organic-inorganic hybrid perovskite nanocrystal particle light emitter according to an embodiment of the present disclosure.

FIG. 67 is a schematic diagram showing a method of manufacturing an organic-inorganic hybrid perovskite nanocrystal particle light emitter according to an embodiment of the present disclosure.

FIG. 68 is a schematic diagram showing an organic-inorganic hybrid perovskite nanocrystal particle light emitter and an inorganic metal halide perovskite nanocrystal particle light emitter according to an embodiment of the present disclosure.

FIG. 69 is a schematic diagram showing a method of manufacturing an organic-inorganic hybrid perovskite nanocrystal particle light emitter substituted with an organic ligand according to an embodiment of the present disclosure.

FIG. 70 is a cross-sectional view illustrating a light emitting layer having a tandem structure according to an embodiment of the present disclosure.

FIG. 71 illustrates energy levels of a light emitting layer having a stacked structure in which first and second light emitting material layers are alternately located according to an embodiment of the present disclosure.

FIG. 72 illustrates energy levels of materials used in a light emitting layer having a stacked structure in which first and second light emitting material layers are alternately located according to an embodiment of the present disclosure.

FIG. 73 illustrates energy levels of constituent layers in a light-emitting device (normal structure) including a light-emitting layer according to an embodiment of the present disclosure.

FIG. 74 illustrates energy levels of constituent layers in a light-emitting device (inverse structure) including a light-emitting layer according to another embodiment of the present disclosure.

FIG. 75 is a schematic structural diagram illustrating a structure of a stacked hybrid light emitting diode according to an embodiment of the present disclosure.

FIG. 76 is a schematic diagram showing the structure of a light emitting device according to an embodiment of the present disclosure (Normal structure).

FIG. 77 is a schematic diagram showing the structure of a light emitting device according to an embodiment of the present disclosure (Inverted structure).

FIG. 78 shows energy levels of a light emitting device in which a first charge transport layer (hole injection layer) of a light emitting device according to an embodiment of the present disclosure is a thin metal halide perovskite thin film (Normal structure).

FIG. 79 shows energy levels of a light emitting device in which a first charge transport layer (hole injection layer) of a light emitting device according to an embodiment of the present disclosure is a thin metal halide perovskite thin film (Inverted structure).

FIG. 80 shows energy levels of a light-emitting device in which a second charge transport layer (electron injection layer) of the light-emitting device according to an embodiment of the present disclosure is a thin film of metal halide perovskite (Normal structure).

FIG. 81 shows energy levels of a light emitting device in which the second charge transport layer (electron injection layer) of the light emitting device according to an embodiment of the present disclosure is a thin metal halide perovskite thin film (Inverted structure).

FIG. 82 shows energy levels of a light emitting device in which the first charge transport layer (hole injection layer) and the second charge transport layer (electron injection layer) of the light emitting device according to an embodiment of the present disclosure are metal halide perovskite thin films (Inverted structure).

FIG. 83 shows energy levels of a light emitting device in which the first charge transport layer (hole injection layer) and the second charge transport layer (electron injection layer) of the light emitting device according to an embodiment of the present disclosure are metal halide perovskite thin films (Normal structure).

FIG. 84 shows energy levels of a light emitting device in which the first charge transport layer (hole injection layer) and the second charge transport layer (electron injection layer) of the light emitting device according to an embodiment of the present disclosure are metal halide perovskite thin films (Inverted structure).

FIG. 85 shows energy levels of a light emitting device in which the first charge transport layer (hole injection layer) and the second charge transport layer (electron injection layer) of the light emitting device according to an embodiment of the present disclosure are metal halide perovskite thin films (Normal structure).

FIG. 86 is a schematic diagram showing a metal halide perovskite-polymer composite film according to another embodiment of the present disclosure.

FIG. 87 are cross-sectional views illustrating a method of sealing a wavelength converting body according to an embodiment of the present disclosure.

FIG. 88 is a cross-sectional view of a light emitting device including a wavelength conversion layer according to an embodiment of the present disclosure.

FIG. 89 is a cross-sectional view of a light emitting device including a wavelength converting body according to an embodiment of the present disclosure.

FIG. 90 is a cross-sectional view schematically illustrating a stretchable wavelength conversion layer according to an embodiment of the present disclosure.

FIG. 91 is a cross-sectional view of a stretchable light emitting device according to an embodiment of the present disclosure.

FIG. 92 is a schematic diagram illustrating a method of manufacturing a stretchable wavelength conversion layer according to an embodiment of the present disclosure.

FIG. 93 is another schematic diagram illustrating a method of manufacturing a stretchable wavelength conversion layer according to an embodiment of the present disclosure.

FIG. 94 is a schematic diagram illustrating a method of manufacturing a stretchable light emitting device according to an embodiment of the present disclosure.

FIG. 95 shows a hybrid wavelength converting body according to an embodiment of the present disclosure.

FIG. 96 is a schematic diagram showing a hybrid wavelength converting body according to another embodiment of the present disclosure.

FIG. 97 is a schematic diagram showing a method of manufacturing a metal halide perovskite nanocrystal particle used as a wavelength conversion particle in a hybrid wavelength converting body according to an embodiment of the present disclosure.

FIG. 98 is a cross-sectional view showing a method of manufacturing a hybrid wavelength converting body using a sealing method according to an embodiment of the present disclosure.

FIG. 99 is a cross-sectional view of a light emitting device including a hybrid wavelength converting body according to an embodiment of the present disclosure.

FIG. 100 is a cross-sectional view of a light emitting device including a hybrid wavelength converting body according to another embodiment of the present disclosure.

FIG. 101 is a cross-sectional view of an encapsulated metal halide perovskite wavelength conversion layer film according to an embodiment of the present disclosure.

FIG. 102 is a schematic diagram showing a method of manufacturing a metal halide perovskite wavelength converting body having a structure in which encapsulated particles are dispersed according to an embodiment of the present disclosure.

FIG. 103 is a schematic diagram showing a method of manufacturing a metal halide perovskite wavelength converting body having a structure in which encapsulated particles are dispersed according to another embodiment of the present disclosure.

FIG. 104 is a schematic diagram showing a method of manufacturing a metal halide perovskite wavelength converting body having a structure in which encapsulated particles are dispersed according to another embodiment of the present disclosure.

FIG. 105 is a schematic diagram showing a method of manufacturing a metal halide perovskite wavelength converting body having a structure in which encapsulated particles are dispersed according to another embodiment of the present disclosure.

FIG. 106 is a schematic diagram of a metal halide perovskite light emitting particle to which a medium-sized organic cation is added according to an embodiment of the present disclosure.

FIG. 107 is a schematic diagram of a metal halide perovskite light emitting particle to which a medium-sized organic cation is added according to another embodiment of the present disclosure.

FIG. 108 is an XRD analysis result according to the content of a medium-sized organic cation (guanidinium) in the metal halide perovskite light emitting particle according to an embodiment of the present disclosure.

FIG. 109 is a photoluminescence analysis result according to the content of medium-sized organic cations (guanidinium) in the metal halide perovskite light emitting particles according to an embodiment of the present disclosure.

FIG. 110 is an analysis result of particle size according to the content of medium-sized organic cations (guanidinium) in the metal halide perovskite light emitting particles according to an embodiment of the present disclosure.

FIG. 111 is a graph of photoluminescence quantum efficiency according to the content of medium-sized organic cations (guanidinium) in the metal halide perovskite light emitting particles according to an embodiment of the present disclosure.

FIG. 112 is a graph of photoluminescence lifetime according to the content of medium-sized organic cations (guanidinium) in the metal halide perovskite light emitting particles according to an embodiment of the present disclosure.

FIG. 113 is an exciton binding energy determined by temperature dependent photoluminescence according to the content of a medium-sized organic cation (guanidinium) in the metal halide perovskite light emitting particle according to an embodiment of the present disclosure. It is a graph showing.

FIG. 114 is a graph showing the stability against UV irradiation of a metal halide perovskite light emitting particle to which a medium organic cation is added according to an embodiment of the present disclosure.

FIG. 115 is a graph showing the stability against thermal decomposition according to the content of medium-sized organic cations (guanidinium) in the metal halide perovskite light emitting particles according to an embodiment of the present disclosure.

FIG. 116 is a graph showing the luminescence efficiency of a light emitting diode according to the content of medium-sized organic cations (guanidinium) in the metal halide perovskite light emitting particles according to an embodiment of the present disclosure.

FIG. 117 is a graph showing changes in lattice constants in crystals according to the content of medium-sized organic cations (guanidinium) in the metal halide perovskite light emitting particles having a mixed cation structure according to an embodiment of the present disclosure.

FIG. 118 is a graph showing the light emission intensity of the perovskite light-emitting particles according to the content of medium-sized organic cations (guanidinium) in the metal halide perovskite light-emitting particles having a mixed cationic structure according to an embodiment of the present disclosure.

FIG. 119 is a graph showing a luminescence intensity and lifetime of metal halide perovskite light emitting particles having a mixed cation structure according to an embodiment of the present disclosure, according to the content of medium-sized organic cations (guanidinium).

FIG. 120 is a graph showing light emission intensity of a polycrystalline thin film including perovskite emission particles according to types of organic cations to be mixed in a metal halide perovskite light emitting particle having a mixed cationic structure according to an embodiment of the present disclosure.

FIG. 121 is a graph showing the luminance of a polycrystalline thin film including perovskite light emitting particles according to types of organic cations to be mixed in the metal halide perovskite light emitting particles having a mixed cationic structure according to an embodiment of the present disclosure.

FIG. 122 is a graph showing current efficiency of a polycrystalline thin film including perovskite light emitting particles according to types of organic cations to be mixed in metal halide perovskite light emitting particles having a mixed cationic structure according to an embodiment of the present disclosure.

FIG. 123 is a graph showings the operational lifetime of a polycrystalline thin film including perovskite light emitting particles according to types of organic cations to be mixed in the metal halide perovskite light emitting particles having a mixed cationic structure according to an embodiment of the present disclosure.

### MODES OF THE DISCLOSURE

Hereinafter, preferred embodiments according to the present disclosure will be described in more detail with reference to the accompanying drawings in order to describe the present disclosure in more detail. However, the present disclosure is not limited to the embodiments described herein and may be embodied in other forms.

In the present specification, terms such as first and second are used to describe various components, and these components are not limited by the above terms, and are used in order to distinguish one component from other components. In addition, the present disclosure is not limited to the particles or configurations described in the present specification, but it should be understood that all changes or equivalents included in the spirit of the present disclosure are also included in the technical scope of the present disclosure.

Components of the accompanying drawings in the present specification may be enlarged or reduced for convenience of description.

In the present specification, reference numerals are limited to each drawing in which the reference numerals are included, and reference numerals included in different drawings may represent different components even if the markings are the same.

This disclosure offers the metal halide perovskite (hereinafter, perovskite) with the structure of ABX₃(3D), A₄BX(0D), AB₂X₅(2D), A₂BX₄(2D), A₂B⁺B³⁺X₆(3D), A₃B₂X₉(2D), or Aₙ₋₁BₙX₃ₙ₊₁ (quasi-2D) where above A is monovalent cation, above B is metal, above X is halogen.

While tolerance factor is defined as

$$t = \frac{{R}_{A} + {R}_{X}}{\sqrt{2} ( {R}_{B} + {R}_{X} )}$$

(R_{A}, R_{B}, R_{X} is the ionic radius of A, B, X respectively), the above mentioned A is the mixture of 1^{st} monovalent cation (A₁) which can provide the tolerance factor smaller than 1.01 when incorporated into perovskite structure, and 2^{nd} monovalent cation (A₂) which can make the tolerance factor larger than 1.01 and smaller than 3. A₂ monovalent cation is incorporated inside the metal halide perovskite crystal and on the surface of the nanocrystal simultaneously.

Moreover, preferably, the perovskite emitters may be colloidal nanocrystal particle dispersed in the solvent or polycrystalline bulk thin films.

Moreover, preferably, the above mentioned 1st monovalent cation (A₁) is at least one or a combination of them selected from a group of methylammonium, formamidinium, Cs, Rb and the above mentioned 2nd monovalent cation (A₂) is at least one or a combination of them selected from a group of ethylammonium, guanidinium, tert-butylammonium, diethylammonium, dimethylammonium, ethane-1.2.-diammonium, imidazolium, n-propylammonium, iso-propylammonium, and pyrrolidinium.

Moreover, preferably, the ratio of 1^{st} monovalent cation (A₁) to total A-site monovalent cation is larger than 5% and smaller than 60%. For example, 5%, 5.5%, 6%, 6.5%, 7%, 7.5%, 8%, 8.3%, 8.5%, 8.7%, 9%, 9.3%, 9.5%, 9.7%, 10%, 10.3%, 10.5%, 10.7%, 11%, 11.3%, 11.5%, 11.7%, 12%, 12.5%, 13%, 13.5%, 14%, 14.5%, 15%, 15.5%, 16%, 16.5%, 17%, 17.5%, 18%, 18.5% 19%, 19.5%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, and 60%. It may be preferably between 7% and 30% respectively. More preferably, it may be between 8% and 20%. More preferably, it may not be less than 9% but not more than 15%.

Moreover, preferably, if the perovskite emitter is nanocrystal particle, the nanocrystal particle can have a crystal size of larger than the Bohr diameter (˜10 nm in MAPbBr₃, ˜7 nm in CsPbBr₃) and less than 30 nm. For example, 7 nm, 7.5 nm, 8 nm, 8.3 nm, 8.5 nm, 8.7 nm, 9 nm, 9.3 nm, 9.5 nm, 9.7 nm, 10 nm, 10.3 nm, 10.5 nm, 10.7 nm, 11 nm, 11.3 nm, 11.5 nm, 11.7 nm, 12 nm, 12.3 nm, 12.5 nm, 12.7 nm, 13 nm, 13.3 nm, 13.5 nm, 13.7 nm, 14 nm, 14.3 nm, 14.5 nm, 14.7 nm, 15 nm, 15.3 nm, 15.5 nm, 15.7 nm, 16 nm, 16.5 nm 17 nm, 17.5 nm, 18 nm, 18.5 nm, 19 nm, 19.5 nm, 20 nm, 21 nm, 22 nm, 23 nm, 24 nm, 25 nm, 26 nm, 27 nm, 28 nm, 29 nm, and 30 nm. It can be preferably not less than 7 nm but not more than 25 nm. More preferably, it may not be less than 10 nm but not more than 20 nm. More preferably, it can be not less than 10 nm but not more than 15 nm. When determining the size of the colloidal particle, a value measured by Transmission Electron Microscopy (TEM) is taken. The light scattering method cannot be distinguished from agglomerated particle agglomerates (aggregates), so it is not an accurate measurement method.

Also preferably, the perovskite light-emitting material may emit light in a region of 200 nm to 1500 nm. More preferably, light in a region of 430 nm to 780 nm may be emitted. More preferably, light in the 450 nm to 650 nm region may be emitted. For example, 200 nm, 250 nm, 300 nm, 350 nm, 400 nm, 410 nm, 420 nm, 430 nm, 440 nm, 450 nm, 455 nm, 460 nm, 465 nm, 470 nm, 475 nm, 480 nm, 485 nm, 490 nm, 500 nm, 510 nm, 520 nm, 525 nm, 530 nm, 535 nm, 540 nm, 545 nm, 550 nm, 560 nm, 570 nm, 580 nm, 590 nm, 600 nm, It may be 610 nm, 620 nm, 625 nm, 630 nm, 635 nm, 640 nm, 650 nm.

Also preferably, the first monovalent (A₁) cation is a combination of methylammonium, formamidinium, and cesium, the second monovalent (A₂) cation is guanidinium, and the guanidinium may be contained in an amount of 5% or more and 60% or less with respect to the total amount of the mixture of methylammonium, formamidinium, cesium and guanidinium. For example, it may be 5%, 5.5%, 6%, 6.5%, 7%, 7.5%, 8%, 8.3%, 8.5%, 8.7%, 9%, 9.3%, 9.5%, 9.7%, 10%, 10.3%, 10.5%, 10.7%, 11%, 11.3%, 11.5%, 11.7%, 12%, 12.5%, 13%, 13.5%, 14%, 14.5%, 15%, 15.5%, 16%, 16.5%, 17%, 17.5%, 18%, 18.5% 19%, 19.5%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%. Preferably it may be 7% or more and 30% or less. More preferably, it may be 8% or more and 20% or less. More preferably, it may be 9% or more and 15% or less.

In addition, preferably, when the perovskite light emitter is a nanocrystal particle, the perovskite light emitting material may convert a wavelength of light generated from an excitation light source into a specific wavelength.

In addition, preferably, when the perovskite light emitting material is a nanocrystal particle, a plurality of organic ligands surrounding the nanocrystal may be further included in the perovskite nanocrystal particle and may be dispersed in an organic solvent.

To achieve the above-mentioned second purpose, this disclosure offers the metal halide perovskite with the structure of ABX₃(3D), A₄BX(0D), AB₂X₅(2D), A₂BX₄(2D), A₂B⁺B³⁺X₆(3D), A₃B₂X₉(2D), or Aₙ₋₁BₙX₃ₙ₊₁ (quasi-2D) where above A is monovalent cation, above B is metal, above X is halogen.

While tolerance factor is defined as

$$t = \frac{{R}_{A} + {R}_{X}}{\sqrt{2} ( {R}_{B} + {R}_{X} )}$$

(R_{A}, R_{B}, R_{X} is the ionic radius of A, B, X respectively), the above mentioned A is the mixture of at least three different 1^{st} monovalent cation (A₁, A₃, A₄) that make the tolerance factor smaller than 1.01 when incorporated into perovskite structure, and the 2^{nd} monovalent cation (A₂) which make the tolerance factor larger than 1.01 and smaller than 3, preferably, smaller than 2. The 1^{st} monovalent cation and the 2^{nd} monovalent cation (A₂) are simultaneously incorporated at the A site evenly inside the metal halide perovskite crystal.

Moreover, preferably, the perovskite emitters may be colloidal nanocrystal particle dispersed in the solvent or polycrystalline bulk thin films.

Moreover, preferably, the above mentioned 1^{st} monovalent cation (A₁, A₃, A₄) is at least one or a combination of them selected from a group of methylammonium, formamidinium, Cs, Rb and the above mentioned 2^{nd} monovalent cation (A₂) is at least one or a combination of them selected from a group of ethylammonium, guanidinium, tert-butylammonium, diethylammonium, dimethylammonium, ethane-1.2.-diammonium, imidazolium, n-propylammonium, iso-propylammonium, and pyrrolidinium.

Moreover, preferably, the ratio of 1^{st} monovalent cation (A₁) at the A site to amount of the total A-site monovalent cations comprising the 1^{st} monovalent cation (A₁) and the 2^{nd} monovalent cation (A₂) is larger than 5% and smaller than 60%. For example, it may be 5%, 6%, 7%, 8%, 9%, 10%, 11%, 12%, 13%, 14%, 15%, 15.1%, 15.2%, 15.3%, 15.4%, 15.5%, 15.6%, 15.7%, 15.8%, 15.9%, 16%, 16.1%, 16.2%, 16.3%, 16.4%, 16.5%, 16.6%, 16.7%, 16.8%, 16.9%, 17%, 17.1%, 17.2%, 17.3%, 17.4%, 17.5%, 17.6%, 17.7%, 17.8%, 17.9%, 18%, 18.1%, 18.2%, 18.3%, 18.4%, 18.5%, 18.6%, 18.7%, 18.8%, 18.9%, 19%, 19.1%, 19.2%, 19.3%, 19.4%, 19.5%, 19.6%, 19.7%, 19.8%, 19.9%, 20%, 20.1%, 20.2%, 20.3%, 20.4%, 20.5%, 20.6%, 20.7%, 20.8%, 20.9%, 21%, 21.1%, 21.2%, 21.3%, 21.4%, 21.5%, 21.6%, 21.7%, 21.8%, 21.9%, 22%, 22.1%, 22.2%, 22.3%, 22.4%, 22.5%, 22.6%, 22.7%, 22.8%, 22.9%, 23%, 23.1%, 23.2%, 23.3%, 23.4%, 23.5%, 23.6%, 23.7%, 23.8%, 23.9%, 24%, 24.1%, 24.2%, 24.3%, 24.4%, 24.5%, 24.6%, 24.7%, 24.8%, 24.9%, 25%, 25.5%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, and 60%. It may be preferably between 5% and 30% respectively. More preferably, it may be between 15% and 25%. More preferably, it may not be less than 18% but not more than 22%.

Moreover, preferably, if the perovskite emitter is nanocrystal particle, the nanocrystal particle can have a crystal size of larger than the Bohr diameter (˜10 nm in MAPbBr₃, ˜7 nm in CsPbBr₃) and less than 30 nm. For example, it may be 7 nm, 7.5 nm, 8 nm, 8.3 nm, 8.5 nm, 8.7 nm, 9 nm, 9.3 nm, 9.5 nm, 9.7 nm, 10 nm, 10.3 nm, 10.5 nm, 10.7 nm, 11 nm, 11.3 nm, 11.5 nm, 11.7 nm, 12 nm, 12.3 nm, 12.5 nm, 21 nm, 22 nm, 23 nm, 24 nm, 25 nm, 26 nm, 27 nm, 28 nm, 29 nm, 30 nm. It can be preferably not less than 7 nm but not more than 25 nm. More preferably, it may not be more than 10 nm but not more than 20 nm. More preferably, it can be not less than 10 nm but not more than 15 nm.

Also preferably, the perovskite light-emitting material may emit light in a region of 300 nm to 1500 nm. More preferably, light in a region of 430 nm to 780 nm may be emitted. More preferably, light in the 450 nm to 650 nm region may be emitted. For example, it may be 400 nm, 410 nm, 420 nm, 430 nm, 440 nm, 450 nm, 455 nm, 460 nm, 465 nm, 470 nm, 475 nm, 480 nm, 485 nm, 490 nm, 500 nm, 510 nm, 520 nm, 525 nm, 530 nm, 535 nm, 540 nm, 545 nm, 550 nm, 560 nm, 570 nm, 580 nm, 590 nm, 600 nm, 610 nm, 620 nm, 625 nm, 630 nm, 635 nm, 640 nm, and 650 nm.

Moreover, preferably, 1^{st} monovalent cation (A₁, A₃, A₄) is a combination containing all of methylammonium, formamidinium, and cesium, the second monovalent cation is guanidinium, and the guanidinium may be 5% or more and 60% or less with respect to the total amount of the 4 kinds of cation mixture of methylammonium, formamidinium, cesium and guanidinium. For example, it can be 5%, 6%, 7%, 8%, 9%, 10%, 11%, 12%, 13%, 14%, 15%, 15.1%, 15.2%, 15.3%, 15.4%, 15.5%, 15.6%, 15.7%, 15.8%, 15.9%, 16%, 16.1%, 16.2%, 16.3%, 16.4%, 16.5%, 16.6%, 16.7%, 16.8%, 16.9%, 17%, 17.1%, 17.2%, 17.3%, 17.4%, 17.5%, 17.6%, 17.7%, 17.8%, 17.9%, 18%, 18.1%, 18.2%, 18.3%, 18.4%, 18.5%, 18.6%, 18.7%, 18.8%, 18.9%, 19%, 19.1%, 19.2%, 19.3%, 19.4%, 19.5%, 19.6%, 19.7%, 19.8%, 19.9%, 20%, 20.1%, 20.2%, 20.3%, 20.4%, 20.5%, 20.6%, 20.7%, 20.8%, 20.9%, 21%, 21.1%, 21.2%, 21.3%, 21.4%, 21.5%, 21.6%, 21.7%, 21.8%, 21.9%, 22%, 22.1%, 22.2%, 22.3%, 22.4%, 22.5%, 22.6%, 22.7%, 22.8%, 22.9%, 23%, 23.1%, 23.2%, 23.3%, 23.4%, 23.5%, 23.6%, 23.7%, 23.8%, 23.9%, 24%, 24.1%, 24.2%, 24.3%, 24.4%, 24.5%, 24.6%, 24.7%, 24.8%, 24.9%, 25%, 25.5%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, It can be 54%, 55%, 56%, 57%, 58%, 59%, 60%. Preferably, it may be 5% or more and 30% or less. More preferably, it may be 15% or more and 25% or less. More preferably, it may be 18% or more and 22% or less.

Also preferably, the first monovalent cation (A₁, A₃, A₄) is a combination of methylammonium, formamidinium, and cesium, the second monovalent cation (A₂) is guanidinium, and the guanidinium may be 1% or more and 20% or less with respect to the total amount of the mixture of four types of cation of methyl ammonium, formamidinium, cesium and guanidinium. For example, it can be 1%, 1.5%, 2%, 2.5%, 3%, 3.5%, 4%, 4.5%, 5%, 5.1%, 5.2%, 5.3%, 5.4%, 5.5%, 5.6%, 5.7%, 5.8%, 5.9%, 6%, 6.1%, 6.2%, 6.3%, 6.4%, 6.5%, 6.6%, 6.7%, 6.8%, 6.9%, 7%, 7.1%, 7.2%, 7.3%, 7.4%, 7.5%, 7.6%, 7.7%, 7.8%, 7.9%, 8%, 8.1%, 8.2%, 8.3%, 8.4%, 8.5%, 8.6%, 8.7%, 8.8%, 8.9%, 9%, 9.1%, 9.2%, 9.3%, 9.4%, 9.5%, 9.6%, 9.7%, 9.8%, 9.9%, 10%, 10.1%, 10.2%, 10.3%, 10.4%, 10.5%, 10.6%, 10.7%, 10.8%, 10.9%, 11%, 11.1%, 11.2%, 11.3%, 11.4%, 11.5%, 11.6%, 11.7%, 11.8%, 11.9%, 12%, 12.1%, 12.2%, 12.3%, 12.4%, 12.5%, 12.6%, 12.7%, 12.8%, 12.9%, 13%, 13.1%, 13.2%, 13.3%, 13.4%, 13.5%, 13.6%, 13.7%, 13.8%, 13.9%, 14%, 14.1%, 14.2%, 14.3%, 14.4%, 14.5%, 14.6%, 14.7%, 14.8%, 14.9%, 15%, 15.1%, 15.2%, 15.3%, 15.4%, 15.5%, 15.6%, 15.7%, 15.8%, 15.9%, 16%, 16.1%, 16.2%, 16.3%, 16.4%, 16.5%, 16.6%, 16.7%, 16.8%, 16.9%, 17%, 17.1%, 17.2%, 17.3%, 17.4%, 17.5%, 17.6%, 17.7%, 17.8%, 17.9%, 18%, 18.1%, 18.2%, 18.3%, 18.4%, 18.5%, 18.6%, 18.7%, 18.8%, 18.9%, 19%, 19.1%, 19.2%, 19.3%, 19.4%, 19.5%, 19.6%, 19.7%, 19.8%, 19.9%, 20%. Preferably it may be 5% or more and 15% or less. More preferably, it may be 7.5% or more and 12.5% or less. More preferably, it may be 9% or more and 11% or less.

The present disclosure includes the perovskite nanocrystal particles for converting a wavelength of light generated from an excitation light source into a specific wavelength; And it provides a metal halide perovskite wavelength conversion material comprising a dispersion medium for dispersing the perovskite nanocrystal particles.

In addition, the present disclosure provides the perovskite light-emitting device comprising a substrate; A first electrode on the substrate; A light emitting layer positioned on the first electrode; And a second electrode positioned on the emission layer, wherein the emission layer is the metal halide perovskite light emitters.

In addition, the present disclosure provides the perovskite light-emitting device comprising a base structure; at least one excitation light source that emits light of a predetermined wavelength located on the base structure; and the perovskite wavelength conversion materials located in the optical path of the excitation light source.

<Metal Halide Perovskite Crystal>

The metal halide perovskite may be a material having a three-dimensional crystal structure, a two-dimensional crystal structure, a one-dimensional crystal structure, or a zero-dimensional crystal structure.

The metal halide perovskite is ABX₃(3D), A₄BX₆(0D), AB₂X₅(2D), A₂BX₄(2D), A₂BX₆(0D), A₂B⁺B³⁺X₆(3D), A₃B₂X₉(2D) or it may include a structure of Aₙ₋₁BₙX₃ₙ₊₁ (quasi-2D) (n is an integer between 2 and 6). A is a monovalent cation, B is a metal material, and X may be a halogen element. The quasi-2D structure may be a Ruddlesden-Popper phase or a Dion-Jacobson phase.

The monovalent cation may be a monovalent organic cation or an alkali metal. For example, the monovalent organic cation is organic ammonium (RNH₃)⁺, organic amidinium derivative (RC(═NR₂)NR₂)⁺, organic guanidinium derivative (R₂NC(═NR₂)NR₂)⁺, organic diammonium (CₓH₂ₓ₋ₙ₊₄)(NH₃)ₙ⁺, ((CₓH₂ₓ₊₁)ₙNH₃)(CH₃NH₃)ₙ⁺, (RNH₃)₂⁺, (CₙH₂ₙ₊₁NH₃)₂⁺, (CF₃NH₃)⁺, (CF₃NH₃)ₙ⁺, ((CₓF₂ₓ₊₁)ₙNH₃)₂(CF₃NH₃)ₙ⁺, ((CₓF₂ₓ₊₁)ₙNH₃)₂⁺ or (CₙF₂ₙ₊₁NH₃)₂⁺ (x or n is an integer of 1 or more, R=hydrocarbon derivative, alkyl, alkyl fluoride derivatives, H, F, Cl, Br, I) and combinations thereof, but are not limited thereto. The alkali metal may be Li⁺, Na⁺, K⁺, Rb⁺, Cs⁺, Fr⁺, and combinations thereof, but is not limited thereto.

In addition, preferably, the organic cations are acetamidinium, azaspironanium, benzene diammonium, benzylammonium, butanediammonium, iso-butylammonium, n-butylammonium, t-butylammonium, cyclohexylammonium, cyclohexylmethylammonium, azobicyclooctanedinium, diethylammonium, N,N-diethylethane diammonium, N,N-diethylpropane diammonium, Dimethylammonium, N,N-dimethylethane diammonium, dimethylpropane diammonium, dodecylammonium, Ethanediammonium, ethylammoniuium, 4-fluoro-benzylammonium, 4-fluoro-phenylethylammonium, 4-fluoro-phenylammonium, formamidinium, guanidinium, hexanediammnium, hexylammonium, imidazolium, 2-methoxyethylammonium, 4-methoxy-phenlylethylammonium, 4-methoxy-phenylammonium, methylammonium, morpholinium, octylammonium, pentylammonium, piperazinediium, piperidinium, propanediammonium, iso-propylammonium, di-iso-propylammonium, n-propylammonium, pyridinium, 2-pyrrolidin-1-ium-1-yethylammonium, Pyrrolidinium, quinclidin-1-ium, 4-trifluoromethyl-benzylammonium, 4-trifluoromethyl ammonium, and quaternary ammonium cations such as benzalkonium chloride, dimethyldioctadecylammonium chloride, trimethylglycine, and choline, and combinations thereof, but is not limited there to.

The B may be a divalent transition metal, a rare earth metal, an alkaline earth metal, a combination of a monovalent metal and a trivalent metal, an organic substance (a monovalent, divalent, trivalent cation), and a combination thereof. In addition, preferably, the divalent transition metal, rare earth metal, and alkaline earth metal are Be²⁺, Mg²⁺, Ca²⁺, Sr²⁺, Ba²⁺, Ra²⁺, Mn²⁺, Fe²⁺, Co²⁺, Ni²⁺, Cu²⁺, Zn²⁺, Ru²⁺, Bi²⁺, Pd²⁺, Cd²⁺, Pt²⁺, Hg²⁺, Ge²⁺, Sn²⁺, Pb²⁺, Se²⁺, Te²⁺, Po²⁺, Eu²⁺, No²⁺, and combinations thereof, but are not limited thereto. The monovalent metal may be Li⁺, Na⁺, K⁺, Rb⁺, Cs⁺, Fr⁺, Ag⁺, Hg⁺, Ti⁺, and combinations thereof, and the trivalent metal is Cr³⁺, Fe³⁺, Co³⁺, Ru³⁺, Rh³⁺, Ir³⁺, Au³⁺, Al³⁺, Ga³⁺, In³⁺, Ti³⁺, As³⁺, Sb³⁺, Bi³⁺, La³⁺, Ce³⁺, Pr³⁺, Nd³⁺, Pm³⁺, Sm³⁺, Eu³⁺, Gd³⁺, Tb³⁺, Dy³⁺, Ho³⁺, Er³⁺, Tm³⁺, Yb³⁺, Am³⁺, Cm³⁺, Bk³⁺, Cf³⁺, Es³⁺, Fm³⁺, Md³⁺, Lr³⁺ and combinations thereof.

In addition, X may be F⁻, Cl⁻, Br⁻, I⁻, At⁻, and combinations thereof.

The metal halide perovskite is ABX₃(3D), A₄BX₆(0D), AB₂X₅(2D), A₂BX₄(2D), A₂BX₆(0D), A₂B⁺B³⁺X₆(3D), A₃B₂X₉(2D) or it may include a structure of Aₙ₋₁BₙX₃ₙ₊₁ (quasi-2D) (n is an integer between 2 and 6), wherein A is a monovalent cation, B is a metal material, and X is a halogen.

The metal halide perovskite may be in the form of a polycrystalline metal halide perovskite bulk thin film, or a metal halide perovskite nanocrystal that can be easily dispersed in a colloidal state in a solution.

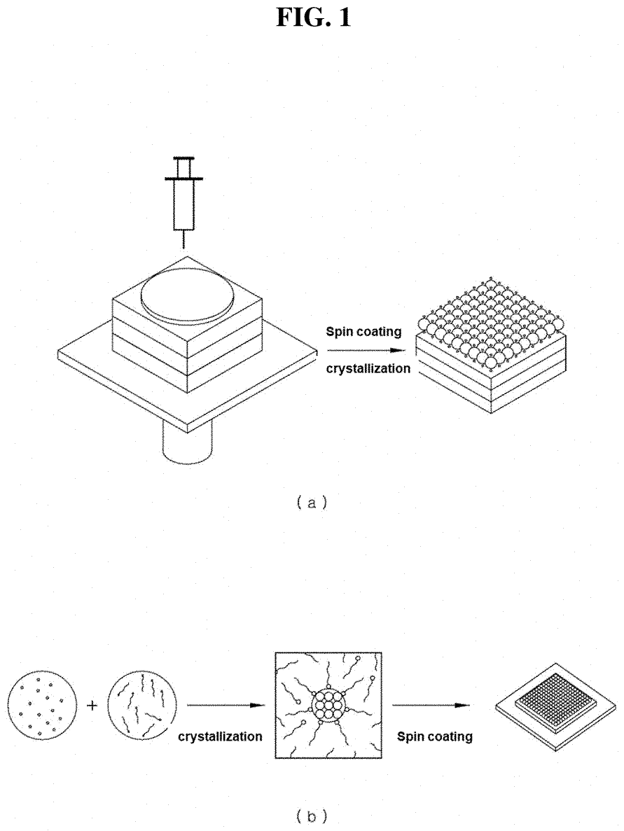

FIG. 1 is a schematic diagram showing the difference between a metal halide perovskite bulk thin film and a metal halide perovskite nanocrystal particle according to an embodiment of the present disclosure.

As shown in FIG. 1, crystallization and thin film formation processes occur at the same time to form the metal halide perovskite bulk thin film by evaporating the solvent in the spin coating process of the transparent ion-type metal halide perovskite precursor solution. do. Therefore, the bulk thin film forms a thin film by directly reacting two or more precursors, and it is greatly affected by thermodynamic parameters such as temperature and surface energy during the thin film formation process. A thin film composed of a large three-dimensional or two-dimensional polycrystal is formed.

However, as shown in FIG. 1, the perovskite nanocrystal particles are first crystallized into nanoparticles in a colloidal solution and then stably dispersed in the solution using a ligand. Because nanocrystal particles are in a state where crystallization is terminated in a solution, when forming a thin film through coating, there is no additional growth of crystals and is not affected by coating conditions, and the nanocrystal particle that are configured to have a size of several nanometers to several tens of nanometers can form a thin film that maintains high luminescence efficiency.

Colloidal solution refers to a dispersion in which solid particles having a size of about 10 μm or less do not aggregate with each other, form a stable mixed solution, and spread in a liquid. The solid particles constituting the colloid correspond to the dispersed phase, and the liquid in which the solid fine particles are dispersed is referred to as a dispersion medium. Similar to colloidal concepts, there may be an aerosol and an emulsion. However, aerosol refers to a state in which liquid droplets or solid particles are dispersed in a gas, and emulsion is a uniformly dispersed state of liquid droplets that is immiscible in other types of liquid. Thus, the aerosol and emulsion have differences from colloids. There are various opinions on the limit of the size of the solid particles constituting the colloidal dispersion system. A dispersion of the corresponding fine particles having a size of 1 nm to 1 μm to be dispersed is referred to as a colloid, and for a size larger than that, it is sometimes referred to separately as a suspension. In this specification, the concept of defining a solid dispersion having a size of 10 μm or less as a colloid is followed, but the difference between dispersion and suspension is classified according to whether or not it precipitates well over time. For example, when precipitation occurs within several hours, it is a suspension, and dispersion is defined as a dispersion that can be dispersed without precipitation for several hours or longer, preferably for several days or longer. The colloidal dispersion may be dispersed in a dispersion medium such as a polymer or ceramic material to form a thin film or film.

### <Metal Halide Perovskite Nanocrystal Particle>

FIG. 2 is a schematic diagram showing a metal halide perovskite nanocrystal particles according to an embodiment of the present disclosure.

The metal halide perovskite nanocrystal may further include a plurality of organic ligands (20) surrounding the halide metal halide perovskite nanocrystal (10). The organic ligands (20) at this time are substances used as surfactants, so it may include alkyl halide, alkyl ammonium halide, amine ligand, carboxylic acid, or phosphonic acid.

Ligand refers to a generic term for an ion or molecule that can be bound to a central atom in a dative complex. The ligand binds to the surface of the nanoparticle and serves to precisely control the shape and size of the nanoparticle. For a detailed description of the ligand, refer to [Journal of the American Chemistry Society, 2013, 135, 49, pp 18536-18548]. The ligand binding to the surface of the nanoparticle may correspond to an L-type ligand, an X-type ligand, or a Z-type ligand according to the mode of binding with the surface of the nanoparticle: an L-type ligand donate two electrons to form a coordinate bond, an X-type ligand donates an electron to the cation site on the surface of a nanoparticle followed by formation of a covalent bond, and the acceptor of two electrons on the surface of a nanoparticle corresponds to the Z-type ligand.

Surfactants are amphiphilic substances that have opposite functional groups of hydrophilicity and hydrophobicity simultaneously in the same molecule, and are adsorbed at the interface between liquid and gas, liquid and liquid, or liquid and solid, so they can play a role in causing various physical phenomena to appear. The role of the surfactant can serve to lower surface tension, emulsify, improve wettability and foamability, or solubilize. Particularly, when the surfactant acts as a ligand by binding to the surface of the nanoparticle through a coordinate bond, the dispersibility of the nanoparticle can be improved. Examples of surfactants include anionic surfactants (e.g., sulfate such as ammonium lauryl sulfate, sodium lauryl sulfate, sodium dodecyl sulfate, sodium laureth sulfate, sodium myreth sulfate), sulfonate such as dioctyl sodium sulfosuccinate, perfluorooctanesulfonate, perfluorobutanesulfonate, linear alkylbenzne sulfates, phosphate esters, carboxylates that include sodium stearate, sodium lauroyl sarcosinate, perfluorononanoate, perfluorooctanoate, cationic surfactants that include primary, secondary, tertiary, quaternary ammonium cation, and quaternary ammonium cations such as benzalkonium chloride, dimethyldioctadecylammonium chloride, trimethylglycine, choline, zwitterionic or amphoteric surfactant having both cations and anions in the same substance, and nonionic surfactants that include long chain alcohols such as fatty alcohols, cetyl alcohol, stearyl alcohol, cetostearyl alcohol (consisting predominantly of cetyl and stearyl alcohols), and oleyl alcohol.

The alkyl halide may be an alkyl-X structure. The halogen element corresponding to X at this time may include Cl, Br, or I. In addition, the alkyl structure at this time includes a primary alcohol having a structure such as acyclic alkyl having a structure of CₙH₂ₙ₊₁, a primary alcohol having a structure such as CₙH₂ₙ₊₁OH, a secondary alcohol, a tertiary alcohol, alkylamine having a structure of alkyl-N (ex. hexadecyl amine, 9-octadecenylamine (1-Amino-9-octadecene, C₁₈H₃₇N), p-substituted aniline, phenyl ammonium or fluorine ammonium, but are not limited thereto.

The amine ligand can be N,N-diisopropylethylethylamine, ethylenediamine, hexamethylenetetraamine, methylamine, hexylamine, oleylamine, N,N,N,N-tetramethylenediamine, triethylamine, diethanolamine, 2,2-(ethylenedioxyl)bis-(ethylamine) may be selected from, but is not limited thereto.

The alkyl ammonium halide (or alkylammonium salt) includes methylammonium chloride, dimethylammonium bromide, and octylammonium bromide, and in some cases, fluoride or acetate may be substituted as a salt in the place of a halide (e.g. ethyl dimethylammonium fluoride, tetrabenzylammonium acetate), but are not limited thereto.

The carboxylic acid include 4,4′-azobis(4-cyanovaleric acid), acetic acid, 5-aminosalicylic acid, acrylic acid, L-aspentic acid, 6-bromohexanoic acid, bromoacetic acid, dichloro acetic acid, ethylenediaminetetraacetic acid, isobutyric acid, itaconic acid, maleic acid, r-maleimidobutyric acid, L-malic acid, 4-nitrobenzoic acid, 1-pyrenecarboxylic acid or oleic acid.

The phosphonic acid includes n-hexylphosphonic acid, n-octylphosphonic acid, n-decylphosphonic acid, n-dodecylphosphonic acid, n-tetradecylphosphonic acid, n-hexadecylphosphonic acid, and n-octadecylphonic acid, but is not limited thereto.

The organic ligand may be in a fluorinated form. For example, the organic ligand include 2-fluorophenylbornic acid, 3,5-diformyl-2-fluorophenylboronic acid, 3-chloro-4-fluorophenylboronic acid, 4-cyano-3-fluprpbenzoic acid, L-Fmoc-3-fluorophenylalanine, L-Fmoc-4-fluorophenylalanine, Methyl 6-fluorochromone-2-carboxylic acid, 4-fluorobenzoic acid, 2-fluorobenzoic acid, 2-fluoro benzylamine, 2-2-fluorocinnamic acid, 2-fluorophenyl isothiocyanate, 4-fluorobenzenesulfonic acid, 4-flurobenzylamine, 4-fluorophenyl isothiocyanate, 4-fluorophenylacetic acid, Fluorocinnamic acid, 3-Fluoro-4-methylphenyl)acetic acid, 3-fluoro-5-isopropoxyphenyl)boronic acid, 3-fluoro-5-methoxycarbonylphenyl)boronic acid, 3-fluoro-5-methylphenyl)boronic acid, 4-fluoro-2-methoxyphenyl)oxoacetic acid, 4-fluoro-3-methoxyphenyl)acetic acid, 4-fluoro-3-methoxyphenyl)boronic acid and a combination thereof, but is not limited thereto.

Also, preferably, the fluorinated organic compound may be in the form of a perfluorinated compound. The perfluorinated compounds include perfluorinated alkyl halides, perfluorinated aryl halide, fluorochloroalkene, perfluoroalcohol, perfluoamine, and perfluorinated carbohydrate. It may be an acid (perfluorocarboxylic acid), perfluorosulfonic acid (perfluorosulfonic acid), or a derivative thereof, but is not limited thereto.

The perfluorinated alkyl halides and perfluorinated aryl halide are trifluoroiodomethane, pentafluoroethyl iodide, and perfluorinated octyl bromide (perfluorooctyl bromide, perflubron), dichlorodifluoromethane, and derivatives thereof, but are not limited thereto.

The fluorochloroalkene may be chlorotrifluoroethylene, dichlorodifluoroethylene, and may be a derivative thereof, but is not limited thereto.

The fluorochloroalkene may be chlorotrifluoroethylene, dichlorodifluoroethylene, and derivatives thereof, but is not limited thereto.

The perfluorocarboxylic acid may be trifluoroacetic acid, heptafluorobutryric acid, pentafluorobenzoic acid, perfluorooctanoic acid, perfluorononanoic acid, and derivatives thereof, but is not limited thereto.

The perfluorosulfonic acid is triflic acid, perfluorobuanesulfonic acid, perfluorobutane sulfonamide, perfluorooctanesulfonic acid, and It may be a derivative thereof, but is not limited thereto.

The ligand may be trioctylphosphine oxide (TOPO), trioctylphosphine (TOP, trioctylphosphine), triethylphosphine oxide, tributylphosphine oxide and their derivative, but is not limited thereto.

Therefore, as described above, the alkyl halide used as a surfactant to stabilize the surface of the metal halide perovskite, which may otherwise precipitate, becomes an organic ligand surrounding the surface of the metal halide perovskite nanocrystal. On the other hand, when the length of the alkyl halide surfactant is short, the size of the formed nanocrystal increases, so it can be formed in excess of 100 nm, further 300 nm or more, and even more than 1 μm, and in the large nanocrystals, there may be a fundamental problem in that due to thermal ionization and delocalization of charge carriers, excitons do not emit light and are separated into free charge carriers and disappear. Accordingly, the size of the metal halide perovskite nanocrystals formed by using an alkyl halide having a predetermined length or longer as a surfactant can be controlled to a predetermined size or less (ie, 100 nm or less, preferably 30 nm or less).

In addition, as the size of the previously used inorganic quantum dots becomes smaller than the exciton Bohr diameter, it is difficult to adjust the size of the quantum dots, and the color purity and spectrum are affected by the size and size distribution, and there is a disadvantage in that the efficiency is rather reduced due to the defect on the crystal surface. In order to solve this problem, it is possible to provide nanocrystal particle having a size larger than the exciton Bohr diameter, which are not affected by the quantum confinement effect and exhibit maximum luminescence efficiency.

The method for deriving the exciton Bohr diameter is described in the [ACS Nano, 2017, 11 (7), pp 6586-6593, AIP Advances, 2018, 8, 025108] papers, their Supporting Information, and references described in this paper [in particular, Nature Physics, 2015, 11, 582; Energy & Environmental Science, 2016, 9, 962; J. Phys. Chem. Lett., 2017, 8, 1851]. As an example, in the case of MAPbBr₃, the exciton Bohr diameter may be about 10 nm. It may be smaller than 10 nm or higher depending on the material. When obtaining physical parameters to be used in such a measurement, it should be obtained within a range that those skilled in the art agree. Recent papers on dielectric constant according to frequency [Advanced Energy Materials, 2017, 7, 1700600; APL Materials, 2019, 7, 010901; According to Advanced Materials, 2019, 31, 1806671], the dielectric constant (εᵣ) should not be used in the range of a dynamic dielectric constant higher than the high frequency (>1,000,000 Hz). It should be determined by considering the static dielectric constant (ε₀=static dielectric constant). A high frequency level of about 10¹⁵ Hz is the range in which the photophysical reaction takes place, and ε_{∞} can be defined in this range. The dielectric constant used to calculate the exciton Bohr diameter should be between ε_{∞} and ε₀. In general, considering that an organic semiconductor material has a dielectric constant of 3-5, the ionic metal halide perovskite material must have a static dielectric constant much larger than these values, and the ionic metal halide perovskite materials possess dielectric constant of 10 or more and 50 or less when measured at room temperature, more preferably 20 or more and 35 or less at room temperature, and may vary depending on the temperature in the range between 20 and 100. The CsPbBr₃ material has a dielectric constant that is almost independent of temperature, but the organic-inorganic hybrid metal halide perovskite has a dependence on temperature. And the measurement should be made with a pure metal halide perovskite thin film without ligands, and the value measured at normal room temperature should be put into the formula. In a typical metal halide perovskite semiconductor in the range of 1 eV to 3.5 eV, it is reasonable that the dielectric constant of the metal halide perovskite has a value that is more than twice the dielectric constant of an organic material. The dielectric constant can be measured through a conventional LCR meter or obtained by fitting with an equivalent circuit after measurement with an impedance spectroscopy equipment. See also Nature Physics, 2015, 11, 582; Energy & Environmental Science, 2016, 9, 962; J. Phys. Chem. Lett., 2017, 8, 1851. As shown in those papers, after obtaining the effective mass and exciton binding energy, dielectric constant can be obtained according to the equation R*=R₀μ/m₀ε₂² (R*=exciton binding energy, R₀=atomic Rydberg constant, m₀=free electron mass, μ=reduced effective mass defined by 1/μ=1/μₕ1+μₑ, mₕ=effective mass of hole, mₑ=effective mass of electron). The effective dielectric constant obtained in this way and reported in AIP Advances, 2018, 8, 025108 is 11.4. At this time, a value of μ=0.117 m₀ was used. At this time, the calculated exciton Bohr radius is 5.16 nm and the exciton Bohr diameter is 10.32 nm (in the paper, it described that the exciton Bohr radius is 4.7 nm, so the exciton Bohr diameter is 9.4 nm, but it is judged as a calculation error.)

The exciton Bohr diameter can be obtained by the value of the effective mass of the metal halide perovskite and Equation 1 below.

$$r = {a}_{0} {ɛ}_{r} \frac{{m}_{0}}{μ} \qquad [ Equation 1 ]$$

Where r is the Bohr exciton radius, a₀ is the Bohr diameter of hydrogen (0.053 nm), εᵣ is the dielectric constant, μ=mₑ×mₕ/(mₑ+mₕ), mₑ is the effective electron mass and mₕ can be the effective hole mass. Here, the Bohr diameter represents twice the Bohr radius.

In addition, ITO/PEDOT:PSS/perovskite film/electron injection layer/cathode structure device was fabricated, and the capacitance (C) value of the perovskite thin film at 1000 Hz was measured through Impedance Spectroscopy. Afterwards C=εᵣε₀ A/d (where A is the device area and d is the thickness), εᵣ is measured, and the reduced effective mass value (μ=0.117 m₀) in the papers of Energy & Environmental Science, 2016, 9, 962 for MAPbBr₃ is used to determine the exciton Bohr diameter which was calculated as 12.4 nm.

Here, the dielectric constant should be measured at room temperature and measured using a pure metal halide perovskite thin film without a ligand, and it may vary depending on the material, but may generally have a value of 7-30, and more preferably between 7-20. However, when it has a value less than 7, it may be due to an error in measurement and thus should be measured with special care. In the case of MAPbBr₃, it may vary depending on the crystal size or the quality of the thin film, but a value between 7 and 20 is a reasonable range. In addition, if different values come out depending on the quality of the thin film, the measured value using the thin film of which the grain size is made as large as possible should be used.

Another way to experimentally determine the exciton Bohr diameter is that the size of the point at which the photoluminescence peak wavelength starts to change rapidly with a function of the size of the nanoparticles is very close to the exciton Bohr diameter, or it can be viewed as the particle size at the point where the full width at half maximum (FWHM) of the photoluminescence spectrum starts to increase. The quantum confinement effect begins at the size of the above exciton Bohr diameter, and particles below this point are called quantum dots. If the particle size becomes smaller in the quantum dot regime and the distribution of the particle size is present, the photoluminescence peak of a particle shifts toward blue color and the emission colors can be changed with the particle size, so the FWHM can be increased because photoluminescence spectrum is measured from ensembles of all the nanoparticles. It is most preferable to measure the size of the particles with a transmission electron microscope (TEM). When measured by the light scattering method, the particle size error is larger. When the particles are agglomerated, it is difficult to analyze the size of one particle, and the size of the aggregated particles is overestimated.

The quantum confinement effect refers to a phenomenon observed when the energy band is affected by a change in the atomic structure of a particle, and the exciton Bohr diameter is the point (the size of the semiconductor particle) at which the quantum confinement effect occurs. In other words, if a quantum dot is a particle of the semiconductor of which the size is an exciton Bohr diameter or less, as the particle size decreases the quantum confinement effect is applied, and accordingly, the “band gap” and the corresponding “emission wavelength (photoluminescence (PL) spectrum)” changes. Therefore, in order to obtain a practical value of the exciton Bohr diameter, it is necessary to find a region where the quantum confinement effect begins, that is, the “point at which the emission wavelength changes according to the size” of the semiconductor particle.

However, even when the particle size is larger than the exciton Bohr diameter, since electron-hole interaction in the semiconductor changes, the band gap and emission wavelength of the semiconductor particle may change. However, since the amount of change in this part is very small, it is commonly referred to as “Weak confinement regime”. On the other hand, the quantum confinement regime in which the band gap varies greatly depending on the size of the quantum dot particles is referred to as a “strong confinement regime”. Therefore, in order to obtain the exciton Bohr diameter, the boundary between the weak confinement regime and the strong confinement regime must be found. Therefore, when the particle size obtained through this experimentally the point at which the PL peak or FWHM rapidly changes (the point at which the straight line drawn along the slope meets when two sharply different slopes meet) matched the value obtained by the above equation within a slight error range (approximately 10%), the exciton Bohr diameter obtained by the formula can be said to be a physically meaningful value.

Referring to FIG. 2, a metal halide perovskite nanocrystal particle (100) according to the present disclosure may include a metal halide perovskite nanocrystal structure (110) that can be dispersed in an organic solvent. The organic solvent at this time may be a polar solvent or a non-polar solvent.

For example, the polar solvent includes acetic acid, acetone, acetonitrile, dimethylformamide, gamma butyrolactone, N-methylpyrrolidone, ethanol or dimethylsulfoxide, and the non-polar solvent includes ethylene dichloride, trichloroethylene, chloroform, chlorobenzene, dichlorobenznene, styrene, dimethylformamide, dimethyl sulfoxide, xylene, toluene, cyclohexane, or isopropyl alcohol, but is not limited thereto.

In addition, the shape structures of the metal halide perovskite nanocrystals may be forms generally used in the field. The shape of the metal halide perovskite nanocrystal may be a 0-dimensional, 1-dimensional or 2-dimensional shape. As an example, it may be in the form of sphere, ellipsoid, cube, hollow cube, pyramid, cylinder, cone, elliptic column, hollow sphere, Janus particle, prism, multipod, polyhedron, nano tube, nano wire, nano fiber or nanoplatelet.

In addition, the size of the crystalline particles may be 1 nm to 10 μm or less. For example, the size of the crystalline particles may be 1 nm, 2 nm, 3 nm, 4 nm, 5 nm, 6 nm, 7 nm, 7.5 nm, 8 nm, 8.5 nm, 9 nm, 9.5 nm, 10 nm, 10.5 nm, 11 nm, 11.5 nm, 12 nm, 12.5 nm, 13 nm, 13.5 nm, 14 nm, 14.5 nm, 15 nm, 16 nm, 17 nm, 18 nm, 19 nm, 20 nm, 25 nm, 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 150 nm, 200 nm, 250 nm, 300 nm, 350 nm, 400 nm, 450 nm, 500 nm, 600 nm, 700 nm, 800 nm, 900 nm, 1 μm, 2 μm, 5 μm, or 10 μm. The size of the particle can be defined as a region that takes the lower value as the minimum value and the larger value as the maximum value out of the two numbers selected above. It is preferably 8 nm or more and 300 nm or less, and more preferably 10 nm or more and 30 nm or less. On the other hand, the size of the crystalline particles at this time means a size that does not take the length of a ligand to be described later into account, that is, the size of the remaining portions excluding the ligand. When the size of the crystalline particles is 1 μm or more, there may be a fundamental problem in that excitons do not undergo luminescence due to thermal ionization and delocalization of charge carriers in a large crystal, but are separated into free charge carriers and disappear. In addition, more preferably, as described above, the size of the crystalline particles may be greater than or equal to exciton Bohr diameter. The thermal ionization and delocalization of the charge carrier may gradually increase when the size of the nanocrystal exceeds 100 nm. If it is more than 300 nm, the phenomenon will occur more significantly, and if it is more than 1 μm, it is completely bulky and is subject to the above phenomenon.

For example, when the crystalline particles are spherical, the diameter of the crystalline particles may be 1 nm to 10 μm. For example, the diameter of the crystalline particles may be 1 nm, 2 nm, 3 nm, 4 nm, 5 nm, 6 nm, 7 nm, 7.5 nm, 8 nm, 8.5 nm, 9 nm, 9.5 nm, 10 nm, 10.5 nm, 11 nm, 11.5 nm, 12 nm, 12.5 nm, 13 nm, 13.5 nm, 14 nm, 14.5 nm, 15 nm, 16 nm, 17 nm, 18 nm, 19 nm, 20 nm, 25 nm, 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 150 nm, 200 nm, 250 nm, 300 nm, 350 nm, 400 nm, 450 nm, 500 nm, 600 nm, 700 nm, 800 nm, 900 nm, 1 μm, 2 μm, 5 μm or 10 μm.

In addition, the band gap energy of the nanocrystal particle may be 1 eV to 5 eV. Preferably, the band gap energy of the nanocrystal particle may be included in 1 eV, 1.1 eV, 1.2 eV, 1.3 eV, 1.4 eV, 1.5 eV, 1.6 eV, 1.7 eV, 1.8 eV, 1.81 eV, 1.82 eV, 1.83 eV, 1.84 eV, 1.85 eV, 1.86 eV, 1.87 eV, 1.88 eV, 1.89 eV, 1.9 eV, 1.91 eV, 1.92 eV, 1.93 eV, 1.94 eV, 1.95 eV, 1.96 eV, 1.97 eV, 1.98 eV, 1.99 eV, 2 eV, 2.01 eV, 2.02 eV, 2.03 eV, 2.04 eV, 2.05 eV, 2.06 eV, 2.07 eV, 2.08 eV, 2.09 eV, 2.1 eV, 2.11 eV, 2.12 eV, 2.13 eV, 2.14 eV, 2.15 eV, 2.16 eV, 2.17 eV, 2.18 eV, 2.19 eV, 2.2 eV, 2.21 eV, 2.22 eV, 2.23 eV, 2.24 eV, 2.25 eV, 2.26 eV, 2.27 eV, 2.28 eV, 2.29 eV, 2.3 eV, 2.31 eV, 2.32 eV, 2.33 eV, 2.34 eV, 2.35 eV, 2.36 eV, 2.37 eV, 2.38 eV, 2.39 eV, 2.4 eV, 2.41 eV, 2.42 eV, 2.43 eV, 2.44 eV, 2.45 eV, 2.46 eV, 2.47 eV, 2.48 eV, 2.49 eV, 2.5 eV, 2.51 eV, 2.52 eV, 2.53 eV, 2.54 eV, 2.55 eV, 2.56 eV, 2.57 eV, 2.58 eV, 2.59 eV, 2.6 eV, 2.61 eV, 2.62 eV, 2.63 eV, 2.64 eV, 2.65 eV, 2.66 eV, 2.67 eV, 2.68 eV, 2.69 eV, 2.7 eV, 2.71 eV, 2.72 eV, 2.73 eV, 2.74 eV, 2.75 eV, 2.76 eV, 2.77 eV, 2.78 eV, 2.79 eV, 2.8 eV, 2.9 eV, 3 eV, 3.1 eV, 3.2 eV, 3.3 eV, 3.4 eV, 3.5 eV, 3.6 eV, 3.7 eV, 3.8 eV, 3.9 eV, 4 eV, 4.1 eV, 4.2 eV, 4.3 eV, 4.4 eV, 4.5 eV, 4.6 eV, 4.7 eV, 4.8 eV, 4.9 eV, and 5 eV.

Accordingly, since the energy band gap is determined according to the constituent material or crystal structure of the nanocrystal particle, light having a wavelength of, for example, 200 nm to 1300 nm may be emitted by controlling the constituent material of the nanocrystal particle. In addition, preferably, the nanocrystal particle may emit ultraviolet, blue, green, red, and infrared light.

The ultraviolet light includes ranges in which a lower value out of two numbers selected among 200 nm, 210 nm, 220 nm, 230 nm, 240 nm, 250 nm, 260 nm, 270 nm, 280 nm, 290 nm, 300 nm, 310 nm, 320 nm, 330 nm, 340 nm, 350 nm, 360 nm, 370 nm, 380 nm, 390 nm, 400 nm, 410 nm, 420 nm, and 430 nm a lower limit and a higher value has an upper limit may be included. The blue light includes ranges in which a lower value of two numbers among 440 nm, 450 nm, 451 nm, 452 nm, 453 nm, 454 nm, 455 nm, 456 nm, 457 nm, 458 nm, 459 nm, 460 nm, 461 nm, 462 nm, 463 nm, 464 nm, 465 nm, 466 nm, 467 nm, 468 nm, 469 nm, 470 nm, 471 nm, 472 nm, 473 nm, 474 nm, 475 nm, 476 nm, 477 nm, 478 nm, 479 nm, and 480 nm, is a lower limit and a higher value of two is an upper limit. The green light is range in which a lower value of two numbers selected among 500 nm, 501 nm, 502 nm, 503 nm, 504 nm, 505 nm, 506 nm, 507 nm, 508 nm, 509 nm, 510 nm, 511 nm, 512 nm, 513 nm, 514 nm, 515 nm, 516 nm, 517 nm, 518 nm, 519 nm, 520 nm, 521 nm, 522 nm, 523 nm, 524 nm, 525 nm, 526 nm, 527 nm, 528 nm, 529 nm, 530 nm, 531 nm, 532 nm, 533 nm, 534 nm, 535 nm, 536 nm, 537 nm, 538 nm, 539 nm, 540 nm, 541 nm, 542 nm, 543 nm, 544 nm, 545 nm, 546 nm, 547 nm, 548 nm, 549 nm, 550 nm, 560 nm, 570 nm, and 580 nm, is a lower limit and a higher value of the two has an upper limit. The red light include ranges in which a lower value of two numbers selected among 590 nm, 600 nm, 601 nm, 602 nm, 603 nm, 604 nm, 605 nm, 606 nm, 607 nm, 608 nm, 609 nm, 610 nm, 611 nm, 612 nm, 613 nm, 614 nm, 615 nm, 616 nm, 617 nm, 618 nm, 619 nm, 620 nm, 621 nm, 622 nm, 623 nm, 624 nm, 625 nm, 626 nm, 627 nm, 628 nm, 629 nm, 630 nm, 631 nm, 632 nm, 633 nm, 634 nm, 635 nm, 636 nm, 637 nm, 638 nm, 639 nm, 640 nm, 641 nm, 642 nm, 643 nm, 644 nm, 645 nm, 646 nm, 647 nm, 648 nm, 649 nm, 650 nm, 660 nm, 670 nm, 680 nm, 690 nm, and 700 nm is a lower limit and a higher value is an upper limit. The infrared light includes ranges in 700 nm, 710 nm, 720 nm, 730 nm, 740 nm, 750 nm, 760 nm, 770 nm, 780 nm, 790 nm, 800 nm, 810 nm, 820 nm, 830 nm, 840 nm, 850 nm, 860 nm, 870 nm, 880 nm, 890 nm, 900 nm, 910 nm, 920 nm, 930 nm, 940 nm, 950 nm, 960 nm, 970 nm, 980 nm, 990 nm, 1000 nm, 1010 nm, 1020 nm, 1030 nm, 1040 nm, 1050 nm, 1060 nm, 1070 nm, 1080 nm, 1090 nm, 1100 nm, 1110 nm, 1120 nm, 1130 nm, 1140 nm, 1150 nm, 1160 nm, 1170 nm, 1180 nm, 1190 nm, 1200 nm, 1210 nm, 1220 nm, 1230 nm, 1240 nm, 1250 nm, 1260 nm, 1270 nm, 1280 nm, 1290 nm, 1300 nm, 1350 nm, 1400 nm, 1450 nm, and 1500 nm is a lower limit and a higher value of the two is an upper limit.

The metal halide perovskite nanocrystal particles according to the present disclosure may provide nanocrystal particle having various band gaps according to substitution of halogen elements.

For example, a nanocrystal particle including a CH₃NH₃PbCl₃ organic-inorganic metal halide perovskite nanocrystal structure may have a band gap energy of about 3.1 eV. In addition, the nanocrystal particle including the CH₃NH₃PbBr₃ organic-inorganic metal halide perovskite nanocrystal structure may have a band gap energy of about 2.3 eV. In addition, the nanocrystal particle including the CH₃NH₃PbI₃ organic-inorganic metal halide perovskite nanocrystal structure may have a band gap energy of about 1.5 eV.

In addition, the metal halide perovskite nanocrystal particles according to the present disclosure may provide nanocrystal particle having various band gaps according to substitution of organic elements.

For example, when n=4 in (CₙH₂ₙ₊₁NH₃)₂PbBr₄, nanocrystal particle having a band gap of about 3.5 eV may be provided. In addition, when n=5, nanocrystal particle having a band gap of about 3.33 eV may be provided. In addition, when n=7, nanocrystal particle having a band gap of about 3.34 eV may be provided. In addition, when n=12, nanocrystal particle having a band gap of about 3.52 eV may be provided.

In addition, the metal halide perovskite nanocrystal particles according to the present disclosure may provide nanocrystal particle having various band gaps according to the substitution of a central metal

For example, a nanocrystal particle including a CH₃NH₃PbI₃ organic-inorganic metal halide perovskite nanocrystal structure may have a band gap energy of about 1.5 eV. In addition, the nanocrystal particle including the CH₃NH₃Sn_{0.3}Pb_{0.7}I₃ organic-inorganic metal halide perovskite nanocrystal structure may have a band gap energy of about 1.31 eV. In addition, the nanocrystal particle including the CH₃NH₃Sn_{0.5}Pb_{0.5}I₃ organic-inorganic metal halide perovskite nanocrystal structure may have a band gap energy of about 1.28 eV. In addition, the nanocrystal particle including the CH₃NH₃Sn_{0.7}Pb_{0.3}I₃ organic-inorganic metal halide perovskite nanocrystal structure may have a band gap energy of about 1.23 eV. In addition, the nanocrystal particle including the CH₃NH₃Sn_{0.9}Pb_{0.1}I₃ organic-inorganic metal halide perovskite nanocrystal structure may have a band gap energy of about 1.18 eV. In addition, the nanocrystal particle including the CH₃NH₃SnI₃ organic-inorganic metal halide perovskite nanocrystal structure may have a band gap energy of about 1.1 eV. In addition, the nanocrystal particle including the CH₃NH₃PbₓSn₁₋ₓBr₃ organic-inorganic metal halide perovskite nanocrystal structure may have a band gap energy of 1.9 eV to 2.3 eV. In addition, the nanocrystal particle including the CH₃NH₃PbₓSn₁₋ₓCl₃ organic-inorganic metal halide perovskite nanocrystal structure may have a band gap energy of 2.7 eV to 3.1 eV.

A schematic diagram 3 is showing a method of manufacturing a metal halide perovskite nanocrystal particle according to an embodiment of the present disclosure.

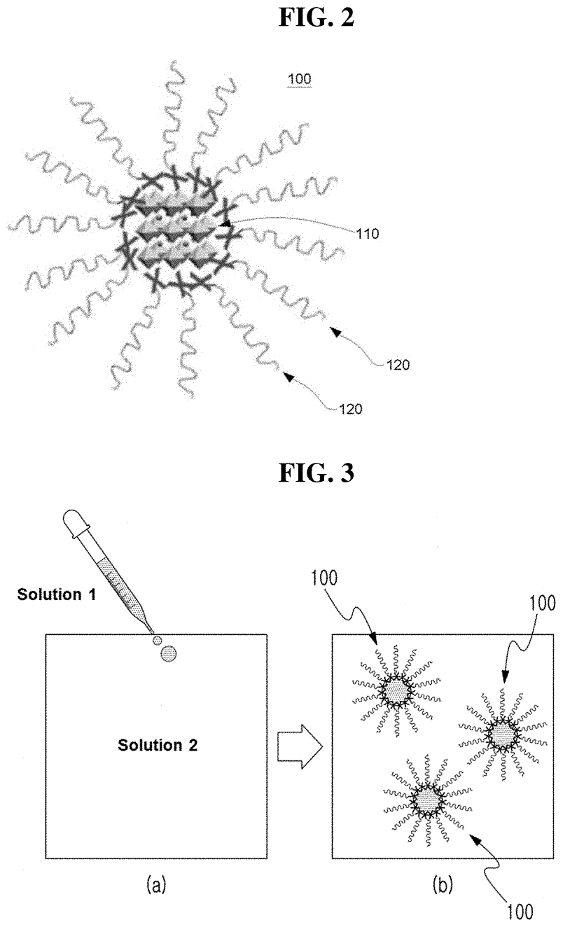

Referring to FIG. 3, in the method for preparing metal halide perovskite nanocrystal particles according to an embodiment of the present disclosure, a first solution in which a metal halide perovskite is dissolved in a polar solvent and a surfactant is dissolved in a non-polar solvent. Preparing a second solution and mixing the first solution with the second solution to form nanocrystal particle.

First, a first solution in which a metal halide perovskite is dissolved in a polar solvent and a second solution in which a surfactant is dissolved in a non-polar solvent are prepared.

The polar solvent at this time may include dimethylformamide, gamma butyrolactone, N-methylpyrrolidone, or dimethylsulfoxide, but is not limited thereto.

The metal halide perovskite may be a material having a three-dimensional crystal structure, a two-dimensional crystal structure, a one-dimensional crystal structure, or a zero-dimensional crystal structure.

The metal halide perovskite is ABX₃(3D), A₄BX₆(0D), AB₂X₅(2D), A₂BX₄(2D), A₂BX₆(0D), A₂B⁺B³⁺X₆(3D), A₃B₂X₉(2D) or it may include a structure of Aₙ₋₁BₙX₃ₙ₊₁ (quasi-2D) (n is an integer between 2 and 6). A is a monovalent cation, B is a metal material, and X may be a halogen element. Specific examples of A, B, and X of the metal halide perovskite are as described in the previous section of <Metal Halide Perovskite Crystal>.

On the other hand, such a metal halide perovskite can be prepared by combining AX and BX₂ in a certain ratio. That is, the first solution may be formed by dissolving AX and BX₂ in a polar solvent at a predetermined ratio. For example, by dissolving AX and BX₂ in a polar solvent in a ratio of 2:1, a first solution in which a metal halide perovskite precursor is dissolved may be prepared.

In addition, the non-polar solvent at this time may include dichloroethylene, trichloroethylene, chloroform, chlorobenzene, dichlorobenzene, styrene, dimethylformamide, dimethylsulfoxide, xylene, toluene, hexane, octadecene, cyclohexene or isopropyl alcohol.

In addition, the surfactant may include an alkyl halide, an amine ligand, and a carboxylic acid or phosphonic acid.

The specific description of the alkyl halide, amine ligand, carboxylic acid, and phosphonic acid is as described in the previous section of <metal halide perovskite nanocrystal particle>.

Next, the first solution is mixed with the second solution to form nanocrystal particle.

In the step of forming nanocrystal particle by mixing the first solution with the second solution, it is preferable to mix the first solution by dropping the first solution into the second solution. At this time, it is preferable to mix by dropping into fine droplets, and to react by dropping several droplets finely from a spray or nozzle. In some cases, the first solution in the beaker may be poured as it is and dropped into the stirred second solution. In addition, the second solution at this time may be stirred. For example, a first solution in which an organic-inorganic metal halide perovskite (OIP) is dissolved is slowly added dropwise to a second solution in which an alkyl halide surfactant is dissolved and then nanocrystal particle are synthesized.

In this case, when the first solution is dropped into the second solution and mixed, organic-inorganic metal halide perovskite (OIP) is precipitated from the second solution due to a difference in solubility. The organic-inorganic metal halide perovskite (OIP) precipitated in the second solution is stabilized by an alkyl halide surfactant to form well-dispersed organic-inorganic metal halide perovskite nanocrystals (OIP-NC). Accordingly, it is possible to prepare metal halide perovskite nanocrystal particles including organic-inorganic metal halide perovskite nanocrystals and a plurality of alkyl halide organic ligands surrounding the organic/inorganic metal halide perovskite nanocrystals.

Meanwhile, the size of the crystalline particles of the organic/inorganic metal halide perovskite can be controlled by controlling the length or shape factor and amount of the alkyl halide surfactant. For example, the shape factor control can control the size through a linear, tapered, or inverted triangular surfactant.

In addition, the metal halide perovskite nanocrystal particles according to an embodiment of the present disclosure may have a core-shell structure.

Hereinafter, a core-shell structured metal halide perovskite nanocrystal particle according to an embodiment of the present disclosure will be described.

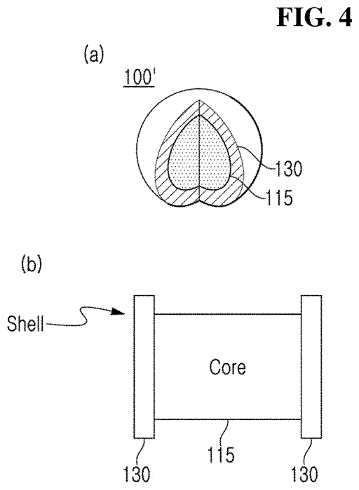

FIG. 4 is a schematic diagram showing a core-shell structured metal halide perovskite nanocrystal particle and an energy band diagram thereof according to an embodiment of the present disclosure.

Referring to FIG. 4(a), a core-shell structured metal halide perovskite nanocrystal particle (100′) according to the present disclosure includes a core (115) and a shell (130) surrounding the core (115). In this case, a material having a band gap larger than that of the core (115) may be used as the material of the shell (130).

At this time, referring to FIG. 4(b), the energy band gap of the shell (130) is larger than the energy band gap of the core (115), so that excitons can be better constrained to the core metal halide perovskite.

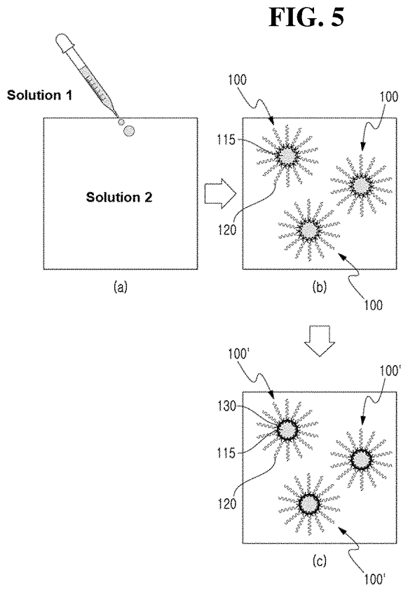

FIG. 5 is a schematic diagram showing a method for producing a metal halide perovskite nanocrystal particle having a core-shell structure according to an embodiment of the present disclosure.

A method for producing a core-shell structured metal halide perovskite nanocrystal particle according to an embodiment of the present disclosure comprises a first solution in which a first metal halide perovskite is dissolved in a polar solvent and alkyl in a non-polar solvent. Preparing a second solution in which at least one surfactant selected from a halide, a carboxylic acid derivative, and an amine derivative is dissolved, and the first solution is mixed with the second solution to obtain a first metal halide solution. It may include forming a core including a perovskite nanocrystal structure, and forming a shell surrounding the core and including a material having a larger band gap than the core.

Referring to FIG. 5(a), a first solution in which a metal halide perovskite is dissolved in a polar solvent is added dropwise to a second solution in which an alkyl halide surfactant is dissolved in a non-polar solvent.

Referring to FIG. 5(b), when the first solution is added to the second solution, metal halide perovskite is precipitated in the second solution due to the difference in solubility, and the precipitated metal halide perovskite includes a well-dispersed metal halide perovskite nanocrystalline core (115) while stabilizing the surface by being surrounded by at least one surfactant selected from an alkyl halide, a carboxylic acid derivative, and an amine derivative. The metal halide perovskite nanocrystal particles (100) are generated. At this time, the nanocrystalline core (115) is surrounded by the alkyl halide organic ligands (120).

When octadecene or hexane is used as a non-polar solvent to prepare the second solution, there is no miscibility with polar solvents such as dimethylsulfoxide, dimethylformamide, gamma butyrolactone, or N-methylpyrrolidone, so it is not mixed at all. Then, the phase separation occurs and thus the reaction does not occur, and the reaction still does not occur even after stirring. When vigorously stirred, an opaque emulsion solution is formed as cloudy as milky, and the color of the perovskite itself is not found. However, when acetone or alcohol such as tert-butanol is added into this solution, a reaction occurs and particles are formed. In this case, the ligands mix with the other solvent through acetone or tert-butanol, thereby causing a reaction surrounding the nanocrystal particle. This method is called the Inverse Nano Emulsion method. If instead of hexane and octadecene, you use a solvent (eg, toluene) that can mix with even a little bit of polar solvent, there is no need to inject additional solvent, and when the first solution is dropped into the second solution, perovskite particles are formed. This case is called the Ligand-Assisted Reprecipitation method. If the first solution is injected into the second solution and injected at a temperature of at least 50 degrees above room temperature, it is called a hot injection method. Typically, the high-temperature injection method is performed in an inert gas atmosphere.

Since the description on FIGS. 5(a) and 5(b) is the same as described above in FIG. 4, a detailed description thereof will be omitted.

Referring to FIG. 5(c), metal halide perovskite nanocrystal particles (100′) having a core-shell structure can be prepared by forming a shell (130) including a material having a larger band gap than the core (115) while surrounding the core (115). The following five methods can be used for the methods of forming such a shell.

As a first method, a shell may be formed using a second metal halide perovskite solution or an inorganic semiconductor material solution. That is, by adding a third solution, in which a second metal halide perovskite having a larger band gap than the first metal halide perovskite or an inorganic semiconductor material is dissolved, to the second solution, a shell may be formed: The shell surrounding the core may include a second metal perovskite nanocrystal, an inorganic semiconductor material or an organic polymer.

For example, while strongly stirring the solution of metal halide perovskites (MAPbBr₃) produced through the inverse nano-emulsion method, ligand-assisted reprecipitation method, and hot injection method as described above, a solution of metal halide perovskite (MAPbCl₃) with a larger band gap than MAPbBr₃, or a solution of inorganic semiconductor materials such as metal sulfide (e.g. PbS and ZnS) or metal oxide or a precursor solution thereof, or a solution of organic polymers such as polyethylene glycol, polyethylene oxide, polyvinylpyrrolidone, polyethyleneimine, polyvinyl alcohol (PVA), polysilazane, acrylate polymer, fluorinated polyvinylidene fluoride (PVDF) polymer, and acrylate-based low-molecular monomer, are slowly added dropwise or several drops into the metal halide perovskite solution. Then, a shell that includes a second metal halide perovskite nanocrystal (MAPbCl₃) or an inorganic semiconductor material may be formed. MA above stands for methyl ammonium.

The core-shell metal halide perovskite nanocrystals are synthesized because the core made of metal halide perovskite and the shell made of metal halide perovskite are mixed to form an alloy or adhere to each other.

Therefore, it is possible to form metal halide perovskite nanocrystal particles of MAPbBr₃/MAPbCl₃ core-shell structure.

In addition, after dispersing an organic polymer dot and an existing inorganic quantum dot (mainly III-V, II-IV semiconductor) in the second solution, the perovskite shell is formed by injecting the perovskite precursor.

As a second method, a shell can be formed using an organic ammonium halide solution. That is, after adding a large amount of the organic ammonium halide solution to the second solution and stirring, a shell having a larger band gap than the core surrounding the core may be formed.

For example, metal halide perovskite produced through the inverse nano-emulsion method, ligand-assisted reprecipitation method, and hot injection method as described above The MACl solution is added to the MAPbBr₃ solution, stirred vigorously, and MAPbBr₃ on the surface is converted to MAPbBr₃₋ₓClₓ by an excessive amount of MACl to form a shell.

Accordingly, metal halide perovskite nanocrystal particles having a MAPbBr₃/MAPbBr₃₋ₓClₓ core-shell structure can be formed.

In addition, a metal halide perovskite (MAPbI₃) solution produced through the inverse nano-emulsion method, ligand-assisted reprecipitation method, and hot injection method as described above. The MABr solution is added to and vigorously stirred, and MAPbI₃ on the surface is converted into MAPbI₃₋ₓBrₓ by an excessive amount of MABr, thereby forming a shell.

Accordingly, metal halide perovskite nanocrystal particles having a MAPbI₃/MAPbI₃₋ₓBrₓ core-shell structure can be formed.

In addition, the MAI solution is added to a solution of metal halide perovskite (MAPbBr₃) produced through the inverse nano-emulsion method, ligand-assisted reprecipitation method, and hot injection method as described above, the solution is vigorously stirred, and thus MAPbBr₃ on the surface is converted into MAPbBr₃₋ₓIₓ by an excessive amount of MAI to form a shell.

Accordingly, metal halide perovskite nanocrystal particles having a MAPbBr₃/MAPbBr₃₋ₓIₓ core-shell structure can be formed. In this case, a red-emitting perovskite can be prepared.

As a third method, the shell can be formed using a pyrolysis/synthesis method. That is, after thermally decomposing the surface of the core by heat-treating the second solution, an organic ammonium halide solution is added to the heat-treated second solution to synthesize the surface again, so that the band gap of the shell is larger than that of the core while the shell surrounds the core.

For example, after heat-treating a solution of metal halide perovskite (MAPbBr₃) produced through the inverse nano-emulsion method as described above, thermally decomposing the surface of the perovskite particle to be converted to PbBr₂, and then adding the MACl solution to the heat-treated solution, the shell can be formed by reacting it again to have MAPbBr₂Cl at the surface. In this case, blue-emitting perovskite particles can be produced.

Accordingly, metal halide perovskite nanocrystal particles having a MAPbBr₃/MAPbBr₂Cl core-shell structure can be formed.

Therefore, the metal halide perovskite nanocrystal particles of the core-shell structure formed according to the present disclosure form a shell with a material having a larger band gap than the core, so that excitons are better confined to the core, and the metal halide perovskite nanocrystal particles are stable in air. It is possible to improve the durability of nanocrystals by using air-stable metal halide perovskite or inorganic semiconductor to prevent the core made of metal halide perovskite from being exposed to air.

As a fourth method, a shell can be formed using an organic semiconductor material solution. That is, in the second solution, an organic semiconductor material having a larger band gap than the metal halide perovskite is previously dissolved, and the first solution in which the above-described first metal halide perovskite is dissolved is added to the second solution. A core including a first metal halide perovskite nanocrystal and a shell including an organic semiconductor material surrounding the core may be formed.

Since the organic semiconductor material adheres to the surface of the core metal halide perovskite, it is possible to synthesize a metal halide perovskite having a core-shell structure.

Therefore, MAPbBr₃-organic semiconductor core-shell structure metal halide perovskite nanocrystal particle light emitters can be formed.

As a fifth method, a shell may be formed using a selective extraction method. That is, by adding a small amount of IPA solvent to the second solution in which the core containing the first metal halide perovskite nanocrystal is formed, MABr is selectively extracted from the surface of the nanocrystal and the surface is formed only with PbBr₂ to surround the core A shell having a larger band gap than the core may be formed.

For example, by adding a small amount of IPA to the metal halide perovskite (MAPbBr₃) solution produced through the inverse nano-emulsion method as described above, only MABr on the nanocrystal surface is selectively dissolved. PbBr₂ shell can be formed by extracting so that only PbBr₂ remains on the surface.

That is, at this time, MABr on the surface of MAPbBr₃ may be removed through selective extraction.

Therefore, it is possible to form a metal halide perovskite nanocrystal particle light emitter having a MAPbBr₃—PbBr₂ core-shell structure.

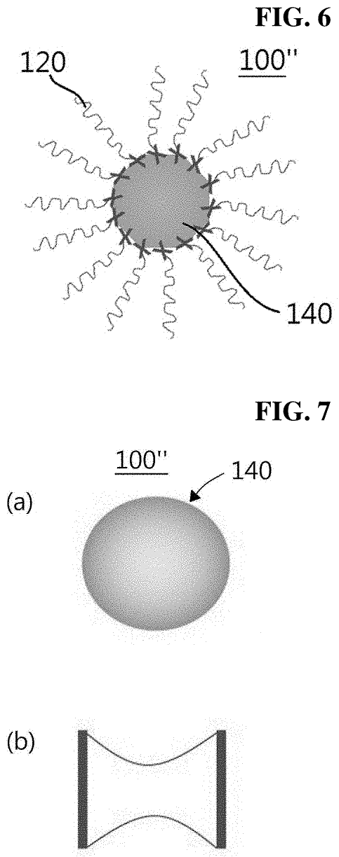

FIG. 6 is a schematic diagram showing a metal halide perovskite nanocrystal particle having a gradient composition structure according to an embodiment of the present disclosure.

FIG. 6, a metal halide perovskite nanocrystal particle (100″) having a structure having a gradient composition according to an embodiment of the present disclosure has a metal halide perovskite nanocrystal structure (140) that can be dispersed in an organic solvent and the nanocrystal structure (140) has a gradient composition structure whose composition changes from the center toward the outside, and the organic solvent at this time may be a polar solvent or a non-polar solvent.

The metal halide perovskite at this time has a structure of ABX₃₋ₘX′ₘ, A₂BX₄₋₁X′₁ or ABX₄₋ₖX′ₖ, wherein A is a monovalent cation, B is a metal material, and X is Br, X′ may be Cl or X may be I, and X′ may be Br. In addition, the m, l, and k values are characterized by increasing from the center of the nanocrystal structure (140) toward the outside.

Accordingly, the energy band gap increases from the center of the nanocrystal structure (140) toward the outside.

For example, the monovalent cation may be a monovalent organic cation or an alkali metal. For example, the monovalent organic cation is organic ammonium (RNH₃⁺), organic amidinium derivative (RC(═NR₂)NR₂)⁺, organic guanidinium derivative (R₂NC(═NR₂)NR₂)⁺, organic diammonium (CₓH₂ₓ₋ₙ₊₄)(NH₃)ₙ⁺, ((CₓH₂ₓ₊₁)ₙNH₃)(CH₃NH₃)ₙ⁺, (RNH₃)₂⁺, (CₙH₂ₙ₊₁NH₃)²⁺, (CF₃NH₃)⁺, (CF₃NH₃)ₙ⁺, ((CₓF₂ₓ₊₁)ₙNH₃)₂(CF₃NH₃)ₙ⁺, ((CₓF₂ₓ₊₁)ₙNH₃)₂⁺ or (CₙF₂ₙ₊₁NH₃)₂⁺ (x, n is an integer of 1 or more, R=hydrocarbon derivative, alkyl, alkyl fluoride derivatives, H, F, Cl, Br, I), and combinations thereof, but are not limited thereto. The alkali metal may be Li⁺, Na⁺, K⁺, Rb⁺, Cs⁺, Fr⁺, and combinations thereof, but is not limited thereto.

Also preferably, the organic cations are acetamidinium, azaspironanium, benzene diammonium, benzylammonium, butanediammonium, isobutylammonium, n-butylammonium, t-butylammonium, cyclohexylammonium, cyclohexylmethylammonium, diazobicyclooctanedinium, Diethylammonium, N,N-diethylethane diammonium, N,N-diethylpropane diammonium, dimethylammonium, N, N-dimethylethane diammonium, dimethylpropane diammonium, dodecylammonium, ethanediammonium, ethylammoniuium, 4-fluoro-benzyl Ammonium, 4-fluoro-phenylethylammonium, 4-fluoro-phenylammonium, formamidinium, Guanidinium, hexanediammnium, hexylammonium, imidazolium, 2-methoxyethylammonium, 4-methoxy-phenylethylammonium, 4-methoxy-phenylammonium, methylammonium, morpholinium, octylammonium, pentylammonium, piperidinium, propanediammonium, iso-propylammonium, di-iso-propylammonium, n-propylammonium, pyridinium, 2-pyrrolidin-1-ium-1-yethylammonium, pyrrolidinium, quinclidin-1-ium, 4-trifluoromethyl-benzylammonium, 4-trifluoromethyl ammonium, and Benzalkonium chloride, Dimethyldioctadecylammonium chloride, Trimethylglycine, quaternary ammonium cation such as Choline, and combinations thereof.

The B may be a divalent transition metal, a rare earth metal, an alkaline earth metal, a monovalent metal, a combination of a trivalent metal, an organic substance (a monovalent, divalent, trivalent cation), and a combination thereof. In addition, preferably, the divalent transition metal, rare earth metal, and alkaline earth metal are Be²⁺, Mg²⁺, Ca²⁺, Sr²⁺, Ba²⁺, Ra²⁺, Mn²⁺, Fe²⁺, Co²⁺, Ni²⁺, Cu²⁺, Zn²⁺, Ru²⁺, Bi²⁺, Pd²⁺, Cd²⁺, Pt²⁺, Hg²⁺, Ge²⁺, Sn²⁺, Pb²⁺, Se²⁺, Te²⁺, Po²⁺, Eu²⁺, No²⁺, and combinations thereof, but are not limited thereto. The monovalent metal may be Li⁺, Na⁺, K⁺, Rb⁺, Cs⁺, Fr⁺, Ag⁺, Hg⁺, Ti⁺, and combinations thereof, and the trivalent metal is Cr³⁺, Fe³⁺, Co³⁺, Ru³⁺, Rh³⁺, Ir³⁺, Au³⁺, Al³⁺, Ga³⁺, In³⁺, Ti³⁺, As³⁺, Sb³⁺, Bi³⁺, La³⁺, Ce³⁺, Pr³⁺, Nd³⁺, Pm³⁺, Sm³⁺, Eu³⁺, Gd³⁺, Tb³⁺, Dy³⁺, Ho³⁺, Er³⁺, Tm³⁺, Yb³⁺, Am³⁺, Cm³⁺, Bk³⁺, Cf³⁺, Es³⁺, Fm³⁺, Md³⁺, Lr³⁺ and combinations thereof.

Meanwhile, the m, l, and k values may gradually increase from the center of the nanocrystal structure toward the outside. Therefore, the energy band gap may gradually increase according to the composition change.

As another example, the m, l, and k values may increase in a stepwise shape from the center of the nanocrystal structure toward the outside. Therefore, according to the composition change, the energy band gap may increase in the form of a step.

In addition, a plurality of organic ligands (120) surrounding the metal halide perovskite nanocrystal structure (140) may be further included. The organic ligand (120) may include an alkyl halide, an amine ligand, and a carboxylic acid or phosphonic acid. Detailed descriptions of the alkyl halide, amine ligand, carboxylic acid, and phosphonic acid are as described in the section of <Metal Halide perovskite nanocrystal particles>.

Therefore, by making the nanocrystal structure into a gradient-alloy type, the contents of the metal halide perovskite present in a large amount outside the nanocrystal structure and the metal halide perovskite present in a large amount inside the nanocrystal structure can be gradually changed. This gradual change in the content in the nanocrystal structure uniformly adjusts the fraction in the nanocrystal structure, reduces surface oxidation, and improves exciton confinement in the metal halide perovskite present in a large amount, thereby increasing luminescence efficiency. Not only that, it can also increase durability-stability.

A method of manufacturing a metal halide perovskite nanocrystal particle having a gradient composition structure according to an embodiment of the present disclosure will be described.

The method for preparing metal halide perovskite nanocrystal particles having a gradient composition structure according to an embodiment of the present disclosure includes preparing a metal halide perovskite nanocrystal particle having a core-shell structure, and forming the metal halide perovskite nanocrystal particles to have a gradient composition through interdiffusion by heat treatment.

First, a core-shell structure of metal halide perovskite nanocrystal particles is prepared. A method of manufacturing a metal halide perovskite nanocrystal particle having a related core-shell structure is the same as described above with reference to FIG. 5, and a detailed description thereof is omitted.

Thereafter, the core-shell structured metal halide perovskite nanocrystal particles may be heat-treated to form a gradient composition through mutual diffusion.

For example, a metal halide perovskite having a core-shell structure is annealed at a high temperature to form a solid solution, and then heat treated to have a gradient composition through interdiffusion.

For example, the heat treatment temperature may be 100° C. to 150° C. Interdiffusion can be induced by annealing at this heat treatment temperature.

A method of manufacturing a metal halide perovskite nanocrystal particle having a gradient composition structure according to another embodiment of the present disclosure includes the steps of forming a first metal halide perovskite nanocrystalline core and forming a second metal halide perovskite nanocrystalline shell having a gradient composition and surrounding the core.

First, a first metal halide perovskite nanocrystalline core is formed. This is the same as the method of forming the nanocrystalline core described above, so a detailed description thereof will be omitted.

Then, a second metal halide perovskite nanocrystalline shell having a gradient composition, surrounding the core, is formed.

The second metal halide perovskite has a structure of ABX₃₋ₘX′ₘ, A₂BX₄₋₁X′₁ or ABX₄₋ₖX′ₖ, wherein A is an organic cationic material, and B may be a metal material. The combination of X and X′ may be selected from F⁻, Cl⁻, Br⁻, I⁻, and At⁻, but the ionic radius of X′ may be smaller than X.

The monovalent cation may be a monovalent organic cation or an alkali metal. For example, the monovalent organic cation is organic ammonium (RNH₃⁺), organic amidinium derivative (RC(═NR₂)NR₂)⁺, organic guanidinium derivative (R₂NC(═NR₂)NR₂)⁺, organic diammonium (CₓH₂ₓ₋ₙ₊₄)(NH₃)ₙ⁺, ((CₓH₂ₓ₊₁)ₙNH₃)(CH₃NH₃)ₙ⁺, (RNH₃)₂⁺, (CₙH₂ₙ₊₁NH₃)²⁺, (CF₃NH₃)⁺, (CF₃NH₃)ₙ⁺, ((CₓF₂ₓ₊₁)ₙNH₃)₂(CF₃NH₃)ₙ⁺, ((CₓF₂ₓ₊₁)ₙNH₃)₂⁺ or (CₙF₂ₙ₊₁NH₃)₂⁺ (x, n is an integer of 1 or more, R=hydrocarbon derivative, alkyl, alkyl fluoride derivatives, H, F, Cl, Br, I), and combinations thereof, but are not limited thereto. The alkali metal may be Li⁺, Na⁺, K⁺, Rb⁺, Cs⁺, Fr⁺, and combinations thereof, but is not limited thereto.

Also preferably, the organic cations are acetamidinium, azaspironanium, benzene diammonium, benzylammonium, butanediammonium, isobutylammonium iso-butylammonium), n-butylammonium, t-butylammonium, cyclohexylammonium, cyclohexylmethylammonium, diazobicyclooctanedinium, diethylammonium, N,N-diethylethane diammonium, N,N-diethylpropane diammonium, dimethylammonium, N, N-dimethylethane diammonium, dimethylpropane diammonium, dodecylammonium, ethanediammonium, ethylammoniuium, 4-fluoro-benzylammonium, 4-fluoro-phenylethylammonium, 4-fluoro-phenylammonium, formamidinium, guanidinium, hexanediammnium, hexylammonium, imidazolium, 2-methoxyethylammonium, 4-methoxy-phenylethylammonium, 4-methoxy-phenylammonium, methylammonium, morpholinium, octylammonium, pentylammonium, pipeazinediium, piperidinium, propanediammonium, Iso-propylammonium, di-iso-propylammonium, n-propylammonium, pyridinium, 2-pyrrolidin-1-ium-1-yethylammonium, pyrrolidinium, quinclidin-1-ium, 4-trifluoromethyl-benzylammonium, 4-trifluoromethyl ammonium, and Benzalkonium chloride, Dimethyldioctadecylammonium chloride, Trimethylglycine, Quaternary ammonium cation such as Choline, and combinations thereof.

The B may be a divalent transition metal, a rare earth metal, an alkaline earth metal, a monovalent metal, a combination of a trivalent metal, an organic substance (a monovalent, divalent, trivalent cation), and a combination thereof. In addition, preferably, the divalent transition metal, rare earth metal, and alkaline earth metal are Be²⁺, Mg²⁺, Ca²⁺, Sr²⁺, Ba²⁺, Ra²⁺, Mn²⁺, Fe²⁺, Co²⁺, Ni²⁺, Cu²⁺, Zn²⁺, Ru²⁺, Bi²⁺, Pd²⁺, Cd²⁺, Pt²⁺, Hg²⁺, Ge²⁺, Sn²⁺, Pb²⁺, Se²⁺, Te²⁺, Po²⁺, Eu²⁺, No²⁺, and combinations thereof, but are not limited thereto. The monovalent metal may be Li⁺, Na⁺, K⁺, Rb⁺, Cs⁺, Fr⁺, Ag⁺, Hg⁺, Ti⁺, and combinations thereof, and the trivalent metal is Cr³⁺, Fe³⁺, Co³⁺, Ru³⁺, Rh³⁺, Ir³⁺, Au³⁺, Al³⁺, Ga³⁺, In³⁺, Ti³⁺, As³⁺, Sb³⁺, Bi³⁺, La³⁺, Ce³⁺, Pr³⁺, Nd³⁺, Pm³⁺, Sm³⁺, Eu³⁺, Gd³⁺, Tb³⁺, Dy³⁺, Ho³⁺, Er³⁺, Tm³⁺, Yb³⁺, Am³⁺, Cm³⁺, Bk³⁺, Cf³⁺, Es³⁺, Fm³⁺, Md³⁺, Lr³⁺ and combinations thereof.

Accordingly, a third solution in which the second metal halide perovskite is dissolved may be added to the second solution while increasing the m, 1 or k value.

That is, the solution in which the composition of ABX₃₋ₘX′ₘ, A₂BX₄₋₁X′₁ or ABX₄₋ₖX′ₖ is controlled is continuously dropped to form a shell whose composition is continuously changed.

FIG. 7 is a schematic diagram showing a metal halide perovskite nanocrystal particle having a structure having a gradient composition and an energy band diagram thereof according to an embodiment of the present disclosure.

Referring to FIG. 7(a), it can be seen that the nanocrystal particle (100″) according to the present disclosure is a metal halide perovskite nanocrystal structure (140) having a gradient composition of varying content. In this case, FIG. 7(b), by changing the composition of the material from the center of the metal halide perovskite nanocrystal structure (140) toward the outside, the energy band gap may be increased from the center to the outside.

Meanwhile, the metal halide perovskite nanocrystal particles according to the present disclosure may be nanocrystal particle of doped metal halide perovskites.

The doped metal halide perovskite contains a structure of ABX₃, A₂BX₄, ABX₄ or Aₙ₋₁BₙX₃ₙ₊₁ (n is an integer between 2 and 6), and a part of A is substituted with A′, or a part of B is substituted with B′, or a part of X is substituted with X′, wherein A and A′ are monovalent cationic materials, and B and B′ are metal materials, and the X and X′ may be halogen elements.

The monovalent cation may be a monovalent organic cation or an alkali metal. For example, the monovalent organic cation is organic ammonium (RNH₃⁺), organic amidinium derivative (RC(═NR₂)NR₂)⁺, organic guanidinium derivative (R₂NC(═NR₂)NR₂)⁺, organic diammonium (CₓH₂ₓ₋ₙ₊₄)(NH₃)ₙ⁺, ((CₓH₂ₓ₊₁)ₙNH₃)(CH₃NH₃)ₙ⁺, (RNH₃)₂⁺, (CₙH₂ₙ₊₁NH₃)²⁺, (CF₃NH₃)⁺, (CF₃NH₃)ₙ⁺, ((CₓF₂ₓ₊₁)ₙNH₃)₂(CF₃NH₃)ₙ⁺, ((CₓF₂ₓ₊₁)ₙNH₃)₂⁺ or (CₙF₂ₙ₊₁NH₃)₂⁺ (x, n is an integer of 1 or more, R=hydrocarbon derivative, alkyl, alkyl fluoride derivatives, H, F, Cl, Br, I), and combinations thereof, but are not limited thereto. The alkali metal may be Li⁺, Na⁺, K⁺, Rb⁺, Cs⁺, Fr⁺, and combinations thereof, but is not limited thereto.

In addition, preferably, the organic cations are acetamidinium, azaspironanium, benzene diammonium, benzylammonium, butanediammonium, iso-butylammonium, n-butylammonium, t-butylammonium, cyclohexylammonium, cyclohexylmethylammonium, diazobicyclooctanedinium, diethylamhtmonium, N,N, diethylpropane diammonium, dimethylammonium, N,N-dimethylethane diammonium, dimethylpropane diammonium, dodecylammonium, ethanediammonium, ethylammoniuium, 4-fluoro-benzylammonium, 4-fluoro-phenylethylammonium, 4-fluoro-phenylammonium, formamidinium, guanidinium, 2 hexanediammonium-methoxyethylammonium, 4-methoxy-phenlylethylammonium, 4-methoxy-phenylammonium, methylammonium, morpholinium, oxtylammonium, pentylammonium, piperazinediium, piperidinium, propanediammonium, iso-propylammonium, di-iso-propylammonium, n-propylammonium, pyridin-1pyridinium, 2-pyrrolidinium, 2-pyrrolidinium-ium-1-yethylammonium, pyrrolidinium, quinclidin-1-ium, 4-trifluoromethyl-benzylammonium, 4-trifluoromethyl ammonium, and Benzalk Quaternary ammonium cation such as onium chloride, dimethyldioctadecylammonium chloride, trimethylglycine, and choline, and combinations thereof, but are not limited thereto.

B and B′ are divalent metals (e.g., transition metal, rare earth metal, alkaline earth metal, post-transition metal, lanthanum group), monovalent metal, trivalent metal, organic material (monovalent, divalent, trivalent cation) And combinations thereof. In addition, preferably, the divalent metal (e.g., transition metal, rare earth metal, alkaline earth metal, post-transition metal, lanthanum group) is Be²⁺, Mg²⁺, Ca²⁺, Sr²⁺, Ba²⁺, Ra²⁺, Mn²⁺, Fe²⁺, Co²⁺, Ni²⁺, Cu²⁺, Zn²⁺, Ru²⁺, Bi²⁺, Pd²⁺, Cd²⁺, Pt²⁺, Hg²⁺, Ge²⁺, Sn²⁺, Pb²⁺, Se²⁺, Te²⁺, Po²⁺, Eu²⁺, No²⁺, and combinations thereof, but are not limited thereto. The monovalent metal may be Li⁺, Na⁺, K⁺, Rb⁺, Cs⁺, Fr⁺, Ag⁺, Hg⁺, Ti⁺, and combinations thereof, and the trivalent metal is Cr³⁺, Fe³⁺, Co³⁺, Ru³⁺, Rh³⁺, Ir³⁺, Au³⁺, Al³⁺, Ga³⁺, In³⁺, Ti³⁺, As³⁺, Sb³⁺, Bi³⁺, La³⁺, Ce³⁺, Pr³⁺, Nd³⁺, Pm³⁺, Sm³⁺, Eu³⁺, Gd³⁺, Tb³⁺, Dy³⁺, Ho³⁺, Er³⁺, Tm³⁺, Yb³⁺, Am³⁺, Cm³⁺, Bk³⁺, Cf³⁺, Es³⁺, Fm³⁺, Md³⁺, Lr³⁺ and combinations thereof. Also, Eu metals can be additionally doped.

In addition, X and X′ may be F⁻, Cl⁻, Br⁻, I⁻, and At⁻, and combinations thereof.

In addition, a portion of A is substituted with A′, a portion of B is substituted with B′, or a portion of X is substituted with X is characterized in that 0.1% to 5%.

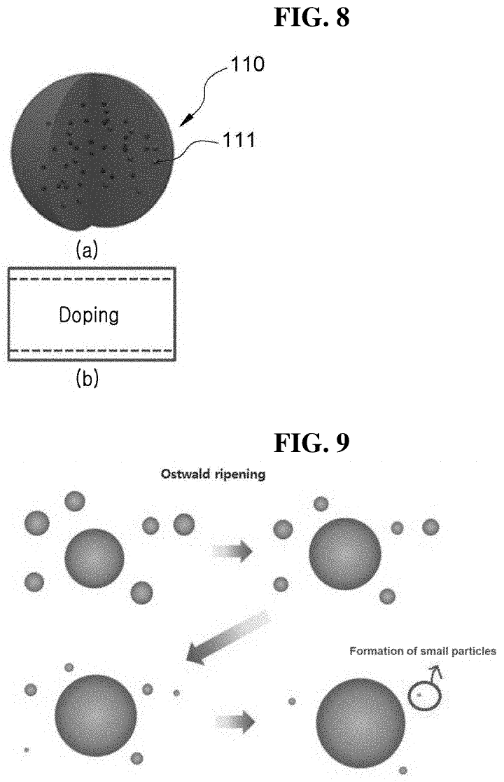

FIG. 8 is a schematic diagram showing a doped metal halide perovskite nanocrystal particle and an energy band diagram thereof according to an embodiment of the present disclosure.

FIG. 8(a) is a partially cut-away schematic diagram of a metal halide perovskite nanocrystal structure (110) doped with a doping element (111). FIG. 8(b) is a band diagram of such a doped metal halide perovskite nanocrystal structure (110).

Referring to FIGS. 8A and 8B, a semiconductor type may be changed to an n-type or a p-type through doping with a metal halide perovskite. For example, when a metal halide perovskite nanocrystal of MAPbI3 is partially doped with Cl, it can be changed to n-type to control electro-optical properties. MA at this time is methyl ammonium.

A doped metal halide perovskite nanocrystal particle according to an embodiment of the present disclosure will be described. A method of manufacturing through an inverse nano-emulsion method or a ligand-assisted reprecipitation method will be described as an example.

First, a first in which a metal halide perovskite doped in a polar solvent is dissolved in a second solution in which at least one surfactant selected from alkyl halides, carboxylic acids and derivatives thereof, and alkylamines and derivatives thereof is dissolved in a non-polar solvent. The solution is added in the form of drops.

The polar solvent at this time may include dimethylformamide, gamma butyrolactone or N-methylpyrrolidone, dimethylsulfoxide, but is limited thereto.

The doped metal halide perovskite contains a structure of ABX₃, A₂BX₄, ABX₄ or Aₙ₋₁BₙX₃ₙ₊₁ (n is an integer between 2 and 6), and a part of A is substituted with A′, or a part of B is substituted with B′, or a part of X is substituted with X′, wherein A and A′ are monovalent cationic materials, and B and B′ are metal materials, and the X and X′ may be halogen elements

For example, the monovalent cation may be a monovalent organic cation is organic ammonium (RNH₃⁺), organic amidinium derivative (RC(═NR₂)NR₂)⁺, organic guanidinium derivative (R₂NC(═NR₂)NR₂)⁺, organic diammonium (CₓH₂ₓ₋ₙ₊₄)(NH₃)ₙ⁺, ((CₓH₂ₓ₊₁)ₙNH₃)(CH₃NH₃)ₙ⁺, (RNH₃)₂⁺, (CₙH₂ₙ₊₁NH₃)₂⁺, (CF₃NH₃)⁺, (CF₃NH₃)ₙ⁺, ((CₓF₂ₓ₊₁)ₙNH₃)₂(CF₃NH₃)ₙ⁺, ((CₓF₂ₓ₊₁)ₙNH₃)₂⁺ or (CₙF₂ₙ₊₁NH₃)₂⁺ (x, n is an integer of 1 or more, R=hydrocarbon derivative, fluorocarbon derivatives, alkyl, alkyl fluoride (fluoroalkyl), H, F, Cl, Br, I), and combinations thereof, but are not limited thereto. The alkali metal may be Li⁺, Na⁺, K⁺, Rb⁺, Cs⁺, Fr⁺, and combinations thereof, but is not limited thereto.

Also preferably, the organic cations are acetamidinium, azaspironanium, benzene diammonium, benzylammonium, butanediammonium, iso-butylammonium, n-butylammonium, t-butylammonium, cyclohexylammonium, cyclohexylmethylammonium, diazobicyclooctanedinium, diethylammonium, N-diethylpropane diammonium, dimethylammonium, N,N-dimethylethane diammonium, dimethylpropane diammonium, dodecylammonium, ethanediammonium, ethylammoniuium, 4-fluoro-benzylammonium, 4-fluoro-phenylethylammonium, 4-fluoro-phenylamidamonium, formamidinium, guanidinium, hexylammonium 2-methoxyethylammonium, 4-methoxy-phenlylethylammonium, 4-methoxy-phenylammonium, methylammonium, morpholinium, oxtylammonium, pentylammonium, piperazinediium, piperidinium, propanediammonium, iso-propylammonium, di-iso-propylammonium, n-propylammonium, pyridinium, 2-pyrrolidinium, 2-pyrrolidinium 1-ium-1-yethylammonium, pyrrolidinium, quinclidin-1-ium, 4-trifluoromethyl-benzylammonium, 4-trifluoromethyl ammonium and derivatives thereof, and may be a combination of these, but is not limited thereto.

B may be a divalent transition metal, a rare earth metal, an alkaline earth metal, a monovalent metal, a combination of a trivalent metal, and a combination thereof. Also preferably, the divalent transition metal, rare earth metal, and alkaline earth metal are Be²⁺, Mg²⁺, Ca²⁺, Sr²⁺, Ba²⁺, Ra²⁺, Mn²⁺, Fe²⁺, Co²⁺, Ni²⁺, Cu²⁺, Zn²⁺, Ru²⁺, Bi²⁺, Pd²⁺, Cd²⁺, Pt²⁺, Hg²⁺, Ge²⁺, Sn²⁺, Pb²⁺, Se²⁺, Te²⁺, Po²⁺, Eu²⁺, No²⁺, and combinations thereof, but are not limited thereto. The monovalent metal may be Li⁺, Na⁺, K⁺, Rb⁺, Cs⁺, Fr⁺, Ag⁺, Hg⁺, Ti⁺, and combinations thereof, and the trivalent metal is Cr³⁺, Fe³⁺, Co³⁺, Ru³⁺, Rh³⁺, I³⁺, Au³⁺, Al³⁺, Ga³⁺, In³⁺, Ti³⁺, As³⁺, Sb³⁺, Bi³⁺, La³⁺, Ce³⁺, Pr³⁺, Nd³⁺, Pm³⁺, Sm³⁺, Eu³⁺, Gd³⁺, Tb³⁺, Dy³⁺, Ho³⁺, Er³⁺, Tm³⁺, Yb³⁺, Am³⁺, Cm³⁺, Bk³⁺, Cf³⁺, Es³⁺, Fm³⁺, Md³⁺, Lr³⁺ and combinations thereof.

In addition, X and X′ may be Cl, Br or I.

In this case, A and A′ are different organic substances, B and B′ are different metals, and X and X′ are different halogen elements. Furthermore, it is preferable to use an element that does not form an alloy with X as the doped X′.

For example, a first solution may be formed by adding CH₃NH₃I, PbI₂, and PbCl₂ to a DMF solvent. In this case, the molar ratio of CH₃NH₃I:PbI₂ and PbCl₂ may be 1:1, and the molar ratio of PbI₂:PbCl₂ may be set to 97:3.

Meanwhile, as an example of the synthesis of AX at this time, when A is CH₃NH₃ and X is Br, CH₃NH₂ (methylamine) and HBr (hydroiodic acid) are dissolved in a nitrogen atmosphere to obtain CH₃NH₃Br through solvent evaporation.

Then, when the first solution is added to the second solution, the doped metal halide perovskite is precipitated from the second solution due to the difference in solubility, and the precipitated doped metal halide perovskite is converted into a well-dispersed doped metal halide perovskite nanocrystal (100) structure while stabilizing the surface while surrounding a large number of at least one type of surfactant selected from an alkyl halide, carboxylic acid and its derivatives (e.g. oleic acid) and amine derivatives (e.g. oleylamine). At this time, the surface of the doped metal halide perovskite nanocrystal particles is surrounded by a plurality of organic ligands (the surfactant also serves as a ligand).

Thereafter, a polar solvent including doped metal halide perovskite nanocrystal particles dispersed in a non-polar solvent in which a surfactant is dissolved is selectively evaporated by heating, or a co-solvent capable of dissolving both a polar solvent and a non-polar solvent (co-solvent) is added to selectively extract a polar solvent including nanocrystal particle from a non-polar solvent to obtain doped metal halide perovskite nanocrystal particles.

On the other hand, when the metal halide perovskite nanoparticles are synthesized in the air (ambient condition), grain boundary creep and defects are formed by moisture in the air, resulting in Ostwald ripening. As a result, small-sized nanocrystal particle are generated, which causes a problem of lowering the color purity.

Thus, for the synthesis of metal halide perovskite nanocrystal particles exhibiting better color purity, preparing a first solution in which metal halide perovskite is dissolved in an aprotic solvent, and a second solution in which a surfactant is dissolved in a protic or aprotic solvent; And mixing the first solution with the second solution in an inert gas atmosphere to form metal halide perovskite nanocrystal particles, wherein when forming metal halide nanocrystal particle in the inert gas atmosphere, the occurrence of Ostwald ripening between the nanocrystal particle is suppressed and the size distribution of the crystalline particles is controlled, it is possible to use a method for controlling the size distribution of metal halide perovskite crystalline particles.

Referring to FIG. 3, the conventional method for preparing metal halide perovskite nanocrystal particles is a method of manufacturing through an inverse nano-emulsion method or a ligand-assisted reprecipitation method. A first solution in which metal halide perovskite precursors are dissolved in a protic solvent and a second solution in which a surfactant is dissolved in a protic solvent or an aprotic solvent is prepared, and the first solution was mixed with the second solution to form nanocrystal particle in the air (ambient condition). In the reverse nanoemulsion, an emulsion is formed in two solvents that are not completely miscible, and a particle-forming reaction is not formed unless acetone or alcohol is additionally added. In the ligand-assisted reprecipitation method, since the two solvents are partially miscible, the particle formation reaction proceeds immediately without an additional solvent. However, depending on the process, a surfactant may be added to the first solution, and some or all of the perovskite precursors may be added to the second solution.

However, in the case of synthesizing metal halide perovskite nanoparticles in the air (ambient condition), grain boundary creep and defects are formed by moisture in the air, and as shown in FIG. 9, Ostwald ripening takes place.

The Ostwald ripening is a theory explaining the principle of the growth of particles dissolved in the form of an emulsion. It means “When the particle size of the emulsion is varied, the relatively small particles continue to decrease, and the large particles gradually increase.”

Conventionally, in the case of synthesis in air, nanocrystal particle having a size of 5 nm or less were generated due to the Ostwald ripening, and the size distribution range of the prepared crystalline particles was too wide, which is the cause of lowering the color purity.

If the nanocrystal particle has a size of less than the Bohr diameter, that is, less than 10 nm, for example, the band gap is changed by the particle size. The Bohr diameter may vary depending on the structure of the material, but since it is generally more than 10 nm, in the case of less than 10 nm, the emission wavelength may be changed even if it has the same metal halide perovskite structure. Therefore, in order to increase the color purity of the metal halide perovskite nanoparticles, it is preferable that the size of the particles is uniform, and it is required to control the size distribution range of the crystalline particles generated therefrom.

When the synthesis atmosphere is adjusted to form nanocrystal particle by mixing the first solution with the second solution under an inert atmosphere, Ostwald ripening does not occur, suppressing the generation of fine nanocrystal particle, and thus, the nanocrystal particle with a size distribution of 10-30 nm larger than the Bohr diameter can be prepared.

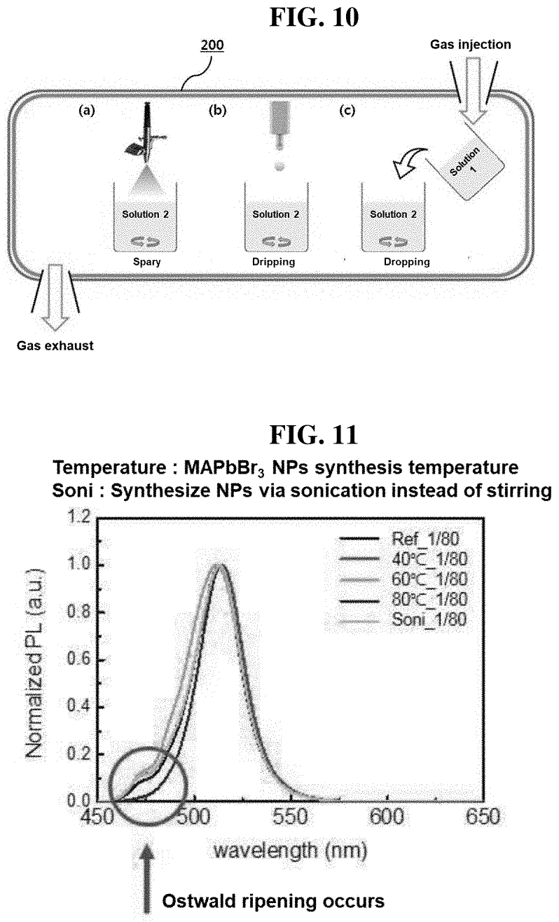

Hereinafter, the present disclosure will be described in more detail with reference to FIG. 10.

As shown in FIG. 10, the method for controlling the size distribution of the metal halide perovskite crystalline particles according to the present disclosure includes preparing a first solution in which metal halide perovskite precursors are dissolved in a polar solvent (including a protic solvent or an aprotic solvent), and a second solution in which a surfactant is dissolved in a at least one solvent selected from a protic solvent, an aprotic solvent, or a non-polar solvent (however, it must be different from the solvent of the first solution); and mixing the first solution with the second solution in an inert gas atmosphere to form metal halide perovskite nanocrystal particles. In the case of forming an inverse nanoemulsion, an additional process of demulsifying the emulsion is required. For this, acetone or alcohols such as tert-butanol can be used.

First, prepare a first solution in which metal halide perovskite precursors are dissolved in an aprotic solvent, and a second solution in which surfactants are dissolved in at least one type of solvent selected from a protic solvent, aprotic, or non-polar solvent.

At this time, the protic solvent may be selected from methanol, ethanol, isopropyl alcohol, tert-butanol, carboxylic acid, water and formic acid, and the aprotic solvent may be selected from dimethylformamide, dimethyl sulfoxide, gamma butyrolactone, N-methylpyrrolidone, acetonitrile, THF (tetrahydrofuran), acetone, and HMPA (hexamethylphosphoramide), but it is not limited thereto. The non-polar solvent may be selected from xylene, octadecene, toluene, hexane, cyclohexene, dichloroethylene, trichloroethylene, chloroform, chlorobenzene, and dichlorobenzene, but it is not limited thereto.

The metal halide perovskite may be a material having a three-dimensional crystal structure, a two-dimensional crystal structure, a one-dimensional crystal structure, or a zero-dimensional crystal structure.

The metal halide perovskite is ABX₃(3D), A₄BX₆(0D), AB₂X₅(2D), A₂BX₄(2D), A₂BX₆(0D), A₂B⁺B³⁺X₆(3D), A₃B₂X₉(2D) or it may include a structure of Aₙ₋₁BₙX₃ₙ₊₁ (quasi-2D) (n is an integer between 2 and 6). A is a monovalent cation, B is a metal material, and X may be a halogen element. Specific examples of A, B, and X of the metal halide perovskite are as described in the section of <Metal Halide Perovskite Crystal>.

On the other hand, such a metal halide perovskite can be prepared by combining AX and BX₂ in a certain ratio. That is, the first solution may be formed by dissolving AX and BX₂ in an aprotic solvent at a predetermined ratio. For example, by dissolving AX and BX₂ in a 1:1 ratio in an aprotic solvent, a first solution, in which ABX₃ metal halide perovskite is dissolved, may be prepared.

In addition, the surfactant may include an alkyl halide, an amine ligand, a carboxylic acid or phosphonic acid, and derivatives thereof. Detailed descriptions of the alkyl halide, amine ligand, carboxylic acid, and phosphonic acid are as described in the section of <Metal Halide perovskite nanocrystal particles>.

Next, the first solution is mixed with the second solution in an inert gas atmosphere to form metal halide perovskite nanocrystal particles.

At this time, the inert gas may be nitrogen (N₂), argon (Ar), or a mixed gas thereof, and any inert gas flow is possibly made if an oxygen concentration is 20 ppm or less. Mixing the first solution with the second solution under the inert gas atmosphere may be performed in a closed space such as a glove box.

In the step of forming nanocrystal particle by mixing the first solution with the second solution, it is preferable that the first solution is added dropwise into the second solution and mixed. In addition, the second solution at this time may be stirred. For example, a first solution in which an organic/inorganic metal halide perovskite (OIP) is dissolved slowly added drop by drop or in several drops to a second solution in which an amine ligand and a carboxylic acid or phosphonic acid surfactant are dissolved and then nanocrystal particle can be synthesized.

In this case, when the first solution is dropped into the second solution and mixed, organic-inorganic metal halide perovskite (OIP) is precipitated from the second solution due to a difference in solubility. The amine-based ligand previously mixed in the second solution adheres to the crystal structure of the metal halide perovskite, thereby reducing the difference in solubility to prevent rapid precipitation of the metal halide perovskite. And carboxylic acid surfactants or phosphonic acid surfactants are adhered to the surface of ionic crystals of organic-inorganic metal halide perovskite (OIP) precipitated from the second solution and stabilizes nanocrystals, and then well-dispersed organic-inorganic metal halide perovskite nanocrystals (OIP-NCs) are produced. Therefore, it is possible to prepare a metal halide perovskite nanocrystal particles including an organic-inorganic metal halide perovskite nanocrystals and a plurality of organic ligands surrounding the organic-inorganic metal halide perovskite nanocrystals.

However, when the first solution and the second solution have very low or no miscibility, recrystallization may not occur, and in this case, a demulsifier may be additionally added.

Tert-butanol and acetone may be used as the demulsifier, but the present disclosure is not limited thereto.

The size distribution of the thus prepared metal halide perovskite crystalline particles can be controlled in the range of 10-30 nm.

The colloidal solution containing the thus prepared metal halide perovskite nanocrystal particles may then be coated to form a thin film.

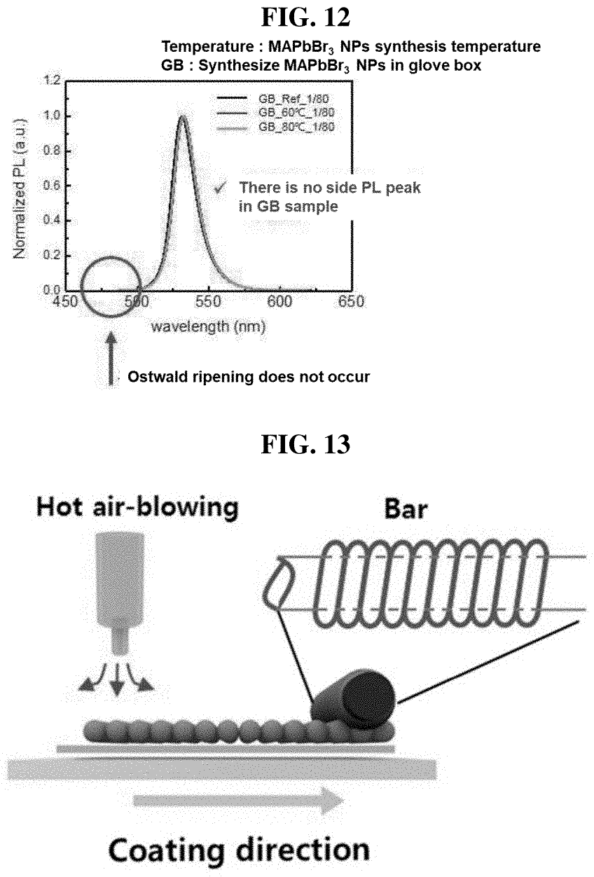

By spin coating a colloidal solution containing metal halide perovskite nanocrystal particles prepared in an inert gas atmosphere according to the method of the present disclosure and metal halide perovskite nanocrystal particles prepared in air according to a conventional manufacturing method, thin films are formed and used to measure the photoluminescence properties. As shown in FIG. 11, in the case of the metal halide perovskite nanocrystal particle thin film produced in air according to the conventional manufacturing method, very small nanoparticles are also generated due to occurrence of Oswald ripening and the region of the emission wavelength was splitted. However, in the case of the metal halide perovskite nanocrystal particle thin film prepared in an inert gas atmosphere according to the present disclosure, as shown in FIG. 12, Oswald ripening does not occur and the single emission band appears, thereby realizing higher color purity.

Therefore, metal halide perovskite nanocrystal particles (organic metal halide perovskite nanocrystal particles or inorganic metal halide perovskite nanocrystal particles) prepared according to the method according to an embodiment of the present disclosure can be applied to various optoelectronics devices.

<Metal Halide Perovskite Nanocrystal Thin Film Production>

In order to apply the metal halide perovskite nanocrystal particles to various optoelectronic devices, it is important to form a uniform thin film. For example, preparing a metal halide perovskite nanocrystal particle dispersed in an organic solvent to form a uniform metal halide perovskite nanocrystal particle thin film; thin films are formed with a randomly selected one among various known methods such as spin coating method, spray method, dip coating method, bar coating method, nozzle printing method, slot-die coating method, gravure printing method, casting method, or Langmuir-Blodgett film method (LB).

When performing the spin coating process, the spin coating speed may be 1000 rpm to 5000 rpm, and the spin coating time may be 15 seconds to 150 seconds. If the spin coating speed falls below 1000 rpm or the spin coating time is shortened within 15 seconds, the thin film may become non-uniform or the solvent may not evaporate.

When forming a thin film through a printing method other than spin coating, the metal halide perovskite nanocrystal particles form a thin film in an already-crystallized state and thus are not affected by the coating speed, the coating environment, and the crystallinity of the underlying substrate layer, compared to polycrystalline bulk metal halide perovskite thin film in which crystallization proceeds during coating, the thin films. However, when a thin film is manufactured using such a printing method, the evaporation rate of the solvent is slow, so the nanocrystal particles are agglomerated and thus large crystals can be formed through recrystallization.

Accordingly, a uniform metal halide perovskite nanocrystal thin film can be manufactured through a printing process combined with an additional method of rapidly drying the printed nanocrystal thin film. It is possible to prevent recrystallization between the metal halide perovskite nanocrystal particles by additionally performing a step of rapidly drying the thin film after the printing process.

Preferably, the solvent remaining after the printing process can be removed through air injection.

FIG. 13 is a schematic diagram of a process of removing a solvent remaining after a bar coating process through air injection according to an embodiment of the present disclosure.

In addition, preferably, referring to FIG. 13, the drying step may be characterized in that high-temperature air is sprayed. It is preferable that the temperature of the sprayed air be 70° C. to 100° C. If the temperature of the air sprayed is out of the above range and less than 70° C., evaporation of the ground solvent may be delayed and recrystallization between the nanocrystal particles may occur. If the temperature of the sprayed air exceeds 100° C., the metal halide perovskite crystal structure vulnerable to heat may be decomposed. It is more preferable to carry out drying.

In the case of drying by applying only a temperature of 70 to 100° C. without blowing air in the drying step, since the drying temperature is lower than the boiling point of the organic solvent used for dispersion of the metal halide perovskite nanocrystal particles (e.g., toluene (110.6° C.), dimethylformamide (153° C.)), the drying speed is slow, so that recrystallization of the nanocrystal particles can be achieved, and it is more preferable to perform rapid drying by adding air injection.

According to another embodiment of the present disclosure, in order to form a uniform metal halide perovskite nanocrystal particle thin film, the forming of the perovskite nanocrystal particle thin film includes an anchoring solution and the organic-inorganic metal halide perovskite. Preparing a solution of organic-inorganic metal halide perovskite nanoparticles containing perovskite nanocrystals, forming an anchoring agent layer by spin coating the anchoring solution on the substrate or the gate insulating film, and the process of forming an anchoring semiconductor layer by spin coating the organic-inorganic metal halide perovskite nanoparticle solution on the agent layer may be included.

Specifically, first, an anchoring solution and an organic/inorganic metal halide perovskite nanoparticle solution including the organic/inorganic metal halide perovskite nanocrystal may be prepared.

The anchoring solution may be a solution containing a resin that imparts adhesiveness exhibiting an anchoring effect. The anchoring solution may be, for example, a 3-mercaptopropionic acid ethanolic solution. The anchoring solution may have a concentration of 7 wt % to 12 wt %.

Thereafter, the anchoring agent layer may be formed by spin coating the anchoring solution on the substrate on which the metal halide perovskite nanocrystal particle thin film is to be formed. When performing the spin coating process, the spin coating speed may be 1000 rpm to 5000 rpm, and the spin coating time may be 15 seconds to 150 seconds. If the spin coating speed falls below 1000 rpm or the spin coating time is shortened within 15 seconds, the thin film may become non-uniform or the solvent may not evaporate.

Thereafter, an organic-inorganic metal halide perovskite nanoparticle solution may be spin-coated on the anchoring agent layer to form a thin film of anchoring metal halide perovskite nanocrystal particles. When the anchoring metal halide perovskite nanocrystal particle thin film is formed using the anchoring solution, a more dense nanocrystal layer may be formed.

Thereafter, a crosslinking agent layer may be formed on the anchoring metal halide perovskite nanocrystal particle thin film. When the crosslinking agent layer is formed, a denser metal halide perovskite nanocrystal layer can be formed, and the length of the ligand is shortened to facilitate the injection of charges into the nanocrystals, thereby increasing the luminescence efficiency and luminance of the light emitting device.

The crosslinking agent above is preferably a crosslinking agent having an X—R—X structure, and as an example, 1,2-ethanedithiol may be used. After preparing a solution by mixing the crosslinking agent in a soluble solvent, it may be spin-coated.

At this time, the steps of spin-coating the organic-inorganic metal halide perovskite nanoparticle solution and forming a crosslinking agent layer on the spin-coated layer of the organic-inorganic metal halide perovskite nanoparticle solution are alternately repeated. Thus, the thickness of the light emitting layer can be adjusted.

In this case, the spin coating speed is preferably 1000 rpm to 5000 rpm, and the spin coating time may be 15 seconds to 150 seconds. If the spin coating speed decreases down to 1000 rpm or less, or if the spin coating time is shortened to 15 seconds or less, the thin film may become non-uniform or the solvent may not evaporate.

<Metal Halide Perovskite Light Emitting Device>

According to an embodiment of the present disclosure, the metal halide perovskite described above may be used in a light emitting device.

In the present specification, the “light-emitting device” may include all devices that emit light such as a light-emitting diode, a light-emitting transistor, a laser, and a polarized light-emitting device.

The light emitting device according to an embodiment of the present disclosure is characterized in that light is emitted from the above-described metal halide perovskite.

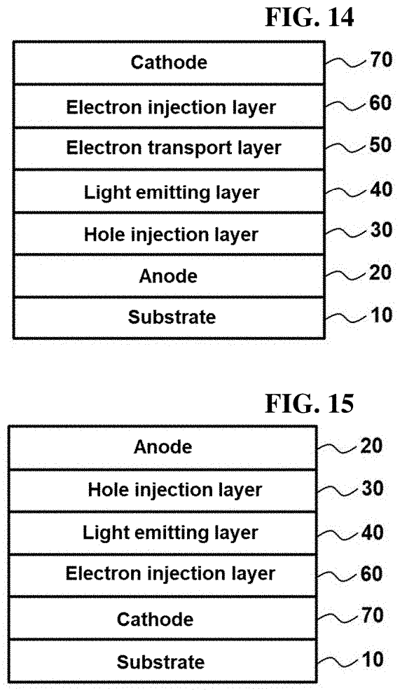

FIGS. 14 and 15 are schematic diagrams showing a light emitting device according to an embodiment of the present disclosure.

FIGS. 14 and 15, the light emitting device according to the present disclosure may include an anode (20) and a cathode (70) and a light emitting layer (40) located between the two electrodes. In addition, preferably, a hole injection layer (30) may be provided between the anode (20) and the light emitting layer (40) to facilitate injection of holes. In addition, an electron transport layer (50) for transporting electrons and an electron injection layer (60) for facilitating injection of electrons may be provided between the light emitting layer (40) and the cathode (70).

In addition, the light emitting device according to the present disclosure may further include a hole transport layer for transporting holes between the hole injection layer (30) and the light emitting layer (40).

In addition, a hole blocking layer (not shown) may be located between the light emitting layer (40) and the electron transport layer (50). In addition, an electron blocking layer (not shown) may be located between the light emitting layer (40) and the hole transport layer. However, the present disclosure is not limited thereto, and the electron transport layer (50) may serve as a hole blocking layer, or the hole transport layer may serve as an electron blocking layer.

The anode (20) may be a conductive metal oxide, a metal, a metal alloy, or a carbon material. Conductive metal oxides include ITO, AZO(Al-doped ZnO), GZO(Ga-doped ZnO), IGZO(In,Ga-doped ZnO), MZO(Mg-doped ZnO), Mo-doped ZnO, Al-doped MgO, Ga-doped MgO, F-doped SnO₂, Nb-doped TiO₂ or CuAlO₂, or a combination thereof. Metals or metal alloys suitable as the anode (20) may be Au and CuI. The carbon material may be graphite, graphene, or carbon nanotubes.

The negative electrode (70) is a conductive film having a lower work function than the positive electrode (20), for example, metals such as aluminum, magnesium, calcium, sodium, potassium, indium, yttrium, lithium, silver, lead, cesium, or these It can be formed using a combination of two or more types of.

The anode (20) and the cathode (70) may be formed using a sputtering method, a vapor deposition method, or an ion beam deposition method. The hole injection layer (30), the hole transport layer, the light emitting layer (40), the hole blocking layer, the electron transport layer (50), and the electron injection layer (60) are independent of each other by a vapor deposition method or a coating method such as spraying and spin coating, dipping, printing, doctor blading, or electrophoresis.

The hole injection layer (30) and/or the hole transport layer are layers having a HOMO level between the work function level of the anode (20) and the HOMO level of the light emitting layer (40), and holes from the anode (20) to the light emitting layer (40). It functions to increase the efficiency of injection or transport.

The hole injection layer (30) or the hole transport layer may include a material commonly used as a hole transport material, and one layer may include different hole transport material layers. Hole transport materials include, for example, mCP (N,Ndicarbazolyl-3,5-benzene); PEDOT:PSS (poly(3,4-ethylenedioxythiophene):polystyrenesulfonate); NPD (N,N′-di(1-naphthyl)-N,N′-diphenylbenzidine); N,N′-Bis(3-methylphenyl)-N,N′-diphenylbenzidine (TPD); DNTPD (N4,N4′-Bis[4-[bis(3-methylphenyl)amino]phenyl]-N4,N4′-diphenyl-[1,1′-biphenyl]-4,4′-diamine); N,N′-diphenyl-N,N′-dinaphthyl-4,4′-diaminobiphenyl; N,N,N′N′-tetra-p-tolyl-4,4′-diaminobiphenyl; N,N,N′N′-tetraphenyl-4,4′-diaminobiphenyl; Porphyrin compound derivatives such as copper(II)1,10,15,20-tetraphenyl-21H,23H-porphyrin; TAPC (1,1-Bis[4-[N,N′-Di(p-tolyl)Amino]Phenyl]Cyclohexane); Triarylamine derivatives such as N,N,N-tri(p-tolyl)amine, 4,4′, 4′-tris[N-(3-methylphenyl)-N-phenylamino]triphenylamine; Carbazole derivatives such as N-phenylcarbazole and polyvinylcarbazole; Phthalocyanine derivatives such as metal-free phthalocyanine and copper phthalocyanine; Starburst amine derivatives; Enaminestilbene derivatives; Derivatives of aromatic tertiary amines and styryl amine compounds; and polysilane. Such a hole transport material may serve as an electron blocking layer.

The hole injection layer (30) may also include a hole injection material. For example, the hole injection layer may include at least one of a metal oxide and a hole injection organic material.

When the hole injection layer (30) includes a metal oxide, the metal oxide is MoO₃, WO₃, V₂O₅, nickel oxide (NiO), copper oxide (Copper(II) Oxide: CuO), copper aluminum oxide (Copper Aluminum Oxide: CAO, CuAlO₂), Zinc Rhodium Oxide: ZRO, ZnRh₂O₄, GaSnO, and metal-sulfide (FeS, ZnS or CuS) doped with one or more metal oxides selected from the group consisting of GaSnO can do.

When the hole injection layer (30) contains a hole injection organic material, the hole injection layer (30) may be formed according to a method arbitrarily selected from a variety of known methods such as a vacuum deposition method, a spin coating method, a casting method, a Langmuir-Blodgett (LB) method, a spray coating method, a dip coating method, a gravure coating method, a reverse offset coating method, a screen printing method, a slot-die coating method, and a nozzle printing method.

The hole-injecting organic material is may include at least one selected from the group consisting of Fullerene (C₆₀), HAT-CN, F₁₆CuPC, CuPC, m-MTDATA [4,4′, 4″-tris(3-methylphenylphenylamino)triphenylamine] (see formula below), NPB [N, N′-Di(1-naphthyl)-N,N′-diphenyl-(1,1′-biphenyl)-4,4′-diamine)], TDATA (see formula below), 2T-NATA (see formula below), Pani/DBSA (Polyaniline/Dodecylbenzenesulfonic acid:Polyaniline/Dodecylbenzenesulfonic acid), PEDOT/PSS (Poly(3,4-ethylenedioxythiophene)/Poly(4-styrenesulfonate):Poly(3,4-ethylenedioxythiophene)/Poly(4-styrenesulfonate)), Pani/CSA (Polyaniline/Camphor sulfonic acid:polyaniline/camphor sulfonic acid) and PANI/PSS (Polyaniline)/Poly(4-styrenesulfonate).

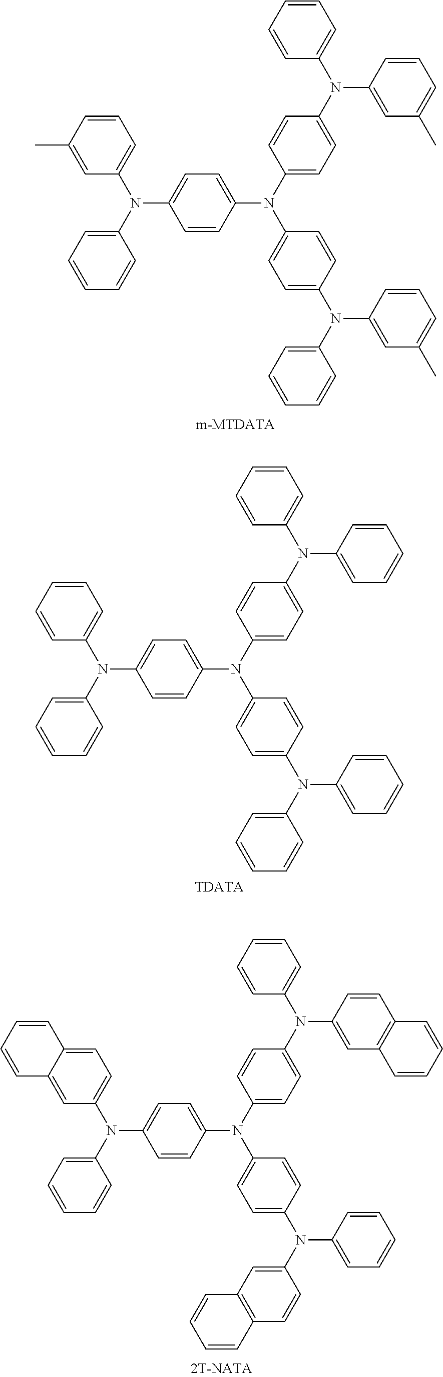

For example, the hole injection layer may be a layer in which the metal oxide is doped into the hole injecting organic material matrix. In this case, the doping concentration is preferably 0.1 wt % to 80 wt % based on the total weight of the hole injection layer.

The hole injection layer may have a thickness of 1 nm to 1000 nm. For example, the thickness of the hole injection layer is 1 nm, 2 nm, 3 nm, 4 nm, 5 nm, 6 nm, 7 nm, 8 nm, 9 nm, 10 nm, 11 nm, 12 nm, 13 nm, 14 nm, 15 nm, 16 nm, 17 nm, 18 nm, 19 nm, 20 nm, 21 nm, 22 nm, 23 nm, 24 nm, 25 nm, 26 nm, 27 nm, 28 nm, 29 nm, 30 nm, 31 nm, 32 nm, 33 nm, 34 nm, 35 nm, 36 nm, 37 nm, 38 nm, 39 nm, 40 nm, 41 nm, 42 nm, 43 nm, 44 nm, 45 nm, 46 nm, 47 10 nm, 48 nm, 49 nm, 50 nm, 51 nm, 52 nm, 53 nm, 54 nm, 55 nm, 56 nm, 57 59 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 101 nm, 102 nm, 103 nm, 104 nm, 105 nm, 106 nm, 107 nm, 108 nm, 109 nm, 110 nm, 111 nm, 112 nm, 113 nm, 114 nm, 115 nm, 116 nm, 117 nm, 118 nm, 119 nm, 120 nm, 125 nm, 130 nm, 135 nm, 140 nm, 145 nm, 150 nm, 155 nm, 160 nm, 165 nm, 170 nm, 175 nm, 180 nm, 185 nm, 190 nm, 195 nm, 200 nm, 210 nm, 220 nm, 230 nm, 240 nm, 250 nm, 260 nm, 270 nm, 280 nm, 290 nm, 300 nm, 310 nm, 320 nm, 330 nm, 340 nm, 350 nm, 360 nm, 370 nm, 380 nm, 390 nm, 400 nm, 450 nm, 500 nm, 550 nm, 600 nm, 650 nm, 700 nm, 750 nm, 800 nm, 850 nm, 900 nm, 950 nm and 1000 nm. The thickness of the hole injection layer can be defined as a region that takes the lower value as the minimum value and the larger value as the maximum value out of the two numbers selected above. Also, preferably, the thickness of the hole injection layer may be 10 nm to 200 nm. When the thickness of the hole injection layer satisfies the above-described region, the driving voltage is not increased, so that a high-quality organic device can be implemented.

In addition, a hole transport layer may be further formed between the light emitting layer and the hole injection layer.

The hole transport layer may include a known hole transport material. For example, the hole transport material that may include at least one selected from the group consisting of (1,3-bis(carbazol-9-yl)benzene (MCP), 1,3,5-tris(carbazol-9-yl)benzene (TCP), 4,4′, 4″-tris(carbazol-9-yl)triphenylamine (TCTA), 4,4′-bis(carbazol-9-yl)biphenyl (CBP), N,N′-bis(naphthalen-1-yl)-N,N′-bis(phenyl)-benzidine (NPB), N,N′-bis(naphthalen-2-yl)-N,N′-bis(phenyl)-benzidine (β-NPB), N,N′-bis(naphthalen-1-yl)-N,N′-bis(phenyl)-2,2′-dimethylbenzidine (α-NPD), Di-[4-(N,N-ditolyl-amino)-phenyl]cyclohexane (TAPC), N,N,N′,N′-tetra-naphthalen-2-yl-benzidine (β-TNB) and N4,N4,N4′,N4′-tetra(biphenyl-4-yl)biphenyl-4,4′-diamine (TPD15), poly(9,9-dioctylfluorene-co-bis-N,N′-(4-butylphenyl)-bis-N,N′-phenyl-1,4-phenylenedi amine) (PFB), poly(9,9′-dioctylfluorene-co-N-(4-butylphenyl)diphenylamine) (TFB), poly(9,9′-dioctylfluorene-co-bis-N,N′-(4-butylphenyl)-bis-N,N′-phenylbenzidine) (BFB), poly(9,9-dioctylfluorene-co-bis-N,N′-(4-methoxyphenyl)-bis-N,N′-phenyl-1,4-phenylenediamine)(PFMO), but it is not limited thereto.

The formula of the hole transport material is summarized in Table 1 below.

TABLE 1

| Name | Formula |
|---|---|
| NPB | 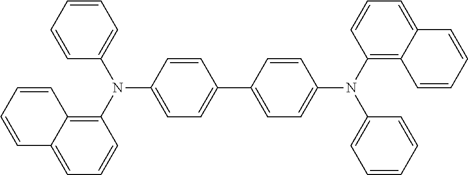 [SMILES: c1ccc(N(c2ccc(-c3ccc(N(c4ccccc4)c4cccc5ccccc45)cc3)cc2)c2cccc3ccccc23)cc1] |
| TCP | 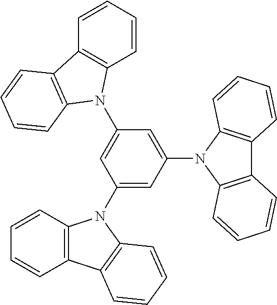 [SMILES: c1ccc2c(c1)c1ccccc1n2-c1cc(-n2c3ccccc3c3ccccc32)cc(-n2c3ccccc3c3ccccc32)c1] |
| CBP | 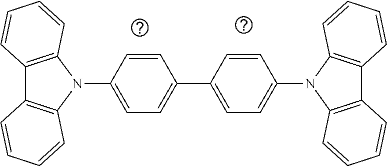 [SMILES: c1ccc2c(c1)c1ccccc1n2-c1ccc(-c2ccc(-n3c4ccccc4c4ccccc43)cc2)cc1] |
| α-NPD | 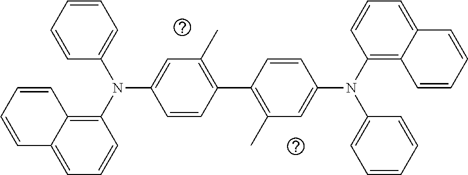 [SMILES: Cc1cc(N(c2ccccc2)c2cccc3ccccc23)ccc1-c1ccc(N(c2ccccc2)c2cccc3ccccc23)cc1C] |
| β-TNB | 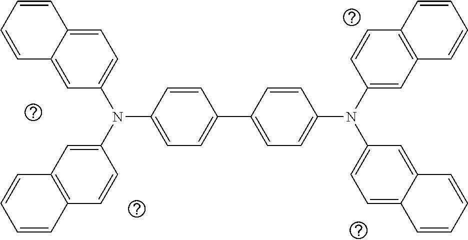 [SMILES: c1ccc2cc(N(c3ccc(-c4ccc(N(c5ccc6ccccc6c5)c5ccc6ccccc6c5)cc4)cc3)c3ccc4ccccc4c3)ccc2c1] |
| MCP | 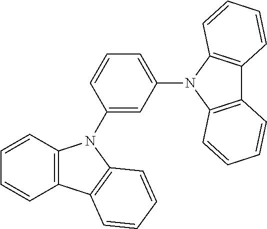 [SMILES: c1cc(-n2c3ccccc3c3ccccc32)cc(-n2c3ccccc3c3ccccc32)c1] |
| TCTA | 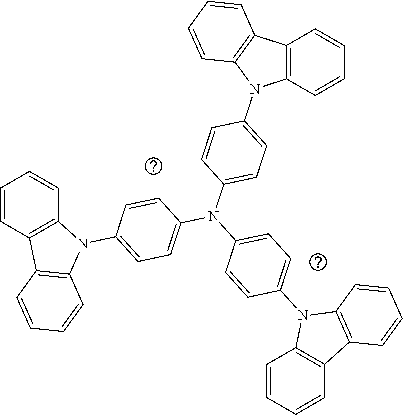 [SMILES: c1ccc2c(c1)c1ccccc1n2-c1ccc(N(c2ccc(-n3c4ccccc4c4ccccc43)cc2)c2ccc(-n3c4ccccc4c4ccccc43)cc2)cc1] |
| β-NPB | 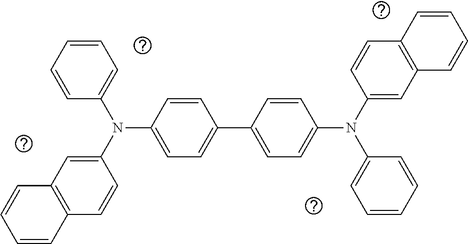 [SMILES: c1ccc(N(c2ccc(-c3ccc(N(c4ccccc4)c4ccc5ccccc5c4)cc3)cc2)c2ccc3ccccc3c2)cc1] |
| TAPC | 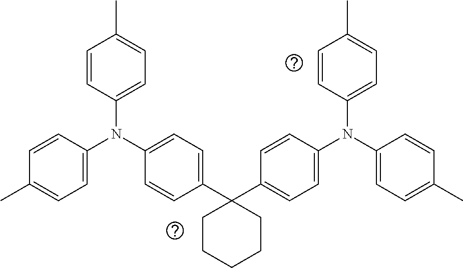 |
| TPD15 | 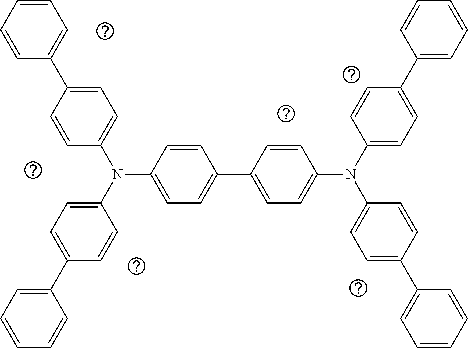 [SMILES: c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1] |

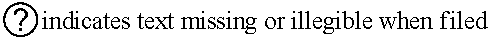

Among the hole transport layers, for example, in the case of TCTA, in addition to the hole transport role, it may play a role of preventing diffusion of excitons from the emission layer.

The thickness of the hole transport layer may be 1 nm to 100 nm. For example, the thickness of the hole transport layer is 1 nm, 2 nm, 3 nm, 4 nm, 5 nm, 6 nm, 7 nm, 8 nm, 9 nm, 10 nm, 11 nm, 12 nm, 13 nm, 14 nm, 15 nm, 16 nm, 17 nm, 18 nm, 19 nm, 20 nm, 21 nm, 22 nm, 23 nm, 24 nm, 25 nm, 26 nm, 27 nm, 28 nm, 29 nm, 30 nm, 31 nm, 32 nm, 33 nm, 34 nm, 35 nm, 36 nm, 37 nm, 38 nm, 39 nm, 40 nm, 41 nm, 42 nm, 43 nm, 44 nm, 45 nm, 46 nm, 47 nm, 48 nm, 49 nm, 50 nm, 51 nm, 52 nm, 53 nm, 54 nm, 55 nm, 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, and 100 nm. The thickness of the hole transport layer can be defined as a region that takes the lower value as the minimum value and the larger value as the maximum value out of the two numbers selected above. In addition, preferably, the thickness of the hole transport layer may be 10 nm to 60 nm. When the thickness of the hole transport layer satisfies the above-described range, light efficiency of the organic light emitting diode may be improved and luminance may be increased.

The electron injection layer (60) and/or the electron transport layer (50) are layers having an LUMO level between the work function level of the cathode (70) and the LUMO level of the emission layer (40), and increase the efficiency of injection or transport of electrons from the cathode (70) to the emission layer (40).

The electron injection layer (60) may be, for example, LiF, NaCl, NaF, CsF, Li₂O, BaO, BaF₂, MgF₂, or Liq (lithium quinolate). In addition, if the electron transport layer and the electron injection layer material are co-deposited to form a doped electron transport layer, the electron injection layer may be replaced.

The electron transport layer (50) may include quinoline derivative, especially tris(8-hydroxyquinoline)aluminum (Alq₃), Bis(2-methyl-8-quinolinolate)-4-(phenylphenolato)aluminium (Balq), bis(10-hydroxybenzo[h]quinolinato)-beryllium (Bebg₂), 2,9-dimethyl-4,7-diphenyl-1,10-phenanthroline (BCP), 4,7-diphenyl-1,10-phenanthroline (Bphen), 2,2′, 2″-(benzene-1,3,5-triyl)-tris(1-phenyl-1H-benzimidazole (TPBI), 3-(4-biphenyl)-4-phenyl-5-tert-butylphenyl-1,2,4-triazole (TAZ), 4-(naphthalen-1-yl)-3,5-diphenyl-4H-1,2,4-triazole (NTAZ), 2,9-bis(naphthalen-2)-yl)-4,7-diphenyl-1,10-phenanthroline (NBphen), Tris(2,4,6-trimethyl-3-(pyridin-3-yl)phenyl)borane (3TPYMB), phenyl-dipyrenylphosphine oxide (POPy₂), 3,3′, 5,5′-tetra[(m-pyridyl)-phen-3-yl]biphenyl (BP4mPy), 1,3,5-tri[(3-pyridyl)-phen-3-yl]benzene (TmPyPB), 1,3-bis[3,5-di(pyridin-3-yl)phenyl]benzene (BmPyPhB), Bis(10-hydroxybenzo[h]quinolinato)beryllium (Bepq₂), Diphenylbis(4-(pyridin-3-yl)phenyl)silane (DPPS) and 1,3,5-tri(p-pyrid-3-yl-phenyl)benzene (TpPyPB), 1,3-bis[2-(2,2′-bipyridine-6-yl)-1,3,4-oxadiazo-5-yl]benzene (Bpy-OXD), 6,6′-bis[5-(biphenyl-4-yl)-1,3,4-oxadiazo-2-yl]-2,2′-bipyridyl (BP-OXD-Bpy), TSPO1(diphenylphosphine oxide-4-(triphenylsilyl)phenyl), TPBi(1,3,5-tris(N-phenylbenzimidazol-2-yl)benzene), tris(8-quinolinorate)aluminum (Alq3), 2,5-diaryl silol derivative (PyPySPyPy), perfluorinated compound (PF-6P), COTs (Octasubstituted cyclooctatetraene).

The formula of the electron transport material is summarized in Table 2 below.

TABLE 2

| Name | Formula |
|---|---|
| A [symbol] | 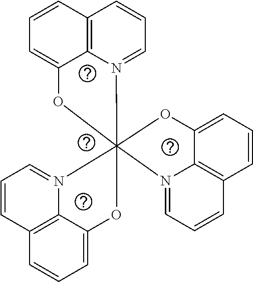 |
| PBD | 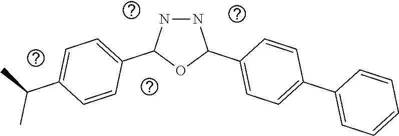 |
| Bphen | 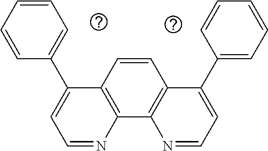 [SMILES: c1ccc(-c2ccnc3c2ccc2c(-c4ccccc4)ccnc23)cc1] |
| Bpy-OXD | 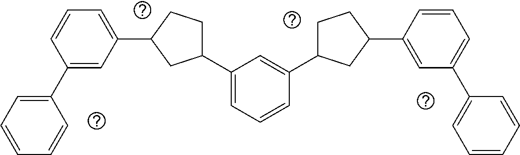 [SMILES: c1ccc(-c2cccc(C3CCC(c4cccc(C5CCC(c6cccc(-c7ccccc7)c6)C5)c4)C3)c2)cc1] |
| TAZ | 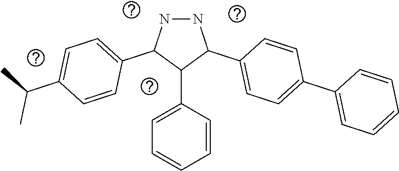 |
| NBphen | 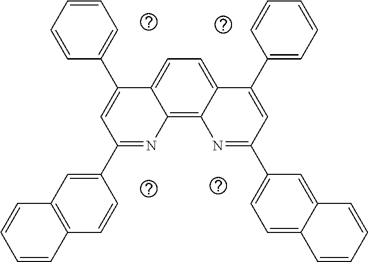 [SMILES: c1ccc(-c2cc(-c3ccc4ccccc4c3)nc3c2ccc2c(-c4ccccc4)cc(-c4ccc5ccccc5c4)nc23)cc1] |
| POPy [symbol] | 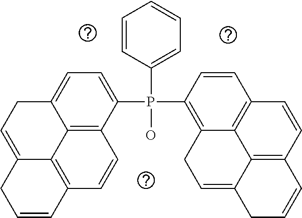 |
| TmPyPB | 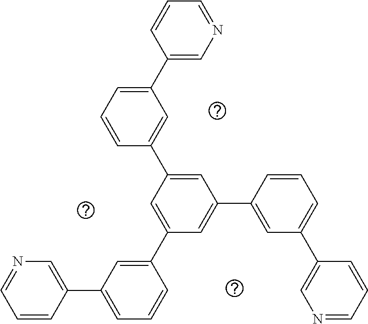 [SMILES: c1cncc(-c2cccc(-c3cc(-c4cccc(-c5cccnc5)c4)cc(-c4cccc(-c5cccnc5)c4)c3)c2)c1] |
| Bebq2 | 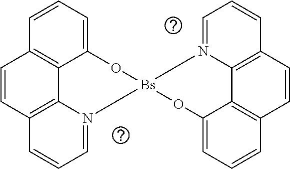 |
| TpPyPR | 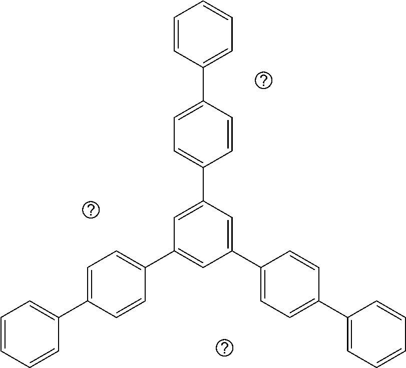 [SMILES: c1ccc(-c2ccc(-c3cc(-c4ccc(-c5ccccc5)cc4)cc(-c4ccc(-c5ccccc5)cc4)c3)cc2)cc1] |
| TPBI [symbol] | 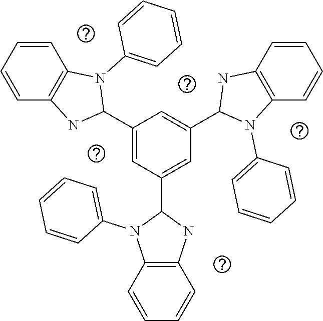 [SMILES: c1ccc(N2c3ccccc3NC2c2cc(C3Nc4ccccc4N3c3ccccc3)cc(C3Nc4ccccc4N3c3ccccc3)c2)cc1] |
| BCP | 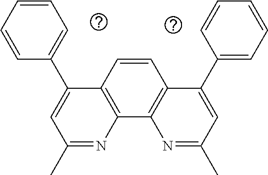 [SMILES: Cc1cc(-c2ccccc2)c2ccc3c(-c4ccccc4)cc(C)nc3c2n1] |
| Balq | 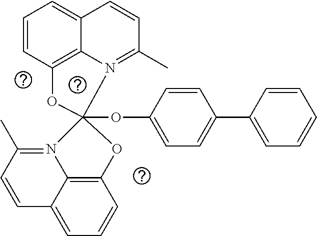 |
| BP-OXD-Bpy | 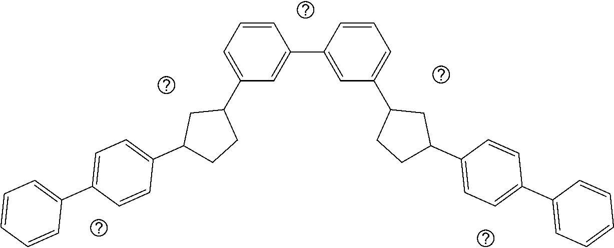 [SMILES: c1ccc(-c2ccc(C3CCC(c4cccc(-c5cccc(C6CCC(c7ccc(-c8ccccc8)cc7)C6)c5)c4)C3)cc2)cc1] |
| NTAZ | 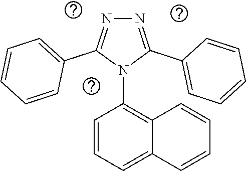 [SMILES: c1ccc(-c2nnc(-c3ccccc3)n2-c2cccc3ccccc23)cc1] |
| 3TPYMB | 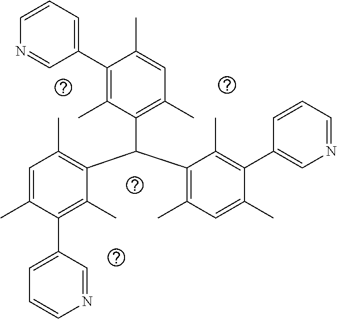 [SMILES: Cc1cc(C)c(C(c2c(C)cc(C)c(-c3cccnc3)c2C)c2c(C)cc(C)c(-c3cccnc3)c2C)c(C)c1-c1cccnc1] |
| BP4mPy | 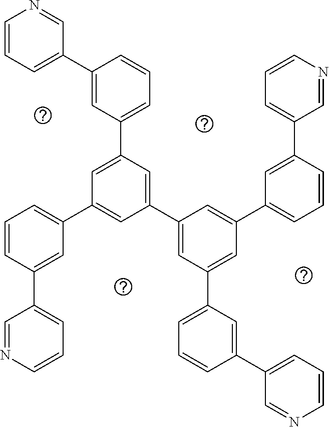 [SMILES: c1cncc(-c2cccc(-c3cc(-c4cccc(-c5cccnc5)c4)cc(-c4cc(-c5cccc(-c6cccnc6)c5)cc(-c5cccc(-c6cccnc6)c5)c4)c3)c2)c1] |
| BmPyPhB | 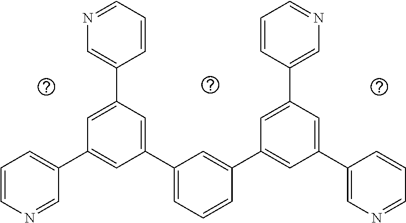 [SMILES: c1cncc(-c2cc(-c3cccnc3)cc(-c3cccc(-c4cc(-c5cccnc5)cc(-c5cccnc5)c4)c3)c2)c1] |
| DPPS | 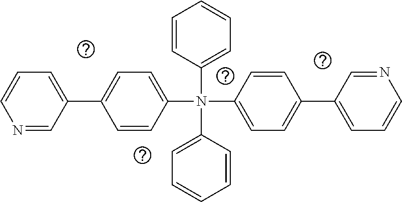 |

[symbol]

indicates data missing or illegible when filed

The thickness of the electron transport layer may be about 5 nm to 100 nm. For example, the thickness of the electron transport layer is 5 nm, 6 nm, 7 nm, 8 nm, 9 nm, 10 nm, 11 nm, 12 nm, 13 nm, 14 nm, 15 nm, 16 nm, 17 nm, 18 nm, 19 nm, 20 nm, 21 nm, 22 nm, 23 nm, 24 nm, 25 nm, 26 nm, 27 nm, 28 nm, 29 nm, 30 nm, 31 nm, 32 nm, 33 nm, 34 nm, 35 nm, 36 nm, 37 nm, 38 nm, 39 nm, 40 nm, 41 nm, 42 nm, 43 nm, 44 nm, 45 nm, 46 nm, 47 nm, 48 nm, 49 nm, 50 nm, 51 nm, 52 nm, 53 nm, 54 nm, 55 nm, 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm. The thickness of the hole injection layer can be defined as a region that takes the lower value as the minimum value and the larger value as the maximum value out of the two numbers selected above. In addition, preferably, the thickness of the electron transport layer may be 15 nm to 60 nm. When the thickness of the electron transport layer satisfies the above-described range, excellent electron transport characteristics can be obtained without an increase in driving voltage.

The electron injection layer (60) may include a metal oxide. Since the metal oxide has n-type semiconductor properties, it has excellent electron transport capability, and further, it is a material that is not reactive to air or moisture, and may be selected from semiconductor materials having excellent transparency in a visible light region.

The electron injection layer (60) may include at least one metal oxide selected from among, for example, aluminum doped zinc oxide (AZO), alkali metal (Li, Na, K, Rb, Cs or Fr) doped AZO, TiOₓ (x is a real number of 1 to 3), indium oxide (In₂O₃), tin oxide (SnO₂), zinc oxide (ZnO), zinc tin oxide, gallium oxide (Ga₂O₃), tungsten oxide (WO₃), aluminum oxide, titanium oxide, vanadium oxide (V₂O₅, vanadium(IV) oxide(VO₂), V₄O₇, V₅O₉, or V₂O₃), molybdenum oxide (MoO₃ or MoOₓ), copper oxide (Copper(II) Oxide: CuO), oxidation Nickel (NiO), copper aluminum oxide (Copper Aluminum Oxide: CAO, CuAlO₂), Zinc Rhodium Oxide: ZRO, ZnRh₂O₄, iron oxide, chromium oxide, bismuth oxide, IGZO (indium-Gallium Zinc Oxide), and ZrO₂, but is not limited thereto. As an example, the electron injection layer (60) may be a metal oxide thin film layer, a metal oxide nanoparticle layer, or a layer including metal oxide nanoparticles in the metal oxide thin film.

The electron injection layer (60) may be formed using a wet process or a vapor deposition method.

As an example of a wet process, when the electron injection layer (60) is formed by a solution method (ex. a sol-gel method), mixture solution which includes at least one of a sol-gel precursor of a metal oxide and a metal oxide in the form of nanoparticles is applied on the substrate (10) and then heat treatment. In this case, the solvent may be removed by heat treatment or the electron injection layer (60) may be crystallized. The method of providing the mixture for the electron injection layer on the substrate (10) may be selected from a known coating method, for example, a spin coating method, a cast method, a Langmuir-Blodgett (LB) method, a spray coating method, a dip coating method, gravure coating method, a reverse offset coating method, a screen printing method, a slot-die coating method, a nozzle printing method, and a dry transfer printing method, but the present disclosure is not limited thereto.

The sol-gel precursor of the metal oxide may include at least one selected from the group consisting of a metal salt (e.g, metal halide, metal sulfate, metal nitrate, metal perchlorate, metal acetate, metal carbonate, etc.), metal salt hydrate, metal hydroxide, metal alkyl, metal alkoxide, metal carbide, metal acetylacetonate, metal acid, metal acid salt, metal acid hydrate, metal sulfide, metal acetate, metal alkanoate, metal phthalocyanine, metal nitride, and metal carbonate.

When the metal oxide is ZnO, the ZnO sol-gel precursor may include at least one selected from the group consisting of zinc sulfate, zinc fluoride, zinc chloride, zinc bromide, zinc iodide, zinc perchlorate, zinc hydroxide (Zn(OH)₂), zinc acetate (Zn(CH₃COO)₂), zinc acetate hydrate (Zn(CH₃(COO)₂.nH₂O), diethyl zinc (Zn(CH₃CH₂)₂), zinc nitrate (Zn(NO₃)₂), zinc nitrate hydrate (Zn(NO₃)₂.nH₂O), zinc carbonate (Zn(CO₃)), zinc acetylacetonate (Zn(CH₃COCHCOCH₃)₂), and zinc acetylacetonate hydrate (Zn(CH₃COCHCOCH₃)₂.nH₂O), but it is not limited thereto.

When the metal oxide is indium oxide (In₂O₃), the In₂O₃ sol-gel precursor may include at least one selected from the group consisting of nitric acid (nH₂O), indium acetate (In(CH₃COO)₂), indium acetate hydrate (In(CH₃(COO)₂.nH₂O), indium chloride (InCl, InCl₂, InCl₃), indium nitrate (In(NO₃)₃), indium nitrate hydrate (In(NO₃)₃.nH₂O), indium acetylacetonate (In(CH₃COCHCOCH₃)₂), and indium acetylacetonate hydrate (In(CH₃COCHCOCH₃)₂.nH₂O).

When the metal oxide is tin oxide (SnO₂), the SnO₂ sol-gel precursor may include at least one selected from the group consisting of tin acetate (Sn(CH₃COO)₂), tin acetate hydrate (Sn(CH₃(COO)₂nH₂O), tin chloride (SnCl₂, SnCl₄), tin chloride hydrate (SnClₙ.nH₂O), tin acetylacetonate (Sn(CH₃COCHCOCH₃)₂), and tin acetylacetonate hydrate (Sn(CH₃COCHCOCH₃)₂.nH₂O).

When the metal oxide is gallium oxide (Ga₂O₃), the Ga₂O₃ sol-gel precursor may include at least one selected from the group consisting of gallium nitrate (Ga(NO₃)₃), gallium nitrate hydrate (Ga(NO₃)₃nH₂O), gallium acetylacetonate (Ga(CH₃COCHCOCH₃)₃)), gallium acetylacetonate hydrate (Ga(CH₃COCHCOCH₃)₃.nH₂O), and gallium chloride (Ga₂Cl₄, GaCl₃).

When the metal oxide is tungsten oxide (WO₃), the WO₃ sol-gel precursor may include at least one selected from the group consisting of tungsten carbide (WC), tungstic acid powder (H₂WO₄), tungsten chloride (WCl₄, WCl₆), tungsten isopropoxide (W(OCH(CH₃)₂)₆), sodium tungstate (Na₂WO₄), sodium tungstate hydrate (Na₂WO₄.nH₂O), ammonium tungstate ((NH₄)₆H₂W₁₂O₄₀), ammonium tungstate hydrate ((NH₄)₆H₂W₁₂O₄₀.nH₂O), and tungsten ethoxide (W(OC₂H₅)₆).

When the metal oxide is aluminum oxide, the aluminum oxide sol-gel precursor may include at least one selected from the group consisting of aluminum chloride (AlCl₃), aluminum nitrate (Al(NO₃)₃), aluminum nitrate hydrate (Al(NO₃)₃.nH₂O), and aluminum butoxide (Al(C₂H₅CH(CH₃)O)).

When the metal oxide is titanium oxide, the titanium oxide sol-gel precursor may include at least one selected from the group consisting of titanium isopropoxide (Ti(OCH(CH₃)₂)₄), titanium chloride (TiCl₄), titanium ethoxide (Ti(OC₂H₅)₄)), and titanium butoxide (Ti(OC₄H₉)₄).

When the metal oxide is vanadium oxide, the sol-gel precursor of vanadium oxide may include at least one selected from the group consisting of vanadium isopropoxide (VO(OC₃H₇)₃), ammonium vanadate (NH₄VO₃), vanadium acetylacetonate (V(CH₃COCHCOCH₃)), vanadium acetylacetonate hydrate (V(CH₃COCHCOCH₃)₃.nH₂O).

When the metal oxide is molybdenum oxide, the molybdenum oxide sol-gel precursor may include at least one selected from the group consisting of molybdenum isopropoxide (Mo(OC₃H₇)₅), molybdenum chloride isopropoxide (MoCl₃(OC₃H₇)₂)), ammonium molybdenate ((NH₄)₂MoO₄), and ammonium molybdenate hydrate ((NH₄)₂MoO₄.nH₂O) may be used.

When the metal oxide is copper oxide, the copper oxide sol-gel precursor may include at least one selected from the group consisting of copper chloride (CuCl, CuCl₂), copper chloride hydrate (CuCl₂.nH₂O), copper acetate (Cu(CO₂CH₃), Cu(CO₂CH₃)₂), copper acetate hydrate. (Cu(CO₂CH₃)₂.nH₂O), copper acetylacetonate (Cu(C₅H₇O₂)₂), copper nitrate (Cu(NO₃)₂), copper nitrate hydrate (Cu(NO₃)₂.nH₂O), copper bromide (CuBr, CuBr₂), copper carbonate (CuCO₃Cu(OH)₂), copper sulfide (Cu₂S, CuS), copper phthalocyanine (C₃₂H₁₆N₈Cu), copper trifluoroacetate (Cu(CO₂CF₃)₂), copper isobutyrate (C,H₁₄CuO₄), copper ethylacetoacetate (C₁₂H₁₈CuO₆)), copper 2-ethylhexanoate ([CH₃(CH₂)₃CH(C₂H,)CO₂]₂Cu), copper fluoride (CuF₂), copper formate hydrate ((HCO₂)₂CuH₂O), copper gluconate (C₁₂H₂₂CuO₁₄), copper hexa fluoroacetylacetonate (Cu(C₅HF₆O₂)₂), copper hexafluoroacetylacetonate hydrate (Cu(C₅HF₆O₂)₂.nH₂O), copper methoxide (Cu(OCH₃)₂), copper neodecanoate (C₁₀H₁₉O₂Cu), copper perchlorate hydrate (Cu(ClO₄)₂6H₂O), copper sulfate (CuSO₄), copper sulfate hydrate (CuSO₄.nH₂O), copper tartrate hydrate ([—CH(OH)CO₂]₂Cu.nH₂O), copper trifluoroacetylacetonate (Cu(C₅H₄F₃O₂)₂), copper trifluoromethanesulfonate ((CF₃SO₃)₂Cu), and tetraamine copper sulfate hydrate (Cu(NH₃)₄SO₄H₂O).

When the metal oxide is iron oxide, the sol-gel precursor of iron oxide may include at least one selected from the group consisting of iron acetate (Fe(CO₂CH₃)₂), iron chloride (FeCl₂, FeCl₃), iron chloride hydrate (FeCl₃nH₂O), iron acetylacetonate (Fe(C₅H₇O₂)₃), iron nitrate hydrate (Fe(NO₃)₃₉H₂O), iron phthalocyanine (C₃₂H₁₆FeN₈), iron oxalate hydrate (Fe(C₂O₄)nH₂O, and Fe₂(C₂O₄)₃ 6H₂O).

When the metal oxide is chromium oxide, the chromium oxide sol-gel precursor may include at least one selected from the group consisting of chromium chloride (CrCl₂, CrCl₃), chromium chloride hydrate (CrCl₃.nH₂O), chromium carbide (Cr₃C₂), chromium acetylacetonate (Cr(C₅H₇O₂)₃), chromium nitrate hydrate (Cr(NO₃)₃.nH₂O), chromium hydroxide (CH₃CO₂)₇Cr₃(OH)₂, and chromium acetate hydrate ([(CH₃CO₂)₂CrH₂O]₂).

When the metal oxide is bismuth oxide, the bismuth oxide sol-gel precursor may include at least one selected from the group consisting of bismuth chloride (BiCl₃), bismuth nitrate hydrate (Bi(NO₃)₃.nH₂O), bismuth acetic acid ((CH₃CO₂)₃Bi), and bismuth carbonate ((BiO)₂CO₃).

When the metal oxide nanoparticles are contained in the mixed solution for the electron injection layer, the average particle diameter of the metal oxide nanoparticles may be 10 nm to 100 nm.

The solvent may be a polar solvent or a non-polar solvent. For example, examples of the polar solvent include alcohols and ketones, and examples of the nonpolar solvent include aromatic hydrocarbons, alicyclic hydrocarbons, and aliphatic hydrocarbon-based organic solvents. As an example, the solvent may include at least one selected from the group consisting of ethanol, dimethylformamide, ethanol, methanol, propanol, butanol, isopropanol. methyl ethyl ketone, propylene glycol (mono)methyl ether (PGM), isopropyl cellulose (IPC), ethylene carbonate (EC), methyl cellosolve (MC), ethyl cellosolve, 2-methoxy ethanol and ethanol amine. But it is not limited thereto.

For example, when forming the electron injection layer (60) made of ZnO, the mixture for the electron injection layer may include zinc acetate dehydrate as a precursor of ZnO, and combination of 2-methoxy ethanol and ethanolamine as solvents but it is not limited thereto.

The heat treatment conditions will vary depending on the type and content of the selected solvent, but it is generally preferably performed within the range of 100° C. to 350° C. and 0.1 hour to 1 hour. When the heat treatment temperature and time satisfy this range, the solvent removal effect is good and the device may not be deformed.

When the electron injection layer (60) is formed using a vapor deposition method, it can be deposited by a variety of known methods such as electron beam deposition, thermal evaporation, sputter deposition, atomic layer deposition, chemical vapor deposition (Chemical vapor deposition). The deposition conditions vary depending on the target compound, the structure of the target layer, and thermal properties, but for example, it is preferred that the deposition temperature range of 25 to 1500° C., specifically 100 to 500° C., and the vacuum degree range of 10⁻¹⁰ to 10⁻³ torr., the deposition rate is performed within the range of 0.01 to 100 Å/sec.

The electron injection layer (60) may have a thickness of 1 nm to 100 nm. For example, the thickness of the electron injection layer is 1 nm, 2 nm, 3 nm, 4 nm, 5 nm, 6 nm, 7 nm, 8 nm, 9 nm, 10 nm, 11 nm, 12 nm, 13 nm, 14 nm, 15 nm, 16 nm, 17 nm, 18 nm, 19 nm, 20 nm, 21 nm, 22 nm, 23 nm, 24 nm, 25 nm, 26 nm, 27 nm, 28 nm, 29 nm, 30 nm, 31 nm, 32 nm, 33 nm, 34 nm, 35 nm, 36 nm, 37 nm, 38 nm, 39 nm, 40 nm, 41 nm, 42 nm, 43 nm, 44 nm, 45 nm, 46 nm, 47 nm, 48 nm, 49 nm, 50 nm, 51 nm, 52 nm, 53 nm, 54 nm, 55 nm, 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, and 100 nm. The thickness of the hole injection layer can be defined as a region that takes the lower value as the minimum value and the larger value as the maximum value out of the two numbers selected above. In addition, preferably, the thickness of the electron injection layer may be 15 nm to 60 nm.

The hole injection layer (30), the hole transport layer, the electron injection layer (60), or the electron transport layer (50) may include materials used in conventional organic light emitting diodes.

The hole injection layer (30), the hole transport layer, the electron injection layer (60) or the electron transport layer (50) may be formed by performing a method arbitrarily selected from a variety of known methods such as a vacuum deposition method, a spin coating method, a spray method, a dip coating method, a bar coating method, a nozzle printing method, a slot-die coating method, a gravure printing method, a cast method, or a Langmuir-Blodgett (LB) method. At this time, the conditions and coating conditions for forming the thin film may vary depending on the target compound, the structure and thermal properties of the target layer.

The substrate (10) serves as a support for the light emitting device and may be a transparent material. In addition, the substrate (10) may be a flexible material or a hard material, and preferably may be a flexible material.

The material of the substrate (10) may include glass, sapphire, quartz, silicon, polyethylene terephthalate (PET), polystyrene (PS), and polyimide (PI), polyvinyl chloride (PVC), polyvinylpyrrolidone (PVP), polyethylene (PE), but it is not limited thereto.

The substrate (10) may be located under the anode (20) or may be located above the cathode (70). In other words, the anode (20) may be formed before the cathode deposition (70) on the substrate, or the cathode (70) may be formed before the anode deposition (20). Accordingly, the light emitting device may have both the normal structure of FIG. 14 and the inverse structure of FIG. 15.

The light emitting layer (40) is formed between the hole injection layer (30) and the electron injection layer (60), and the holes (h) introduced from the anode (20) and the electrons (e) introduced from the cathode (70) are combined. As a result, excitons are formed, and light is emitted while the excitons transition to a ground state, thereby causing light emission.

In the light emitting device according to the present disclosure, the light emitting layer (40) is characterized in that it includes the metal halide perovskite described above.

The metal halide perovskite may be a material having a three-dimensional crystal structure, a two-dimensional crystal structure, a one-dimensional crystal structure, or a zero-dimensional crystal structure.

The metal halide perovskite is ABX₃(3D), A₄BX₆(0D), AB₂X₅(2D), A₂BX₄(2D), A₂BX₆(0D), A₂B⁺B³⁺X₆(3D), A₃B₂X₉(2D) or it may include a structure of Aₙ₋₁BₙX₃ₙ₊₁ (quasi-2D) (n is an integer between 2 and 6). A is a monovalent cation, B is a metal material, and X may be a halogen element. Specific examples of A, B, and X of the metal halide perovskite are as described in the section of <Metal Halide Perovskite Crystal>.

<Metal Halide Perovskite Light-Emitting Transistor>

In particular, when the light-emitting device is a light-emitting transistor, it may have a higher color purity than a conventional organic semiconductor-based light-emitting transistor, and field-effect mobility and on/off ratio are increased, so that switching properties can be improved, and manufacturing costs can be reduced.

The metal halide perovskite light emitting transistor includes a gate electrode, a semiconductor layer, a gate insulating film located between the semiconductor layer and the gate electrode, and a light emitting transistor including a source electrode and a drain electrode electrically connected to the semiconductor layer. In this case, it may be characterized in that it has a semiconductor layer including a metal halide perovskite.

The substrate may be used as a support for an electrode, a semiconductor layer, or the like formed on the substrate, and any substrate used in a known organic light emitting transistor may be used. The substrate, for example, may be a metal substrate such as carbon (C), iron (Fe), chromium (Cr), manganese (Mn), nickel (Ni), titanium (Ti), molybdenum (Mo), or stainless steel (SUS), or may be a plastic substrate such as polyethylene naphthalate (PEN), polyethylene terephthalate (PET), polyphenylene sulfide (PPS), polyallylate, polyimide, and polyester. polyetherimide (PEI), polyacrylate (PAR), or polycarbonate, or a glass substrate, but it is not limited thereto.

The source electrode, the drain electrode, and the gate electrode may include at least one selected from the group consisting of a metal, a conductive polymer, a carbon material-doped semiconductor, and a combination thereof. For example, gold (Au), platinum (Pt), chromium (Cr), molybdenum (Mo), nickel (Ni), aluminum (Al), graphene or an alloy thereof, or indium tin oxide (ITO) or at least one material selected from inorganic oxide materials such as indium zinc oxide (IZO).

The gate insulating layer is formed between the gate electrode and the semiconductor layer for stability of the light emitting transistor, and may include at least one selected from the group consisting of a carboxyl group (—COOH), a hydroxyl group (—OH), a thiol group (—SH), and a trichlorosilane group (—SiCl₃), and may be made of at least one material selected from the group consisting of self-assembled molecules including any one selected from the group consisting of metals, insulating polymers, inorganic oxides, polymer electrolytes, and combinations thereof.

The metal halide perovskite used in the semiconductor layer may be a material having a three-dimensional crystal structure, a two-dimensional crystal structure, a one-dimensional crystal structure, or a zero-dimensional crystal structure.

The metal halide perovskite is ABX₃(3D), A₄BX₆(0D), AB₂X₅(2D), A₂BX₄(2D), A₂BX₆(0D), A₂B⁺B³⁺X₆(3D), A₃B₂X₉(2D) or Aₙ₋₁BₙX₃ₙ₊₁ (quasi-2D) (n is an integer between 2 and 6) may be included. A is a monovalent cation, B is a metal material, and X may be a halogen element. Specific examples of A, B, and X of the metal halide perovskite are as described in <Metal Halide Perovskite Crystal>.

The metal halide perovskite light emitting transistor may have a bottom-gate/top-contact, a bottom-gate/bottom-contact, a top-gate/top-contact, or a top-gate/bottom-contact structure.

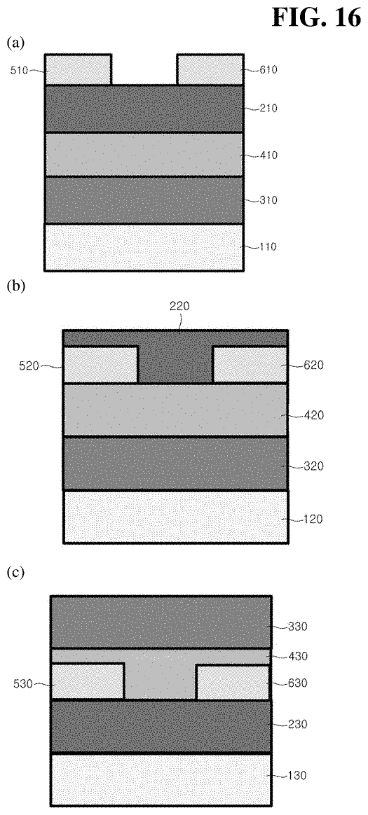

Referring to FIG. 16(a), in an embodiment of the present disclosure, the metal halide perovskite light emitting transistor may have a bottom gate/top-contact structure. Specifically, the gate electrode (310) and the gate insulating layer (410) may be sequentially located on the substrate (110), and the semiconductor layer (210) including a metal halide perovskite may be located on the gate insulating layer (410). Also, a source electrode (510) and a drain electrode (610) may be located on the semiconductor layer (210) to be electrically connected to the semiconductor layer (210)

Referring to FIG. 16(b), in another embodiment of the present disclosure, the metal halide perovskite light emitting transistor may have a bottom-gate/bottom-contact structure. Specifically, the gate electrode (320) and the gate insulating layer (420) may be sequentially located on the substrate (120), and the source electrode (520) and the drain electrode (620) may be located on the gate insulating layer (420). So as to be electrically connected to the source electrode (520) and the drain electrode (620), the semiconductor layer (220) including a metal halide perovskite may be located as covering the source electrode (520) and the drain electrode (620) form on the gate insulating layer (420).

Referring to FIG. 16(c), in another embodiment of the present disclosure, the metal halide perovskite light emitting transistor may have a top-gate/top-contact structure. Specifically, a semiconductor layer (230) including a metal halide perovskite may be located on the substrate (130), and the source electrode (530) and the drain electrode (630) can be located on the semiconductor layer (230) to be electrically connected to the semiconductor layer (230). A gate insulating layer (430) may be located to cover the source electrode (530) and the drain electrode (630), and the gate electrode (330) may be located on the gate insulating layer (430).

Referring to FIG. 16(d), in another embodiment of the present disclosure, the metal halide perovskite light emitting transistor may have a top gate/bottom-contact structure. Specifically, the source electrode (540) and the drain electrode (630) may be located on the substrate (140), and the source electrode (540) may be electrically connected to the drain electrode (630). A semiconductor layer (240) including a metal halide perovskite may be located to cover the (540) and the drain electrode (630). A gate insulating layer (440) may be located on the semiconductor layer (240), and a gate electrode (340) may be located on the gate insulating layer (440).

As described above, in the metal halide perovskite light emitting transistor of the present disclosure, a semiconductor layer including a metal halide perovskite can be applied to various structures.

It may further include at least one of an electron transport layer and a hole transport layer located above or below the semiconductor layer.

FIG. 19(a) to 19(d) are schematic diagrams showing the structure of a metal halide perovskite light emitting transistor according to another embodiment of the present disclosure.

Specifically, FIG. 19(a) to 19(d) show an upper or lower part of the semiconductor layer in the light emitting transistor in the case of a metal halide perovskite light emitting transistor having a bottom-gate/top-contact structure of the present disclosure. It may further include at least one of the electron transport layer and the hole transport layer.

Referring to FIG. 19(a), in an embodiment of the present disclosure, an electron transport layer (750) may be further located under the semiconductor layer (250). Specifically, the gate electrode (350) and the gate insulating layer (450) are sequentially located on the substrate (150), the electron transport layer (750) may be located first before the semiconductor layer (250) is located on the gate insulating layer (450), and a semiconductor layer (250) including the metal halide perovskite may be located on the electron transport layer (750). Thereafter, a source electrode (550) and a drain electrode (650) may be located on one end and the other end of the semiconductor layer (250) so that they are electrically connected to the semiconductor layer (250) on the semiconductor layer (250).

Referring to FIG. 19(b) in another embodiment of the present disclosure, a hole transport layer (860) may be further located under the semiconductor layer (260). Specifically, the gate electrode (360) and the gate insulating layer (460) are sequentially located on the substrate (160), the hole transport layer (860) is located on the gate insulating layer (460) before the semiconductor layer (260) is located on the gate insulating layer (260), and a semiconductor layer (260) including metal halide perovskite may be located on the hole transport layer (860). Thereafter, a source electrode (560) and a drain electrode (660) may be located on one end and the other end of the semiconductor layer (260) so that they are electrically connected to the semiconductor layer (260) on the semiconductor layer (260).

Referring to FIG. 19(c), in another embodiment of the present disclosure, an electron transport layer (770) may be located under the semiconductor layer (270), and a hole transport layer (870) may be further located on the semiconductor layer (270). Specifically, the gate electrode (370) and the gate insulating layer (470) are sequentially located on the substrate (170), and the electron transport layer (770) is first located on the gate insulating layer (470) and, and the semiconductor layer (270) including a metal halide perovskite may be located on the electron transport layer (770). Thereafter, a hole transport layer (870) may be located on the semiconductor layer (270), and a source electrode (570) and a drain electrode (670) may be located at one end and the other end of the hole transport layer (870).

Referring to FIG. 19(d), in another embodiment of the present disclosure, a hole transport layer (880) is located under the semiconductor layer (280), and an electron transport layer (780) is further located on the semiconductor layer (280). Specifically, a gate electrode (380) and a gate insulating layer (480) are sequentially located on the substrate (180), a hole transport layer (880) is first located on the gate insulating layer (480), and the semiconductor layer (280) including a metal halide perovskite may be located on the hole transport layer (880). Thereafter, an electron transport layer (780) may be located on the semiconductor layer (280), and a source electrode (580) and a drain electrode (680) may be located at one end and the other end of the electron transport layer (780).

According to an embodiment, even when the metal halide perovskite light emitting transistor has a bottom-gate/bottom-contact, top-gate/top-contact, or top-gate/bottom-contact structure in addition to the above-described bottom-gate/top-contact structure, as shown in FIGS. 19(a) and 19(d), at least one of an electron transport layer and a hole transport layer may be located above or below the semiconductor layer.

The metal halide perovskite may have a polycrystalline or single crystal structure.

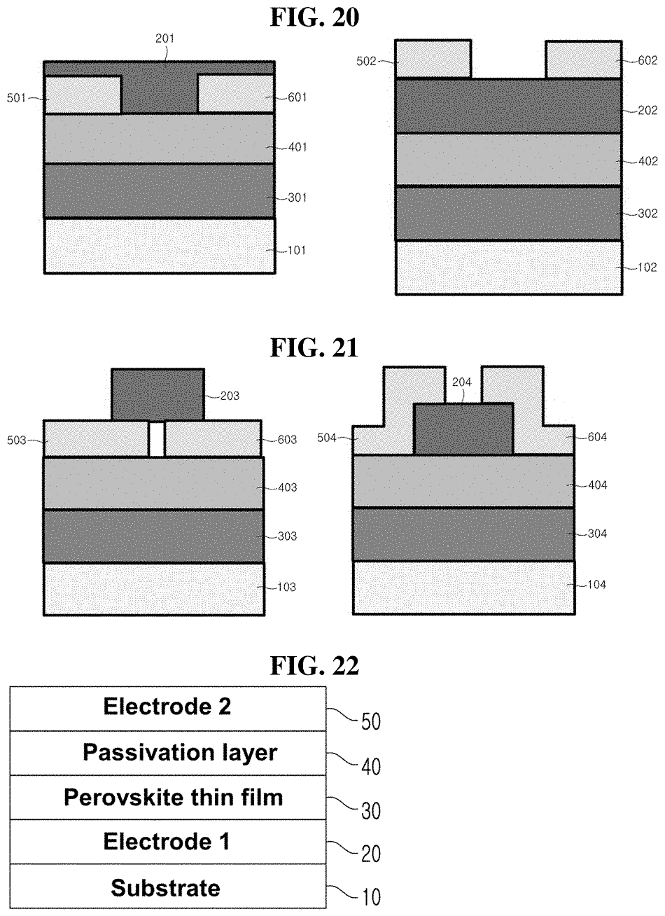

FIGS. 20(a) and 20(b) are schematic diagrams illustrating a light emitting transistor in which a semiconductor layer including a metal halide perovskite having a polycrystalline structure is located according to an embodiment of the present disclosure.

Referring to FIG. 20(a), a semiconductor layer including a metal halide perovskite having a polycrystalline structure according to an embodiment of the present disclosure may be located in a bottom-gate/bottom-contact structure. Specifically, the gate electrode (301) and the gate insulating film (401) are sequentially located on the substrate (101), and the source electrode (501) and the drain electrode (601) may be located at one end and the other end of the gate insulating film (401). A semiconductor layer (201) including a metal halide perovskite having the polycrystalline structure may be located to cover the source electrode (501) and the drain electrode (601) so that it is electrically connected to the source electrode (501) and the drain electrode (601).

Referring to FIG. 20(b), a semiconductor layer including a metal halide perovskite having a polycrystalline structure according to another embodiment of the present disclosure may be located in a bottom-gate/top-contact structure. Specifically, a gate electrode (302) and a gate insulating film (402) are sequentially located on a substrate (102), and a semiconductor layer (202) including a metal halide perovskite having the polycrystalline structure on the gate insulating film (402) can be deployed. Thereafter, a source electrode (502) and a drain electrode (602) are formed at one end and the other end of the semiconductor layer (202) so that the semiconductor layer (202) is electrically connected to the source electrode (502) and the drain electrode (602) can be placed.

FIG. 21(a) to 21(b) are schematic diagrams showing a light emitting transistor in which a semiconductor layer including a metal halide perovskite having a single crystal structure is located according to another embodiment of the present disclosure.

Referring to FIG. 21(a), a semiconductor layer including a metal halide perovskite having a single crystal structure according to another embodiment of the present disclosure may be located in a bottom-gate/bottom-contact structure. Specifically, the gate electrode (302) and the gate insulating film (403) are sequentially located on the substrate (103), and the source (503) electrode and the drain electrode (603) are located at one end and the other end of the gate insulating film (403). A semiconductor layer (203) including metal halide perovskite having polycrystalline structure can be located on the source electrode (503) and the drain electrode (603) to be electrically connected to the source electrode (503) and the drain electrode (603).

Referring to FIG. 21(b), a semiconductor layer including a metal halide perovskite having a single crystal structure according to another embodiment of the present disclosure may be located in a bottom-gate/top-contact structure. Specifically, a semiconductor layer (204) including a metal halide perovskite having the single crystal structure in the upper center of the gate electrode (304) and the gate insulating film (404) are sequentially located on the substrate (104) may be located. Thereafter, the source electrode (504) and a drain electrode (604) may be positioned in a form in which a portion of one end and the other end region of the semiconductor layer (204) is electrically connected to the source electrode (504) and the drain electrode (604).

As described above, when a semiconductor layer including a metal halide perovskite having a single crystal structure is located in a bottom-gate/top-contact structure, the channel length may be 1 μm or less.

Hereinafter, a method of manufacturing a metal halide perovskite light emitting transistor according to an embodiment of the present disclosure will be described.

The method of manufacturing the metal halide perovskite light emitting transistor includes a step forming a semiconductor layer made of a nanocrystalline thin film in which a metal halide perovskite nanocrystal is formed on a substrate or the gate insulating layer by coating a solution containing perovskite nanoparticles based on a method of manufacturing a transistor commonly used in the art.

The solution containing the organic-inorganic metal halide perovskite nanoparticles in which the metal halide perovskite nanocrystals are formed is a first solution in which a metal halide perovskite is dissolved in a protic solvent. A second solution in which an alkyl halide surfactant is dissolved in an aprotic solvent may be prepared, and the first solution may be mixed with the second solution to form nanoparticles.

The protic solvent at this time may include dimethylformamide, gamma butyrolactone or N-methylpyrrolidone, dimethylsulfoxide, but is limited thereto.

The metal halide perovskite may be a material having a polycrystalline or single crystal structure.

On the other hand, such a metal halide perovskite can be prepared by combining AX and BX₂ in a certain ratio. That is, the first solution may be formed by dissolving AX and BX₂ in a protic solvent at a predetermined ratio. For example, a first solution in which A₂BX₃ metal halide perovskite is dissolved may be prepared by dissolving AX and BX₂ in a ratio of 2:1 in a protic solvent.

In addition, the aprotic solvent at this time is dichloroethylene, trichloroethylene, chloroform, chlorobenzene, dichlorobenzene, styrene, dimethylformamide, dimethylsulfoxide, xylene, toluene, cyclohexene or isopropyl alcohol May include, but is not limited to.

The surfactant may include the aforementioned alkyl halide, amine ligand, carboxylic acid or phosphonic acid.

Then, the first solution may be mixed with the second solution to form nanoparticles. Mixing the first solution with the second solution to form nanoparticles may be mixing the first solution drop by drop into the second solution. In addition, the second solution at this time may be stirred. For example, nanoparticles may be synthesized by slowly adding a second solution in which organic-inorganic metal halide perovskite (OIP) is dissolved in a second solution in which a surfactant being strongly stirred is dissolved.

In this case, when the first solution is dropped into the second solution and mixed, organic-inorganic metal halide perovskite (OIP) is precipitated from the second solution due to a difference in solubility. The organic-inorganic metal halide perovskite (OIP) precipitated in the second solution is stabilized by an alkyl halide surfactant to form well-dispersed organic-inorganic metal halide perovskite nanocrystals (OIP-NC). Accordingly, it is possible to prepare an organic-inorganic hybrid metal halide perovskite nanoparticle including an organic-inorganic metal halide perovskite nanocrystal and a plurality of alkyl halide organic ligands surrounding the organic-inorganic metal halide perovskite nanocrystal.

Meanwhile, the size of the organic-inorganic metal halide perovskite nanocrystal particles can be controlled by controlling the length or shape factor and amount of the alkyl halide surfactant. For example, the shape factor control can control the size through a linear, tapered, or inverted triangular surfactant.

That is, the metal halide perovskite nanoparticles according to the present disclosure can be prepared through an inverse nano-emulsion method.

Meanwhile, as an example of the synthesis of AX at this time, when A is CH₃NH₃ and X is Br, CH₃NH₂ (methylamine) and HBr (hydroiodic acid) are dissolved in a nitrogen atmosphere to obtain CH₃NH₃Br through solvent evaporation. When the first solution is added to the second solution, a metal halide perovskite is precipitated in the second solution due to a difference in solubility, and the precipitated metal halide perovskite is surrounded by an alkyl halide surfactant while stabilizing the surface, it may be to generate metal halide perovskite nanoparticles including well-dispersed metal halide perovskite nanocrystals. At this time, the surface of the metal halide perovskite nanocrystal may be surrounded by organic ligands that are alkyl halide.

Thereafter, metal halide perovskite nanoparticles can be obtained by selectively evaporating by applying heat to a protic solvent including the metal halide perovskite nanoparticles, or selectively extracting protic solvent including nanoparticles from the aprotic solvent by adding a co-solvent that can dissolve both a protic solvent and aprotic solvent.

In another embodiment of the present disclosure, the forming of the semiconductor layer may include mixing an organic semiconductor with a solution containing the organic-inorganic metal halide perovskite nanoparticles to form an organic-inorganic metal halide perovskite-organic semiconductor solution. And forming a semiconductor layer by spin coating the organic-inorganic metal halide perovskite-organic semiconductor solution on the substrate or the gate insulating layer.

Specifically, in the process of forming a semiconductor layer by spin coating the organic-inorganic metal halide perovskite-organic semiconductor solution, the semiconductor layer may include an organic layer on the substrate or the gate insulating layer; The semiconductor layer and the organic-inorganic metal halide perovskite nanoparticles may be sequentially stacked and self-organization.

Specifically, first, the organic-inorganic metal halide perovskite-organic semiconductor solution may be prepared by mixing an organic semiconductor with a solution containing the organic-inorganic metal halide perovskite nanoparticles. The organic semiconductor is tris(8-quinolinorate)aluminum (Alq₃), TAZ, TPQ1, TPQ2, Bphen (4,7-diphenyl-1,10-phenanthroline (4,7-diphenyl-1,10-phenanthroline)), BCP, BeBq₂, BAlq, CBP (4,4′-N,N′-dicarbazole-biphenyl), 9,10-di(naphthalen-2-yl) anthracene (ADN), TCTA (4, 4′, 4″-tris(N-carbazolyl)triphenylamine), TPBI(1,3,5-tris(N-phenylbenzimidazol-2-yl)benzene(1,3,5-tris(Nphenylbenzimidazol) e-2-yl)benzene)), TBADN (3-tert-butyl-9,10-di(naphth-2-yl) anthracene), and E3, but may be one or more selected from the group consisting of, but is not limited thereto.

Thereafter, the organic-inorganic metal halide perovskite-organic semiconductor solution may be spin-coated to form a semiconductor layer. In this case, the spin coating speed is preferably 1000 rpm to 5000 rpm, and the spin coating time may be 15 seconds to 150 seconds. If the spin coating speed drops to 1000 rpm or less, or if the spin coating time is shortened to 15 seconds or less, the thin film may become non-uniform or the solvent may not evaporate.

Accordingly, the semiconductor layer of the present disclosure may form a nanocrystalline thin film of organic-inorganic metal halide perovskite nanoparticles including organic-inorganic metal halide perovskite nanocrystals on the substrate or the gate insulating layer.

As described above, when the semiconductor layer is formed, exciton-exciton annihilation, which may occur because the nanocrystals are closely located in the existing metal halide perovskite nanocrystal layer, can be prevented. In addition, by using an organic host or co-host having a bipolar characteristic, the electron-hole recombination zone can be widened, thereby preventing exciton-exciton annihilation. Accordingly, roll-off that occurs when the metal halide perovskite light emitting transistor is driven at high luminance can be reduced.

In another embodiment of the present disclosure, the forming of the semiconductor layer includes forming a self-assembled monomolecular film on a member for depositing a semiconductor layer, and the organic-inorganic metal halide perovskite nanoparticles on the self-assembled monomolecular film forming an organic-inorganic metal halide perovskite nanoparticle layer by spin coating a solution containing, and forming the organic-inorganic metal halide perovskite nanoparticle layer on the substrate or the gate insulating layer using a stamp process can be included.

Specifically, first, a self-assembled monolayer may be formed on the semiconductor layer deposition member. In this case, a member made of silicon may be used as the member for depositing the semiconductor layer. In more detail, an octadecyltrichlorosilane (ODTS) treated wafer obtained by dipping a silicon native wafer in an ODTS solution may be used.

Thereafter, a solution including the organic-inorganic metal halide perovskite nanoparticles may be spin-coated on the self-assembled monolayer to form an organic-inorganic metal halide perovskite nanoparticle layer. Then, the organic-inorganic metal halide perovskite nanoparticle layer may be formed on the second semiconductor layer deposition member using a stamp. The stamp may be prepared by curing polydimethylsiloxane (PDMS) on a silicon wafer.

In the case of forming the semiconductor layer as described above, the problems related to substrate sensitivity, and stacking processes of large-area assembly (Large-area assembly) and layer-by-layer (layer-by-layer) in the existing wet processes can be solved by forming the organic-inorganic metal halide perovskite nanoparticle layer through the stamping process, In the metal halide perovskite light emitting transistor, the order of performing the steps of forming each of the substrate, the gate electrode, the gate insulating layer, the source electrode, and the drain electrode may be changed according to the structure of the transistor to be manufactured. Specifically, the structure of the metal halide perovskite light emitting transistor implemented in the embodiment of the present disclosure is a bottom-gate/top-contact structure, a bottom-gate/bottom-contact structure, a top-gate/top-contact structure, or it may be a top-gate/bottom-contact structure.

Specifically, in an embodiment of the method of manufacturing the metal halide perovskite light emitting transistor, prior to the forming of the semiconductor layer, the step of sequentially forming the gate electrode and the gate insulating layer on a substrate is performed. It may further include, and after the step of forming the semiconductor layer, forming a source electrode and a drain electrode electrically connected to the semiconductor layer at one end and the other end of the semiconductor layer. Specifically, this may be a method of manufacturing a metal halide perovskite light emitting transistor having a bottom-gate/top-contact structure as shown in FIG. 16(a).

In another embodiment of the method of manufacturing the metal halide perovskite light emitting transistor, before the step of forming the semiconductor layer, the steps of sequentially forming the gate electrode, the gate insulating film, and a source electrode and a drain electrode electrically connected to one end and the other end of the semiconductor layer are further included. Specifically, this may be a method of manufacturing a metal halide perovskite light emitting transistor having a bottom-gate/bottom-contact structure as shown in FIG. 16(b).

In another embodiment of the method of manufacturing the metal halide perovskite light emitting transistor, before the step of forming the semiconductor layer, the steps of forming a source electrode and a drain electrode electrically connected to the semiconductor layer at one end and the other end of the semiconductor layer on a substrate is further included, and after forming the semiconductor layer, the steps of sequentially forming the gate insulating layer and the gate electrode on the semiconductor layer is further included. Specifically, this may be a method of manufacturing an organic-inorganic hybrid metal halide perovskite light emitting transistor having a top-gate/bottom-contact structure, as shown in FIG. 16(d).

In each of the above-described embodiments, forming the gate electrode, the gate insulating film, the source electrode, and the drain electrode may be performed using at least one method selected from organic nanowire lithography, drop casting, and spin coating. dip coating, e-beam evaporation, thermal evaporation, printing, soft lithography, and sputtering.

In an embodiment of the present disclosure, the forming of the gate electrode, the gate insulating layer, the source electrode, and the drain electrode may be performed using the organic nanowire lithography method. Specifically, in the organic nanowire lithography, the steps include forming an organic wire or organic-inorganic hybrid wire mask pattern having a circular or elliptical cross section on a pattern forming member, forming a target material layer on the mask pattern and removing the mask pattern to leave the target material layer in a region where the mask pattern is not formed. Here, the target material layer may be a material layer for forming a gate electrode that is a target to be formed.

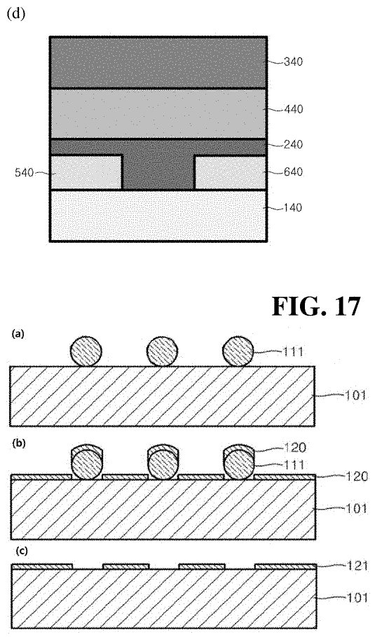

FIG. 17(a) to 17(c) are schematic diagrams showing an organic nanowire lithography process sequence according to an embodiment of the present disclosure.

First, as shown in FIG. 17A, an organic wire or organic-inorganic hybrid wire mask pattern (111) having a circular or elliptical cross section may be formed on the pattern forming member (101)

Thereafter, referring to FIG. 17B, a target material layer (120) may be formed on the mask pattern (111). The target material layer may be formed on the mask pattern (111) and on the pattern forming member (101) as shown in FIG. 17B.

Then, when the mask pattern (111) is removed, the target material layer (121) may remain in a region where the mask pattern is not formed, as shown in FIG. 17C. Using this method, the gate electrode, the gate insulating film, the source electrode, and the drain electrode included in the metal halide perovskite light emitting transistor of the present disclosure may be formed.

The organic wire or organic-inorganic hybrid wire mask pattern having a circular or elliptical cross section may include electric field assisted robotic nozzle printing, direct tip drawing, and meniscus-guided direct writing, melt spinning, wet spinning, dry spinning, gel spinning, or electrospinning.

Specifically, this may be performed using an electric field assisted robotic nozzle printer device as disclosed in Korean Patent Registration No. 10-1407209.

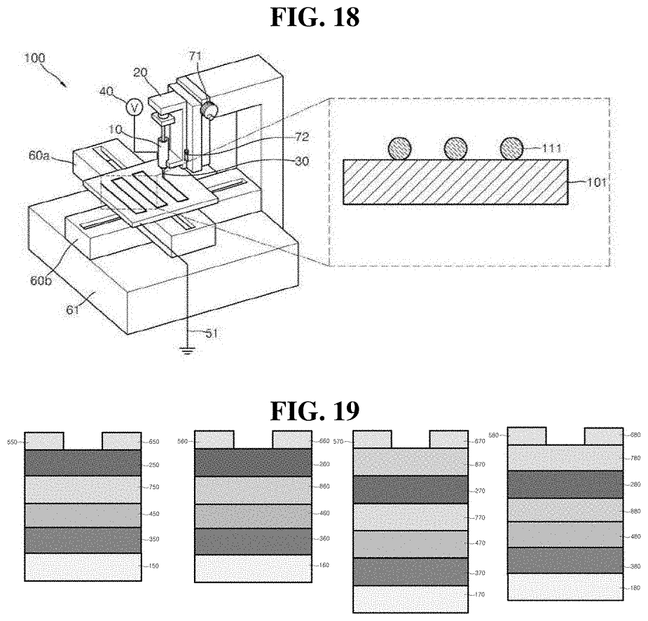

FIG. 18 is a schematic diagram of an electric field assisted robotic nozzle printer.

Referring to FIG. 18, the electric field auxiliary robotic nozzle printing device (100) includes a solution storage device (10) for supplying a solution for discharging, and a nozzle (30) for discharging a solution supplied from the solution storage device (10). A voltage application device (40) for applying a high voltage to the nozzle (30), a flat and movable collector (50) in which an organic wire or an organic-inorganic hybrid wire formed by being discharged from the nozzle (30) is aligned, the collector (50) a robot stage (60) installed below and capable of moving the collector 50 in the x-y direction (horizontal direction), the nozzle (30) and the collector (50) in the z direction (vertical direction) a micro-distance adjuster that adjusts the distance between the micro-distance adjusters, and a stone table (61) located under the robot stage (60) to maintain a plan view of the collector (50) and suppress vibrations generated during the operation of the robot stage (60). It may be to use the included electric field assisted robotic nozzle printer (100).

The above-described organic nanowire lithography may be performed using the electric field assisted robotic nozzle printer. Specifically, as shown in FIG. 17, an organic wire or an organic-inorganic hybrid wire mask pattern (111) may be formed on the substrate (101).

Meanwhile, according to an embodiment, before or after the step of forming the semiconductor layer, the step of forming at least one of an electron transport layer and a hole transport layer above or below the semiconductor layer may be further included.

Specifically, in an embodiment of the present disclosure, in the case of a metal halide perovskite light emitting transistor having a bottom-gate/top-contact structure, as shown in FIG. 19(a), the electron transport layer is formed under the semiconductor layer. It may further include forming. Alternatively, as shown in FIG. 19(b), the step of forming a hole transport layer under the semiconductor layer may be further included. Alternatively, the step of forming an electron transport layer under the semiconductor layer and forming a hole transport layer over the semiconductor layer as shown in FIG. 19(c) may be further included. Alternatively, as shown in FIG. 19(d), forming a hole transport layer under the semiconductor layer and forming an electron transport layer over the semiconductor layer may be further included.

In detail, the electron transport layer may be formed according to a method arbitrarily selected from a variety of known methods such as a vacuum deposition method, a spin coating method, a casting method, and an LB method.

As the electron transport layer material, a known electron transport material may be used. For example, the electron transport layer is a quinoline derivative, in particular tris(8-hydroxyquinoline)aluminum: Alq₃, Bis(2-methyl-8-quinoli nolate)-4-(phenylphenolato)aluminium: Balq, bis(10-hydroxybenzo[h]quinolinato)-beryllium: Bebq₂), 2,9-dimethyl-4,7-diphenyl-1,10-phenanthroline: BCP, 4,7-diphenyl-1,10-phenanthroline: Bphen, 2,2,2 (benzene-1,3,5-triyl)-Tris(1-phenyl-1H-benzimidazole): TPBI, 3-(4-Biphenyl)-4-(phenyl-5-tert-butylphenyl-1,2,4-triazole: TAZ), 4-(naphthalen-1-yl)-3,5-diphenyl-4H-1,2,4-triazole: NTAZ), 2,9-bis(naphthalen-2-yl)-4,7-diphenyl-1,10-phenanthroline: NBphen), tris(2,4,6-trimethyl-3-(pyridin-3-yl)phenyl)borane: 3TPYMB), phenyl-dipyrenylphosphine oxide: POPy₂, 3,3,5,5 Tetra[(m-pyridyl)-phen-3-yl]biphenyl: BP4mPy, 1, 3,5-tri[(3-pyridyl)-phen-3-yl]benzene: TmPyPB), 1,3-Bis[3,5-di(pyridin-3-yl)phenyl]benzene: BmPyPhB, bis(10-hydroxybenzo) [h]quinolinato)beryllium: Bepq₂), diphenylbis(4-(pyridin-3-yl)phenyl)silane: DPPS and 1,3,5-tri(p-pyrid-3-yl-phenyl)benzene: TpPyPB, 1,3-bis[2-(2,2bipyridin-6-yl)-1,3,4-oxadiazo-5-yl]benzene: Bpy-OXD, 6,6 bis[5-(biphenyl-4-yl)-1,3,4-oxadiazo-2-yl]-2,2bipyridyl: BP-OXD-Bpy, and the like, but are not limited thereto.

In addition, the hole transport layer may be formed according to a method arbitrarily selected from among various known methods such as a vacuum deposition method, a spin coating method, a cast method, and an LB method. At this time, in the case of selecting the vacuum deposition method, the deposition conditions vary depending on the target compound, the structure of the target layer, and thermal properties, but for example, the deposition temperature range of 100° C. to 500° C., 10⁻¹⁰ to 10⁻³ torr The vacuum degree may be selected within a range of a deposition rate of 0.01 Å/sec to 100 Å/sec. On the other hand, in the case of selecting the spin coating method, the coating conditions are different depending on the target compound, the structure of the target layer, and thermal properties, but the coating speed range of 2000 rpm to 5000 rpm, the heat treatment temperature of 80 to 200° C. (after coating heat treatment temperature for solvent removal) may be selected within the range.

The hole transport layer material may be selected from materials capable of better transporting holes than hole injection. The hole transport layer may be formed using a known hole transport material. For example, the hole transport layer may be an amine-based material having an aromatic condensed ring or a triphenyl amine-based material.

More specifically, the hole transporting material is, 1,3-bis(carbazol-9-yl)benzene(MCP), 1,3,5-tris(carbazol-9-yl)benzene(TCP), 4,4′, 4″-tris(carbazol-9-yl)triphenylamine(TCTA), 4,4′-bis(carbazol-9-yl)biphenyl(CBP), N,N′-bis(naphthalen-1-yl)-N,N′-bis(phenyl)benzidine(NPB), N,N′-bis(naphthalen-2-yl))-N,N′-bis(phenyl)-benzidine(β-NPB), N,N′-bis(naphthalen-1-yl)-N,N′-bis(phenyl)-2,2′-dimethylbenzidine(αNPD), (Di-[4-(N,N-ditolyl-amino)-phenyl]cyclohexane(TAPC), N,N,N′,N′-tetra-naphthalen-2-yl-benzidine(β-TNB) and N4,N4,N4′,N4′-tetra(biphenyl-4-yl)biphenyl-4,4′-diamine(TPD15), poly(9,9-dioctylfluorene-co-bis-N,N′-(4-butylphenyl)-bis-N,N′-phenyl-1,4-phenylenedi amine)(PFB), poly(9,9′-dioctylfluorene-co-N-(4-butylphenyl)diphenylamine)(TFB), poly(9,9′-dioctylfluorene-co-bis-N,N′-(4-butylphenyl)-bis-N,N′-phenylbenzidine)(BFB), poly(9,9-dioctylfluorene-co-bis-N,N′-(4-methoxyphenyl)-bis-N,N′-phenyl-1,4-phenylenediamine) (PFMO), and the like may be exemplified, but the present disclosure is not limited thereto.

The thickness of the hole transport layer may be 5 nm to 100 nm, for example, 10 nm to 60 nm. When the thickness of the hole transport layer satisfies the above-described range, excellent hole transport characteristics can be obtained without an increase in driving voltage.

<Metal Halide Perovskite Light Emitting Device Including Passivation Layer>

According to still another embodiment of the present disclosure, the light emitting device may include a passivation layer capable of reducing defects in the metal halide perovskite thin film and eliminating charge imbalance.

Metal halide perovskite nanocrystal particles having improved properties that can be applied to various electronic devices exhibit improved luminescence efficiency by confining excitons to a very small size. In addition, even a bulk polycrystalline film having a very small grain size may exhibit improved luminescence efficiency through exciton confinement. However, the metal halide perovskite light emitting layer shows relatively low luminescence efficiency because surface defects still exist, and it causes charge carrier imbalance in the light emitting device to show low luminescence efficiency. Accordingly, by further including a passivation layer in the light emitting device, defects of the metal halide perovskite thin film may be reduced and charge imbalance may be eliminated.

A metal halide perovskite light emitting device comprising a passivation layer according to the present disclosure includes a metal halide perovskite thin film as a light emitting layer, and a passivation layer is formed on the metal halide perovskite thin film.

FIG. 22 is a schematic diagram showing a metal halide perovskite light emitting device according to an embodiment of the present disclosure.

Referring to FIG. 22, the metal halide perovskite light emitting device according to the present disclosure includes a substrate (10), a first electrode (20), a metal halide perovskite thin film (30), a passivation layer (40), and a second electrode (50).

The form of the metal halide perovskite nanocrystal may be a form commonly used in the art. The shape of the metal halide perovskite nanocrystal may be a 0-dimensional, 1-dimensional or 2-dimensional shape. As an example, it may be in the form of a sphere, an ellipsoid, a hollow cube, a pyramid, a cylinder, a cone, an elliptic column, hollow sphere, Janus particle, prism, multipod, polyhedron, nano tube, nano wire, nano fiber or nanoplatelet.

In addition, the size of the metal halide perovskite crystalline particles may be 1 nm to 10 μm or less. For example, 1 nm, 2 nm, 3 nm, 4 nm, 5 nm, 6 nm, 7 nm, 7.5 nm, 8 nm, 8.5 nm, 9 nm, 9.5 nm, 10 nm, 10.5 nm, 11 nm, 11.5 nm, 12 nm, 12.5 nm, 13 nm, 13.5 nm, 14 nm, 14.5 nm, 15 nm, 16 nm, 17 nm, 18 nm, 19 nm, 20 nm, 25 nm, 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 150 nm, 200 nm, 250 nm, 300 nm, 350 nm, 400 nm, 450 nm, 500 nm, 600 nm, 700 nm, 800 nm, 900 nm, 1 μm, 2 μm, 5 μm, or 10 μm. The size of the particle can be defined as a region with the lower value as the minimum value and the larger value as the maximum value among the two numbers selected above. It is preferably 8 nm or more and 300 nm or less, and more preferably 10 nm or more and 30 nm or less. On the other hand, the size of the crystalline particles at this time means a size that does not take into account the length of a ligand to be described later, that is, the size of the remaining portions excluding the ligand. When the size of the crystalline particles is 1 μm or more, there is a fundamental problem in that excitons do not emit light due to thermal ionization and delocalization of charge carriers in a large crystal, but are separated into free charges and disappeared. I can. In addition, more preferably, as described above, the size of the crystalline particles may be greater than or equal to a exciton Bohr diameter. The thermal ionization and delocalization of the charge carrier may gradually appear when the size of the nanocrystal exceeds 100 nm. If it is more than 300 nm, the phenomenon will appear more, and if it is more than 1 μm, it is completely bulky and is subject to the above phenomenon.

For example, when the nanocrystal particle are spherical, the diameter of the nanocrystal particle may be 1 nm to 10 μm. Preferably diameter may be 1 nm, 3 nm, 5 nm, 6 nm, 7 nm, 8 nm, 9 nm, 10 nm, 11 nm, 12 nm, 13 nm, 14 nm, 15 nm, 16 nm, 17 nm, 18 nm, 19 nm, 20 nm, 25 nm, 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 150 nm, 200 nm, 250 nm, 300 nm, 350 nm, 400 nm, 450 nm, 500 nm, 600 nm, 700 nm, 800 nm, 900 nm, 1 μm, 2 μm, 5 μm or 10 μm.

In addition, the band gap energy of the nanocrystal particle may be 1 eV to 5 eV. Preferably, the band gap energy of the nanocrystal particle is 1 eV, 1.1 eV, 1.2 eV, 1.3 eV, 1.4 eV, 1.5 eV, 1.6 eV, 1.7 eV, 1.8 eV, 1.81 eV, 1.82 eV, 1.83 eV, 1.84 eV, 1.85 eV, 1.86 eV, 1.87 eV, 1.88 eV, 1.89 eV, 1.9 eV, 1.91 eV, 1.92 eV, 1.93 eV, 1.94 eV, 1.95 eV, 1.96 eV, 1.97 eV, 1.98 eV, 1.99 eV, 2 eV, 2.01 eV, 2.02 eV, 2.03 eV, 2.04 eV, 2.05 eV, 2.06 eV, 2.07 eV, 2.08 eV, 2.09 eV, 2.1 eV, 2.11 eV, 2.12 eV, 2.13 eV, 2.14 eV, 2.15 eV, 2.16 eV, 2.17 eV, 2.18 eV, 2.19 eV, 2.2 eV, 2.21 eV, 2.22 eV, 2.23 eV, 2.24 eV, 2.25 eV, 2.26 eV, 2.27 eV, 2.28 eV, 2.29 eV, 2.3 eV, 2.31 eV, 2.32 eV, 2.33 eV, 2.34 eV, 2.35 eV, 2.36 eV, 2.37 eV, 2.38 eV, 2.39 eV, 2.4 eV, 2.41 eV, 2.42 eV, 2.43 eV, 2.44 eV, 2.45 eV, 2.46 eV, 2.47 eV, 2.48 eV, 2.49 eV, 2.5 eV, 2.51 eV, 2.52 eV, 2.53 eV, 2.54 eV, 2.55 eV, 2.56 eV, 2.57 eV, 2.58 eV, 2.59 eV, 2.6 eV, 2.61 eV, 2.62 eV, 2.63 eV, 2.64 eV, 2.65 eV, 2.66 eV, 2.67 eV, 2.68 eV, 2.69 eV, 2.7 eV, 2.71 eV, 2.72 eV, 2.73 eV, 2.74 eV, 2.75 eV, 2.76 eV, 2.77 eV, 2.78 eV, 2.79 eV, 2.8 eV, 2.9 eV, 3 eV, 3.1 eV, 3.2 eV, 3.3 eV, 3.4 eV, 3.5 eV, 3.6 eV, 3.7 eV, 3.8 eV, 3.9 eV, 4 eV, 4.1 eV, 4.2 eV, 4.3 eV, 4.4 eV, 4.5 eV, 4.6 eV, 4.7 eV, 4.8 eV, 4.9 eV, 5 eV. Band gap energy of the nanocrystal particle can be defined as a region that takes the lower value as the minimum value and the larger value as the maximum value out of the two numbers selected above.

Accordingly, since the energy band gap is determined according to the constituent material or crystal structure of the nanocrystal particle, light having a wavelength of, for example, 200 nm to 1300 nm may be emitted by controlling the constituent material of the nanocrystal particle. In addition, preferably, the nanocrystal particle may emit ultraviolet, blue, green, red, and infrared light.

The ultraviolet light can be defined as a region that takes the lower value as the minimum value and the larger value as the maximum value out of the two numbers selected among 200 nm, 210 nm, 220 nm, 230 nm, 240 nm, 250 nm, 260 nm, 270 nm, 280 nm, 290 nm, 300 nm, 310 nm, 320 nm, 330 nm, 340 nm, 350 nm, 360 nm, 370 nm, 380 nm, 390 nm, 400 nm, 410 nm, 420 nm, and 430 nm. The blue light can be defined as a region that takes the lower value as the minimum value and the larger value as the maximum value out of the two numbers selected among 440 nm, 450 nm, 451 nm, 452 nm, 453 nm, 454 nm, 455 nm, 456 nm, 457 nm, 458 nm, 459 nm, 460 nm, 461 nm, 462 nm, 463 nm, 464 nm, 465 nm, 466 nm, 467 nm, 468 nm, 469 nm, 470 nm, 471 nm, 472 nm, 473 nm, 474 nm, 475 nm, 476 nm, 477 nm, 478 nm, 479 nm, 480 nm, 490 nm. The green light can be defined as a region that takes the lower value as the minimum value and the larger value as the maximum value out of the two numbers selected among 500 nm, 501 nm, 502 nm, 503 nm, 504 nm, 505 nm, 506 nm, 507 nm, 508 nm, 509 nm, 510 nm, 511 nm, 512 nm, 513 nm, 514 nm, 515 nm, 516 nm, 517 nm, 518 nm, 519 nm, 520 nm, 521 nm, 522 nm, 523 nm, 524 nm, 525 nm, 526 nm, 527 nm, 528 nm, 529 nm, 84 530 nm, 531 nm, 532 nm, 533 nm, 534 nm, 535 nm, 536 nm, 537 nm, 538 nm, 539 nm, 540 nm, 541 nm, 542 nm, 543 nm, 544 nm, 545 nm, 546 nm, 547 nm, 548 nm, 549 nm, 550 nm, 560 nm, 570 nm, and 580 nm. The red light can be defined as a region that takes the lower value as the minimum value and the larger value as the maximum value out of the two numbers selected among 590 nm, 600 nm, 601 nm, 602 nm, 603 nm, 604 nm, 605 nm, 606 nm, 607 nm, 608 nm, 609 nm, 610 nm, 611 nm, 612 nm, 613 nm, 614 nm, 615 nm, 616 nm, 617 nm, 618 nm, 619 nm, 620 nm, 621 nm, 622 nm, 623 nm, 624 nm, 625 nm, 626 nm, 627 nm, 628 nm, 629 nm, 630 nm, 631 nm, 632 nm, 633 nm, 634 nm, 635 nm, 636 nm, 637 nm, 638 nm, 639 nm, 640 nm, 641 nm, 642 nm, 643 nm, 644 nm, 645 nm, 646 nm, 647 nm, 648 nm, 649 nm, 650 nm, 660 nm, 670 nm, 680 nm, 690 nm, 700 nm. The infrared light can be defined as a region that takes the lower value as the minimum value and the larger value as the maximum value out of the two numbers selected among 700 nm, 710 nm, 720 nm, 730 nm, 740 nm, 750 nm, 760 nm, 770 nm, 780 nm, 790 nm, 800 nm, 810 nm, 820 nm, 830 nm, 840 nm, 850 nm, 860 nm, 870 nm, 880 nm, 890 nm, 900 nm, 910 nm, 920 nm, 930 nm, 940 nm, 950 nm, 960 nm, 970 nm, 980 nm, 990 nm, 1000 nm, 1010 nm, 1020 nm, 1030 nm, 1040 nm, 1050 nm, 1060 nm, 1070 nm, 1080 nm, 1090 nm, 1100 nm, 1110 nm, 1120 nm, 1130 nm, 1140 nm, 1150 nm, 1160 nm, 1170 nm, 1180 nm, 1190 nm, 1200 nm, 1210 nm, 1220 nm, 1230 nm, 1240 nm, 1250 nm, 1260 nm, 1270 nm, 1280 nm, 1290 nm, 1300 nm.

A passivation layer (40) is formed on the metal halide perovskite thin film (30).

The metal halide perovskite thin film (30) exhibits relatively low luminescence efficiency due to the presence of surface defects, and low luminescence efficiency due to charge carrier imbalance in the light emitting device. Accordingly, there is a need for a method capable of eliminating defects in a metal halide perovskite thin film and eliminating charge imbalance in a light emitting device.

Accordingly, in a light emitting device including a metal halide perovskite thin film as a light emitting layer, the present disclosure is characterized in that a passivation layer is formed on the metal halide perovskite thin film.

In the metal halide perovskite light emitting device according to the present disclosure, the passivation layer may include one or more compounds of Formulas 1 to 4 below.

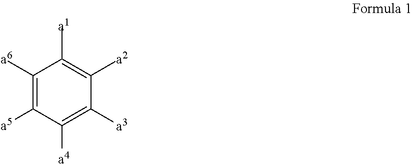

In Formula 1, a₁ to a₆ are H, CH₃ or CH₂X, wherein at least three of a₁ to a₆ are CH₂X, and X is a halogen element.

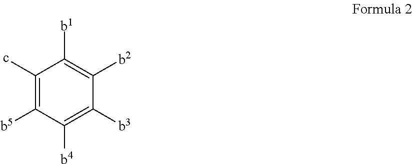

(In the above chemical formula 2

b1 to b5 are halogen elements

c is,

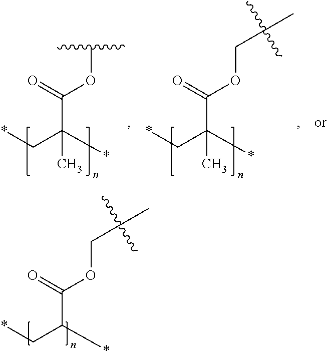

At this time, n is an integer from 1 to 100)

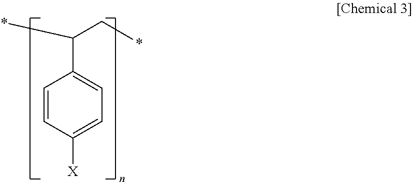

(In the above chemical formula 3

X is a halogen element

n is an integer from 1 to 100)

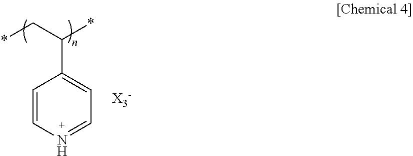

The compounds of chemical formulas 1 to 4 are organic compounds containing halogen, and defects in the emissive layer can be stabilized by compensating for the deficiency of halogen in the metal halide perovskite crystal.

Preferably, the compounds forming the passivation layer can be selected from the group consisting of poly(4-vinylpyridinium tribromide), (1,3,5-tris(bromomethyl)benzene), 2,4,6-tris(bromomethyl) mesitylene (TBMM), 1,2,4,5-tetrakis. (Bromomethyl)benzene, hexakis (bromomethyl)benzene, poly(pentabromophenylmethacrylate), poly(pentabromobenzylmethacrylate), poly(pentabromobenzyl acrylate) and poly(4-bromostyrene), more preferably 2,4,6-tris(bromomethyl)mesitylene(TBMM) can be used.

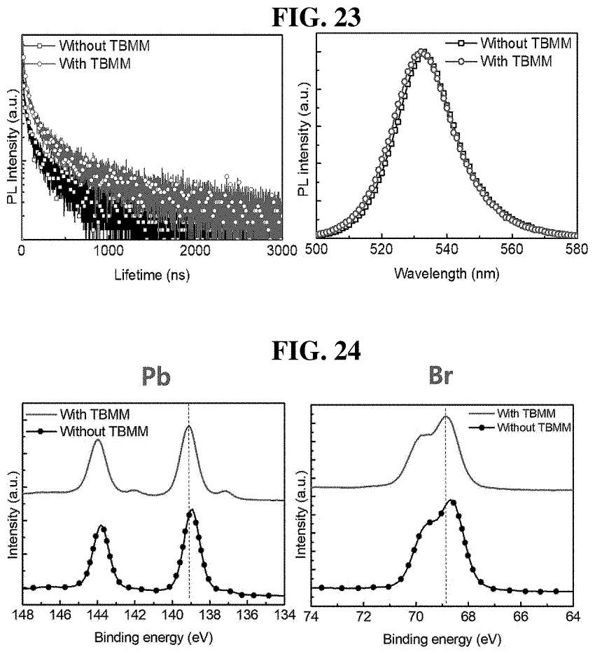

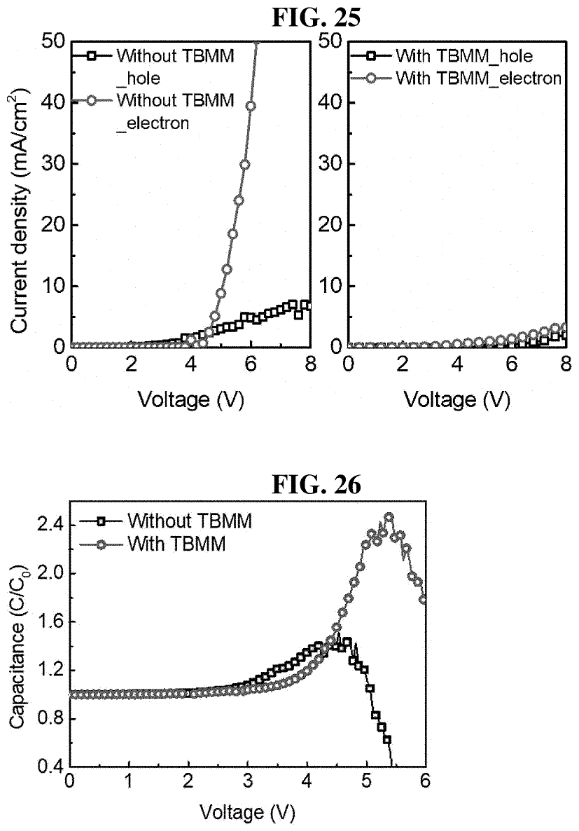

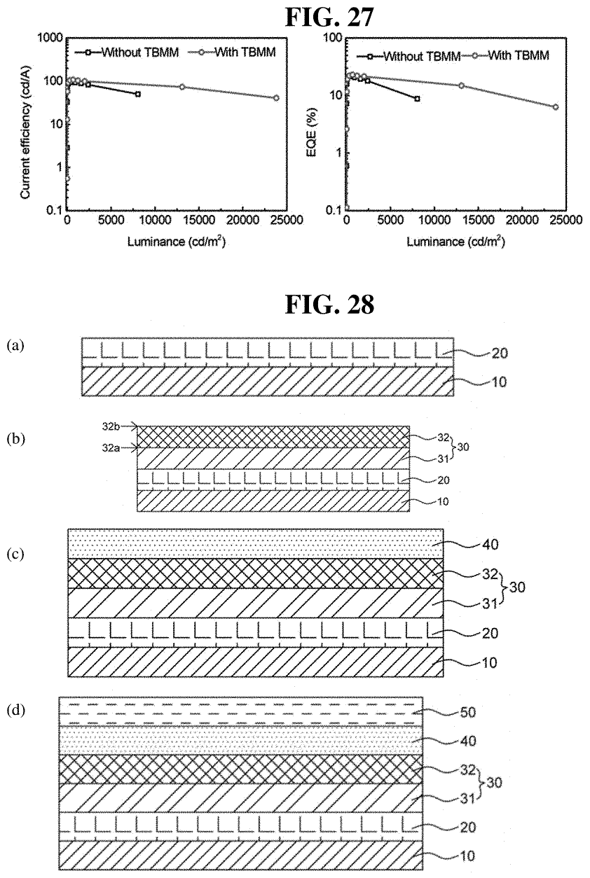

In one embodiment of the present disclosure, as a result of measuring the light emission characteristics before and after coating the TBMM thin film, which is one of the compounds of the chemical formula 1, on the metal halide perovskite nanocrystal particle emissive layer, after the TBMM thin film was coated, the photoluminescence (PL) lifetime becomes longer (see FIG. 23), the binding energy of the metal halide perovskite element increases (see FIG. 24), and the current densities of holes and electrons become similar. It was confirmed that the charge imbalance in the element was eliminated (see FIG. 25), the maximum capacitance was increased (see FIG. 26), and the luminescence efficiency and maximum brightness were improved (see FIG. 27).

Therefore, when the passivation layer is formed on the metal halide perovskite thin film, the luminescence efficiency and the photoluminescence (PL) lifetime can be improved.

The thickness of the passivation layer (40) is preferably 1 to 100 nm, but if the thickness of the passivation layer exceeds 100 nm, charge injection is reduced due to insulation characteristics.

The passivation layer may be coated by performing spin coating, bar coating, spray coating, slot die coating, gravure coating, blade coating, screen printing, nozzle printing, inkjet printing, electrohydrodynamic jet printing, electrospray or electrospinning.

On the other hand, in one embodiment of the present disclosure, if the first electrode (20) is used as an anode, the second electrode (50) is used as a cathode, and if the first electrode (20) is used as a cathode, the second electrode (50) can be used as an anode.

The first electrode (20) or second electrode (50) can be formed by physical vapor deposition (PVD), chemical vapor deposition (CVD), sputtering, pulsed laser deposition (PLD), evaporation method, electron beam evaporation, atomic layer deposition (ALD) and molecular beam epitaxy vapor deposition (MBE).

On the other hand, in the light-emitting diodes according to the embodiment of the present disclosure, when the first electrode (20) is the anode, and the second electrode (50) is the cathode, as shown in FIG. 22, a hole injection layer (23) and hole transport for facilitating hole injection between the first electrode (20) and the metal halide perovskite thin film (emissive layer) (30) can be provided as a hole transport layer. Further, an electron transport layer (43) for transporting electrons and an electron injection layer for facilitating electron injection may be provided between the passivation layer (40) and the second electrode (50).

In addition, a hole blocking layer (not shown) can be placed between the metal halide perovskite thin film (emissive layer) (30) and the electron transport layer (43). Further, an electron blocking layer (not shown) can be arranged between the metal halide perovskite thin film (emissive layer) (30) and the hole transport layer. However, the present disclosure is not limited to this, and the electron transport layer (43) can perform the role of the hole blocking layer, or the hole transport layer can also perform the role of the electron blocking layer.

The hole injection layer (23) and/or the hole transport layer have a HOMO level between the work function level of the first electrode (anode) (20) and the HOMO level of the metal halogen perovskite thin film (emissive layer) (30), and functions to improve the injection or transport efficiency of holes into the metal halide perovskite thin film (emissive layer) (30) from the first electrode (anode) (20).

The hole injecting layer (23) or the hole transporting layer can include materials commonly used as hole transporting materials, and one layer can comprise different hole transporting material layers. The hole transport material is, for example, mCP (N, Ndicarbazolyl-3,5-benzene); PEDOT:PSS (poly(3,4-ethylenedioxythiophene):polystyrenesulfonate); NPD (N, N′-di(1-naphthyl))-N, N′-diphenylbenzidine); N, N′-diphenyl-N, N′-di(3-methylphenyl)-4,4′-diaminobiphenyl (TPD); DNTPD (N4, N4′-Bis[4-[bis(3-methylphenyl)amino]phenyl]-N4, N4′-diphenyl-[1,1′-biphenyl]-4,4′-diamine); N, N′-diphenyl-N, N′-Dinaphthyl-4,4′-diaminobiphenyl; N, N, N′N′-tetra-p-tolyl-4,4′-diaminobiphenyl; N, N, N′N′-tetraphenyl-4,4′-Diaminobiphenyl; derivatives of porphyrin compounds such as copper (II) 1,10,15,20-tetraphenyl-21H, 23H-popyrin; TAPC (1,1-Bis[4-[N, N′-Di)(P-tolyl)Amino]Phenyl]Cyclohexane); Triallylamine derivatives such as N, N, N-tri(p-tolyl)amine, 4,4′, 4′-tris[N-(3-methylphenyl)-N-phenylamino]triphenylamine; carbazole derivatives such as N-phenylcarbazole and polyvinylcarbazole; phthalocyanine derivatives such as metal-free phthalocyanine, copper phthalocyanine; starburstamine derivatives; enamine stilbene derivatives; derivatives of tertiary aromatic amine and styrylamine compounds; and polysilanes and the like. These hole transporters can also perform the role of an electron blocking layer.

The hole blocking layer serves to prevent triplet excitons or holes from diffusing in the direction of the second electrode (cathode) (50) and can be selected randomly among the known hole blocking material. For example, an oxadiazole derivative, a triazole derivative, a phenanthroline derivative, TSPO1 (diphenylphosphine oxide-4-(triphenylsilyl)phenyl) and the like can be used.

The electron injecting layer and/or the electron transporting layer (43) have a LUMO level between the work function level of the second electrode (cathode) (50) and the LUMO level of the metal halogen perovskite thin film (emissive layer) (30), and functions to increase the efficiency of electron injection or transport into the metal halide perovskite thin film (emissive layer) (30) from the second electrode (cathode) (50).

The electron injection layer can be, for example, LiF, NaCl, CsF, Li₂O, BaO, BaF₂, or Liq (lithium quinol rate).

The electron transport layer (43) includes TSPO1 (diphenylphosphine oxide-4-(triphenylsilyl)phenyl), TPBi (1,3,5-tris(N-phenylbenzimidazol-2-yl)benzene), tris (8).-Kinolinolate)Aluminum (Alq3), 2,5-diarylsilrol derivative (PyPySPyPy), Perfluorolineted compound (PF-6P), COTs (Octasubstituted cyclooctatetraene), TAZ (see chemical formula below), Bphen (4,7-It can be diphenyl-1,10-phenanthroline (4,7-diphenyl-1,10-phenanthroline), BCP (see chemical formula below), or BAlq (see chemical formula below).

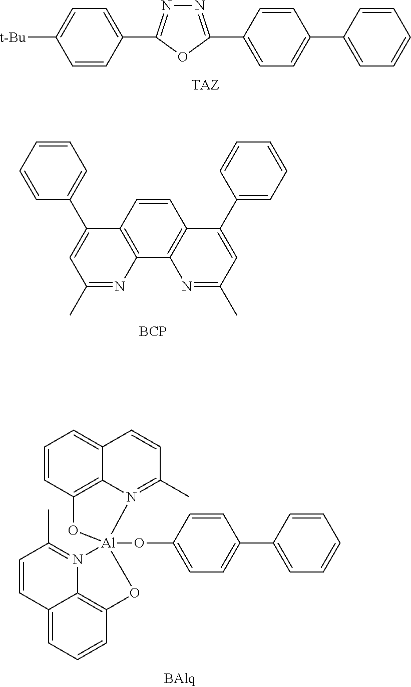

The present disclosure also comprises a method for manufacturing a metal halide perovskite light-emitting diode containing a passivation layer.

The method to producing a metal halide perovskite light-emitting diodes according to the present disclosure includes a step of forming a first electrode on a substrate; a step of forming a metal halide perovskite thin film on the first electrode; a step of forming a passivation layer containing one or more compounds of the chemical formulas 1 to 4 on the metal halide perovskite thin film; a step of forming a second electrode on the passivation layer.

Hereinafter, a method for manufacturing a metal halide perovskite light-emitting diodes including a passivation layer according to an embodiment of the present disclosure will be described with reference to the structure of FIG. 22.

First, the substrate (10) is prepared.

Next, a first electrode (20) may be formed on the substrate (10). The first electrode may be formed using a vapor deposition method or a sputtering method.

Next, a metal halide perovskite thin film (30) can be formed on the first electrode (20). The metal halide perovskite has a structure of ABX₃, A₂BX₄, A₃BX₅, A₄BX₆, ABX₄ or Aₙ₋₁BₙX₃ₙ₊₁ (n is an integer between 2 and 6), where A is an organic ammonium ion, organic amidinium ion, organic phosphonium ion, alkali metal ion or derivatives thereof, the above B contains transition metals, rare earth metals, alkaline earth metals, organic materials, inorganic materials, ammonium, derivatives thereof or combinations thereof, the above X is a halogen ion or a combination of different halogen ions.

The metal halide perovskite thin film (30) can be a thin film composed of a bulk polycrystalline thin film or nanocrystal particle, and the nanocrystal particle have a core-shell structure or a structure having a gradation composition.

These metal halide perovskite thin films (30) are can be formed by using bar-coating, spray coating, slot-die coating, gravure coating, blade coating, screen printing, nozzle printing, inkjet printing, electrohydrodynamic-jet printing, electrospray, electrospinning.

Next, a passivation layer (40) may be formed on the metal halide perovskite thin film (30). The passivation layer preferably includes at least one compound of formulas 1 to 4, and specifically, the compound constituting the passivation layer is (1,3,5-tris(bromomethyl)benzene), 2,4,6-tris(bromomethyl)mesitylene (TBMM), 1,2,4,5-tetrakis(bromomethyl)benzene, hexakis(bromomethyl)benzene, poly(pentabromophenyl methacrylate), poly(pentabromobenzyl methacrylate), poly(pentabromobenzyl acrylate), poly(4-bromostyrene) and poly(4-vinylpyridinium tribromide).

It is preferable that the thickness of the passivation layer (40) is 1 to 100 nm, and if the thickness of the passivation layer exceeds 100 nm, there is a problem that charge injection decreases due to insulating properties.

The passivation layer (40) is formed using spin coating, bar coating, spray coating, slot die coating, gravure coating, blade coating, screen printing, nozzle printing, inkjet printing, electrohydrodynamic jet printing, electrospray or electrospinning.

A second electrode (50) can be formed on the passivation layer (40). These two electrodes (50) can be formed by using a vapor deposition method or a sputtering method.

Further, in one embodiment of the present disclosure, the method for producing the metal halide perovskite light-emitting diodes can include a step of forming a first electrode on a substrate; a step of forming a hole injection layer on the first electrode; a step of forming a metal halide perovskite thin film as a emissive layer on the hole injection layer; a step of forming a passion layer containing one or more compounds of the chemical formulas 1 to 4 on the metal halide perovskite thin film; a step of forming an electron transport layer on the passivation layer; a step of forming a second electrode on the electron transport layer.

Such hole injection layers or electron transport layers can be formed by conducting spin coating, dip coating, thermal or spray deposition.

In the metal halide perovskite light-emitting diodes prepared as described above, a passivation layer composed of one or more compounds of formulas 1 to 4 is formed on the metal halide perovskite thin film. By removing defects and matching charge imbalance in the device, the maximum efficiency and maximum luminance of the light-emitting diodes including the metal halide perovskite thin film are improved.

<Metal Halide Perovskite Light-Emitting Diodes Including Exciton Buffer Layer>

According to an embodiment of the present disclosure, the metal halide perovskite light-emitting diodes can include an exciton buffer layer.

FIG. 28(a) to 28(d) is a schematic diagram showing a method of manufacturing light-emitting diodes including an exciton buffer layer according to an embodiment of the present disclosure. In FIG. 28(a) to 28(d), a metal halide perovskite is described, but an inorganic halide metal halide perovskite may be applied in the same manner as the description of the metal halide perovskite.

With reference to FIG. 28 (a), the first electrode (20) is formed on the substrate (10).

The description of the substrate and the first electrode will be omitted because they are as described above.

With reference to FIG. 28 (b), an exciton buffer layer (30) containing a conductive material and a fluorine-based material having a surface energy lower than that of the conductive material is formed on the first electrode (20) described above.

In this case, the above-described exciton buffer layer (30) is a form in which the conductive layer (31) including the conductive material described above and the surface buffer layer (32) including the fluorine-based material described above are sequentially stacked as shown in FIG. 28(b).

The conductive material described above can contain at least one of a group consisting of conductive polymers, metal carbon nanotubes, graphene, reduced graphene oxide, metal nanowires, semiconductor nanowires, metal grids, metal quantum dots and conductive oxides.

The conductive polymers described above include polythiophene, polyaniline, polypyrrole, polystyrene, sulfonated polystyrene, poly(3,4-ethylenedioxythiophene), self-doped conductive polymers, derivatives thereof, or combinations thereof. The above-described derivative may mean that it may further include various sulfonic acids and the like.

For example, the conductive polymer described above can contain at least one of a group consisting of Pani:DBSA (Polyaniline/Dodecylbenzenesulfonic acid, see the following formula), PEDOT:PSS (Poly(3,4-thylenedioxythiophene)/Poly(4-styrenesulfonate), see the following formula), Pani:CSA (Polyaniline/Camphor sulfonic acid:polyaniline/camphorsulfonic acid) and PANI:PSS (Polyaniline)/Poly(4-styrenesulfonate), but is not limited thereto.

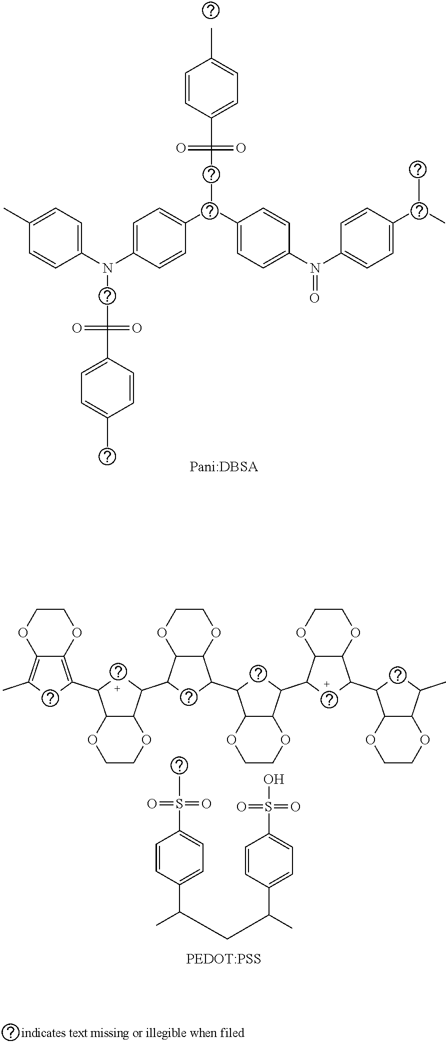

The R can be an H or C₁-C₁₀ alkyl group.

The self-doping conductive polymer can have a degree of polymerization of 10 to 10,000,000 and can have repeating units represented by chemical formula 5 below:

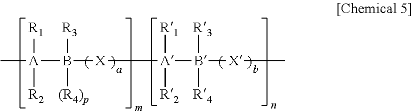

In formula 5, 0<m<10,000,000, 0<n<10,000,000, 0≤a≤20, ≤b≤20;

At least one of R₁, R₂, R₃, R′₁, R′₂, R′₃ and R′₄ contains an ionic group, and A, B, A′ and B′ are independently selected from C, Si, Ge, Sn, or Pb;

R₁, R₂, R₃, R′₁, R′₂, R′₃ and R′₄ are independently selected from the group consisting of hydrogen, halogen, nitro group, substituted or unsubstituted amino group, cyano group, substituted or unsubstituted C₁-C₃₀ alkyl groups, substituted or unsubstituted C₁-C₃₀ alkoxy groups, substituted or unsubstituted C₆-C₃₀ aryl groups, substituted or unsubstituted C₆-C₃₀ arylalkyl groups, substituted or unsubstituted C₆-C₃₀ aryloxy groups, substituted or unsubstituted C₂-C₃₀ heteroaryl groups, substituted or unsubstituted C₂-C₃₀ heteroarylalkyl groups, substituted or unsubstituted C₂-C₃₀ heteroaryloxy group, substituted or unsubstituted C₅-C₃₀ cycloalkyl group, substituted or unsubstituted C₅-C₃₀ heterocycloalkyl group, substituted or unsubstituted C₁-C₃₀ alkyl ester group, substituted or substituted C₆-C₃₀ allyl ester groups, and hydrogen or halogen elements are selectively bonded to the carbon in the above chemical formula;

R₄ is composed of a conjugated conductive polymer chain,

X and X′ are each independently selected from the a simple bond, O, S, a substituted or unsubstituted C₁-C₃₀ alkylene group, a substituted or unsubstituted C₁-C₃₀ heteroalkylene group, a substituted or unsubstituted C₆-C₃₀ arylene group, a substituted or unsubstituted C₆-C₃₀ arylalkylene group, a substituted or unsubstituted C₂-C₃₀ heteroarylene group, a substituted or unsubstituted C₂-C₃₀ heteroarylalkylene group, a substituted or unsubstituted C₅-C₂₀ cycloalkylene group, a substituted or unsubstituted C₅-C₃₀ heterocycloalkylene group aryl ester group, and hydrogen or a halogen element may be optionally bonded to carbon in the above formula.

For example, the ionic group is an anionic group selected from the group consisting of PO₃, SO₃, COO, I, CH₃COO, and a metal ion selected from Na, K, Li, Mg, Zn, Al, and H, NH₄, CH₃(—CH₂—)ₙO (n is a natural number of 1 to 50) selected from the group consisting of organic ions and may include a cationic group paired with the anionic group.

For example, in the self-doped conductive polymer of Formula 100, at least one of each of R₁, R₂, R₃, R′₁, R′₂, R′₃ and R′₄ may be fluorine, or a group substituted with fluorine, but is not limited thereto.

Specific examples of the conductive polymer are as follows but are not limited thereto.

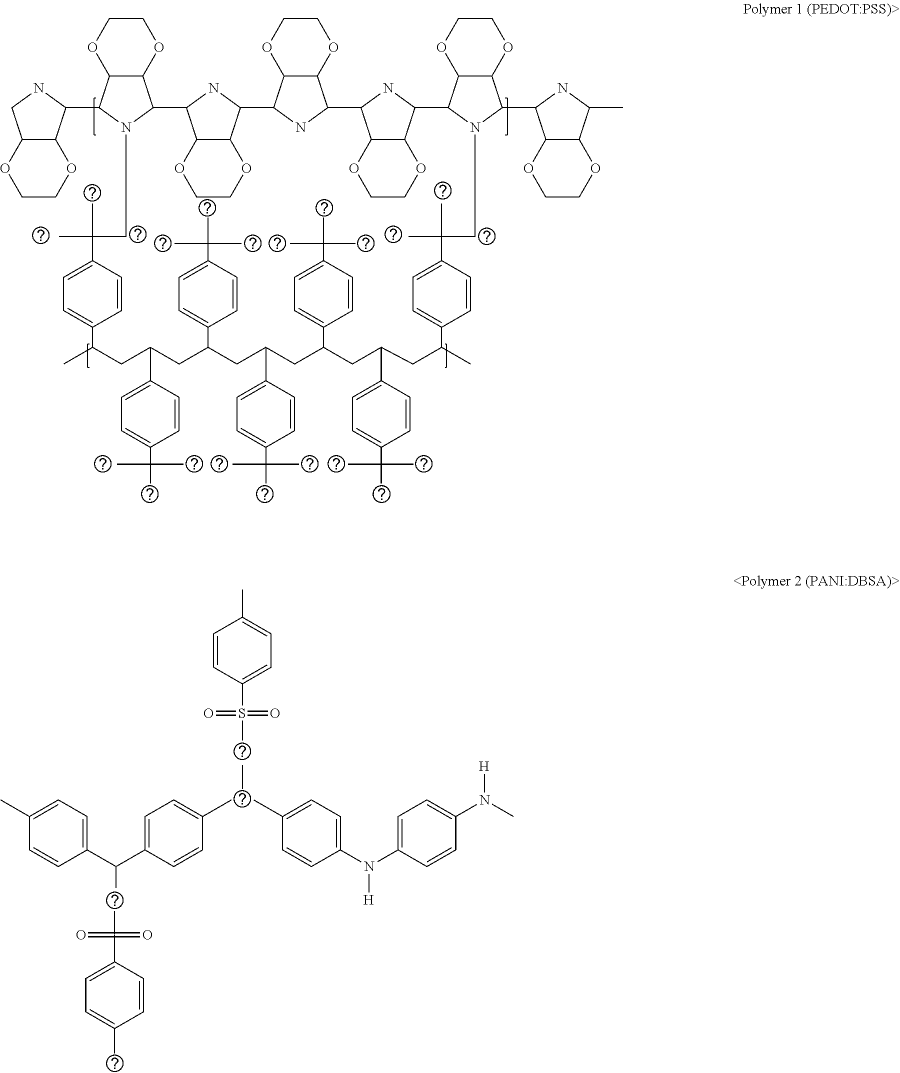

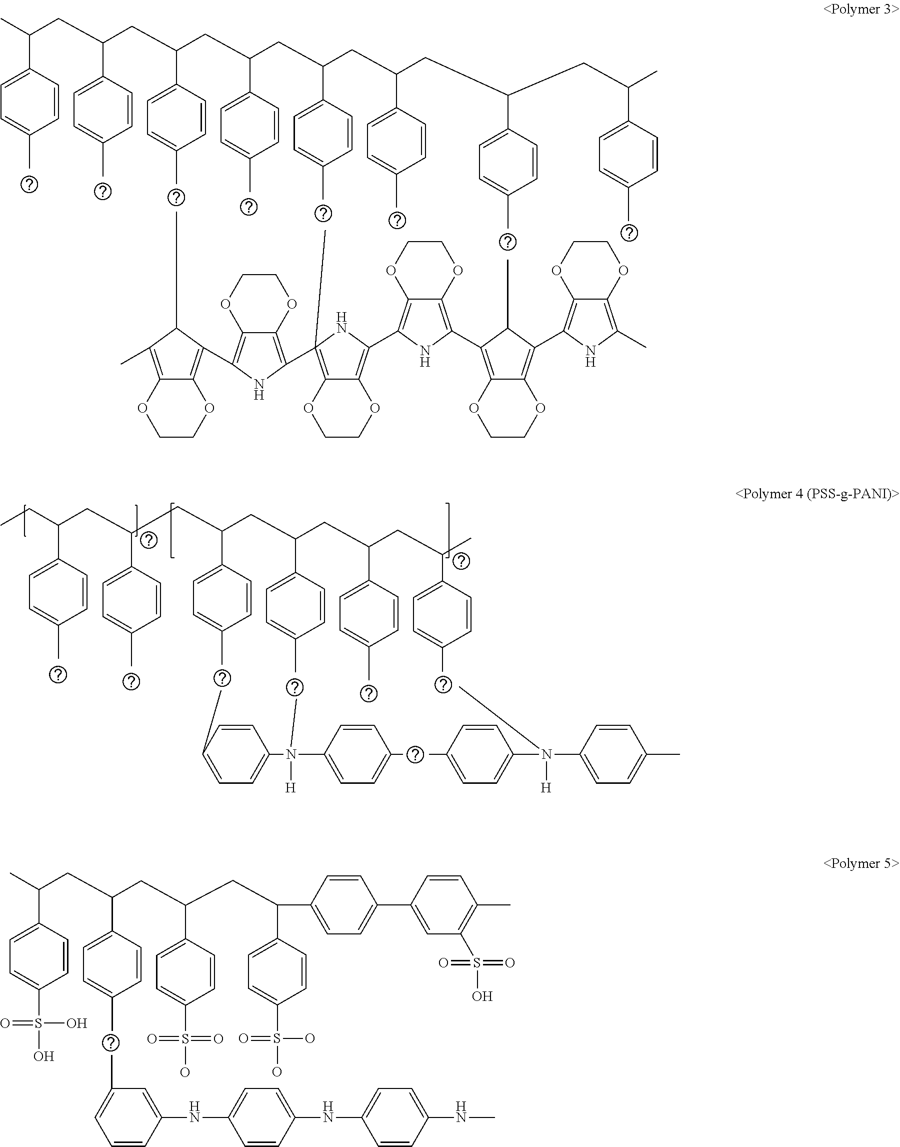

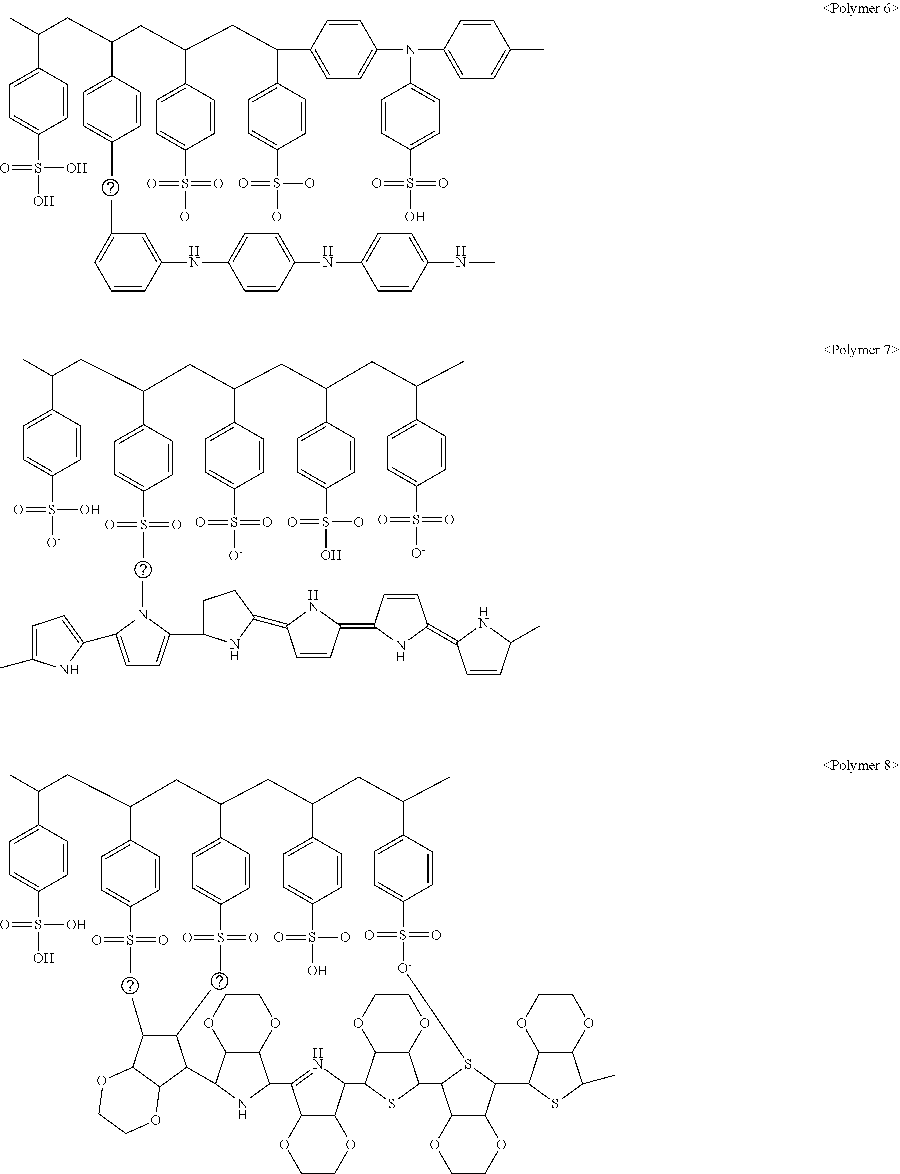

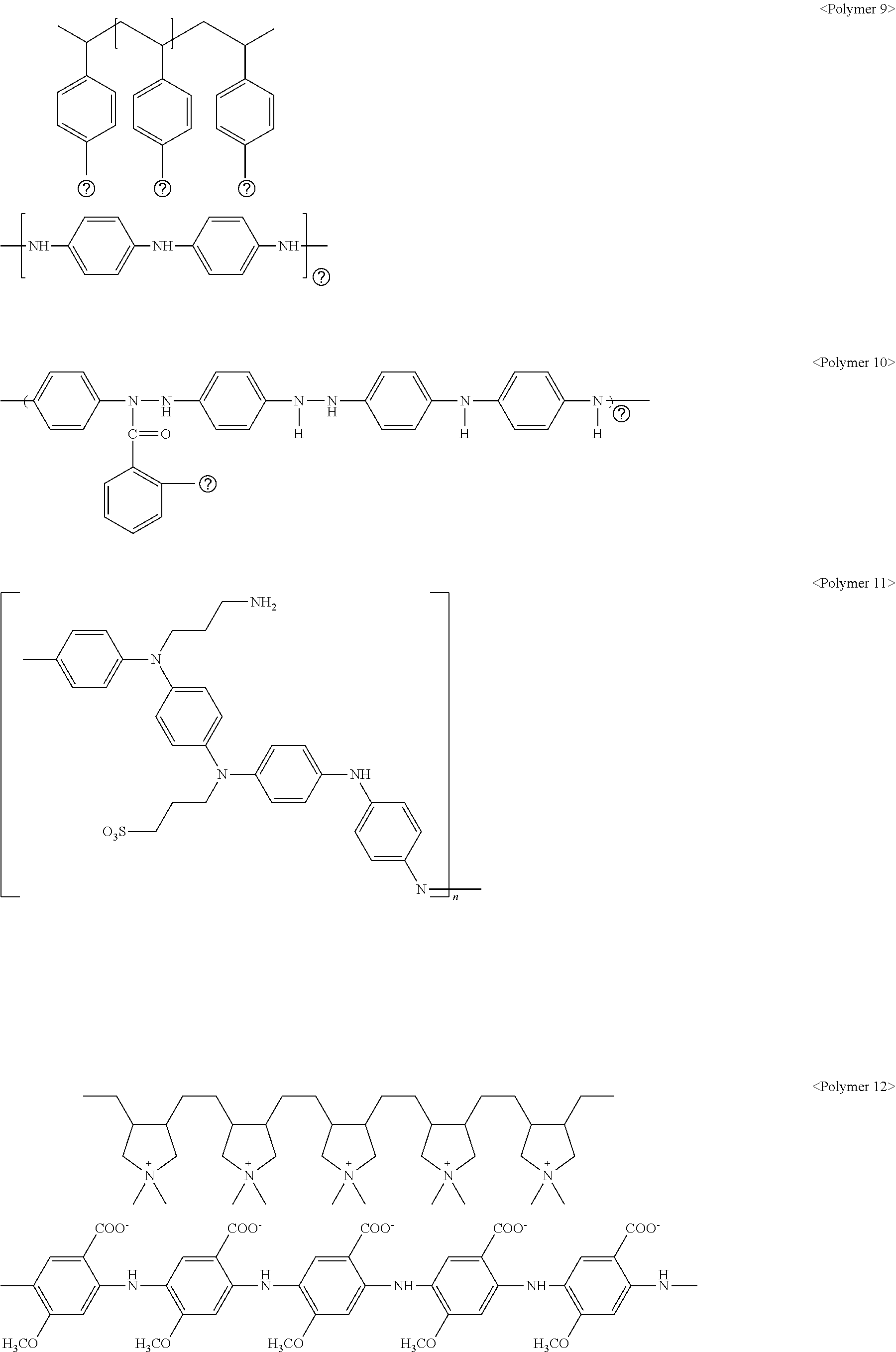

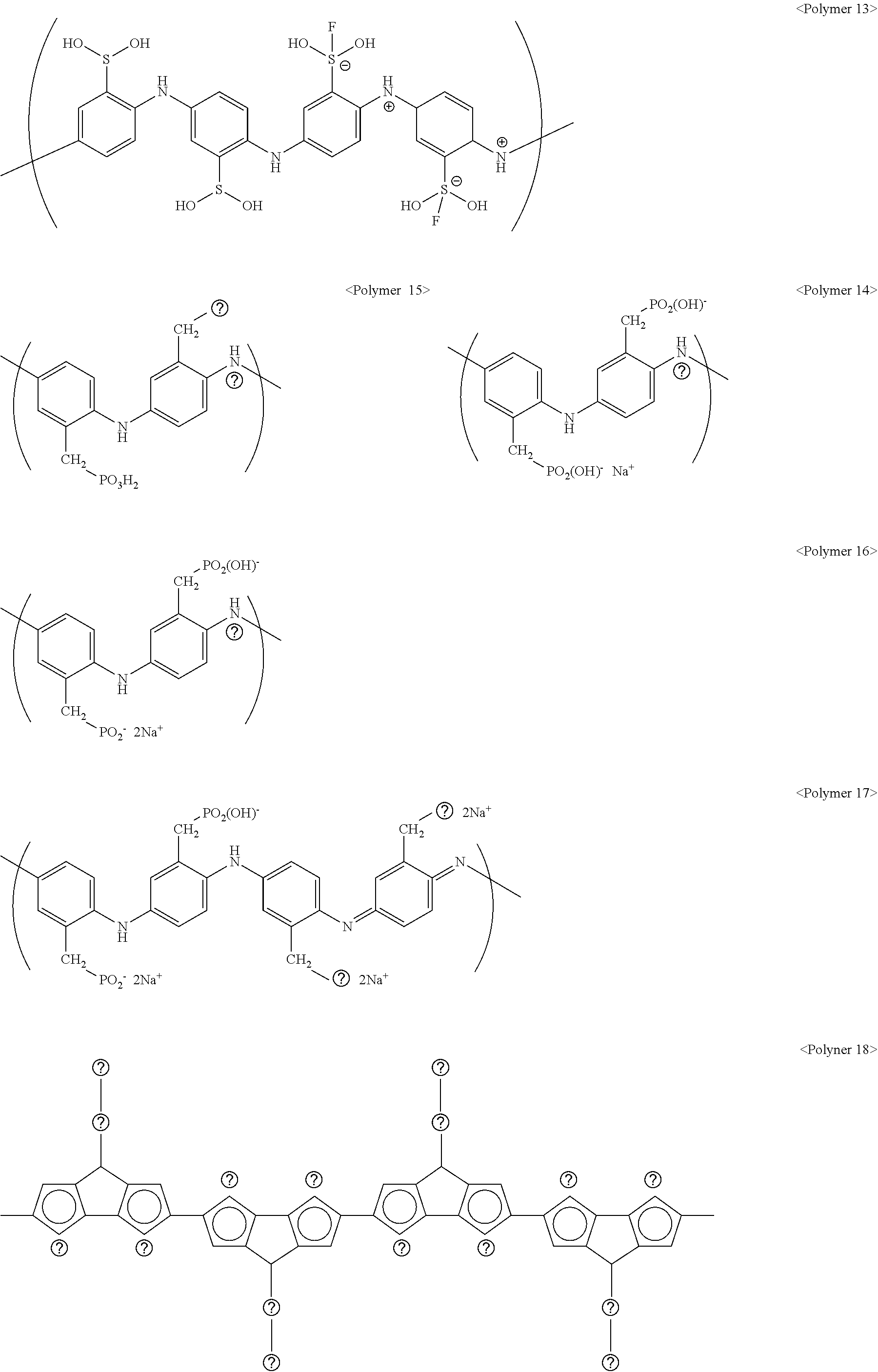

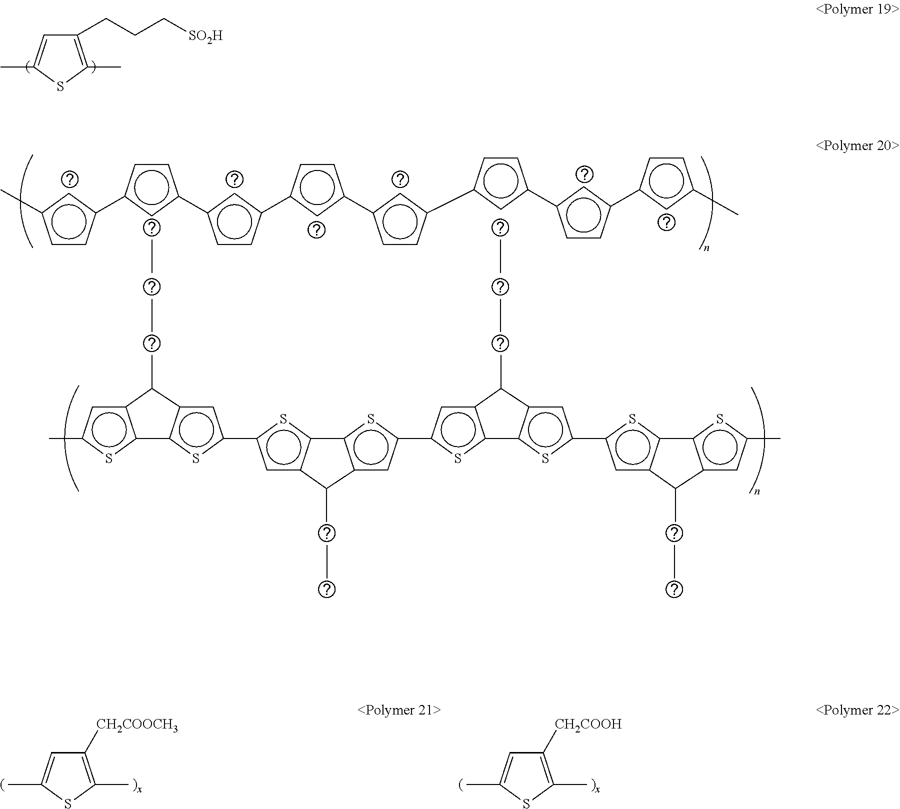

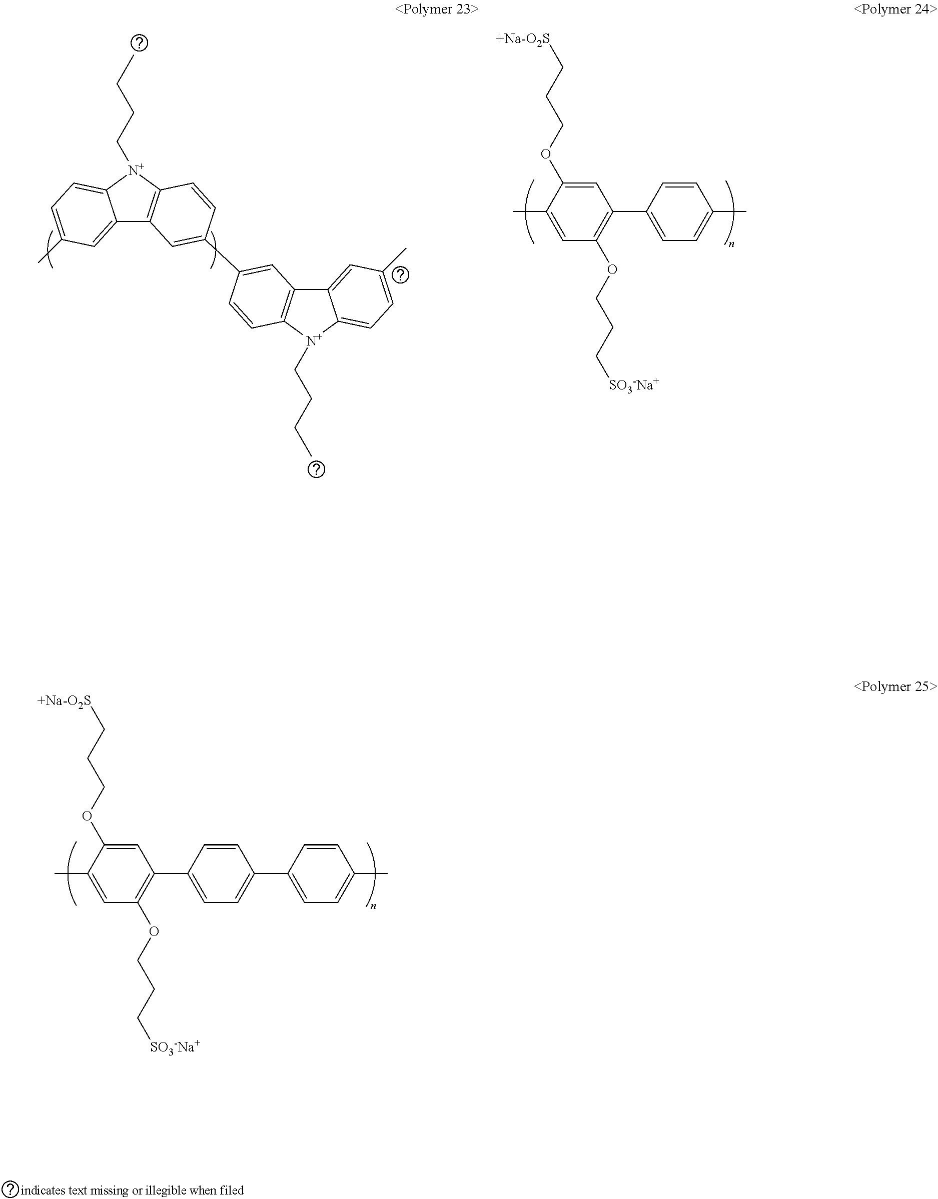

Specific examples of non-replaceable alkyl in this specification include methyl, ethyl, profile, isobutyl, sec-butyl, tert-butyl, pentyl, iso-amyl, hexyl, and one or more of the hydrogen atoms contained in the aforementioned alkyl can be substituted to halogen atoms, hydroxyl, nitro group, cyano group (—NH₂, —NH(R), —N(R′)(R″) and R′ and R″ independently of each other), amidino, hydrazine, hydrazone, carboxyl, sulphonic acid, phosphoric acid alkyl of C₁-C₂₀, halogenated alkyl of C₁-C₂₀, alkenyl of C₁-C₂₀, alkynyl of C₁-C₂₀, heteroalkyl of C₁-C₂₀ arylliche of C₁-C₂₀, a heteroalkyl of C₆-C₂₀, a heteroalkyl of C₆-C₂₀, or a heteroarylalkyl of C₆-C₂₀.

The heteroalkyl group of the present specification means that at least one of the carbon atoms in the main chain of the above-described alkyl group, preferably 1 to 5 carbon atoms, is substituted with a hetero atom such as an oxygen atom, a sulfur atom, a nitrogen atom, or a number of atoms.

Aryl groups herein refer to a carbocycle aromatic system that includes multiple aromatic rings, and the rings described above can be attached or fused together in a pendant method. Specific examples of the aryl group include aromatic groups such as phenyl, naphthyl, and tetrahydronaphthyl, and one or more hydrogen atoms of the above-mentioned aryl group can be substituted with the same substituents as in the case of the above-mentioned alkyl group.

The heteroaryl groups herein contain one, two or three heteroatoms selected from N, O, P or S, means a ring aromatic system having 5 to 30 ring atoms in which the remaining ring atoms are C, and the rings described above can be attached or fused together in a pendant manner. Then, one or more hydrogen atoms of the heteroaryl group described above can be substituted with the same substituents as in the case of the alkyl group described above.

Alkoxy group in this statement indicates radical-O-alkyl herein, and the alkyl is as defined above. Specific examples include methoxy, ethoxy, propoxy, isobutyloxy, sec-butyloxy, pentyloxy, iso-amyloxy, hexyloxy and the like, and one or more hydrogen atoms of the above-mentioned alkoxy group mentioned above can be substituted to above-mentioned substituents similar to those for alkyl groups.

The substituent heteroalkoxy group used in the present disclosure is essentially a heteroalkoxy group, except that one or more heteroatoms, such as oxygen, sulfur or nitrogen, can be present in the alkyl chain, it has the above-mentioned meaning of alkoxy, for example, CH₃CH₂OCH₂CH₂O—, C₄H₉OCH₂CH₂OCH₂CH₂O— and CH₃O(CH₂CH₂O)ₙH and so on.

Arylalkyl groups herein are aryl groups as defined above, meaning that some of the hydrogen atoms have been replaced with lower alkyl radicals such as methyl, ethyl, propyl and the like. For example, there are benzyl, phenylethyl and the like. One or more hydrogen atoms of the allyl alkyl group described above can be substituted with the same substituents as in the case of the alkyl group described above.

The heteroarylalkyl group of the present specification means that some of the hydrogen atoms of the heteroaryl group are substituted with a lower alkyl group, and the definition of heteroaryl among the heteroarylalkyl groups is as described above. At least one hydrogen atom in the aforementioned heteroarylalkyl group may be substituted with the same substituent as in the case of the aforementioned alkyl group.

The aryloxy in this specification refers to radical-O-aryl, where the aryl is defined above. Specific examples include phenoxy, naphtoxy, antracenyloxy, phenantrenyloxy, fluoreneyloxy, indenyloxy, etc., and one or more of the aryloxy's hydrogen atoms can be replaced by a replacement as in the case of the aforementioned alkyl group.

The heteroaryloxy group herein refers to a radical-O-heteroaryl, at this time, heteroaryl is as defined above.

Specific examples of the heteroaryloxy group in the present specification include a benzyloxy, a phenylethyloxy group, and the like, and one or more hydrogen atoms of the heteroaryloxy group can be substituted with a substituent same as in the case of the above-mentioned alkyl group.

The cycloalkyl group herein means a monocyclic system in which 1 of 5 to 30 carbon atoms is used. At least one or more hydrogen atoms of the above-mentioned cycloalkyl groups can be substituted with the same substituents as in the case of the above-mentioned alkyl groups.

The heterocycloalkyl groups herein contain one, two or three heteroatoms selected from N, O, P or S, with the remaining ring atoms being C from 5 to 5 ring atoms. 1 of 30 means a monocyclic system. One or more hydrogen atoms of the cycloalkyl group described above can be substituted with the same substituents as in the case of the alkyl group described above.

The alkyl ester group herein means a functional group that is ester-bonded to an alkyl group, where the alkyl group is as defined above.

The heteroalkyl ester group of the present specification means a functional group in which an ester group is bonded to the heteroalkyl group, and the heteroalkyl group described above is as defined above.

Aryl ester group herein means a functional group to which an allyl group and an ester group are attached, where the aryl group is as defined above.

The heteroaryl ester group herein means a functional group to which a heteroaryl group and an ester group are bonded, at which time the heteroaryl group is as defined above.

The amino group used in the present disclosure means —NH₂, —NH(R) or —N(R′)(R″), where R′ and R″ are independent of each other and have a carbon number of from 1 to 10.

The halogens herein are fluorine, chlorine, bromine, iodine, or astatine, of which fluorine is particularly preferred.

The aforementioned metallic carbon nanotubes may be either refined metallic carbon nanotubes themselves or carbon nanotubes with metal particles (e.g., Ag, Au, Cu, Pt particles, etc.) attached to the inner and/or outer walls of carbon nanotubes.

The above-mentioned graphene is possible to have a graphene single layer having a thickness of about 0.34 nm, a few layers graphene having a structure in which 2 to 10 graphene single layers are laminated, or a graphene multilayer structure having a structure in which a larger number of graphene single layers are stacked than the above-described a few layers graphene.

The metal nanowires and semiconductor nanowires described above include, for example, Ag, Au, Cu, Pt NiSix (NickelSilicide) nanowires and nanowire with two or more composites of these (eg alloys or core-shell composites), but are not limited thereto.

Alternatively, the semiconductor nanowires described above are Si, Ge, B, or N-doped Si nanowires, B, or N-doped Ge nanowires and composites of two or more of these (eg. Alloy or core-shell structures, etc.), but are not limited to thereto.

The aforementioned metal nanowires and semiconductor nanowires can be between 5 nm and 100 nm in diameter and can be between 500 nm and 100 μm in length, depending on the manufacturing method of the aforementioned metal nanowires and semiconductor nanowires.

The above-described metal grid is formed by using Ag, Au, Cu, Al, Pt and their alloys to form a mesh-shaped metal line crossing each other, and can have a line width of 100 nm to 100 μm, and the length is not limited. The above-described metal grid may be formed to protrude on the first electrode or may be inserted into the first electrode to form a recessed shape.

The metal quantum dots mentioned above can be selected from Ag, Au, Cu, Pt and two or more composites quantum dots of these (eg. alloys or core-shell structures), but it is not limited to this.

On the surface of the metal nanowires, semiconductor nanowires, and metal nanodots described above, at least one portion (here, Z₁₀₀, Z₁₀₁, Z₁₀₂, and Z₁₀₃ described above are independently bonded to hydrogen, halogen atoms, substituted or unsubstituted C₁-C₂₀ alkyl groups or substituted or unsubstituted C₁-C₂₀ alkoxy group) labeled as —S(Z₁₀₀) and —Si(Z₁₀₁)(Z₁₀₂)(Z₁₀₃). At least one moiety represented by —S(Z₁₀₀) and —Si(Z₁₀₁)(Z₁₀₂)(Z₁₀₃) described above is a self-assembled moiety, and through the moiety described above, a metal nanowire, a semiconductor nanowire and the bonding between metal nanodots or metal nanowires, semiconductor nanowires, and the bonding force between the metal nanodots and the first electrode (210) may be strengthened, thereby further improving electrical properties and mechanical strength.

The conductive oxide described above can be any of ITO (indium tin oxide), IZO (indium zinc oxide), SnO₂ and InO₂.

The steps of forming the above-mentioned conductive layer (31) on the above-mentioned first electrode (20) are spin coating method, casting method, quantity Langmuir-blog jet method, inkjet printing, nozzle printing method, slot die coating method, doctor blade coating method, screen printing method, dip coating method, gravure printing method, physical transfer method, spray coating method), chemical vapor deposition method or thermal evaporation method

Further, the above-mentioned conductive material is mixed with a solvent to produce a mixed solution, which is then applied onto the above-mentioned first electrode (10), and then heat-treated is conducted by removing the above-mentioned solvent. At this time, the above-mentioned solvent can be polar solvent, and, for example, may include at least one selected from the group consisting of water, alcohol (methanol, ethanol, n-propanol, 2-propanol, n-butanol, etc.), formic acid, nitromethane. Acetic acid, ethylene glycol, glycerol, normal methylpyrrolidone (NMP, n-Methyl-2-Pyrrolidone), N-dimethylacetamide, dimethylformamide (DMF, dimethylformamide), dimethyl sulfoxide (DMSO, dimethyl sulfoxide), tetrahydrofuran (THF, tetrahydrofuran), ethyl acetate (EtOAc, ethyl acetate), acetone (acetone), and acetonitrile (MeCN, acetonitrile).

When the above-mentioned conductive layer (31) contains metal carbon nanotubes, the metal carbon nanotubes may be grown on the above-mentioned first electrode (20), or the carbon nanotubes dispersed in a solvent may be printed by a solution-based printing method (eg. Spray coating method, spin coating method, dip coating method, empty coating method, reverse offset coating method, screen printing method, slot-die coating method)

When the above-mentioned conductive layer (31) contains a metal grid, metal is vacuum-deposited on the above-mentioned first electrode (20) to form a metal film, and then various mesh shapes are formed by a photolithography, or printing method (eg, spray coating method, spin coating method, dip coating method, empty coating method, reverse offset coating method, screen printing method, slot die coating method) by patterning the metal precursor or metal particles in a solvent).

The above-mentioned conductive layer (31) mainly plays a role of improving the conductivity in the above-mentioned exciton buffer layer (30), and additionally adjusts scattering, reflection, and absorption to improve optical extraction, and improving mechanical strength by imparting flexibility.

The surface buffer layer (32) described above contains a fluorinated material. At this time, the above-mentioned fluorine-based material is preferably a fluorine-based material having a lower surface energy than the above-mentioned conductive material, and can have a surface energy of 30 mN/m or less.

Further, the above-mentioned fluorine-based material can have a hydrophobicity larger than that of the above-mentioned conductive polymer.

At this time, the concentration of the above-described fluorine-based material on the second surface (32b) opposite to the above-described first surface (32a) can be lower than the concentration of the above-described fluorine-based material on the first surface (32a) close to the above-described conductive layer (31) in the above-described surface buffer layer (32).

This means that the work function of the second surface (32b) of the surface buffer layer (32) described above can be 5.0 eV or higher. As an example, of the surface buffer layer (32) described above, the work function measured on the second surface (32b) can be 5.0 to 6.5 eV, but is not limited to this.

The fluorinated material described above can be a fluorinated ionomer or a fluorinated ionomer containing at least one F. In particular, when the above-mentioned fluorine-based material is a fluorinated ionomer, the thickness of the buffer layer can be formed to be thick, preventing phase separation between the conductive layer (31) and the surface buffer layer (32) and making it more uniform and allows the formation of an extensive exciton buffer layer. (30).

The above-mentioned fluorinated material can contain at least one ionomer selected from the group consisting of ionomers having the structures of the following chemical formulas 6 to 17.

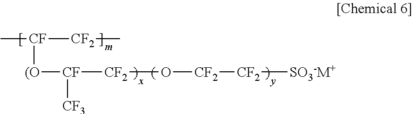

In the above equation, m is a number from 1 to 10,000,000, x and y are independently numbers from 0 to 10, and M is Na⁺, K⁺, Li⁺, H⁺, CH₃(CH₂)ₙNH₃⁺ (n is an integer from 0 to 50), NH₄⁺, NH₂⁺, NHSO₂CF₃⁺, CHO⁺, C₂H₅OH⁺, CH₃OH⁺, RCHO⁺ (R is CH₃(CH₂)ₙ—; n is 0 to 50 Integer) is shown.

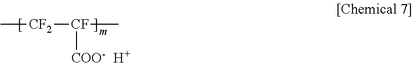

In the above equation, m is a number from 1 to 10,000,000.

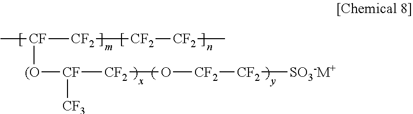

In the above equations, m and n are 0<m≤10,000,000, 0≤n<10,000,000, x and y are independently numbers from 0 to 20, and M is Na⁺, K⁺, Li⁺, H⁺, CH₃(CH₂)ₙNH₃⁺ (n is an integer from 0 to 50), NH₄⁺, NH₂⁺, NHSO₂CF₃⁺, CHO⁺, C₂H₅OH⁺, CH₃OH⁺, RCHO⁺ (R is CH₃(CH₂))ₙ—; n is an integer from 0 to 50).

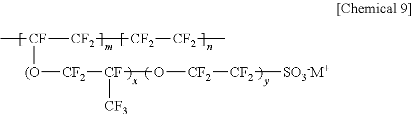

In the equations above, m and n are 0<m≤10,000,000, 0≤n<10,000,000, x and y are independently numbers from 0 to 20, and M is Na⁺, K⁺, Li⁺, H⁺, CH₃(CH₂)ₙNH₃⁺ (n is an integer from 0 to 50), NH₄⁺, NH₂⁺, NHSO₂CF₃⁺, CHO⁺, C₂H₅OH⁺, CH₃OH⁺, RCHO⁺ (R is CH₃(CH₂))ₙ—; n is an integer from 0 to 50)

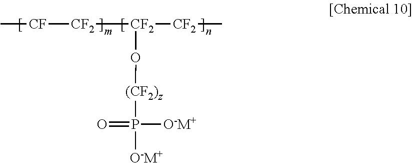

In the above equation, m and n are 0<m≤10,000,000, 0≤n<10,000,000, x and y are independently numbers from 0 to 20, and M is Na⁺, K⁺, Li⁺, H⁺, CH₃(CH₂)ₙNH₃⁺ (n is an integer from 0 to 50), NH₄⁺, NH₂⁺, NHSO₂CF₃⁺, CHO⁺, C₂H₅OH⁺, CH₃OH⁺, RCHO⁺ (R is CH₃(CH₂))ₙ—; n is an integer from 0 to 50).

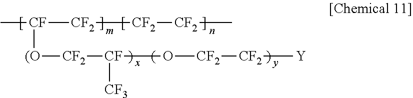

In the above equations, m and n are 0<m≤10,000,000, 0≤n<10,000,000, x and y are independently numbers from 0 to 20, and M is Na⁺, K⁺, Li⁺, H⁺, CH₃(CH₂)ₙNH₃⁺ (n is an integer from 0 to 50), NH₄⁺, NH₂⁺, NHSO₂CF₃⁺, CHO⁺, C₂H₅OH⁺, CH₃OH⁺, RCHO⁺ (R is CH₃(CH₂)ₙ—; n is an integer from 0 to 50).

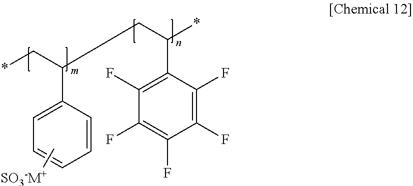

In the above equations, m and n are 0<m≤10,000,000, 0≤n<10,000,000, x and y are independently numbers from 0 to 20, and M is Na⁺, K⁺, Li⁺, H⁺, CH₃(CH₂)ₙNH₃⁺ (n is an integer from 0 to 50), NH₄⁺, NH₂⁺, NHSO₂CF₃⁺, CHO⁺, C₂H₅₀H⁺, CH₃OH, RCHO⁺ (R is CH₃(CH₂)ₙ—; n is an integer from 0 to 50).

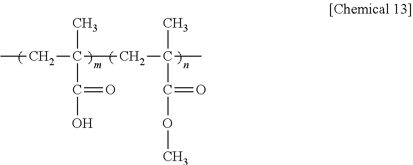

In the above equation, m and n are 0<m≤10,000,000 and 0≤n<10,000,000.

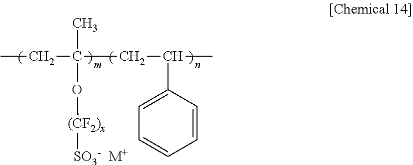

In the above equation, m and n are 0<m≤10,000,000, 0≤n<10,000,000, x and y are independently numbers from 0 to 20, and M is Na⁺, K⁺, Li⁺, H⁺, CH₃(CH₂)ₙNH₃⁺ (n is an integer from 0 to 50), NH₄⁺, NH₂⁺, NHSO₂CF₃⁺, CHOP, C₂H₅OH⁺, CH₃OH⁺, RCHO⁺ (R is CH₃(CH₂)ₙ—; n is an integer from 0 to 50).

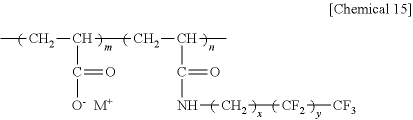

In the equations above, m and n are 0<m≤10,000,000, 0≤n<10,000,000, x and y are independent numbers from 0 to 20, and M is Na⁺, K⁺, Li⁺, H⁺, CH₃(CH₂)ₙNH₃⁺ (n is an integer from 0 to 50), NH₄⁺, NH₂⁺, NHSO₂CF₃⁺, CHO⁺, C₂H₅OH⁺, CH₃OH, RCHO⁺ (R is CH₃(CH₂)ₙ—; n is an integer from 0 to 50).

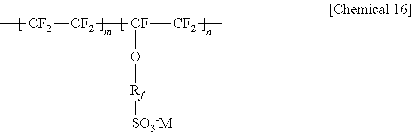

In the equations above, m and n are 0<m≤10,000,000, 0≤n<10,000,000, x and y are independently numbers from 0 to 20, and M is Na⁺, K⁺, Li⁺, H⁺, CH₃(CH₂)ₙNH₃⁺ (n is an integer from 0 to 50), NH₄⁺, NH₂⁺, NHSO₂CF₃⁺, CHOP, C₂H₅OH⁺, CH₃OH⁺, RCHO⁺ (R is CH₃(CH₂)ₙ—; n is an integer from 0 to 50).

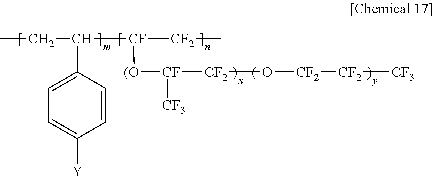

In the equations described above, m and n are 0<m≤10,000,000, 0≤n<10,000,000, x and y are independently numbers from 0 to 20, and M is Na⁺, K⁺, Li⁺, H⁺, CH₃(CH₂)ₙNH₃+(n is an integer from 0 to 50), NH₄⁺, NH₂⁺, NHSO₂CF₃⁺, CHOP, C₂H₅OH⁺, CH₃OH⁺, RCHO⁺ (R is CH₃ (R is CH₃(R)CH₂)ₙ—; n is an integer from 0 to 50).

Further, the above-mentioned fluorine-based material can contain at least one ionomer or low fluoride molecule selected from the group consisting of ionomers or low fluoride molecules having the structures of the following chemical formulas 18 to 22.

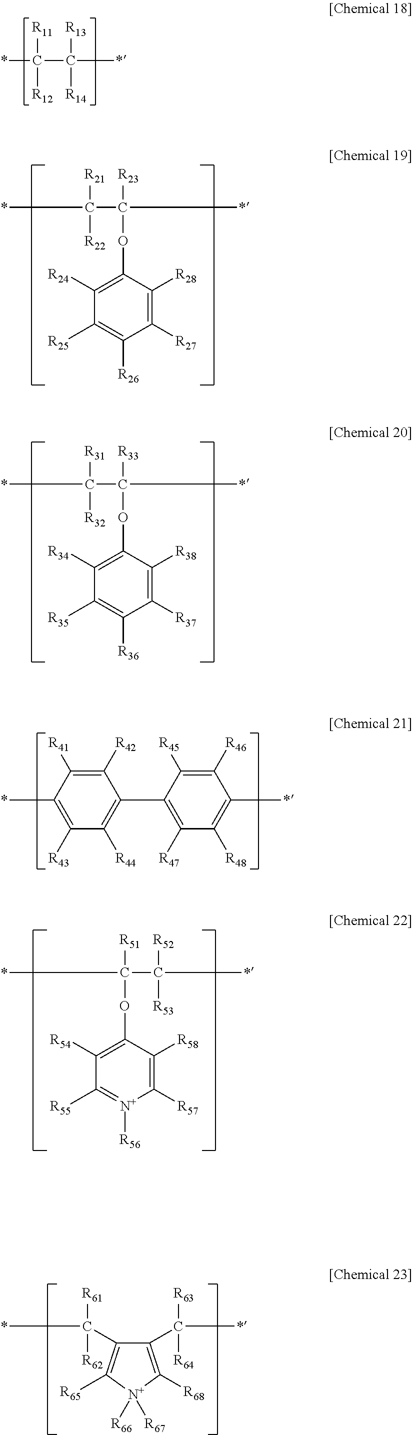

R₁₁ to R₁₄, R₂₁ to R₂₈, R₃₁ to R₃₈, R₄₁ to R₄₈, R₅₁ to R₅₈ and R₆₁ to R₆₈ are independently be chosen among, hydrogen, —F, C₁-C₂₀ alkyl groups and C₁-C₂₀ alkoxy groups, at t least one —F substituted C₁-C₂₀ alkyl group, at least one —F substituted C₁-C₂₀ alkoxy group, Q₁, —O—(CF₂CF(CF₃)—O)n-(CF₂)m-Q₂ (where n and m are independent of each other and are integers from 0 to 20, n⁺ m is greater than or equal to 1) and —(OCF₂CF₂)x-Q₃ (where x is an integer from 1 to 20)),

The aforementioned Q₁ to Q₃ are ionic groups, the aforementioned ionic groups include anionic groups and cationic groups, the aforementioned anionic groups are selected from PO₃²⁻, SO₃⁻, COO⁻, I⁻, CH₃COO⁻, and the aforementioned cations include at least one of metal ions and organic ions, the aforementioned metal ion is selected from Na⁺, K⁺, Li⁺, Mg²⁺, Zn²⁺, and Al³⁺, and the aforementioned organic ion is H⁺, CH₃(CH₂)nNH₃⁺ (n is an integer of 0 to 50), NH₄⁺, NH₂⁺, NHSO₂CF₃⁺, CHO⁺, C₂H₅OH⁺,

at least one of R₁₁ to R₁₄, at least one of R₂₁ to R₂₈, at least one of R₃₁ to R₃₈, and of R₄₁ to R₄₈ at least one, at least one of R₅₁ to R₅₈ and at least one of R₆₁ to R₆₈ are selected from —F, at least one —F substituted C₁-C₂₀ alkyl group, at least one —F substituted C₁-C₂₀ alkoxy groups —O—(CF₂CF(CF₃)—O)n-(CF₂)m-Q₂ and —(OCF₂CF₂)x-Q₃.)

X-M^{f}ₙ-M^{h}ₘ-M^{a}ᵣ-G. [Chemical 24]

(in the chemical formula 24,

X is the end;

M^{f}ₙ represents unit derived from fluorinated monomers obtained from perfluoropolyether alcohol, polyisocyanate and isocyanate reactive-non-fluorinated monomer.

M^{h}ₘ represents a unit derived from a non-fluorinated monomer;

M^{a}ᵣ represents the unit having a silyl group represented by —Si(Y₄)(Y₅)(Y₆);

Y₄, Y₅ and Y₆ described above represent substituted or unsubstituted C₁-C₂₀ alkyl groups, substituted or unsubstituted C₆-C₃₀ aryl groups or hydrolyzable substituents independently of each other, as described above. At least one of Y₄, Y₅ and Y₆ was the hydrolyzable substituents mentioned above;

G is an organic group of 1 containing residues of the chain transfer agent;

n is a number from 1 to 100;

, m is a number between 0 and 100;

r is a number between 0 and 100;

n+m+r is at least 2).

The thickness of the surface buffer layer (32) described above can be 1 nm to 500 nm. For example, the thickness of the surface buffer layer mentioned above may include a range in which a lower value of two numbers, has a lower limit value and a higher value of two numbers has an upper limit value among 1 nm, 2 nm, 3 nm, 4 nm, 5 nm, 6 nm, 7 nm, 8 nm, 9 nm, 10 nm, 11 nm, 12 nm, 13 nm, 14 nm, 15 nm, 16 nm, 17 nm, 18 nm, 19 nm, 20 nm, 21 nm, 22 nm, 23 nm, 24 nm, 25 nm, 26 nm, 27 nm, 28 nm, 29 nm, 30 nm, 31 nm, 32 nm, 33 nm, 34 nm, 35 nm, 36 nm, 37 nm, 38 nm, 39 nm, 40 nm, 41 nm, 42 nm, 43 nm, 44 nm, 45 nm, 46 nm, 47 nm, 48 nm, 49 nm, 50 nm, 51 nm, 52 nm, 53 nm, 54 nm, 55 nm, 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 101 nm, 102 nm, 103 nm, 104 nm, 105 nm, 106 nm, 107 nm, 108 nm, 109 nm, 110 nm, 111 nm, 112 nm, 113 nm, 114 nm, 115 nm, 116 nm, 117 nm, 118 nm, 119 nm, 120 nm, 125 nm, 130 nm, 135 nm, 140 nm, 145 nm, 150 nm, 155 nm, 160 nm, 165 nm, 170 nm, 175 nm, 180 nm, 185 nm, 190 nm, 195 nm, 200 nm, 210 nm, 220 nm, 230 nm, 240 nm, 250 nm, 260 nm, 270 nm, 280 nm, 290 nm, 300 nm, 310 nm, 320 nm, 330 nm, 340 nm, 350 nm, 360 nm, 370 nm, 380 nm, 390 nm, 400 nm, 450 nm, 500 nm. Also, preferably, the thickness of the surface buffer layer described above can be 10 nm to 200 nm. If the thickness of the surface buffer layer (32) described above satisfies the above range, excellent work function characteristics, transparency and flexibility characteristics can be provided.

The above-mentioned surface buffer layer (32) can be formed by producing a mixed solution containing the above-mentioned fluorine-based material and solvent on the above-mentioned conductive layer (31) and then heat-treating the mixed solution.

The exciton buffer layer (30) thus formed can have a thickness of 1 nm to 500 nm. Among 1 nm, 2 nm, 3 nm, 4 nm, 5 nm, 6 nm, 7 nm, 8 nm, 9 nm, 10 nm, 11 nm, 12 nm, 13 nm, 14 nm, 15 nm, 16 nm, 17 nm, 18 nm, 19 nm, 20 nm, 21 nm, 22 nm, 23 nm, 24 nm, 25 nm, 26 nm, 27 nm, 28 nm, 29 nm, 30 nm, 31 nm, 32 nm, 33 nm, 34 nm, 35 nm, 36 nm, 37 nm, 38 nm, 39 nm, 40 nm, 41 nm, 42 nm, 43 nm, 44 nm, 45 nm, 46 nm, 47 nm, 48 nm, 49 nm, 50 nm, 51 nm, 52 nm, 53 nm, 54 nm, 55 nm, 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 101 nm, 102 nm, 103 nm, 104 nm, 105 nm, 106 nm, 107 nm, 108 nm, 109 nm, 110 nm, 111 nm, 112 nm, 113 nm, 114 nm, 115 nm, 116 nm, 117 nm, 118 nm, 119 nm, 120 nm, 125 nm, 130 nm, 135 nm, 140 nm, 145 nm, 150 nm, 155 nm, 160 nm, 165 nm, 170 nm, 175 nm, 180 nm, 185 nm, 190 nm, 195 nm, 200 nm, 210 nm, 220 nm, 230 nm, 240 nm, 250 nm, 260 nm, 270 nm, 280 nm, 290 nm, 300 nm, 310 nm, 320 nm, 330 nm, 340 nm, 350 nm, 360 nm, 370 nm, 380 nm, 390 nm, 400 nm, 450 nm, 500 nm, a range in which a lower value of the two numbers is a lower limit value and a higher value is an upper limit value may be included. Also, preferably, the thickness of the surface buffer layer described above can be 10 nm to 100 nm. If the thickness of the exciton buffer layer described above satisfies the above range, excellent work function characteristics, transparency and flexibility characteristics can be provided.

The conductivity can be improved as the conductive layer (31) described above is formed, and at the same time, the surface energy can be lowered as the surface buffer layer (32) described above is formed. Thereby, the light emission characteristic can be maximized.

The above-described surface buffer layer (32) may further include at least one additive selected from the group consisting of carbon nanotubes, graphene, reduced graphene oxide, metal nanowires, metal carbon nanodots, semiconductor quantum dots, semiconductor nanowires, and metal nanodots. When the above-described additive is further included, it is possible to maximize the conductivity improvement of the exciton buffer layer (30) described above.

Further, the surface buffer layer (32) described above may further contain a cross-linking agent containing a Bis(phenyl azide) material. When the above-mentioned cross-linking agent is further contained in the above-mentioned surface buffer layer (32), composition separation due to time and device drive can be prevented. This can improve the stability and reproducibility of the light-emitting diodes, which reduces the resistance and work function of the exciton buffer layer (30) described above.

The above-mentioned bisphenyl azide-based material can be a bisphenyl azide-based material having the following chemical formula 25.

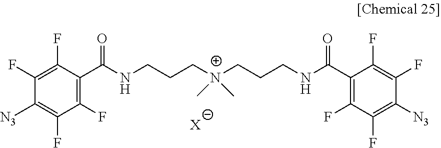

The steps of forming the above-mentioned surface buffer layer (32) on the above-mentioned conductive layer (31) can use spin coating method, casting method, Langmuir-blodgett method, inkjet printing method, nozzle printing method, slot die coating method, doctor blade coating method, screen printing method, dip coating method, gravure printing method, reverse-offset printing method, spray coating method, chemical vapor deposition method or thermal evaporation method process.

However, in the step of forming the exciton buffer layer (30) described above, the conductive layer (31) and the surface buffer layer (32) may be sequentially deposited as described above, but after preparing a mixed solution by mixing the above-described conductive material and the above-described fluorine-based material in a solvent, it can be formed through a process of heat treatment by applying the mixed solution to the above-described first electrode.

In this case, by heat-treating the above-mentioned mixed solution, the conductive layer (31) and the surface buffer layer (32) are sequentially assembled and formed on the above-mentioned first electrode (20). This has the advantage that the process can be simplified.

The above-mentioned fluorine-based material can be a material having a solubility of 90% or more, for example, 95% or more in a polar solvent. Examples of the aprotic solvents mentioned above include water, alcohols (methanol, ethanol, n-propanol, 2-propanol, n-butanol, etc.), ethylene glycol, glycerol, dimethylformamide (DMF), dimethyl sulfoxide (DMSO), acetone, but they are not limited to these.

The exciton buffer layer (30) described above may further contain a cross-linking agent.

By adding a cross-linking agent to the exciton buffer layer (30) described above, it is possible to prevent phase separation of constituent materials from occurring depending on the time and drive of the device. Further, it is possible to prevent the efficiency of the exciton buffer layer (30) from being lowered due to the use of a solvent or the like during the formation of the surface buffer layer (32) described above. Therefore, the stability and reproducibility of the device can be improved.

The cross-linking agent described above can include at least one functional group selected from the group consisting of amine groups (—NH₂), thiol groups (—SH), and carboxyl groups (—COO—).

The above-mentioned cross-linking agents can include at least one selected from the group consisting of include bisphenyl azide-based materials, diaminoalkane-based materials, dithiol-based materials, dicarboxylate, and ethylene glycol, ethylene glycol di(meth)acrylate derivatives, methylenebis azine derivatives, and divinylbenzene (DVB).

A hole transport layer (not shown) can be formed on the exciton buffer layer (30) described above. The hole transport layer described above can be formed based on a method arbitrarily selected from various known methods such as a vacuum deposition method, a spin coating method, a casting method, and an LB method. At this time, when the vacuum vapor deposition method is selected, the vapor deposition conditions differ depending on the target compound, the structure and thermal properties of the target layer, and the like, for example, the vapor deposition temperature range of 100 to 500, vacuum range from 10⁻¹⁰ to 10⁻³ torr, the deposition rate range of 100 Å/sec can be selected. On the other hand, when the spin coating method is selected, the coating conditions vary depending on the target compound, the structure and thermal properties of the target layer, but the coating rate range from 2000 rpm to 5000 rpm and annealing temperature(heat treatment temperature for removing solvent after coating) from 80 to 200° C. can be selected.

Y₁, which is the value of the work function of the first surface (32a) of the surface buffer layer (32) of the exciton buffer layer (30) described above, can be in the range of 4.6 to 5.2, for example, 4.7 to 4.9. Y₂, which is the value of the work function of the second surface (32b) of the surface buffer layer (32) of the exciton buffer layer (30) described above, can be equal to or smaller than the work function of fluorine-based material contained in the surface buffer layer (32) described above. For example, the aforementioned Y₂ can be in the range of 5.0 to 6.5, for example 5.3 to 6.2, but is not limited thereto.

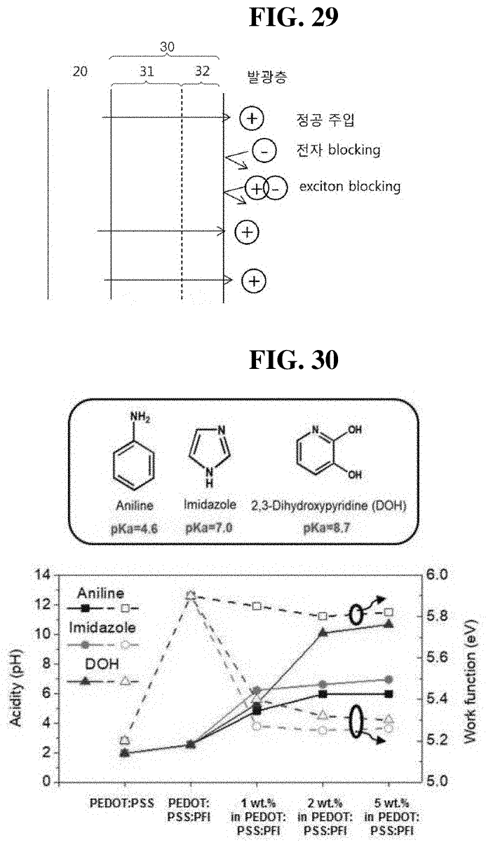

FIG. 29 shows the effect of the exciton buffer layer (30) according to the embodiment of the present disclosure.

Referring to FIG. 29, it can be seen that the exciton buffer layer (30) according to an embodiment of the present disclosure improves hole injection efficiency, performs an electron blocking role, and suppresses quenching of excitons.

<Metal Halide Perovskite Light-Emitting Diodes Containing an Acidity-Controlled Conductive Polymer>

According to an embodiment of the present disclosure, the metal halide perovskite light-emitting diodes can include a conductive polymer composition containing a fluorine-based material and a basic material.

Also, preferably, the conductive polymer composition is basic materials or a fluorinated materials, and it is possible that the material was neutralized at pH 4.0 to 10.0 with a work function of 5.8 eV or higher.

Also, preferably, the conductive polymer composition is added by the addition of a basic material. The surface roughness of the thin film may have decreased below 2 nm.

In addition, preferably, the conductive polymer includes polythiophene, polyaniline, polypyrrole, polystyrene, polyethylenedioxythiophene, polyacetylene, polyphenylene, polyphenylvinylene, polycarbazole, and copolymers which contains at least two different repeating units of these, their derivatives, or blends of two or more of them.

Further, preferably, the above-mentioned fluorine-based material can be an ionomer represented by the following chemical formula 26.

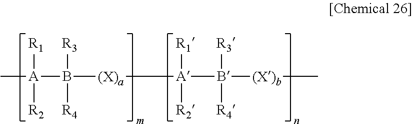

(in the above chemical formula 26,

0<m≤10,000,000, 0≤n<10,000,000, 0≤a≤20, 0≤b≤20;

A, B, A′ and B′ are independently selected from the group consisting of C, Si, Ge, Sn, and Pb;

R₁, R₂, R₃, R₄, R₁′, R₂′, R₃′ and R₄′ can be independently selected from the group consisting of hydrogen, halogen, nitro groups, substituted or unsubstituted amino groups, cyano groups, substituted or non-substituted, substituted C₁-C₃₀ alkyl groups, substituted or unsubstituted C₁-C₃₀ heteroalkyl groups, substituted or unsubstituted C₁-C₃₀ alkoxy groups, substituted or unsubstituted C₁-C₃₀ heteroalkryl groups, substituted or unsubstituted, substituted C₆-C₃₀ aryl groups, substituted or substituted C₆-C₃₀ arylalkyl groups, substituted or unsubstituted C₆-C₃₀ aryloxy groups, substituted or unsubstituted C₂-C₃₀ heteroaryl groups, substituted or unsubstituted C₂-C₃₀ heteroarylalkyl groups, substituted or substituted C₂-C₃₀ heteroaryloxy groups, substituted or unsubstituted C₅-C₂₀ cycloalkyl groups, substituted or unsubstituted C₂-C₃₀ heterocycloalkyl groups, substituted or unsubstituted C₁-C₃₀ alkyl ester groups, substituted or unsubstituted C₁-C₃₀ heteroalkyl ester groups, substituted or unsubstituted C₆-C₃₀ aryl ester groups, substituted or unsubstituted C₂-C₃₀ heteroaryl ester groups, at least one or more of R₁, R₂, R₃, and R₄ are ionic groups or contain ionic groups;

X and X′ are independently selected from the group consisting of simple bond, O, S, substituted or unsubstituted C₁-C₃₀ alkyl group, substituted or unsubstituted C₁-C₃₀ heteroalkyl group, substituted or unsubstituted C₆-C₃₀ Allyl group, substituted or unsubstituted C₆-C₃₀ arylalkyl group, substituted or unsubstituted C₂-C₃₀ heteroaryl groups, substituted or unsubstituted C₂-C₃₀ heteroarylalkyl groups, substituted or unsubstituted C₅-C₂₀ cycloalkyl groups, substituted or unsubstituted C₅-C₃₀ heterocycloalkyl groups, substituted or unsubstituted C₆-C₃₀ aryl ester group and a heteroaryl ester group of substituted or unsubstituted C₂-C₃₀.

However, when n is 0, at least one or more of R₁, R₂, R₃, and R₄ are hydrophobic functional groups containing halogen elements or containing hydrophobic functional groups).

The basic material can be an amine compound having a pKa of 4 to 6, a pyridine compound, and specifically, one or more selected from the group consisting of the amine compound such as naphthylamine, n-Allylaniline, etc. aminobiphenyl(4-Aminobiphenyl), toluidine (o-Toluidine), aniline (Aniline), quinoline (Quinoline), dimethylaniline (N, N,-Diethyl aniline), and pyridine (Pyridine).

FIG. 30 is a graph showing the effects of acidity and work function when a basic additive is added to PEDOT:PSS:PFI, which is a conductive polymer and a hole injection layer.

As shown in FIG. 30, when aniline is added to PEDOT:PSS:PFI, the acidity decreases (pH increases), and it can be confirmed that an influence of work function is small compared to other basic additives.

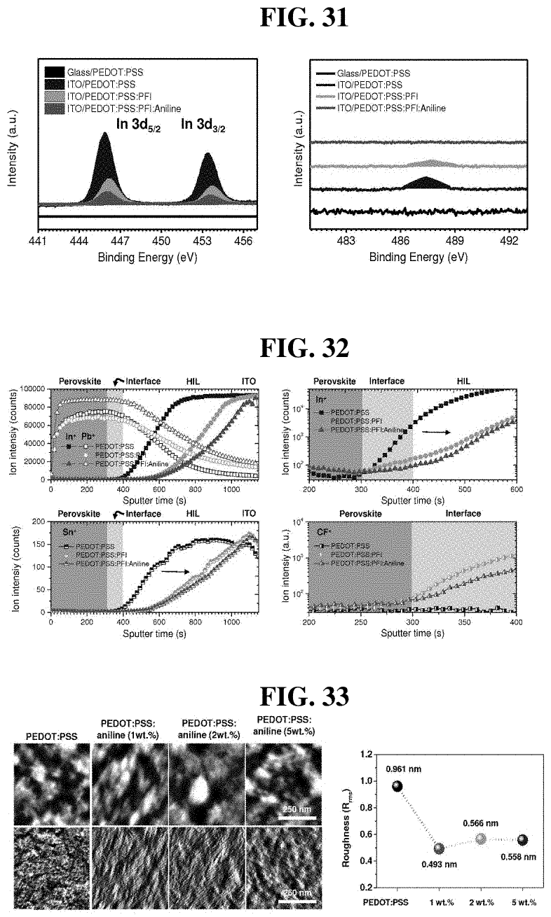

FIG. 31 is a graph of the change in strength according to binding energy when aniline is spin-coated on the ITO electrode in PEDOT:PSS:PFI for a conductive polymer hole injection layer according to an embodiment of the present disclosure.

As shown in FIG. 31, when PEDOT:PSS:PFI:aniline was formed on ITO, the amount of In⁺ and Sn⁺ ions detected on the surface was confirmed to be significantly lower than that of PEDOT:PSS.

FIG. 32 shows a graph which shows the ion intensity at the interface with a halide perovskite emissive layer for the conductive polymer hole injection layer according to an embodiment of the present disclosure, in which a hole injection layer and a metal are formed when aniline is formed on an ITO electrode in PEDOT:PSS:PFI.

As shown in FIG. 32, it was confirmed that when PEDOT:PSS:PFI:aniline was deposited on ITO, the amount of In⁺ and Sn⁺ ions detected on the surface decreased and the diffusion was delayed.

FIG. 33 shows the surface roughness of the formed thin film according to the amount of aniline added when aniline is added to PEDOT:PSS in the conductive polymer hole injection layer according to an embodiment of the present disclosure.

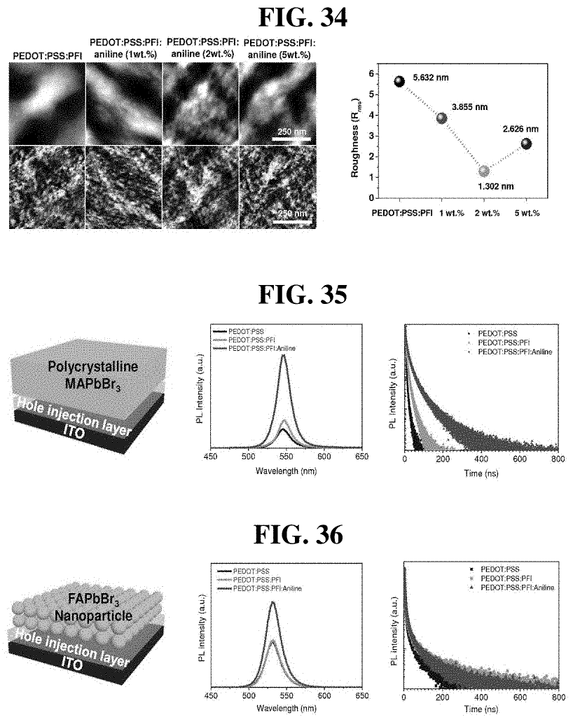

FIG. 34 shows the surface roughness of the formed thin film according to the amount of aniline added when aniline is added to PEDOT:PSS:PFI in the conductive polymer hole injection layer according to an embodiment of the present disclosure.

As shown in FIGS. 33 and 34, it was confirmed that the surface roughness of the thin film was reduced when aniline was added to PEDOT:PSS or PEDOT:PSS:PFI.

FIG. 35 is a graph showing emission intensity and photoluminescence (PL) lifetime of metal halide perovskite in a polycrystalline metal halide perovskite layer/PEDOT:PSS:PFI:aniline/ITO electrode according to an embodiment of the present disclosure.

FIG. 36 is a graph showing the emission intensity and photoluminescence (PL) lifetime of the metal halide perovskite nanoparticle layer/PEDOT:PSS:PFI:aniline/ITO electrode according to the embodiment of the present disclosure.

As shown in FIGS. 35 and 36, the photoluminescence (PL) of the polycrystalline metal halide perovskite thin film and the metal halide perovskite nanoparticle thin film on PEDOT:PSS:PFI:aniline is increased, and the photoluminescence (PL) lifetime is increased.

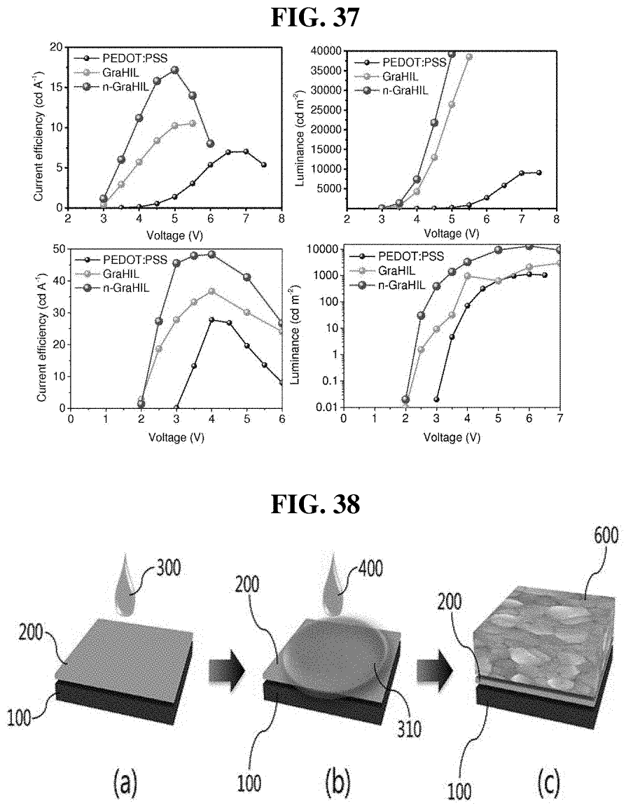

FIG. 37 is a graph showing the efficiency of a polycrystalline metal halide perovskite device and a metal halide perovskite nanoparticle device using a PEDOT:PSS:PFI:aniline hole injection layer according to an embodiment of the present disclosure.

As shown in FIG. 37, the higher efficiency of a polycrystalline metal halide perovskite device and a metal halide perovskite nanoparticle device using PEDOT:PSS:PFI:aniline as a hole injection layer was obtained compared to the device using PEDOT:PSS as a hole injection layer.

Therefore, the PEDOT:PSS:PFI:aniline hole injection layer according to the present disclosure can reduce the acidity and improve the stability of the lower electrode and the upper metal halide perovskite thin film, and therefore the efficiency and stability of the metal halide perovskite light-emitting diodes can be improved.

<Light-Emitting Diodes Including Graphene Barrier>

According to another embodiment of the present disclosure, when an electrode dissociated by an acid is used for the light emitting diodes, a graphene barrier layer can be further included. The electrode material including indium-tin oxide (ITO), which is mainly used as an oxide transparent electrode material for light-emitting diodes, has a property of being dissociated by acid. Generally, a PEDOT:PSS conductive polymer is mainly used as a hole injection layer on the upper part of the indium-tin oxide electrode. However, PEDOT:PSS has a high pH (˜pH 2) and dissolves ITO which is vulnerable to acid, and when the dissolved indium and tin ions are diffused into the upper emissive layer, exciton quenching can occur and reduce the efficiency of the light-emitting diodes. In particular, when the emissive layer of the light-emitting diodes is a metal halide perovskite, the efficiency of the metal halide perovskite light-emitting diodes can be significantly reduced due to the long exciton diffusion length. In order to improve the characteristic that these light emission characteristics are lowered, the optoelectronic device can include a graphene barrier layer.

Further, preferably, the light-emitting diodes containing the graphene barrier is a first electrode and a second electrode facing each other; a emissive layer formed between the first electrode and the second electrode; the first electrode contains a PEDOT:PSS hole transport layer formed between the emissive layer and the emissive layer, and the first electrode is an electrode dissociated by acid, and graphene barrier layer formed between the first electrode and the PEDOT:PSS hole transport layer.

The first electrode (20) is made of a material having a conductive property as an electrode (anode) into which holes are injected. The material constituting the first electrode (20) can be a conductive metal oxide, a metal, a metal alloy, or a carbon material. Conductive metal oxides include indium tin oxide (ITO), fluorine tin oxide (FTO), antimony tin oxide (ATO), fluorine-doped tin oxide (FTO), SnO₂, ZnO, or a combination thereof. Suitable metals or metal alloys as anodes can be Au and CuI. For carbon material, it can be graphite, graphene, or carbon nanotubes.

The graphene barrier layer is located on the first electrode.

The graphene barrier layer (12) is a carbon allotrope forming a two-dimensional plane in the shape of a hexagonal lattice of carbon atoms. The graphene not only exhibits excellent electrical and optical properties, but also has a very dense carbon lattice structure and is attracting much attention as a sealing material.

Graphene can prevent acid dissociation as a barrier layer for electrodes such as ITO that are vulnerable to acid by preventing the movement of ions in the acid.

At this time, the thickness of the graphene barrier layer can be 0.1 nm to 100 nm, but is not limited thereto.

Further, the graphene barrier layer laminated on the electrode dissociated by an acid can be composed of a single layer or a plurality of layers of two or more layers.

Hereinafter, the present disclosure provides a method for manufacturing a optoelectronic device including a graphene barrier layer laminated on an electrode which dissociates from acid.

The method for producing an optoelectronic device includes a step of forming a graphene barrier layer on an electrode dissociated by an acid. For example, when the optoelectronic device is a light emitting diodes including PEDOT:PSS hole transport layer, a step of forming a graphene barrier layer on the first electrode dissociated by acid; a step of forming a PEDOT:PSS hole transport layer on the graphene barrier layer; a step of forming the emissive layer; a step of forming the second electrode on the emissive layer can be included, but the present disclosure is not limited to these, and depending on the type of the optoelectronic device, after a step of forming the graphene barrier layer on top of the electrode dissociated by an acid, methods known in the industry can be used.

Therefore, in the present disclosure, the steps of forming a graphene barrier layer on the electrodes dissociated by an acid will be mainly described.

The above step of forming the graphene barrier layer on the electrode dissociated by an acid include the step of forming the graphene layer on the catalytic metal layer; the step of forming the polymer layer on the above graphene layer; the step of forming a polymer layer/graphene layer thin film by removing the catalytic metal layer and the step of transferring the polymer layer/graphene layer thin film onto an electrode dissociated by an acid and removing the polymer layer.

Hereinafter, the details will be described step by step.

First, a graphene layer is formed on the catalyst metal layer.

At this time, the catalyst metal layer includes any one or a combination of two or more selected from the group consisting of copper (Cu), nickel (Ni), germanium (Ge), cobalt (Co), iron (Fe), gold (Au), palladium (Pd), aluminum (Al)), Chromium (Cr), Magnesium (Mg), Molybdenum (Mo), Ruthenium (Rh), Silicon (Si), Tantal (Ta), Titanium (Ti), Tungsten (W), Uran, Vanadium (V) and Zirconium (Zr).

The catalyst metal layer can be vacuum-deposited on a substrate with a thickness of 100 nm to 50 μm.

At the stage of forming a graphene layer on the catalyst metal layer, a carbon precursor placed on the catalyst metal layer deposited for 1 second to 5 days in the range from 200° C. to 2000° C. in an inert atmosphere or a vacuum atmosphere by using a chemical vapor deposition method.

The carbon precursor can be a carbon-containing hydrocarbon in the form of a gas or solid.

Next, a polymer layer is formed on the graphene layer.

The polymer used in the industry may be used for the above polymer layer, and for example, polymethyl methacrylate (PMMA) may be used, but it is not limited thereto.

The polymer layer can be formed by coating a polymer solution dissolved in a solvent on the graphene layer. A polymer layer/graphene layer/catalyst metal layer thin film is formed in the formation of the polymer layer.

Next, the catalytic metal layer is removed to form a polymer layer/graphene layer thin film.

The removal of the catalyst metal layer can be performed by immersing the polymer layer/graphene layer/catalyst metal layer thin film in a metal etching solution.

Next, the above polymer layer/graphene layer thin film is transferred on the electrodes dissociated by the acid, and the graphene barrier layer can be formed on the electrodes dissociated by the acid by removing the polymer layer.

Specifically, the polymer layer/graphene layer thin film formed in the metal etching solution is scooped out by electrode substrate which is dissociated by acid, and form polymer layer/graphene layer/electrode, and immersing the polymer layer in a polymer layer removing solution such as acetone to remove the polymer layer, a graphene barrier layer can be formed on the electrode dissociated by the acid.

At this time, the thickness of the graphene barrier layer can be 0.1 nm to 100 nm.

The graphene barrier layer can be formed as a single layer, and by repeating the graphene barrier layer formation step, a plurality of layers having two or more layers can be formed.

At this time, it is desirable that the graphene barrier layer is within the 10 layers, but if the graphene barrier layer exceeds the 10 layers, the operating voltage of light-emitting diodes can increase, and the efficiency of light-emitting diodes can decrease, which is driven due to the insulating characteristics of the graphene barrier layer.

In an optoelectronic device manufactured in this manner, the chemically stable graphene barrier layer protects the electrode vulnerable to the acid, so that the stability and durability of the electrode are improved even in an acidic environment.

In an optoelectronic device containing an acid-containing PEDOT:PSS-based hole injection layer, wherein the chemically stable graphene barrier layer protects the vulnerable electrode, the exciton quenching by the PEDOT:PSS-based hole injection layer can be prevented and a highly efficient light-emitting diodes can be manufactured.

<Organic-Assisted Nanocrystal Pinning Process for Producing High-Efficiency Metal Halide Perovskite Light-Emitting Devices>

When the above-described metal halide perovskite is a polycrystalline bulk metal halide perovskite, when forming a polycrystalline bulk metal halide perovskite in the emissive layer, an emissive layer can be formed using a two-step process by additionally applying an organic solution in which a small amount of organic small molecule dissolved in organic solvent before the solvent of the emissive layer is removed.

FIG. 38 shows a method of dropping and coating an organic small molecule solution before the solvent of the emissive layer evaporates while the metal halide perovskite emissive layer according to the embodiment of the present disclosure is coated. It is a schematic diagram which shows the organic material assisted nanocrystal pinning process.

With reference to FIG. 38, a method for manufacturing a metal halide perovskite light-emitting diodes including the metal halide perovskite emissive layer coated by the method of dropping the organic small molecule material solution for coating the metal halide perovskite emissive layer according to the embodiment of the present disclosure (organic material auxiliary nanocrystal pinning step) will be described.

In the present specification, the above-mentioned “organic-assisted nanocrystal pinning step”, after starting to apply a metal halide perovskite solution on a substrate and before the solvent is completely evaporated, that is, before the color of the thin film changes due to crystallization, preferably, means a process of dropping a low-molecular organic molecule solution or applying jet printing in the form of drop-on-demand within 1 to 200 seconds after the start of metal halide perovskite coating, and exhibits the effect of reducing the size of the metal halide nanocrystals during coating. The metal halide perovskite luminescence layer (600) can be formed by coating the small molecular organic solution, a key feature of this disclosure, by dropping it before it dries.

First, a metal halide perovskite solution (300) and an organic small molecule solution (400) can be prepared. Next, the metal halide perovskite solution (300) can be applied and coated on the substrate. At this time, the coating methods including spin-coating, Dip coating, Shear coating, Bar coating, Slot-die coating, and Inkjet Printing, Nozzle printing, Electrohydrodynamic jet printing or spray coating can be used.

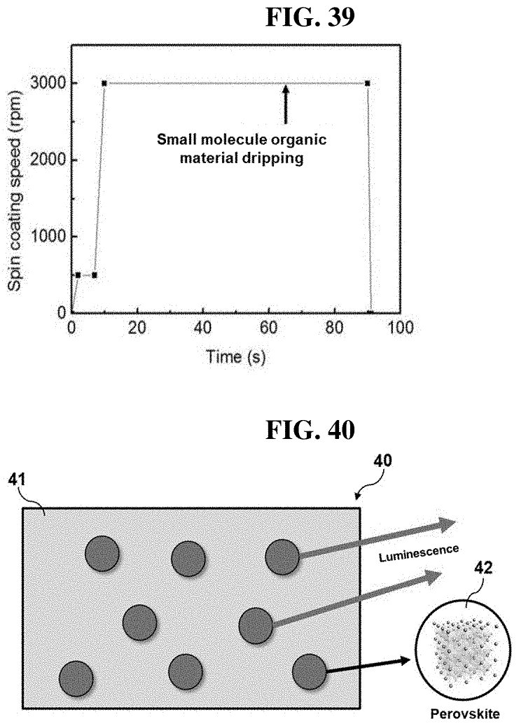

Then, in the middle of the coating, the organic small molecule solution is dropped into a small number of droplets (dripping) or droplets are sprayed through a printer device (jetting or spraying), and then a thin film with controlled size of lead halide perovskite crystal is formed. At this time, when the metal halide perovskite film in which the organic small molecule material is generally distributed is formed and crystallized, metal halide perovskite emissive layer (600) which the organic small molecule material is located at the grain boundary or the surface can be formed [see FIG. 39 (c)].

The low-molecular organic material solution (400) is preferably performed after coating the metal halide perovskite solution on a substrate, and before the solvent is completely evaporated (that is, before the color of the thin film is changed due to crystallization). For example, the organic small molecule solution (400) can be dropped within 1 to 200 seconds after the start of the metal halide perovskite coating, preferably.

After the start of metal halide perovskite coating, the time to drop the low molecular organic material solution (400) include a range where the two numbers of 1 second, 2 seconds, 3 seconds, 4 seconds, 5 seconds, 6 seconds, 7 seconds, 8 seconds, 9 seconds, 10 seconds, 11 seconds, 12 seconds, 13 seconds, 14 seconds, 15 seconds, 16 seconds after the start of coating, 17 seconds, 18 seconds, 19 seconds, 20 seconds, 21 seconds, 22 seconds, 23 seconds, 24 seconds, 25 seconds, 26 seconds, 27 seconds, 28 seconds, 29 seconds, 30 seconds, 31 seconds, 32 seconds, 33 seconds. Seconds, 34 seconds, 35 seconds, 36 seconds, 37 seconds, 38 seconds, 39 seconds, 40 seconds, 41 seconds, 42 seconds, 43 seconds, 44 seconds, 45 seconds, 46 seconds, 47 seconds, 48 seconds, 49 seconds, 50 seconds, 51 seconds, 52 seconds, 53 seconds, 54 seconds, 55 seconds, 56 seconds, 57 seconds, 58 seconds, 59 seconds, 60 seconds, 61 seconds, 62 seconds, 63 seconds, 64 seconds, 65 seconds, 66 seconds, 67 seconds, 68 seconds, 69 seconds, 70 seconds, 71 seconds, 72 seconds, 73 seconds, 74 seconds, 75 seconds, 76 seconds, 77 seconds, 78 seconds, 79 seconds, 80 seconds, 85 seconds, 90 seconds, 95 seconds 100 seconds, 110 seconds, 120 seconds, 130 seconds, 140 seconds, 150 seconds, 160 seconds, 170 seconds, 180 seconds, 190 seconds, and 200 seconds, has the lower number is lower limit and the higher value has the upper limit. The metal halide perovskite is as described above, and detailed description thereof will be omitted.

For example, the metal halide perovskite solution (300) can be prepared by mixing AX and BX₂ and dissolving them in a polar organic solvent. At this time, the polar organic solvent can be dimethyl sulfoxide or dimethyl formamide. For example, CH₃NH₃Br and PbBr₂ can be mixed at a ratio of 1.05:1 and dissolved in dimethyl sulfoxide (DMSO) at 40 wt % to produce the above metal halide perovskite solution (300), CH₃NH₃PbBr₃.

The above organic small molecule matter can be n-type organic small molecule matter when the metal halide perovskite material of the above metal halide perovskite emissive layer (600) has a p-type attribute. It is not limited to this.

The organic small molecule matter can be an n-type organic matter capable of playing a role in electron transfer. For example, an n-type organic small molecule material can be added to the metal halide perovskite emissive layer (600) of p-type CH₃NH₃PbBr₃. The small organic material can include pyridines, —CN, —F or Oxadizole. For example, the organic small molecule material includes TPBI (2,4,6-tris(2-N-phenylbenzimidazolyl)benzene), TmPyPB (2,4,6-Tri (m-pyrid-3-yl-phenyl)benzene), BmPyPB (1,3-bis(3,5-dipyrid-3-yl-phenyl)benzene), BCP (2,9-dimethyl-4,7-diphenyl-1,10-phenanthroline), PBD (2-(4)-biphenylyl)-5-phenyl-1,3,4-oxadiazole), Alq₃ (Tris-(8-hydroxyquinoline)aluminum), BAlq (aluminum (III) bis(2-methyl-8-quinolinato)-4-phenylphenolate), Bebg₂ (bis(10 hydroxybenzo[h]quinolinato) beryllium), or OXD-7 (bis[2-(4-tert-butylphenyl)-1,3,4-oxadiazo-5-yl]benzene).

The organic small molecule matter can have a molecular weight of 10 to 1000.

The organic small molecule material can be a material such as the electron transfer layer (not shown) described above.

The “organic small molecule material” coated on the metal halide perovskite emissive layer (600) is an important feature of the present disclosure and is located at the grain boundaries of the metal halide perovskite crystal structure, and reduce the interaction of crystals, prevents large. In addition, the n-type small-molecular-weight organic material is located at the metal halide perovskite crystal grain boundary so that the p-type metal halide perovskite emissive layer (600) has intrinsic properties to improve the electrical properties. It improves and helps to balance electrons and holes well. Therefore, by adding the organic small molecule material according to the present disclosure to the grain boundary inside the metal halide perovskite emissive layer (600), the crystal grain size of the metal halide perovskite is reduced, and the metal halide perovskite defect is eliminated. It is passivation and has the effect of eliminating the electron-hole imbalance caused by non-polar electrical properties and overcoming the application limits of the metal halide perovskite light-emitting diodes.

The organic small molecule matter can be a p-type organic small molecule matter when the metal halide perovskite material of the metal halide perovskite emissive layer (600) has the n-type attribute. But it is not limited to this. The above p-type organic small molecule materials are TAPC (di-[4-(N, N-ditolyl-amino)-phenyl]cyclohexane) or TCTA (4,4′4″-tri (N-carbazolyl)triphenylamine)). But it is not limited to this.

The above small molecular organic matter solution (400) may be prepared by dissolving small molecular organic matter in a non-polar organic solvent. The above non-polar organic solvents may be limited to chloroform, chlorobenzene, toluene, dichloroethane, dichloromethane, ethyl acetate or xylene, but may not be limited to this.

The concentration of the organic small molecule solution (400) can be 0.001 wt % to 5 wt %. For example, the concentrations of the organic small molecule solution (400) are 0.001 wt %, 0.1 wt %, 0.2 wt %, 0.3 wt %, 0.4 wt %, 0.5 wt %, 0.6 wt %, 0.7 wt %, 0.8 wt %, 0.9 wt %, 1 wt %, 1.1 wt %, 1.2 wt %, 1.3 wt %, 1.4 wt %, 1.5 wt %, 1.6 wt %, 1.7 wt %, 1.8 wt %, 1.9 wt %, 2 wt %, 2.05 wt %, 2.1 wt %, 2.15 wt %, 2.2 wt %, 2.25 wt %, 2.3 wt %, 2.35 wt %, 2.4 wt %, 2.45 wt %, 2.5 wt %, 2.55 wt %, 2.6 wt %, 2.65 wt %, 2.7 wt %, 2.75 wt %, 2.8 wt %, 2.85 wt %, 2.9 wt %, 2.95 wt %, 3 wt %, 3.1 wt %, 3.2 wt %, 3.3 wt %, 3.4 wt %, 3.5 wt %, 3.6 wt % %, 3.7 wt %, 3.8 wt %, 3.9 wt %, 4 wt %, 4.1 wt %, 4.2 wt %, 4.3 wt %, 4.4 wt %, 4.5 wt %, 4.6 wt %, 4.7 wt %, 4.8 wt %, 4.9 wt % and 5 wt %. The range in which the lower value of two of the above is the lower limit, and the higher value is the upper limit can be included.

If the concentration of the above organic small molecule solution exceeds the above range and is less than 0.001 wt %, the effect of trap passivation by the organic small molecule material and the balance of electrons and holes may not be exhibited. When the above concentration is 5 wt % or more, organic small molecule materials that have not entered the metal halide perovskite grain boundaries are thickly accumulated on the surface (>20 nm), and the efficiency of the device can be reduced. Preferably, the device may be efficient if the surface organic small molecule material must have a thickness of 10 nm or less.

The thickness of the metal halide perovskite emissive layer (600) can be 10 nm to 900 nm.

With reference to FIG. 38, in the process of coating the metal halide perovskite solution, the organic small molecule solution can be dropped.

FIG. 39 is a graph showing a point in time when a low molecular weight organic material solution is dropped while the metal halide perovskite emission layer is coated when the metal halide perovskite emission layer is manufactured according to an embodiment of the present disclosure.

For example, the organic small molecule solution can be dropped within 1 to 200 seconds after the start of the metal halide perovskite coating, preferably after the start of the metal halide perovskite coating, 1 second, 2 seconds, 3 seconds, 4 seconds, 5 seconds, 6 seconds, 7 seconds, 8 seconds, 9 seconds, 10 seconds, 11 seconds, 12 seconds, 13 seconds, 14 seconds, 15 seconds, 16 seconds, 17 seconds, 18 seconds, 19 seconds, 20 seconds, 21 seconds, 22 seconds, 23 seconds, 24 seconds, 25 seconds, 26 seconds, 27 seconds, 28 seconds, 29 seconds, 30 seconds, 31 seconds, 32 seconds, 33 seconds, 34 seconds, 35 seconds, 36 seconds, 37 seconds, 38 seconds, 39 seconds, 40 seconds, 41 seconds, 42 seconds, 43 seconds, 44 seconds, 45 seconds, 46 seconds, 47 seconds, 48 seconds, 49 seconds, 50 seconds, 51 seconds, 52 seconds, 53 seconds, 54 seconds, 55 seconds, 56 seconds, 57 seconds, 58 seconds, 59 seconds, 60 seconds, 61 seconds, 62 seconds, 63 seconds, 64 seconds, 65 seconds, 66 seconds, 67 seconds, 68 seconds, 69 seconds, 70 seconds, 71 seconds, 72 seconds, 73 seconds, 74 seconds, 75 seconds, 76 seconds, 77 seconds, 78 seconds, 79 seconds, 80 seconds, 85 seconds, 90 seconds, 95 seconds, 100 seconds, 110 seconds, 120 seconds, 130 seconds, 140 seconds, 150 seconds, 160 seconds, 170 seconds, 180 seconds, 190 seconds, and 200 seconds, and it can include the range in which the lower value of the two numbers above is the lower limit and the higher value is the upper limit.

For example, the organic small molecule solution can be dropped between 60 and 70 seconds after the metal halide perovskite solution is applied onto the substrate and spin coating begins.

The following chemical formula shows the structural formula of the organic small molecule material according to the present disclosure.

With reference to the equations described above, the organic small molecule matter can be an n-type organic matter capable of playing a role in electron transfer. For example, an n-type organic small molecule material can be added to the metal halide perovskite emissive layer of p-type CH₃NH₃PbBr₃. The small organic matter can include pyridines, —CN, —F or Oxadizole. For example, the organic small molecule matter includes TPBI (2,4,6-tris(2-N-phenylbenzimidazolyl)benzene), TmPyPB (2,4,6-Tri (m-pyrid-3-yl-phenyl)benzene), BmPyPB (1,3-bis(3,5-dipyrid-3-yl-phenyl)benzene), BCP (2,9-dimethyl-4,7-diphenyl-1,10-phenanthroline), PBD (2-(4)-biphenylyl)-5-phenyl-1,3,4-oxadiazole), Alq₃ (Tris-(8-hydroxyquinoline)aluminum), BAlq (aluminum (III) bis(2-methyl-8-quinolinato)-4-phenylphenolate), Bebg₂ (bis(10-hydroxybenzo[h]quinolinato) beryllium), or OXD-7 (bis[2-(4-tert-butylphenyl)-1,3,4-oxadiazo-5-yl]benzene)

The organic small molecule matter can have a molecular weight of 10 to 1000.

On the other hand, when the metal halide perovskite material of the metal halide perovskite emissive layer has an n-type attribute, a p-type organic small molecule material can be used, but is not limited thereto. For example, the organic small molecule matter can be TAPC (di-[4-(N, N-ditolyl-amino)-phenyl]cyclohexane) or TCTA (4,4′4″-tri (Ncarbazolyl)triphenylamine).

On the other hand, the organic small molecule matter can be a bipolar organic small molecule matter. The bipolar organic small molecule matter can be CBP (4,4′-N, N′-dicarbazole-biphenyl).

On the other hand, the organic small molecule matter has a molecular weight of 10-1000;

It is a benzene derivative containing an N atom; It is a benzene derivative containing an S atom; It is selected from benzene derivatives containing two or more atoms selected from N, S and O atoms,

The compound containing a thiol group (—SH) group may be characterized in that it is represented by R—SH or HS—R—SH (R is an alkyl group, an aryl group, or a mixture of an alkyl group and an aryl group).

Benzene derivatives containing the N atom are Pyridine, Quinoline, Isoquionoline, Pyrazine, Quinoxaline, Acridine, Pyrimidine, Quinazoline, Pyridazine, Cinnoline, Phthalazine, 1,2,3-Triazine, 1,2,4-Triazine,1,3,5-Triazine, Pyrrole, Pyrazole, Indole, Isoindole, Imidazole, Benzimidazol, Purine, Adenine, or Indazole may be included, but are not limited thereto.

The benzene derivative containing the S atom can include, but is not limited to Thiophene, Benzothiophene or benzo[c]thiophene.

The benzene derivative containing an O atom can include, but is not limited to, Furan, Benzofuran or Isobenzofuran.

The group of benzene derivatives contain a plurality of atoms selected from the above N, S, and O atoms includes Oxazole, Benzoxazole, Benzisoxazole, Isoxazole, Thiazole, Guanine, Hypoxanthine, Xanthine, Theobromine, Caffeine, Uric acid, Isoguanine, and Cytosine. Thymine, Uracil or Benzothiazole, but is not limited to this.

Further, the organic small molecule additive can further include a group of benzene derivatives. The group of the benzene derivatives can include Benzene, Naphthalene or Anthracene.

When the metal halide perovskite emissive layer contains an organic small molecule additive, the organic small molecule additive locates at the crystal grain boundary in the process of growing metal halide perovskite crystalline particles on the emissive layer. By being located at the boundary, it is possible to prevent the extinction of the exciton occurring at the grain boundary and restrain the exciton. Further, the organic small molecule additive prevents the growth of metal halide perovskite crystalline particles during the formation of the metal halide perovskite thin film, thereby forming the crystalline particles of the metal halide perovskite thin film containing no organic small molecule additive. It is possible to induce the growth of crystalline particles having a relatively small size, which can increase the binding force of the exciton existing inside the metal halide perovskite crystalline particles.

<Metal Halide Perovskite Light-Emitting Diodes Using Metal Halide Perovskite-Organic Small Molecule Host Mixed Emissive Layer>

Also preferably, in the metal halide perovskite light-emitting diodes according to the present disclosure, the emissive layer is a metal halide perovskite-organic small molecular host mixed emissive layer in which a metal halide perovskite and an organic small molecule host are co-deposited.

The metal halide perovskite emissive layer used in the metal halide perovskite light-emitting diodes is mainly manufactured through a solution process. However, the above solution step has the disadvantages that the uniformity of the thin film formed is low, the thickness cannot be easily adjusted, and the materials that can be mixed are limited by the characteristics of the solvent.

For metal halide perovskite luminescent devices, the biggest inhibitor to performance is uneven thin films. For thin-film devices consisting of laminated thin films, the unevenness of the thin film is one of the factors that significantly degrade the performance of the element by breaking the charge balance and generating leakage current. In particular, the uniformity of the thin film is crucial for the performance of metal halide perovskite luminescence devices, as metal halide perovskite varies greatly in the morphology of the thin film depending on the thin film formation conditions and surrounding environment. An example of uneven thin films is a common spin-coating process that forms CH₃NH₃PbBr₃, the problem is that without additional nanocrystal pinning process, the thin films are formed in isolated crystal form due to spontaneous crystallization. [Science 2015, 350, 1222].

However, when using the nanocrystal pinning process, the film quality of the thin film can be highly dependent on the experimental environment, so even if the same process is used, the deviation of the film quality is large. In addition, since the film quality of the thin film is improved only in the region where the nanocrystal is formed, there can be a limit in realizing a large-area device.

The location of the electron-hole recombination zone in the device, i.e. the emission spectrum of the device, can be influenced by the thickness of the emissive layer and is the energy level of the material used. It can depend on the position.

However, by depositing the metal halide perovskite and the organic small molecule host, a uniform thin film can be formed, the thickness of the thin film can be easily adjusted, and the metal halide perovskite crystal to be formed can be formed. Due to the smaller size, exciton or charge carrier can be spatially constrained to improve luminescence efficiency. Further, by adjusting the mixing ratio of the metal halide perovskite and the organic small molecule host to adjust the energy level, the degree of energy transfer can be adjusted to adjust the emission wavelength, and the emission wavelength can be adjusted. The electron-hole recombination zone can be adjusted to improve the efficiency of electroluminescence.

FIG. 40 shows a metal halide perovskite-organic according to an embodiment of the present disclosure. It is a sectional drawing which shows the small molecule host mixed emissive layer.

Referring to FIG. 40, the metal halide perovskite-organic small molecule host mixed emissive layer according to the present disclosure contains the metal halide perovskite (42) as a guest in the organic small molecule host (41).

In the case of a light-emitting layer deposited with a metal halide perovskite (42) and an organic small molecule host (41), the energy transfer behavior changes based on the energy level of the material. That is, the energy transfer may occur in the metal halide perovskite on the organic small molecule host, or vice versa, and the emission may occur in the metal halide perovskite (the metal halide perovskite acts as a guest), and the organic small molecule. It can also occur in (small organic molecules act as guests).

Therefore, the energy level of the material used to regulate the position of luminescence is very important.

In the present specification, the energy level means the magnitude of energy. Therefore, even when the energy level is displayed in the minus (−) direction from the vacuum level, the energy level is interpreted to mean the absolute value of the energy value. For example, it means the distance from the HOMO energy level and vacuum level of an organic small molecule host to the highest occupied molecular orbital. It also means the distance from the LUMO energy level and vacuum level of the organic small molecule host to the lowest unoccupied molecular orbital.

In the present specification, the CBM (conduction band minimum) of the metal halide perovskite refers to the lowest stage of the conduction band of the material, and the VBM (valence band maximum) of the metal halide perovskite refers to the uppermost stage of the material household appliances. The difference between the above CBM and VBM is called the bandgap.

In the present specification, the measurement of the HOMO energy level of the organic small molecule host and the VBM of the metal halide perovskite irradiates the surface of the thin film with UV, and at this time, the ejected electrons are detected to detect the material. UPS (UV photoelectron spectroscopy) that measures the ionization potential can be used. Alternatively, the HOMO energy level is measured by using CV (cyclic voltammetry), which measures the oxidation potential via voltage sweep after dissolving the material to be measured in a solvent together with the electrolytic solution. can do. In addition, a PYSA (Photoemission Yield Spectrometer in Air) method for measuring the ionization potential in the atmosphere can be used using an AC-3 (RKI) machine.

In the present specification, the LUMO energy level of the organic small molecule host and the CBM of the metal halide perovskite can be obtained by measuring inverse photoelectron spectroscopy (IPES) or electrochemical reduction potential. IPES is a method of determining the LUMO energy level by irradiating an electron beam onto a thin film and measuring the light emitted at this time. In addition, in the measurement of the electrochemical reduction potential, a reduction potential may be measured through a voltage sweep after dissolving a material to be measured in a solvent together with an electrolyte. Alternatively, the LUMO energy level can be calculated using the HOMO energy level and the singlet energy level obtained by measuring the UV absorption level of the target material.

Specifically, the HOMO energy level of the present specification was measured via an AC-3 (RKI) measuring instrument after vacuum-depositing the target material on an ITO substrate with a thickness of 50 nm or more. For the LUMO energy level, after measuring the absorption spectrum (abs.) and the photoluminescence (PL) of the manufactured sample, the edge energies of each spectrum are calculated, and the difference is seen in the band gap (Eg). Then, the LUMO energy level was calculated by subtracting the band gap difference from the HOMO energy level measured by AC-3.

It is an object of the present disclosure that light emission occurs in the metal halide perovskite (42). Therefore, the metal halide perovskite-organic small molecule host mixed emissive layer is characterized by using an organic small molecule host (41) as a host and a metal halide perovskite (42) as a guest. It is desirable to use a bandgap of energy levels of the organic small molecule host (41) used as a host larger than the bandgap of the metal halide perovskite used as a guest.

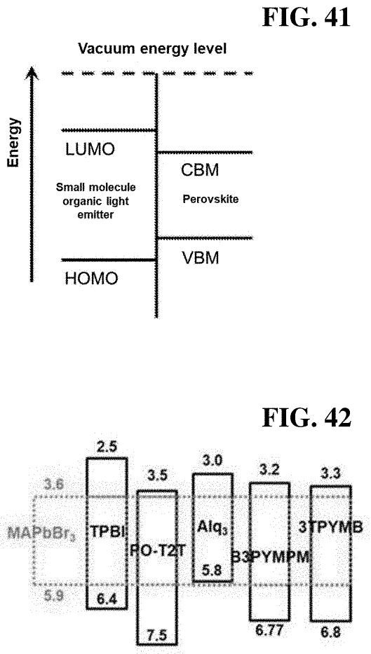

That is, as shown in FIG. 41, it is desirable to use that the HOMO energy level of the organic small molecule host is lower than the VBM of the metal halide perovskite, and the LUMO energy level of the organic small molecule host is higher than the CBM of the metal halide perovskite.

An example of such an organic small molecule host is shown in FIG. 42.

FIG. 42 shows the energy levels of the metal halide perovskite-organic small molecule host mixed emissive layer according to one embodiment of the present disclosure.

Explaining with reference to FIG. 42, when the metal halide perovskite according to the embodiment of the present disclosure is used as MAPbBr₃, the VBM of the above MAPbBr₃ is (−) 5.9 and the CBM is (−) 3.6. At this time, TBPI has a HOMO energy level of (−) 6.4, which is lower than the VBM of the metal halide perovskite (MAPbBr₃), and a LUMO energy level of (−) 2.5, which is higher than the CBM of the metal halide perovskite (MAPbBr₃). It can be used in the metal halide perovskite-organic small molecule host mixed emissive layer according to the present disclosure.

As shown in FIG. 42, the organic host with low molecular weight used in the metal halide perovskite-low molecular weight organic host mixed light emitting layer according to the present disclosure can use 1,3,5-Tris(1-phenyl-1H-benzimidazol-2-yl)benzene (TPBI), 2,4,6-Tris[3-(diphenylphosphinyl)phenyl]-1,3,5-triazine (PO-T2T), Tris(8-hydroxyquinolinato)aluminium (Alq₃), 4,6-Bis(3,5-di(pyridin-3-yl)phenyl)-2-methylpyrimidine (B3PYMPM), Tris(2,4,6-triMethyl-3-(pyridin-3-yl)phenyl)borane (3TPYMB), 4,4′-Bis(N-carbazolyl)-1,1′-biphenyl (CBP), 9,10-di(2-naphthyl)anthracene (ADN), (Tris(4-carbazoyl-9-ylphenyl)amine) TCTA, (1,1-Bis[(di-4-tolylamino)phenyl]cyclohexane) TAPC, (2-tert-butyl-9,10-di(2-naphthyl)anthracene) TBAD, E3, (Bis(10-hydroxybenzo[h]quinolinato)beryllium) BeBq₂, but is not limited thereto.

In this case, the mixing ratio of the metal halide perovskite and the organic low molecular weight host may be 0.01 wt % to 49.99 based on the mass ratio of the metal halide perovskite to the sum of the weights of the metal halide perovskite and the organic low molecular weight host, but is not limited thereto. The mass ratio may include a range in which a lower value of two numbers among 0.01 wt %, 0.2 wt %, 0.4 wt %, 0.6 wt %, 0.8 wt %, 1 wt %, 1.2 wt %, 1.4 wt %, 1.6 wt %, 1.8 wt %, 2 wt %, 2.2 wt %, 2.4 wt %, 2.6 wt %, 2.8 wt %, 3 wt %, 3.1 wt %, 3.2 wt %, 3.3 wt %, 3.4 wt %, 3.5 wt %, 3.6 wt %, 3.7 wt %, 3.8 wt %, 3.9 wt %, 4 wt %, 4.1 wt %, 4.2 wt %, 4.3 wt %, 4.4 wt %, 4.5 wt %, 4.6 wt %, 4.7 wt %, 4.8 wt %, 4.9 wt %, 5 wt %, 5.2 wt %, 5.4 wt %, 5.6 wt %, 5.8 wt %, 6 wt %, 6.2 wt %, 6.4 wt %, 6.6 wt %, 6.8 wt %, 7 wt %, 7.2 wt %, 7.4 wt %, 7.6 wt %, 7.8 wt %, 8 wt %, 8.2 wt %, 8.4 wt %, 8.6 wt %, 8.8 wt %, 9 wt %, 9.2 wt %, 9.4 wt %, 9.6 wt %, 9.8 wt %, 10 wt %, 10.5 wt %, 11 wt %, 11.5 wt %, 12 wt %, 12.5 wt %, 13 wt %, 13.5 wt %, 14 wt %, 14.5 wt %, 15 wt %, 15.5 wt %, 16 wt %, 16.5 wt %, 17 wt %, 17.5 wt %, 18 wt %, 18.5 wt %, 19 wt %, 19.5 wt %, 20 wt %, 21 wt %, 22 wt %, 23 wt %, 24 wt %, 25 wt %, 26 wt %, 27 wt %, 28 wt %, 29 wt %, 30 wt %, 31 wt %, 32 wt %, 33 wt %, 34 wt %, 35 wt %, 36 wt %, 37 wt %, 38 wt %, 39 wt %, 40 wt %, 41 wt %, 42 wt %, 43.127 wt %, 44 wt %, 45 wt %, 46 wt %, 47 wt %, 48 wt %, 49 wt %, 49.99 wt % is a lower limit and a higher value is an upper limit.

If the mass ratio of the metal halide perovskite to the sum of the weights of the metal halide perovskite and the organic small molecule host outside the above range is less than 0.01 wt %, the energy transfer of the metal halide perovskite is not effectively performed in the organic small molecule host, so that the organic small molecule host may glow.

It is preferable that the metal halide perovskite-organic low-molecular host mixed light emitting layer according to the present disclosure is formed by a vapor deposition method. The deposition method may include vacuum deposition, thermal deposition, flash deposition, laser deposition, chemical vapor deposition, atomic layer deposition, physical vapor deposition, physical-chemical co-evaporation deposition, sequential vapor deposition, solution process-assisted thermal deposition, etc.

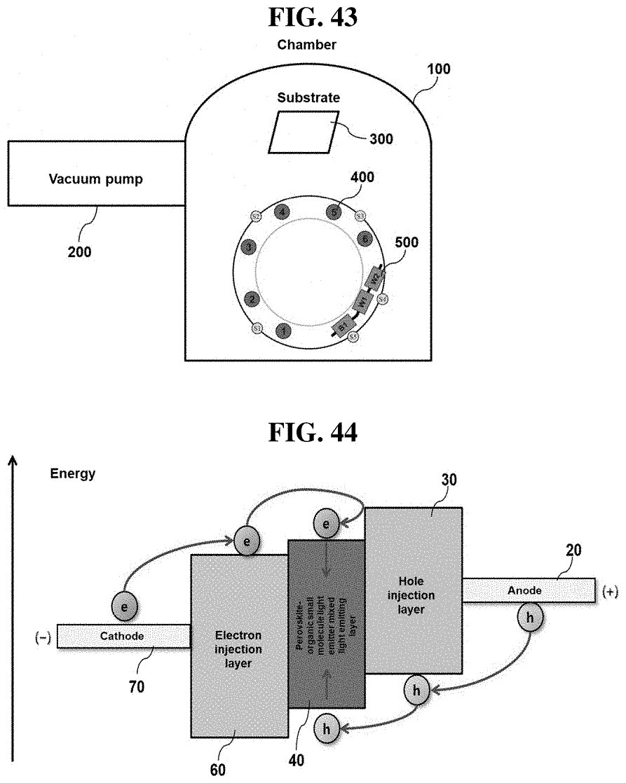

Vacuum evaporation may be preferably used as the deposition method, and in this case, the vacuum evaporation may be performed in high vacuum and low vacuum. In an embodiment of the present disclosure, a light emitting layer was formed using the vacuum evaporator shown in FIG. 43.

Referring to FIG. 43 in detail, the vacuum evaporator includes a chamber(100) and a vacuum pump(200), and in the chamber(100), a substrate(300) on which a substrate to be deposited is placed, and a metal halide. A crucible containing the perovskite precursor material(400) and the organic low molecular weight host material(500), and a heat source for heating the crucible is provided under the crucible. In the vacuum evaporation method, after placing the substrate(300) on the upper side in the chamber(100) of the vacuum evaporator and loading the metal halide perovskite precursor material(400) and the organic low molecular weight host material(500) at the bottom, when each material is heated with an electron beam or the like in a vacuum state, the vaporized materials are synthesized while being deposited on the substrate to form a metal halide perovskite-organic low-molecular host mixed light emitting layer.

The metal halide perovskite-organic low-molecular host mixture light emitting layer according to the present disclosure can form a uniform thin film by co-depositing the metal halide perovskite and an organic low-molecular host, and electroluminescence efficiency can be improved by controlling an energy level and an electron-hole recombination zone.

FIG. 44 shows energy levels of layers in a light-emitting device (normal structure) including a metal halide perovskite-organic low-molecular host mixed light-emitting layer according to an embodiment of the present disclosure.

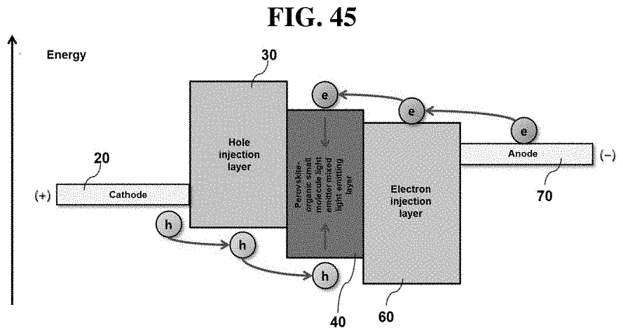

FIG. 45 shows energy levels of layers in a light-emitting device (inverted structure) including a metal halide perovskite-organic low-molecular host mixed light-emitting layer according to an embodiment of the present disclosure.

FIGS. 44 and 45, it is desirable that the energy level of VBM of the light emitting layer (40) is lower than that of the HOMO of the hole injection layer, and higher than the energy level of the HOMO of the electron transport layer in the light emitting device according to the present disclosure. When having such an energy level, if a forward bias is applied to the light emitting device, it becomes easy for holes(h) from the anode(20) to flow into the light emitting layer (40) through the hole injection layer (30).

In addition, it is desirable that the energy level of CBM of the light emitting layer is lower than the energy level of LUMO of the hole injection layer, and is preferably higher than the energy level of LUMO of the electron transport layer in the light emitting device according to the present disclosure. When having such an energy level, when a forward bias is applied to the light emitting device, it becomes easy for electrons(e) from the cathode(70) to flow into the light emitting layer (40) through the electron injection layer (60).

The metal halide perovskite is as described above, and a detailed description thereof will be omitted.

<Metal Halide Perovskite Light Emitting Device Including Multidimensional Hybrid Light Emitting Layer>

According to another embodiment of the present disclosure, the emission layer may include a multidimensional metal halide perovskite hybrid emission layer.

FIG. 46 is an example of a structure of a light emitting diode including a multidimensional metal halide perovskite hybrid light emitting layer according to an embodiment of the present disclosure.

As shown in FIG. 46, the above multi-dimensional metal halide perovskite hybrid luminescence layer may be formed by sequentially deposition or simultaneous deposition of metal halide perovskite bulk polycrystalline films and metal halide perovskite nanocrystal particles films.

The multidimensional metal halide perovskite hybrid light emitting layer according to the disclosure can induce surface passivation of nanocrystal particle through a metal halide perovskite bulk polycrystal, and improve device luminescence efficiency by confining excitons in the nanocrystal particle by coexisting metal halide perovskite nanocrystal particles and metal halide perovskite bulk polycrystals in the light emitting layer.

In addition, a light emitting layer composed of two layers can be produced by forming a metal halide perovskite nanocrystal particle layer on the metal halide perovskite bulk polycrystals in the multidimensional metal halide perovskite hybrid light emitting layer according to the present disclosure, and it is possible to implement a multicolor light-emitting device that is difficult to implement in a single-layer light-emitting layer device.

In this case, the multidimensional metal halide perovskite hybrid light emitting layer (30) may partially serve as the hole injection layer (25). Metal halide perovskite is a promising material as a light emitter, but basically, it has high potential as a charge carrier such as high carrier mobility and long carrier diffusion length, so its role can be extended not only to light emission but also to charge transport. In addition, since the energy level can be easily adjusted through a method such as halide ion exchange in a unit lattice of a metal halide perovskite, it can be used for injecting electric charges into various light emitting layers.

Accordingly, in the multidimensional metal halide perovskite hybrid light emitting layer of the present disclosure, the metal halide perovskite bulk polycrystals having high charge mobility and long exciton diffusion length functions as a charge transport layer. Also, since the metal halide perovskite nanocrystal particles can also serve as a charge transport layer, it is possible to improve device efficiency by promoting hole injection or electron injection into the light emitting layer.

The method of forming the multi-dimensional metal halide perovskite hybrid light emitting layer may be formed by various methods, and a specific method will be described in detail in the following manufacturing method section.

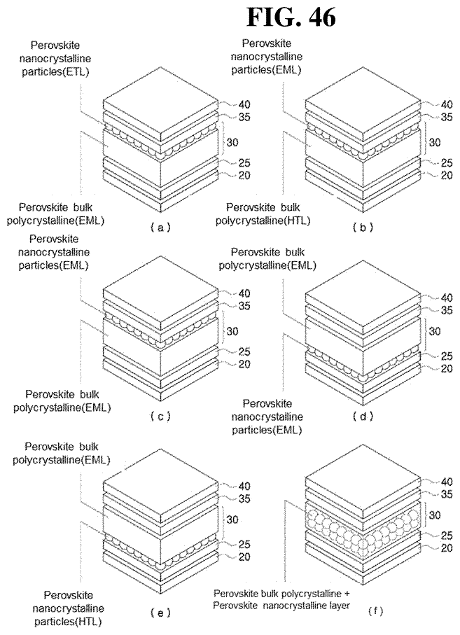

(a) Dripping Method (FIG. 47(a))

The steps of forming the multidimensional metal halide perovskite hybrid light emitting layer (30) include

Preparing a metal halide perovskite bulk polycrystalline precursor (1) solution and a metal halide perovskite nanocrystal particle (2) solution;

Applying the metal halide perovskite bulk polycrystalline precursor (1) solution onto the first electrode (20) or the hole injection layer; and

The step of coating the bulk polycrystalline material (1) and the metal halide perovskite nanocrystal particles (2) together before the coating of the metal halide perovskite bulk polycrystalline precursor (1) solution is completed, the metal halide perovskite nanocrystal particle (2) solution is dropped to the metal halide perovskite.

First, a metal halide perovskite bulk polycrystalline precursor (1) solution and a metal halide perovskite nanocrystal particle (2) solution are prepared.

The metal halide perovskite bulk polycrystal (1) and the metal halide perovskite nanocrystal particles (2) may use a material having the same chemical structure or a material having a different chemical structure.

The metal halide perovskite bulk polycrystalline precursor (1) solution can be prepared by dissolving the metal halide perovskite in a polar solvent (first solution).

In this case, the polar solvent may be selected from dimethylformamide, dimethyl sulfoxide, γ-butyrolactone, N-methylpyrrolidone, and isopropyl alcohol, but is not limited thereto.

Since the metal halide perovskite is the same as described above, a detailed description will be omitted.

On the other hand, such a metal halide perovskite can be prepared by combining AX and BX₂ in a certain ratio. That is, the first solution may be formed by dissolving AX and BX₂ in a polar solvent at a predetermined ratio. For example, a first solution in which ABX₃ metal halide perovskite is dissolved may be prepared by dissolving AX and BX₂ in a 1:1 ratio in a polar solvent.

The metal halide perovskite nanocrystal particles (2) solution above include

The step for preparing a first solution in which a metal halide perovskite is dissolved in a polar solvent and a second solution in which a surfactant is dissolved in a non-polar solvent or a polar solvent; and

The step for mixing the first solution with the second solution to form metal halide perovskite nanocrystal particles.

First, since the method of preparing the first solution is the same as the method of preparing the metal halide perovskite bulk polycrystalline precursor solution described above, a detailed description will be omitted.

The second solution is prepared by dissolving a surfactant in a non-polar solvent or a polar solvent.

The non-polar solvent may be selected from methanol, ethanol, tert-butanol, xylene, toluene, hexane, cyclohexene, dichloroethylene, trichloroethylene, chloroform, chlorobenzene, xylene, toluene, hexane, cyclohexene and dichlorobenzene In addition, the polar solvent may be selected from dimethylformamide, dimethyl sulfoxide, γ-butyrolactone, N-methylpyrrolidone, and isopropyl alcohol, but is not limited thereto.

The surfactant may include an amine ligand, an organic acid, an organic ammonium ligand, or an inorganic ligand.

The amine ligand may be selected from N,N-diisopropylethylethylamine, ethylenediamine, hexamethylenetetraamine, methylamine, hexylamine, oleylamine, N,N,N,N-tetramethylenediamine, triethylamine, diethanolamine, 2,2-(ethylenedioxyl)bis-(ethylamine), but is not limited thereto.

The organic acid may be selected from carboxylic acid and phosphonic acid, and carboxylic acid 4,4′-Azobis(4-cyanovaleric acid), acetic acid, 5-aminosalicylic acid, acrylic acid, L-a spentic acid, 6-bromohexanoic acid, bromoacetic acid, dichloroacetic acid, e thylenediaminetetraacetic acid, isobutyric acid, itaconic acid, maleic acid, r-maleimidobutylic acid, L-malic acid, 4-nitrobenzoic acid, 1-pyrenecarboxylic acid, hexanoic acid, octanoic acid, decanoic acid, undecanoic acid, dodecanoic acid, hexadecenoic acid, octadecanoic acid and oleic acid,

Phosphonic acid can be selected from n-hexylphosphonic acid, n-octylphosphonic acid, n-decylphosphonic acid, n-dodecylphosphonic acid, n-tetradecylphosphonic acid, n-hexadecylphosphonic acid, n-octadecylphonic acid.

The organic ammonium ligand is a ligand of the structure of alkyl-X, and the alkyl can be selected from an acyl alkyl(CₙH₂ₙ₊₁); a polyhydric alcohol(CₙH₂ₙ₊₁OH) including a primary alcohol, a secondary alcohol, and a tertiary alcohol; Alkylamine (alkyl-N) including hexadecyl amine, 9-octadecenylamine, and 1-amino-9-octadacene (C₁₉H₃₇N); It is selected from the group consisting of p-substituted aniline, phenyl ammonium and fluorine ammonium, and X may be Cl, Br or I.

The above first solution can be mixed with the above second solution to form nanocrystal particle, such as spraying, dripping finely drop by drop, dropping at a time, etc. first solution into second solution. In addition, the second solution at this time may be stirred. For example, a second solution containing strongly stirring amine ligands, organic acids (carboxylic acid or phosphonic acid), organic ammonium ligands, or inorganic ligand surfactants with vigorous stirring can be slowly added with a drop of a second solution containing an inorganic halide perovskite (OIP).

In this case, when the first solution is dropped into the second solution and mixed, organic-inorganic metal halide perovskite (OIP) is precipitated from the second solution due to a difference in solubility. The ligand (amine-based ligand) mixed in advance in the second solution adheres to the crystal structure of the metal halide perovskite, thereby reducing the difference in solubility to prevent rapid precipitation of the metal halide perovskite. In addition, a carboxylic acid surfactant or phosphonic acid surfactant attaches to the surface from the second solution, stabilizing the nanocrystals, creating a well-dispersed inorganic metal halide perovskite nanocrystal (OIP-NC). Accordingly, it is possible to prepare a metal halide perovskite nanocrystal particle including an organic-inorganic metal halide perovskite nanocrystal and surrounding the organic-inorganic metal halide perovskite nanocrystal surrounded by a plurality of inorganic or organic ligands.

However, when the compatibility between the first solution and the second solution is high, recrystallization may not occur, and in this case, a demulsifier may be additionally added.

Tert-butanol may be used as the demulsifier, but is not limited thereto.

The thus prepared metal halide perovskite nanocrystal particle solution is a colloidal solution in which metal halide perovskite nanocrystal particles are dispersed in a solvent.

At this time, since the metal halide perovskite nanocrystal particle solution contains unreacted materials and can be re-dissolved by a polar solvent containing the generated nanocrystal particle, in order to maintain the shape of the nanocrystal particle, the step of separating only the nanocrystal particle by centrifugation or the like and redispersing them in a non-polar solvent may be additionally performed.

In addition, the form of the metal halide perovskite nanocrystal may be a form generally used in the relevant field. The shape of the metal halide perovskite nanocrystal may be a 0-dimensional, 1-dimensional or 2-dimensional shape. As an example, it may be in the form of a sphere, an ellipsoid, a hollow cube, a pyramid, a cylinder, a cone, an elliptic column, hollow sphere, Janus particle, prism, multipod, polyhedron, nano tube, nano wire, nano fiber or nanoplatelet.

In addition, the size of the crystalline particles may be 1 nm to 10 μm or less. For example, it can be 1 nm, 2 nm, 3 nm, 4 nm, 5 nm, 6 nm, 7 nm, 7.5 nm, 8 nm, 8.5 nm, 9 nm, 9.5 nm, 10 nm, 10.5 nm, 11 nm, 11.5 nm, 12 nm, 12.5 nm, 13 nm, 13.5 nm, 14 nm, 14.5 nm, 15 nm, 16 nm, 17 nm, 18 nm, 19 nm, 20 nm, 25 nm, 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 150 nm, 200 nm, 250 nm, 300 nm, 350 nm, 400 nm, 450 nm, 500 nm, 600 nm, 700 nm, 800 nm, 900 nm, 1 μm, 2 μm, 5 μm, or 10 μm. The size of the particle can be defined as a range in which the lower value among the two numbers selected from the above is taken as the minimum value and the larger value as the maximum value. It is preferably 8 nm or more and 300 nm or less, and more preferably 10 nm or more and 30 nm or less. On the other hand, the size of the crystalline particles at this time means a size that does not take into account the length of a ligand to be described later, that is, the size of the remaining portions excluding the ligand. When the size of the crystalline particles is 1 μm or more, there is a fundamental problem in that excitons do not emit light due to thermal ionization and delocalization of charge carriers in a large crystal, but are separated into free charges and disappear. In addition, more preferably, as described above, the size of the crystalline particles may be greater than or equal to a Bohr diameter. The thermal ionization and delocalization of the charge carrier may gradually appear when the size of the nanocrystal exceeds 100 nm. If it is more than 300 nm, the phenomenon will appear more, and if it is more than 1 μm, it is completely bulky and is subject to the above phenomenon. For example, when the nanocrystal particles are spherical, the diameter of the nanocrystal particle may be 1 nm to 10 μm. It may preferably be 1 nm, 3 nm, 5 nm, 6 nm, 7 nm, 8 nm, 9 nm, 10 nm, 11 nm, 12 nm, 13 nm, 14 nm, 15 nm, 16 nm, 17 nm, 18 nm, 19 nm, 20 nm, 25 nm, 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 150 nm, 200 nm, 250 nm, 300 nm, 350 nm, 400 nm, 450 nm, 500 nm, 600 nm, 700 nm, 800 nm, 900 nm, 1 μm, 2 μm, 5 μm, or 10 um.

In addition, the band gap energy of the nanocrystal particle may be 1 eV to 5 eV. Preferably, the band gap energy of the nanocrystal particle may include a range in which the lower value of two numbers of 1 eV, 1.1 eV, 1.2 eV, 1.3 eV, 1.4 eV, 1.5 eV, 1.6 eV, 1.7 eV, 1.8 eV, 1.81 eV, 1.82 eV, 1.83 eV, 1.84 eV, 1.85 eV, 1.86 eV, 1.87 eV, 1.88 eV, 1.89 eV, 1.9 eV, 1.91 eV, 1.92 eV, 1.93 eV, 1.94 eV, 1.95 eV, 1.96 eV, 1.97 eV, 1.98 eV, 1.99 eV, 2 eV, 2.01 eV, 2.02 eV, 2.03 eV, 2.04 eV, 2.05 eV, 2.06 eV, 2.07 eV, 2.08 eV, 2.09 eV, 2.1 eV, 2.11 eV, 2.12 eV, 2.13 eV, 2.14 eV, 2.15 eV, 2.16 eV, 2.17 eV, 2.18 eV, 2.19 eV, 2.2 eV, 2.21 eV, 2.22 eV, 2.23 eV, 2.24 eV, 2.25 eV, 2.26 eV, 2.27 eV, 2.28 eV, 2.29 has a lower limit and a higher value has an upper limit.

Accordingly, since the energy band gap is determined according to the constituent material or crystal structure of the nanocrystal particle, light having a wavelength of, for example, 200 nm to 1300 nm may be emitted by controlling the constituent material of the nanocrystal particle. In addition, preferably, the nanocrystal particle may emit ultraviolet, blue, green, red, and infrared light.

The ultraviolet light may include a range in which a low value of two numbers of 200 nm, 210 nm, 220 nm, 230 nm, 240 nm, 250 nm, 260 nm, 270 nm, 280 nm, 290 nm, 300 nm, 310 nm, 320 nm, 330 nm, 340 nm, 350 nm, 360 nm, 370 nm, 380 nm, 390 nm, 400 nm, 410 nm, 420 nm, 430 nm has a lower limit and a high value has an upper limit. The blue light may include a range in which a low value of two numbers of 440 nm, 450 nm, 451 nm, 452 nm, 453 nm, 454 nm, 455 nm, 456 nm, 457 nm, 458 nm, 459 nm, 460 nm, 461 nm, 462 nm, 463 nm, 464 nm, 465 nm, 466 nm, 467 nm, 468 nm, 469 nm, 470 nm, 471 nm, 472 nm, 473 nm, 474 nm, 475 nm, 476 nm, 477 nm, 478 nm, 479 nm, 480 nm, 490 nm has a lower limit and a high value has an upper limit. The green light may include a range in which a low value of two numbers of 500 nm, 501 nm, 502 nm, 503 nm, 504 nm, 505 nm, 506 nm, 507 nm, 508 nm, 509 nm, 510 nm, 511 nm, 512 nm, 513 nm, 514 nm, 515 nm, 516 nm, 517 nm, 518 nm, 519 nm, 520 nm, 521 nm, 522 nm, 523 nm, 524 nm, 525 nm, 526 nm, 527 nm, 528 nm, 529 nm, 530 nm, 531 nm, 532 nm, 533 nm, 534 nm, 535 nm, 536 nm, 537 nm, 538 nm, 539 nm, 540 nm, 541 nm, 542 nm, 543 nm, 544 nm, 545 nm, 546 nm, 547 nm, 548 nm, 549 nm, 550 nm, 560 nm, 570 nm, 580 nm has a lower limit and a high value has an upper limit. The red light may include a range in which a low value of two numbers of 590 nm, 600 nm, 601 nm, 602 nm, 603 nm, 604 nm, 605 nm, 606 nm, 607 nm, 608 nm, 609 nm, 610 nm, 611 nm, 612 nm, 613 nm, 614 nm, 615 nm, 616 nm, 617 nm, 618 nm, 619 nm, 620 nm, 621 nm, 622 nm, 623 nm, 624 nm, 625 nm, 626 nm, 627 nm, 628 nm, 629 nm, 630 nm, 631 nm, 632 nm, 633 nm, 634 nm, 635 nm, 636 nm, 637 nm, 638 nm, 639 nm, 640 nm, 641 nm, 642 nm, 643 nm, 644 nm, 645 nm, 646 nm, 647 nm, 648 nm, 649 nm, 650 nm, 660 nm, 670 nm, 680 nm, 690 nm, 700 nm has a lower limit and a high value has an upper limit. The infrared light may include a range in which a low value of two numbers of 700 nm, 710 nm, 720 nm, 730 nm, 740 nm, 750 nm, 760 nm, 770 nm, 780 nm, 790 nm, 800 nm, 810 nm, 820 nm, 830 nm, 840 nm, 850 nm, 860 nm, 870 nm, 880 nm, 890 nm, 900 nm, 910 nm, 920 nm, 930 nm, 940 nm, 950 nm, 960 nm, 970 nm, 980 nm, 990 nm, 1000 nm, 1010 nm, 1020 nm, 1030 nm, 1040 nm, 1050 nm, 1060 nm, 1070 nm, 1080 nm, 1090 nm, 1100 nm, 1110 nm, 1120 nm, 1130 nm, 1140 nm, 1150 nm, 1160 nm, 1170 nm, 1180 nm, 1190 nm, 1200 nm, 1210 nm, 1220 nm, 1230 nm, 1240 nm, 1250 nm, 1260 nm, 1270 nm, 1280 nm, has a lower limit and a high value has an upper limit.

Next, the metal halide perovskite bulk polycrystalline (1) precursor solution is coated on the first electrode.

In this case, the first electrode may be rotated so that the precursor solution of the metal halide perovskite bulk polycrystalline (1) is evenly applied on the first electrode.

The rotation speed is preferably 1000 rpm to 8000 rpm, if it is less than 1000 rpm, there is a problem in forming a uniform thin film, and if it exceeds 8000 rpm, there is a problem in uniform crystal growth as evaporation of the solvent is remarkably accelerated, and there is a problem in forming a multidimensional metal halide perovskite light emitting layer because it is difficult to inject the metal halide perovskite nanocrystal particles.

In one embodiment of the present disclosure, while rotating the first electrode at 3000 rpm, the metal halide perovskite bulk polycrystalline (1) precursor solution was coated.

The coating may consist of spin coating, bar coating, nozzle printing, spray printing, slot die coating, gravure printing, inkjet printing, screen printing, electrohydrodynamic jet printing, or electrospray.

Next, before the coating of the metal halide perovskite bulk polycrystalline (1) precursor solution is completed, the metal halide perovskite nanocrystal particles (2) solution is injected (dripping) and the metal halide perovskite bulk polycrystalline and metal halide perovskite nanocrystal particles are coated together.

At this time, the time when the coating of the metal halide perovskite bulk polycrystalline (1) precursor solution is completed is the time when solvent of the metal halide perovskite bulk polycrystalline (1) precursor solution is evaporated and crystallization is completed, and it is different for each material, but it takes about 80 seconds. Therefore, it is preferable to drop the metal halide perovskite nanocrystal particle (2) solution within 1 second to 200 seconds after the coating of the metal halide perovskite bulk polycrystalline precursor solution.

In an embodiment of the present disclosure, the metal halide perovskite nanocrystal particles (2) solution is dropped and coated together 20 seconds after the application of the metal halide perovskite bulk polycrystalline (1) precursor solution.

After coating, heat treatment may be performed to increase the density of the thin film.

The heat treatment may be performed at 80-120° C.

In an embodiment of the present disclosure, the hybrid thin film was heat-treated at 90° C. for 10 minutes.

In this hybrid thin film coated with a metal halide perovskite bulk polycrystalline (1) precursor solution and a metal halide perovskite nanocrystal particle (2) solution, metal halide perovskite nanocrystals act as a crystallization seed, providing many crystallization sites, which induces the granular structure of the thin film, as shown in FIG. 5. Accordingly, compared to the conventional single-dimensional light emitting layer made of only one-dimensional material, it is possible to exhibit remarkably improved light emission intensity.

The thickness of the formed multidimensional metal halide perovskite hybrid light emitting layer may be 10 nm to 10 μm.

In addition, the emission wavelength of the multidimensional metal halide perovskite hybrid emission layer may be 200 nm to 1300 nm.

In addition, the band gap energy of the multidimensional metal halide perovskite hybrid light emitting layer may be 1 eV to 5 eV.

(b) Over-Coating Method (FIG. 47(b))

In addition, the steps of forming the multidimensional metal halide perovskite hybrid light emitting layer (30) include

Preparing a metal halide perovskite bulk polycrystalline (1) precursor solution and a metal halide perovskite nanocrystal particle solution;

Forming a metal halide perovskite bulk polycrystalline thin film by coating and coating the metal halide perovskite bulk polycrystalline (1) precursor solution on a first electrode or a hole injection layer; And

Coating the metal halide perovskite nanocrystal particles (2) solution on the formed metal halide perovskite bulk polycrystalline (1) thin film.

In the case of metal halide perovskite nanocrystal particles (2), since the crystals are dispersed in an anti-solvent that does not dissolve and are used to make a thin film, it is possible to coat on the previously formed metal halide perovskite bulk polycrystalline film (1).

Since the steps of preparing the metal halide perovskite bulk polycrystalline (1) precursor solution and the metal halide perovskite nanocrystal particle (2) solution are the same as described above, a detailed description will be omitted.

Next, a metal halide perovskite bulk polycrystalline (1) precursor solution is coated on the hole injection layer.

The difference between the overcoating method and the above-described dripping method is that the dripping method is to make a thin film by coating a solution of low-dimensional metal halide perovskite nanocrystal particles (2) before the high-dimensional metal halide perovskite bulk polycrystal (1) thin film is formed, however, the overcoating method is to coat metal halide perovskite nanocrystal particles (2) after forming a metal halide perovskite bulk polycrystalline film (1).

At this time, in order to evenly apply the metal halide perovskite bulk polycrystal (1) precursor solution onto the first electrode (20) or the hole injection layer (25), the first electrode (20) or the hole injection layer (25) can be rotated. Likewise, even when applying the metal halide perovskite nanocrystal particles (2) solution, the object to be coated may be rotated in order to apply evenly.

The rotational speed is preferably 1000 to 8000 rpm. If it is less than 1000 rpm, there is a problem in forming a uniform thin film, and if it exceeds 8000 rpm, evaporation of the solvent is remarkably accelerated, resulting in a problem in uniform crystal growth.

The coating may be made of spin coating, bar coating, nozzle printing, spray printing, slot die coating, gravure printing, inkjet printing, screen printing, electrohydrodynamic jet printing, or electrospray.

In addition, after coating the nanocrystal particle (2), a purification process for removing impurities from the nanocrystal particle (2) solution may be additionally performed, and the purification process may be performed by applying a non-polar solvent.

In an embodiment of the present disclosure, impurities were removed by applying the non-polar solvent while rotating at a speed of 3000 rpm for 20 seconds.

After coating, heat treatment may be performed to increase the density of the thin film.

The heat treatment may be performed at 80 to 120° C.

In an embodiment of the present disclosure, the prepared metal halide perovskite bulk polycrystalline (1) thin film and the metal halide perovskite nanocrystal particle (2) layer were heat-treated at 90° C. for 10 minutes.

The multidimensional metal halide perovskite hybrid light emitting layer manufactured by the overcoating method according to an embodiment of the present disclosure exhibits remarkably improved light emission intensity compared to the conventional single-dimensional light emitting layer made of only one-dimensional material, When a light emitting device is fabricated using metal halide perovskite bulk polycrystals and nanocrystal particle having different emission wavelength as a light emitting layer, electroluminescence can be generated in both wavelength.

(c) Polycrystalline Vacuum Deposition Method (FIG. 47(c))

In addition, the steps of forming the multidimensional metal halide perovskite hybrid light emitting layer (30) include

Preparing a solution of a metal halide perovskite bulk polycrystalline (1) precursor and a metal halide perovskite nanocrystal particle (2);

Forming a metal halide perovskite nanocrystal particle (2) layer by coating the metal halide perovskite nanocrystal particle (2) solution on the first electrode (20) or the hole injection layer (25); and

The vacuum deposition of the metal halide perovskite bulk polycrystalline (1) precursor on the formed metal halide perovskite nanocrystalline (2) layer.

(d) Simultaneous Deposition Method (FIG. 47(d))

In addition, the step of forming the multidimensional metal halide perovskite hybrid light emitting layer (30)

can be performed by simultaneous vacuum deposition of metal halide perovskite bulk polycrystalline (1) precursors and metal halide perovskite nanocrystal particles (2).

For the polycrystalline vacuum deposition method or simultaneous deposition method, the deposition method can be selected from co-deposition, thermal deposition, flash deposition, laser deposition, chemical vapor deposition, atomic layer deposition, physical vapor deposition, physical-chemical co-evaporation deposition, sequential vapor deposition, solution process solution process-assisted thermal deposition and spray deposition.

<Metal Halide Perovskite Light Emitting Device Including a Metal Halide Perovskite Light Emitting Layer Having a 3D/2D Core-Shell Crystal Structure>

According to another embodiment of the present disclosure, the emission layer may be a metal halide perovskite film having a 3D/2D core-shell crystal structure.

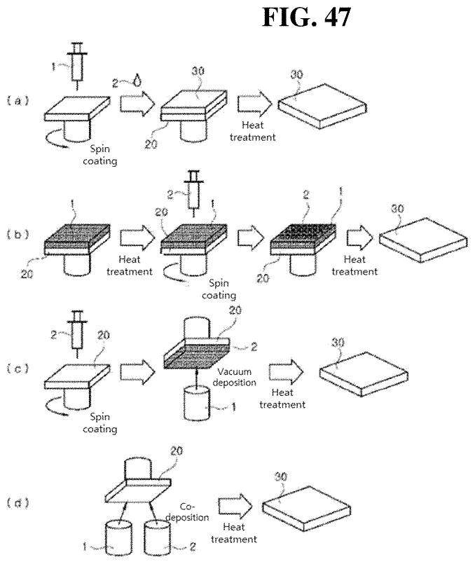

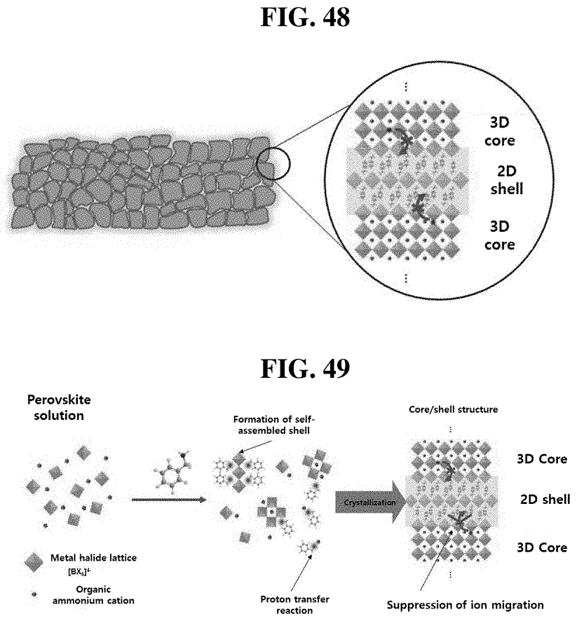

FIG. 48 shows a core/shell structure of a metal halide perovskite film according to an embodiment of the present disclosure.

Referring to FIG. 48, the metal halide perovskite film according to the present disclosure is composed of a core made of a three-dimensional metal halide perovskite crystal, and a shell made of a two-dimensional metal halide perovskite surrounding the core.

The core is composed of a three-dimensional metal halide perovskite crystal of ABX₃ or A′₂Aₙ₋₁BX₃ₙ₊₁ (n is an integer of 2 to 100), and the shell surrounding the core consists of a two-dimensional metal halide perovskite of Y₂Aₘ₋₁BX₃ₘ₊₁ (m is an integer of 1 to 100) including phenylalkanamine compound(Y) of Chemical Formula 1.

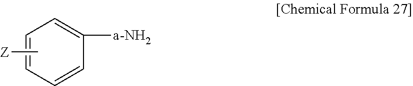

(In Chemical Formula 27, a is a unsubstituted or amine-substituted linear or branched alkyl of C₁ to C₁₀, and Z is F or CF₃.)

A and A′ may be monovalent cations, B may be a metal material, and X may be a halogen element.

The monovalent cation may be a monovalent organic cation or an alkali metal. For example, the monovalent organic cation may be organic ammonium (RNH₃)⁺, organic amidinium derivative (RC(═NR₂)NR₂)⁺, organic guanidinium derivative (R₂NC(═NR₂)NR₂)⁺, organic diammonium (CₓH₂ₓ₋ₙ₊₄)(NH₃)ₙ⁺, ((CₓH₂ₓ₊₁)ₙNH₃)(CH₃NH₃)ₙ⁺, (RNH₃)₂⁺, (CₙH₂ₙ₊₁NH₃)₂⁺, (CF₃NH₃)⁺, (CF₃NH₃)ₙ⁺, ((CₓF₂ₓ₊₁)ₙNH₃)₂(CF₃NH₃)ₙ⁺, ((CₓF₂ₓ₊₁)ₙNH₃)₂⁺ or (CₙF₂ₙ₊₁NH₃)₂⁺ (x, n is an integer greater than or equal to 1, R=hydrocarbon derivative, H, F, Cl, Br, I) and combinations thereof, but is not limited thereto. The alkali metal may be Li⁺, Na⁺, K⁺, Rb⁺, Cs⁺, Fr⁺, and combinations thereof, but is not limited thereto.

In addition, preferably, the organic cations may be acetamidinium, azaspironanium, benzene diammonium, benzylammonium, butanediammonium, iso-butylammonium, n-butylammonium, t-butylammonium, cyclohexylammonium, cyclohexylmethylammonium, diazobicyclooctanedinium, diethylammonium, N,N-diethylethane diammonium, dimethylammonium, dodecylammonium, ethanediammonium, ethylammoniuium, 4-fluoro-benzylammonium, 4-fluoro-phenylethylammonium, 4-fluoro-phenylammonium, formamidinium, guanidinium, hexanediammnium, hexylammonium, imidazolium, 2-methoxyethylammonium, 4-methoxy-phenylethylammonium, 4-methoxy-phenylammonium, methylammonium, morpholinium, octylammonium, pentylammonium, piperazinediium, piperidinium, propanediammonium, iso-propylammonium, di-isopropylammonium, n-propylammonium, Pyridinium, 2-pyrrolidin-1-ium-1-yethylammonium, pyrrolidinium, quinclidin-1-ium), 4-trifluoromethyl-benzylammonium, 4-trifluoromethyl ammonium, quaternary ammonium cations such as benzalkonium chloride, dimethyldioctadecylammonium chloride, trimethylglycine, choline and combinations thereof, but are not limited thereto.

The B may be a divalent transition metal, a rare earth metal, an alkaline earth metal, a monovalent metal, a combination of a trivalent metal, an organic substance (a monovalent, divalent, trivalent cation), and a combination thereof. In addition, preferably, the divalent transition metal, rare earth metal, and alkaline earth metal may be Be²⁺, Mg²⁺, Ca²⁺, Sr²⁺, Ba²⁺, Ra²⁺, Mn²⁺, Fe²⁺, Co²⁺, Ni²⁺, Cu²⁺, Zn²⁺, Ru²⁺, Pd²⁺, Cd²⁺, Pt²⁺, Hg²⁺, Ge²⁺, Sn²⁺, Pb²⁺, Se²⁺, Te²⁺, Po²⁺, Bi²⁺, Eu²⁺, No²⁺, and combinations thereof, but are not limited thereto. Monovalent metal may be Li⁺, Na⁺, K⁺, Rb⁺, Cs⁺, Fr⁺, Ag⁺, Hg⁺, Ti⁺, and combinations thereof, trivalent metal may be Cr³⁺, Fe³⁺, Co³⁺, Ru³⁺, Rh³⁺, Ir³⁺, Au³⁺, Al³⁺, Ga³⁺, In³⁺, Ti³⁺, As³⁺, Sb³⁺, Bi³⁺, La³⁺, Ce³⁺, Pr³⁺, Nd³⁺, Pm³⁺, Sm³⁺, Eu³⁺, Gd³⁺, Tb³⁺, Dy³⁺, Ho³⁺, Er³⁺, Tm³⁺, Yb³⁺, Lu³⁺, Ac³⁺, Am³⁺, Cm³⁺, Bk³⁺, Cf³⁺, Es³⁺, Fm³⁺, Md³⁺, Lr³⁺, and combinations thereof.

In addition, X may be F⁻, Cl⁻, Br⁻, I⁻, At⁻, and combinations thereof.

The three-dimensional metal halide perovskite crystal may be a metal halide perovskite, and the crystal structure of the metal halide perovskite has a central metal (B) in the center, and has a face centered cubic (FCC) structure. 6 halogen elements (X) are located on all surfaces of the hexahedron, and 8 of A or A′ (organic ammonium, organic phosphonium, or alkali metal) are located at all vertices of the hexahedron. Also, all sides of the hexahedron are orthogonal, and it includes not only a cubic structure with the same horizontal length, vertical length, and height, but also a tetragonal structure having the same horizontal length and vertical length but different heights.

The phenylalkanamine compound (Y) of Formula 27 acts as an ion transfer inhibitor and forms a self-assembled shell upon crystallization by reacting with metal halide perovskite ions through a proton transfer reaction in a solvent.

Examples of the phenylalkalamine compound of Formula 27 used in the present disclosure may include phenylmethanamine, (4-fluorophenyl)methanamine, (4-(trifluoromethyl)phenyl)methanamine, 2-phenylethanamine, 1-phenylpropan-2-amine, 1-phenylpropane-1-Amine, 1-phenylethane-1,2-diamine, 2-(4-fluorophenyl)ethanamine, 1-(4-fluorophenyl)propan-2-amine, 1-(4-fluorophenyl)propane-1-amine, 1-(4-fluorophenyl)ethane-1,2-diamine, 2-(4-(trifluoromethyl)phenyl)ethanamine, 1-(4-(trifluoromethyl)phenyl)Propan-2-amine, 1-(4-(trifluoromethyl)phenyl)propan-1-amine, 3-phenylpropan-1-amine, 4-phenylbutan-2-amine, 1-phenylbutan-2-amine, 1-phenylbutan-1-amine, 3-phenylpropane-1,2-diamine, 3-(4-fluorophenyl)propane-1-Amine, 4-(4-fluorophenyl)butan-2-amine, 1-(4-fluorophenyl)butan-1-amine, 4-phenylbutan-1-amine, 5-phenylpentan-2-amine, 1-phenylpentan-3-amine, 1-phenylpentan-1-amine, 4-(4-fluorophenyl)butan-1-amine, 1-(4-fluorophenyl)pentan-3-amine, 1-(4-fluorophenyl)pentan-1-amine, 5-phenylpentan-1-amine, 1-phenylhexan-1-amine, 1-phenylhexan-2-amine, 1-phenylhexan-3-amine, 6-phenylhexan-2-amine, 1-(4-fluorophenyl)hexan-1-amine, 1-(4-fluorophenyl)hexan-3-amine, 6-phenylhexan-1-amine and 1-phenylheptane-1-amine but are not limited thereto.

The chemical structures in the example of compounds that may be used in the phenylalkanamine compound (Y) of the above chemical formula 27 are summarized as shown in Table 3.

TABLE 3

| Ex- | | |
|---|---|---|
| ample | Name | Structure |
| 1 | Phenylmethanamine | 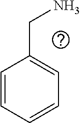 |
| 2 | (4- fluorophenyl) methanamine | 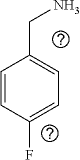 |
| 3 | (4- (trifluoromethyl) phenyl)methanamine | 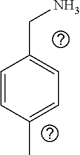 |
| 4 | 2- phenylethanamine | 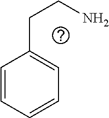 |
| 5 | 1- phenylpropane- 2-amine | 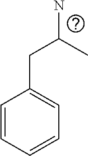 |
| 6 | 1- phenylpropan- 1-amine | 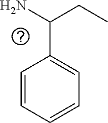 |
| 7 | 1- phenylethane- 1,2-diamine | 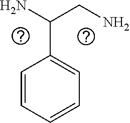 |
| 8 | 2-(4- fluorophenyl) ethanamine | 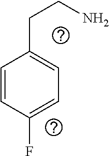 |
| 9 | 1-(4- fluorophenyl) propane- 2-amine | 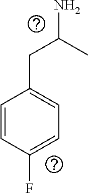 |
| 10 | 1-(4- fluorophenyl) propane- 1-amine | 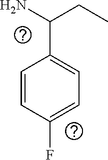 |
| 11 | 1-(4- fluorophenyl) ethane- 1,2-diamine | 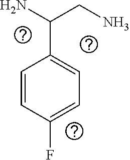 [SMILES: NCC(N)c1ccc(F)cc1] |
| 12 | 2-(4- (trifluoromethyl) phenyl) ethanamine | 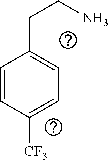 [SMILES: NCCc1ccc(C(F)(F)F)cc1] |
| 13 | 1-(4- (trifluoromethyl) phenyl) propane- 2-amine | 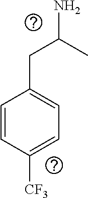 |
| 14 | 1-(4- (trifluoromethyl) phenyl) propane- 1-amine | 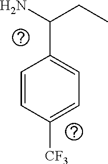 |
| 15 | 3- phenylpropane- 1-amine | 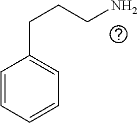 |
| 16 | 4- phenylbutane- 2-amine | 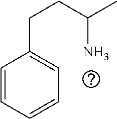 |
| 17 | 1- phenylbutane- 2-amine | 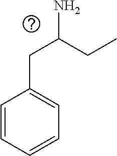 |
| 18 | 1- phenylbutane- 1-amine | 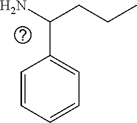 |
| 19 | 3- phenylpropane- 1,2-diamine | 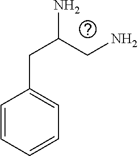 [SMILES: NCC(N)Cc1ccccc1] |
| 20 | 3-(4- fluorophenyl) propane- 1-amine | 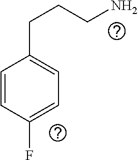 [SMILES: NCCCc1ccc(F)cc1] |
| 21 | 4-(4- fluorophenyl) butane-2- amine | 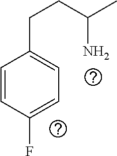 |
| 22 | 1-(4- fluorophenyl) butane-1- amine | 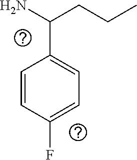 |
| 23 | 4- phenylbutane- 1-amine | 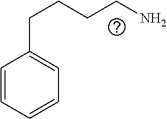 [SMILES: NCCCCc1ccccc1] |
| 24 | 5- phenylpentane- 2-amine | 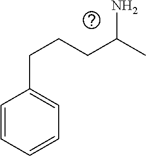 |
| 25 | 1- phenylpentane- 3-amine | 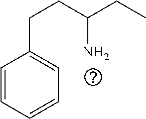 |
| 26 | 1- phenylpentane 1-amine | 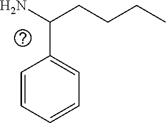 |
| 27 | 4-(4- fluorophenyl) butane-1- amine | 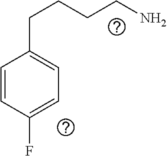 [SMILES: NCCCCc1ccc(F)cc1] |
| 28 | 1-(4- fluorophenyl) pentane- 3-amine | 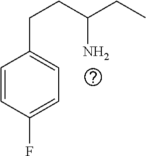 |
| 29 | 1-(4- fluorophenyl) pentane-1- amine | 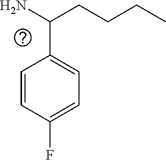 |
| 30 | 5- phenylpentane- 1-amine | 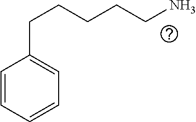 [SMILES: NCCCCCc1ccccc1] |
| 31 | 1- phenylhenxane- 1-amine | 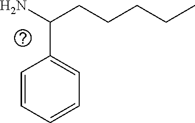 |
| 32 | 1- phenylhenxane- 2-amine | 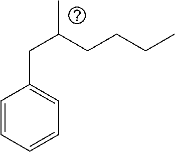 |
| 33 | 1- phenylhenxane- 3-amine | 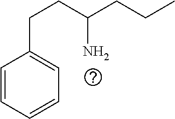 |
| 34 | 5- phenylhenxane- 2-amine | 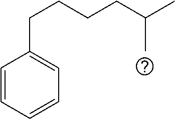 |
| 35 | 1-(4- fluorophenyl) hexane- 1-amine | 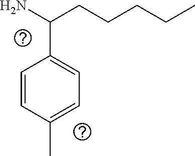 |
| 36 | 1-(4- fluorophenyl) hexane- 3-amine | 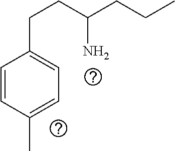 |
| 37 | 6- phenylhexane- 1-amine | 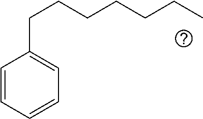 |
| 38 | 1- phenylheptane- 1-amine | 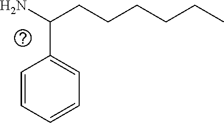 |

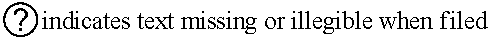

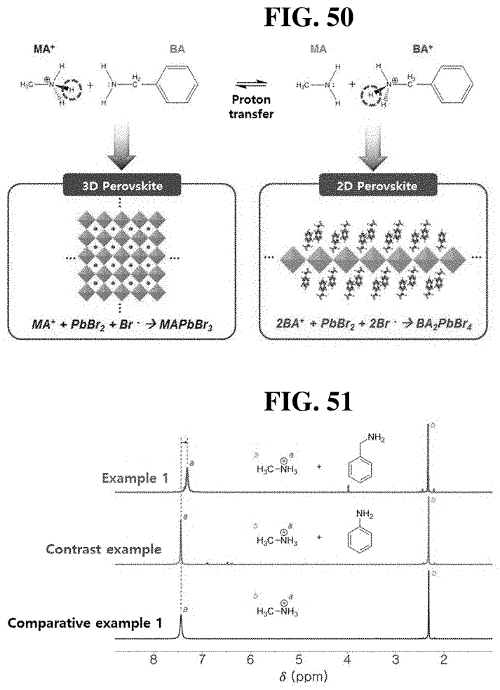

FIG. 49 shows a mechanism for forming a core/shell structure of a metal halide perovskite film according to an embodiment of the present disclosure, FIG. 50 shows a mechanism of forming each of the core and shell structures of a metal halide perovskite film according to an embodiment of the present disclosure.

When organic ammonium is present in an ionic state in the metal halide perovskite bulk precursor solution, when a phenylalkanamine additive is added to the solution, the organic ammonium and phenylalkanamine immediately act as acids and bases, respectively, and the acid-base Through the reaction, protons are transferred from the organic ammonium to the phenylalkanamine additive. The phenylalkanamine added at this time is a strong basic substance having a basicity (pK_{b}) of 7 or less, more preferably 5 or less, and changes to the phenylalkanammonium form through an immediate proton transfer reaction with most of the organic ammonium, as shown in FIG. Likewise, it exists in an active state capable of participating in the formation of 2D metal halide perovskite crystals. This proton transfer reaction can be confirmed by the fact that the proton of the methylammonium ion at 7.4 ppm on the chemical transfer position in the nuclear magnetic resonance spectroscopy spectrum of FIG. 51 moved to 7.2 ppm after adding a small amount of phenylmethylamine.

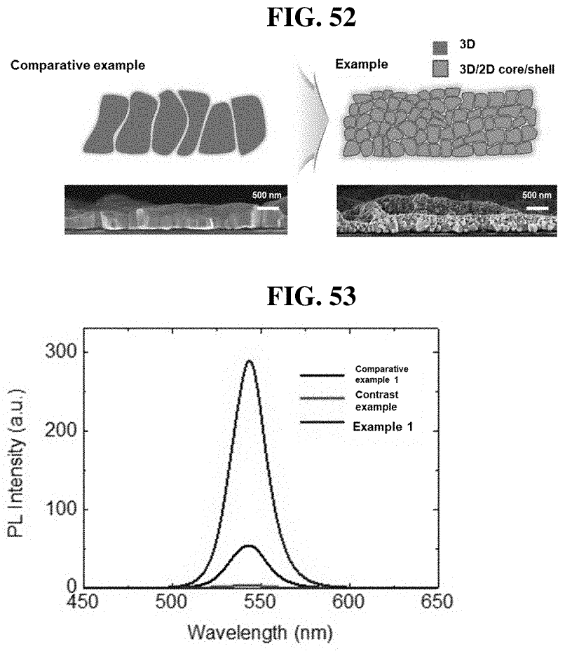

On the other hand, as a control example, the structure is similar to that of phenylalkanamine, but in the case of phenylamine (aniline) having no alkyl group between phenyl and amine, it exhibits weak basicity with a basicity of 7 or more, and the organic metal halide perovskite precursor solution Ammonium and acid-base reaction does not occur, so proton transfer does not occur (see FIG. 51). As a result, it is not possible to participate in the formation of the 2D metal halide perovskite shell, and thus no improvement in luminescence efficiency and lifetime or a two-dimensional crystal structure is observed (see FIG. 52 to 54).

Therefore, in order to obtain a metal halide perovskite having a 3D/2D core-shell crystal structure of the present disclosure, a proton transfer reaction of organic ammonium in the metal halide perovskite is essential, and such a proton transfer reaction is performed between a phenyl group and an amine. By including an alkyl group, it can be achieved through the addition of a phenylalkanamine compound having strong basicity.

The size of the crystal of the metal halide perovskite having a 3D/2D core-shell crystal structure in the metal halide perovskite film according to the present disclosure may be 10 nm to 1 μm, but is not limited thereto.

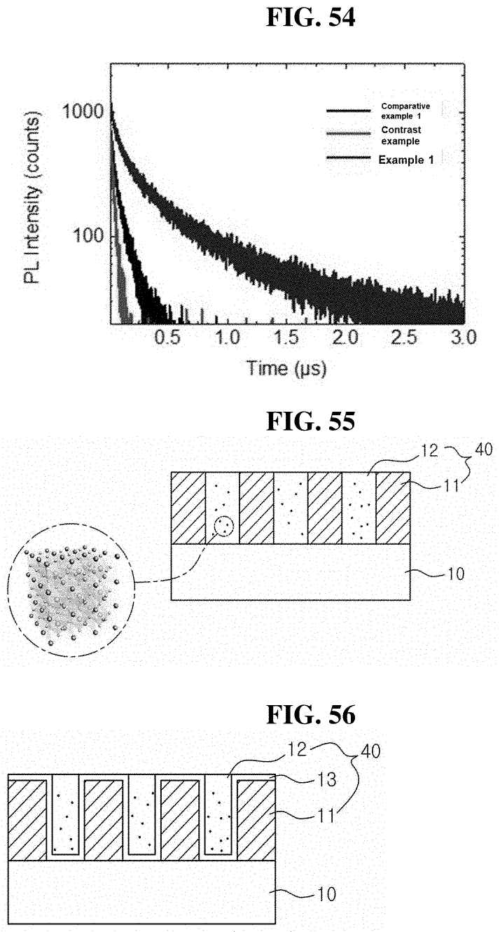

Metal halide perovskite films with 3D/2D core-shell crystalline structure according to disclosure were shown to have approximately six times more luminous intensity than bulk nanocrystal metal halide perovskite films with original 3D crystal structure, as well as significantly improved light efficiency and lifetime(see FIGS. 53 and 54). Therefore, the metal halide perovskite film having a 3D/2D core-shell crystal structure according to the present disclosure can be usefully used in a light emitting layer of a light emitting device.

In addition, the present disclosure provides a method of manufacturing a metal halide perovskite film having a 3D/2D core-shell crystal structure.

The method of manufacturing a metal halide perovskite film having a 3D/2D core-shell crystal structure of the present disclosure comprises preparing a mixed solution by adding a phenylalkanamine compound of Formula 1 to a metal halide perovskite bulk precursor solution (S100) and a metal halide perovskite film having a 3D/2D core-shell crystal structure by coating and coating a mixed solution of the metal halide perovskite bulk precursor solution and a phenylalkanamine compound on a member for applying a light emitting layer It includes a step (S200) of manufacturing.

Hereinafter, the present disclosure will be described step by step.

First, S100 is a step of preparing a mixed solution of a metal halide perovskite bulk precursor solution and a phenylalkanamine compound.

The metal halide perovskite bulk precursor solution may be formed by dissolving AX and BX₂ in a solvent at a predetermined ratio. For example, a metal halide perovskite bulk precursor solution in which ABX₃ metal halide perovskite is dissolved may be prepared by dissolving AX and BX₂ in an aprotic solvent at a ratio of 1.06:1.

In addition, the metal halide perovskite bulk precursor solution may be formed by mixing at least one of AX and A′X and BX₂ in an aprotic solvent.

The solvent used to prepare the metal halide perovskite bulk precursor solution is dimethylformamide, γ-butyrolactone, N-methylpyrrolidone, or dimethylsulfoxide, and combinations thereof.

The concentration of the metal halide perovskite bulk precursor solution may be 0.01M to 1.5M. If the concentration of the metal halide perovskite bulk precursor solution is less than 0.01M, the substrate to be coated cannot be completely covered due to the low concentration, and the flatness is lowered, making the device behavior impossible due to leakage current when applied as a light emitting device. And, if it exceeds 1.5M, there is a problem in that the crystals are aggregated in the process of forming a thin film due to a high concentration, resulting in a large crystal and a rough surface.

Since the description of the phenylalkanamine compound is the same as described above, it will be omitted to avoid redundant description.

In the mixed solution of the metal halide perovskite bulk precursor solution and the phenylalkanamine compound, the phenylalkanamine compound can be mixed in a ratio of 0.1 mol. % to 20 mol. % with respect to the metal halide perovskite bulk precursor solution. For example, the mixing ratio of the phenylalkanamine compound to the metal halide perovskite bulk precursor solution may include a range in which the lower value of two numbers of 0.1 mol. %, 0.5 mol. %, 1 mol. %, 1.5 mol. %, 2 mol. %, 2.5 mol. %, 3 mol. %, 3.1 mol. %, 3.2 mol. %, 3.3 mol. %, 3.4 mol. %, 3.5 mol. %, 3.6 mol. %, 3.7 mol. %, 3.8 mol. %, 3.9 mol. %, 4 mol. %, 4.1 mol. %, 4.2 mol. %, 4.3 mol. %, 4.4 mol. %, 4.5 mol. %, 4.6 mol. %, 4.7 mol. %, 4.8 mol. %, 4.9 mol. %, 5 mol. %, 5.1 mol. %, 5.2 mol. %, 5.3 mol. %, 5.4 mol. %, 5.5 mol. %, 5.6 mol. %, 5.7 mol. %, 5.8 mol. %, 5.9 mol. %, 6 mol. %, 6.1 mol. %, 6.2 mol. %, 6.3 mol. %, 6.4 mol. %, 6.5 mol. %, 6.6 mol % is a lower limit and the higher value is an upper limit.

If the phenylalkanamine compound is mixed in less than 0.1 mol. %, a self-assembled shell may not be formed, and if the phenylalkanamine compound is mixed in more than 20 mol. %, the 2D metal halide perovskite structure is formed. Since the formation is dominant, there may be a problem in that a core-shell structure in which the 2D metal halide perovskite surrounds only the crystal surface while maintaining the 3D metal halide perovskite crystal is not obtained. In addition, since the amount of residual organic ammonium changed to an amine state through a proton transfer reaction increases, charge injection and transfer when applied as a light emitting device, and efficient luminescent recombination may be hindered.

The phenylalkanamine compound receives protons from organic ammonium ions in the metal halide perovskite bulk precursor solution and changes into a cationic form.

Next, step S200 is a fabricating a metal halide perovskite film having a 3D/2D core-shell crystal structure by coating a mixed solution of the metal halide perovskite bulk precursor solution and a phenylalkanamine compound on a light emitting layer.

The member for applying the light emitting layer may be a substrate, an electrode, or a semiconductor layer. The substrate, electrode, or semiconductor layer may be a substrate, electrode, or semiconductor layer that can be used in a light emitting device. In addition, the member for applying the light emitting layer may have a form in which a substrate/electrode is sequentially stacked or a form in which a substrate/electrode/semiconductor layer is sequentially stacked.

Since the description of the substrate, the electrode, or the semiconductor layer is the same as described above, detailed information will be omitted.

Coating methods can be selected from groups of spin-coating, bar-coating, nozzle printing, spray-coating, slot-coating, Gravure-printing, inkjet printing, screen printing, electrohydrodynamic jet printing, and electrospray, but are not limited thereto.

After coating, the above phenylalkanamine compounds in the form of cations upon crystallization react with metal ions and halogen ions in a solution of metal halide perovskite bulk precursors to form a two-dimensional self-assembled shell, resulting in metal halide perovskite crystals in core-shell structures.

<Self-Assembled Polymer-Metal Halide Perovskite Light Emitting Device Including a Metal Halide Perovskite Light Emitting Layer>

According to another embodiment of the present disclosure, the emission layer may include a self-assembled polymer-metal halide perovskite emission layer.

FIG. 55 is a schematic diagram of a self-assembled polymer-metal halide perovskite light emitting layer according to an embodiment of the present disclosure.

Referring to FIG. 55, the self-assembled polymer-metal halide perovskite light emitting layer (40) according to the present disclosure may be formed on the member (10) for applying the light emitting layer, and the self-assembled polymer (11) forming a pattern and a metal halide perovskite nanocrystal particle layer (12) formed inside the pattern of the self-assembled polymer (11).

In the self-assembled polymer-metal halide perovskite light emitting layer according to the present disclosure, the self-assembled polymer (11) is composed of two or more polymers, and the two or more polymers are covalently linked through one end of a chain. As a tangible polymer, molecules in the polymer can spontaneously express a periodic structure in the form of a cylinder at the nanoscale level due to intermolecular interactions. By removing a specific polymer from the self-assembled polymer expressed in the cylindrical structure, periodic patterns can be formed, and the patterns are made of a metal halide perovskite material by binding metal halide perovskite nanocrystal particles. Since the luminescence efficiency can be improved and the ion migration phenomenon between the metal halide perovskite crystals can be blocked, stability can be improved, and the emission wavelength of the light emitting diode can be shifted toward blue (blue-shift). PLQY and luminance can be improved.

These self-assembled polymers 11 include PEO(Polyethylene oxide), PS(Polystyrene), PCL(Polycaprolactone), PAN(Polyacrylonitrile), PMMA(Poly(methyl methacrylate)), polyimide, PVDF(Poly(vinylidene fluoride)), a random copolymer consisting of two or more polymers selected from the group consisting of PVK (Poly(n-vinylcarbazole)), PVC (Polyvinylchloride), and their respective derivatives, an alternating copolymer, and a block copolymer A block copolymer or a graft copolymer may be used, but is not limited thereto.

The self-assembled polymer may be composed of a single layer or multiple layers of two or more depending on the thickness of the light emitting layer to be formed.

For the self-assembly polymer-metal halide perovskite luminous layer according to this disclosure, the pattern of the above self-assembly macromolecules (11) may be formed by removing a particular composition of polymer from a mass of two or more types expressed in a cylinder-shaped structure.

At this time, the width of the formed self-assembled polymer pattern is preferably 10 to 100 nm, more preferably 10 to 30 nm.

In the self-assembled polymer-metal halide perovskite light emitting layer according to the present disclosure, the metal halide perovskite nanocrystal particles constituting the metal halide perovskite nanocrystal particle layer (12) are a light emitter forming a light emitting layer, it may include a metal halide perovskite or metal halide perovskite nanocrystals or colloidal nanoparticles that can be dispersed in an organic solvent.

Since the metal halide perovskite is the same as described above, a detailed description will be omitted.

Meanwhile, as shown in FIG. 56, an organic material layer (13) surrounding the self-assembled polymer (11) may be further included between the patterned self-assembled polymer (11) and the metal halide perovskite nanocrystal particle region (12).

When the width of the pattern of the self-assembled polymer (11) is too wide, the organic material layer (13) may be coated on the self-assembled polymer (11) to reduce the width.

The organic material layer (13) may be formed of a polymer having the same composition as that of the self-assembled polymer, or other polymers. As an example, the organic material used in the organic material layer is PEO(Polyethylene oxide), PS(Polystyrene), PCL(Polycaprolactone), PAN(Polyacrylonitrile), PMMA(Poly(methyl methacrylate)), polyimide, polythiophene, polyaniline, polypyrrole, poly(3,4-ethylenedioxythiophene), PVDF(Poly(vinylidene fluoride)), PVK (Poly(n-vinylcarbazole)), PVC (Polyvinylchloride), and a mixture of two or more selected from the group consisting of, but is not limited thereto.

The organic material layer (13) may be formed on the self-assembled polymer pattern by a deposition method used in the industry, for example, the chemical vapor deposition (CVD) or thermal deposition method (thermal deposition) can be used, but it is not limited thereto.

The thickness of the organic material layer (13) may be adjusted according to the width of the formed self-assembled polymer pattern, and may be, for example, 1 to 20 nm, but is not limited thereto.

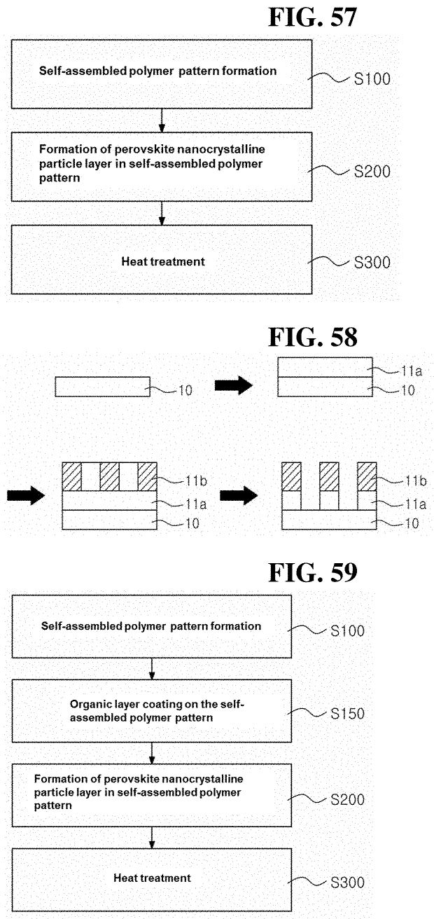

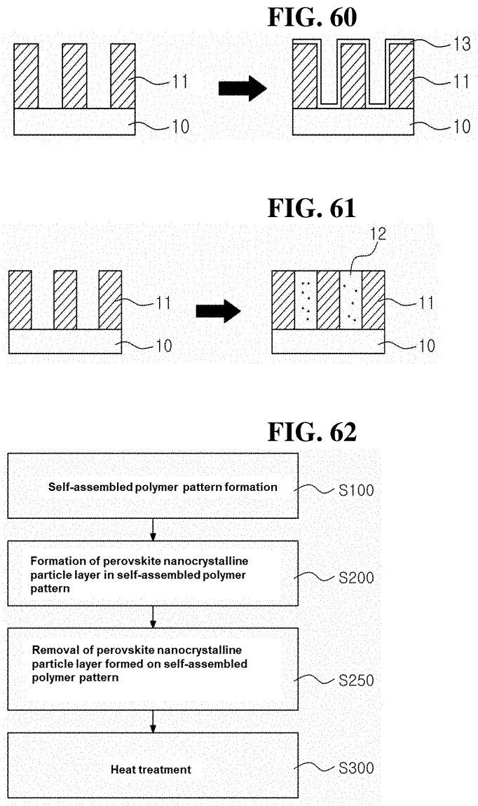

Meanwhile, the luminous layer according to this disclosure may include an organic layer (13) surrounding the self-assembled polymer (11) between the patterned self-assembled polymer (11) and the metal halide perovskite nanocrystal particle (12) as shown in FIG. 57.

When the width of the pattern of the self-assembled polymer (11) is too wide, the organic material layer (13) may be coated on the self-assembled polymer (11) to reduce the width.

The organic material layer (13) may be formed of a polymer having the same composition as that of the self-assembled polymer, or other polymers. As an example, the organic material used in the organic material layer is PEO(Polyethylene oxide), PS(Polystyrene), PCL(Polycaprolactone), PAN(Polyacrylonitrile), PMMA(Poly(methyl methacrylate)), polyimide, polythiophene, polyaniline, polypyrrole, poly(3,4-ethylenedioxythiophene), PVDF(Poly(vinylidene fluoride)), PVK (Poly(n-vinylcarbazole)), PVC (Polyvinylchloride), and a mixture of two or more selected from the group consisting of, but is not limited thereto.

The organic material layer (13) may be formed on the self-assembled polymer pattern by a deposition method used in the industry, for example, the chemical vapor deposition (CVD) or thermal deposition method (thermal deposition) can be used, but it is not limited thereto.

The thickness of the organic material layer (13) may be adjusted according to the width of the formed self-assembled polymer pattern, and may be, for example, 1 to 20 nm, but is not limited thereto.

Hereinafter, a method of manufacturing a metal halide perovskite light emitting device including the self-assembled polymer light emitting layer will be described.

FIG. 57 is a flow chart illustrating a method of manufacturing a self-assembled polymer-metal halide perovskite light emitting layer according to an embodiment of the present disclosure.

Referring to FIG. 57, a method of manufacturing a self-assembled polymer-metal halide perovskite light-emitting layer of the present disclosure includes forming a self-assembled polymer pattern on a member for applying the light-emitting layer (S100).

Preparing a light emitting layer by forming a metal halide perovskite nanocrystal particle layer in the prepared self-assembled polymer pattern (S200); and

Heat-treatment of the light emitting layer (S300).

Hereinafter, the present disclosure will be described step by step.

First, step S100 is a step of forming a self-assembled polymer pattern on a member for applying a light emitting layer.

In the above step, a member for applying a light emitting layer is first prepared.

The member for applying the light emitting layer may be a substrate, an electrode, or a semiconductor layer. The substrate, electrode, or semiconductor layer may be a substrate, electrode, or semiconductor layer that can be used in a light emitting device. In addition, the member for applying the light emitting layer may have a form in which a substrate/electrode is sequentially stacked or a form in which a substrate/electrode/semiconductor layer is sequentially stacked.

Since the description of the substrate, the electrode, or the semiconductor layer is the same as described above, a detailed description will be omitted.

Next, a self-assembled polymer pattern is formed on the member for applying the emission layer.

In the self-assembled polymer used in the present disclosure, since two or more kinds of polymers are formed in a cylinder shape on the member for applying the light emitting layer through a self-assembly reaction, a pattern can be formed by removing one of the polymers in the form of a cylinder.

As an example, as shown in FIG. 58, a random copolymer thin film (11a) is formed on the substrate (10), and after forming a PS-b-PMMA block copolymer thin film (11b) having a cylinder shape on the random copolymer thin film (11a), in the cylindrical block copolymer obtained through self-assembly, the PMMA cylinder portion and the random copolymer thin film under the PMMA may be selectively etched to form a self-assembled polymer pattern in which only the PS portion is left.

In addition, as shown in FIG. 59, after forming the self-assembled polymer pattern, a step (S150) of forming the organic material layer (13) on the self-assembled polymer (11) on which the pattern is formed may be additionally performed.

When the width of the pattern of the self-assembled polymer (11) is too wide, the organic material layer (13) may be coated on the self-assembled polymer (11) as shown in FIG. 61 to reduce the width.

The organic material layer (13) may be formed of a polymer having the same composition as that of the self-assembled polymer, or other polymers. As an example, the organic material used in the organic material layer is PEO(Polyethylene oxide), PS(Polystyrene), PCL(Polycaprolactone), PAN(Polyacrylonitrile), PMMA(Poly(methyl methacrylate)), polyimide, polythiophene, polyaniline, polypyrrole, poly(3,4-ethylenedioxythiophene), PVDF(Poly(vinylidene fluoride)), PVK (Poly(n-vinylcarbazole)), PVC (Polyvinylchloride), and a mixture of two or more selected from the group consisting of, but is not limited thereto.

The organic material layer (13) may be formed on the self-assembled polymer pattern by a deposition method used in the industry, for example, the chemical vapor deposition (CVD) or thermal deposition method (thermal deposition) can be used, but it is not limited thereto.

The thickness of the organic material layer (13) may be adjusted according to the width of the formed self-assembled polymer pattern, and may be, for example, 1 to 20 nm, but is not limited thereto.

Next, S200 is a step of preparing a light emitting layer by forming a metal halide perovskite nanocrystal particle layer in the prepared self-assembled polymer pattern.

The methods of forming the metal halide perovskite nanocrystal particle layer include Preparing a first solution in which a metal halide perovskite is dissolved in a protic solvent; And

And forming a metal halide perovskite nanocrystal particle layer by putting the first solution into a self-assembled polymer pattern.

First, a first solution in which a metal halide perovskite is dissolved in a protic solvent is prepared. The method of preparing the first solution in which the metal halide perovskite is dissolved in the protic solvent is the same as described above, and thus a detailed description thereof will be omitted.

Next, the first solution is put into a self-assembled polymer pattern to form a metal halide perovskite nanocrystal particle layer.

Specifically, as shown in FIG. 61, the first solution is located in a self-assembled polymer pattern pierced in a cylindrical shape. As a batch method, a solution may be dropped drop by drop or a solution process may be used, but is not limited thereto.

The solution process includes spin-coating, bar coating, slot-die coating, gravure-printing, nozzle printing, and ink-jet printing. printing), screen printing, electrohydrodynamic jet printing, and electrospray.

In addition, metal halide perovskite nanoparticles may be prepared through an inverse nano-emulsion method.

Specifically, preparing a first solution in which a metal halide perovskite is dissolved in a protic solvent and a second solution in which an alkyl halide surfactant is dissolved in an aprotic solvent; And

The first solution may be mixed with the second solution and located in a self-assembled polymer pattern to form a metal halide perovskite nanocrystal layer.

At this time, the preparation of the first solution in which the protic solvent and the metal halide perovskite are dissolved is as described above.

At this time, when preparing the second solution, the aprotic solvent is dichloroethylene, trichloroethylene, chloroform, chlorobenzene, dichlorobenzene, styrene, dimethylformamide, dimethyl sulfoxide, xylene, toluene, cyclohexene or It may include, but is not limited to, isopropyl alcohol.

In addition, the alkyl halide surfactant may have a structure of alkyl-X. The halogen element corresponding to X at this time may include Cl, Br, or I. In addition, the alkyl structure at this time includes a primary alcohol having a structure such as acyclic alkyl having a structure of CₙH₂ₙ₊₁, a primary alcohol having a structure such as CₙH₂ₙ₊₁OH, a secondary alcohol, and a tertiary alcohol, alkylamine having a structure of alkyl-N (ex. Hexadecyl amine, 9-Octadecenylamine 1-Amino-9-octadecene (C₁₉H₃₇N)), p-substituted aniline and phenyl ammonium ammonium) and fluorine ammonium.

Meanwhile, a carboxylic acid (COOH) surfactant may be used instead of the alkyl halide surfactant.

For example, surfactants may contain 4,4′-Azobis(4-cyanovaleric acid), acetic acid, 5-aminosalicylic acid, acrylic acid, L-Aspentic acid, 6-Bromohexanoic acid, bromoacetic acid, dichloroacetic acid, ethylenediaminetetraacetic acid, isobutyric acid, itaconic acid, maleic acid, r-maleimidobutylic acid, L-Malic acid, 4-Nitrobenzoic acid, 1-Pyrenecarboxylic acid or oleic acid, but are not limited to this.

In the step of forming a metal halide perovskite nanocrystal layer by mixing the first solution with the second solution and placing it in a self-assembled polymer pattern, it is preferable to mix the first solution dropwise with the second solution. Further, the second solution may be stirred. For example, nanoparticles may be synthesized by slowly adding a second solution in which an organic/inorganic metal halide perovskite (OIP) is dissolved in a second solution in which an alkyl halide surfactant is dissolved, which is being stirred strongly.

In this case, when the first solution is dropped into the second solution and mixed, organic-inorganic metal halide perovskite (OIP) is precipitated from the second solution due to a difference in solubility. The organic-inorganic metal halide perovskite (OIP) precipitated in the second solution is stabilized by an alkyl halide surfactant to form well-dispersed organic-inorganic metal halide perovskite nanocrystals (OIP-NC). Accordingly, a solution including organic-inorganic metal halide perovskite nanocrystals and a plurality of alkyl halide organic ligands surrounding the organic-inorganic metal halide perovskite nanoparticles can be prepared.

Thereafter, the solution containing the metal halide perovskite nanoparticles may be located in a self-assembled polymer pattern pierced in a cylindrical shape using the method described above to form a metal halide perovskite nanocrystal layer.

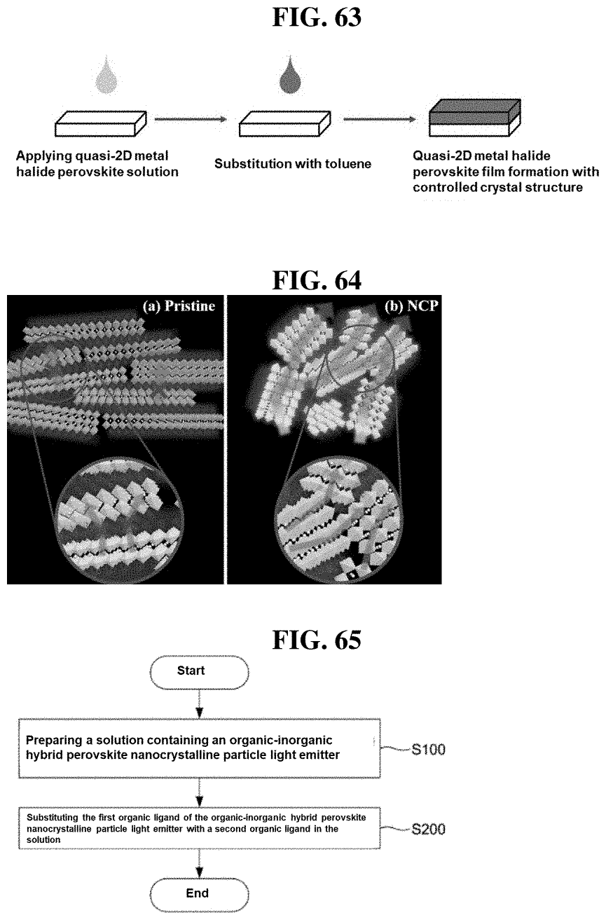

However, as shown in FIG. 62, when the metal halide perovskite nanocrystal particle layer is formed on the polymer pattern beyond the height of the polymer pattern, an additional step (S250) can be taken to remove the metal perovskite nanocrystal particle layer formed on the above polymer pattern as shown in FIG. 63.

Removal of the metal halide perovskite nanocrystal particle layer formed on the above polymer pattern can be performed by dropping the solvent dissolving metal halide perovskite onto the metal halide perovskite nanocrystal particle layer, dissolving only the metal halide perovskite nanocrystal particle layer at the top of the self-assembled polymer pattern.

Next, S300 is a step of heat-treating the prepared light emitting layer.

The heat treatment may be performed at 60 to 80° C. for 5 to 15 minutes.

Through the heat treatment, the solvent is evaporated so that the metal halide perovskite nanocrystal particles are strongly bonded to the polymer pattern.

In an embodiment of the present disclosure, CsBr and PbBr₂ are dissolved in dimethyl sulfoxide (DMSO) to spin coat the metal halide perovskite solution on a patterned substrate, and heat-treated for 10 minutes at 70° C. to fabricate a self-assembled polymer-metal halide perovskite light-emitting layer.

The self-assembled polymer-metal halide perovskite light-emitting layer prepared by the above method binds the metal halide perovskite nanocrystal particles in the self-assembled polymer pattern, thereby shifting the light emission wavelength toward blue, the luminescence efficiency of the metal halide perovskite material can be improved, and the self-assembled polymer located between the metal halide perovskite nanocrystal particle layers can prevent the ion migration between the metal halide perovskite nanocrystal particle layers. Therefore, stability can be improved.

<Metal Halide Perovskite Light Emitting Device Including a Quasi-2D Metal Halide Perovskite Light Emitting Layer with a Controlled Nanocrystal Structure>

According to another embodiment of the present disclosure, when the light-emitting layer has a quasi-two-dimensional structure, it may include a quasi-two-dimensional metal halide perovskite light-emitting layer with a controlled nanocrystal structure. The quasi-2D structure may be a Ruddlesden-Popper phase or a Dion-Jacobson phase.

Recently, high luminescence efficiency has been reported through charge transfer and charge constraints from two-dimensional metal halide perovskite with high bandgap to three-dimensional metal halide perovskite with low bandgap by forming Quasi-2-dimensional metal halide perovskite. However, since the metal halide perovskite crystal is randomly crystallized without the tendency of the crystal orientation and distribution, it is difficult to control the dimensional distribution, so in order to manufacture a high-efficiency light emitting device, additional interfacial treatment must be performed to reduce interfacial quenching and balance charge. There are drawbacks. Therefore, it is necessary to develop a new process for an effective energy structure capable of controlling the number and distribution of dimensions of the metal halide perovskite emission layer and preventing charge dissociation.

The quasi-two-dimensional metal halide perovskite light emitting layer with a controlled nanocrystal structure according to the present disclosure may be produced by method for producing quasi-two-dimensional metal halide perovskite film with controlled crystal structure including forming a quasi-two-dimensional structure metal halide perovskite film by coating a quasi-two-dimensional structure metal halide perovskite solution on a substrate and a step of controlling a multi-phase structure of the quasi-2D structure metal halide perovskite crystals in a three-dimensional structure by doping a solvent having a boiling point of at least 100° C. on the quasi-2D structure metal halide perovskite film. (FIG. 63, FIG. 64)

In addition, preferably, the solvent may be one or more selected from the group consisting of toluene, xylene, butanol, pentanol, hexanol, heptanol, octanol, octane and decane, or a combination thereof, but is not limited thereto.

Also preferably, the quasi-2D metal halide perovskite may include a single-phase structure of A′₂Aₙ₋₁BₙX₃ₙ₊₁ or a multi-phase structure having different n values. The quasi-2D structure may be a Ruddlesden-Popper phase or a Dion-Jacobson phase.

Also preferably, A and A′ may be monovalent cations, B may be a metal material, and X may be a halogen element.

The monovalent cation may be a monovalent organic cation or an alkali metal. For example, the monovalent organic cation may be Organic ammonium (RNH₃)⁺, organic amidinium derivative (RC(═NR₂)NR₂)⁺, organic guanidinium derivative (R₂NC(═NR₂)NR₂)⁺, organic diammonium (CₓH₂ₓ₋ₙ₊₄)(NH₃)ₙ⁺, ((CₓH₂ₓ₊₁)ₙNH₃)(CH₃NH₃)ₙ⁺, (RNH₃)₂⁺, (CₙH₂ₙ₊₁NH₃)₂⁺, (CF₃NH₃)⁺, (CF₃NH₃)ₙ⁺, ((CₓF₂ₓ₊₁)ₙNH₃)₂(CF₃NH₃)ₙ⁺, ((CₓF₂ₓ₊₁)ₙNH₃)₂⁺ or (CₙF₂ₙ₊₁NH₃)₂⁺ (x, n is an integer greater than or equal to 1, R=hydrocarbon derivative, H, F, Cl, Br, I) and combinations thereof, but is not limited thereto. The alkali metal may be Li⁺, Na⁺, K⁺, Rb⁺, Cs⁺, Fr⁺, and combinations thereof, but is not limited thereto.

In addition, preferably, the organic cations may be acetamidinium, azaspironanium, benzene diammonium, benzylammonium, butanediammonium, iso-butylammonium, n-butylammonium, t-butylammonium, cyclohexylammonium, cyclohexylmethylammonium, diazobicyclooctanedinium, diethylammonium, N,N-diethylethane diammonium, dimethylammonium, dodecylammonium, ethanediammonium, ethylammoniuium, 4-fluoro-benzylammonium, 4-fluoro-phenylethylammonium, 4-fluoro-phenylammonium, formamidinium, guanidinium, hexanediammnium, hexylammonium, imidazolium, 2-methoxyethylammonium, 4-methoxy-phenylethylammonium, 4-methoxy-phenylammonium, methylammonium, morpholinium, octylammonium, pentylammonium, piperazinediium, piperidinium, propanediammonium, iso-propylammonium, di-isopropylammonium, n-propylammonium, Pyridinium, 2-pyrrolidin-1-ium-1-yethylammonium, pyrrolidinium, quinclidin-1-ium), 4-trifluoromethyl-benzylammonium, 4-trifluoromethyl ammonium, quaternary ammonium cations such as benzalkonium chloride, dimethyldioctadecylammonium chloride, trimethylglycine, choline and combinations thereof, but are not limited thereto.

The B may be a divalent transition metal, a rare earth metal, an alkaline earth metal, a monovalent metal, a combination of a trivalent metal, an organic substance (a monovalent, divalent, trivalent cation), and a combination thereof. In addition, preferably, the divalent transition metal, rare earth metal, and alkaline earth metal may be Be²⁺, Mg²⁺, Ca²⁺, Sr²⁺, Ba²⁺, Ra²⁺, Mn²⁺, Fe²⁺, Co²⁺, Ni²⁺, Cu²⁺, Zn²⁺, Ru²⁺, Pd²⁺, Cd²⁺, Pt²⁺, Hg²⁺, Ge²⁺, Sn²⁺, Pb²⁺, Se²⁺, Te²⁺, Po²⁺, Bi²⁺, Eu²⁺, No²⁺, and combinations thereof, but are not limited thereto. Monovalent metal may be Li⁺, Na⁺, K⁺, Rb⁺, Cs⁺, Fr⁺, Ag⁺, Hg⁺, Ti⁺, and combinations thereof, trivalent metal may be Cr³⁺, Fe³⁺, Co³⁺, Ru³⁺, Rh³⁺, Ir³⁺, Au³⁺, Al³⁺, Ga³⁺, In³⁺, Ti³⁺, As³⁺, Sb³⁺, Bi³⁺, La³, Ce³⁺, Pr³⁺, Nd³⁺, Pm³⁺, Sm³⁺, Eu³⁺, Gd³⁺, Tb³⁺, Dy³⁺, Ho³⁺, Er³⁺, Tm³⁺, Yb³⁺, Lu³⁺, Ac³⁺, Am³⁺, Cm³⁺, Bk³⁺, Cf³⁺, Es³⁺, Fm³⁺, Md³⁺, Lr³⁺, and combinations thereof.

In addition, X may be F⁻, Cl⁻, Br⁻, I⁻, At⁻, and combinations thereof.

In addition, preferably, the solvent used in the preparation of the quasi-two-dimensional structure metal halide perovskite solution is dimethylformamide, gamma-butyrolactone, and N-methylpyrrolidone or dimethylsulfoxide, and combinations thereof.

Also preferably, the concentration of the quasi-two-dimensional structure metal halide perovskite solution may be 0.01M to 0.5M.

In addition, preferably, the coating method can be selected from a group of spin coatings, bar coatings, nozzle printing, spray coatings, slot die coatings, gravure printing, inkjet printing, screen printing, electrohydrodynamic jet printing and electrospray.

<Ligand Substitution of Metal Halide Perovskite Nanocrystal Particles for High-Efficiency Light Emitting Devices>

If the above luminous layer is a metal halide perovskite nanocrystal particle, the organic ligand surrounding the metal halide perovskite nanocrystal particle can be replaced with a shorter ligand or a ligand containing phenyl or fluoride to produce more light-efficient nanocrystal particle light emitter and light-emitting devices.

This section describes the method of manufacturing metal halide perovskite nanocrystal particle emitter replaced with organic ligands according to an embodiment of the present disclosure.

FIG. 65 is a flow chart illustrating a method of manufacturing a metal halide perovskite nanocrystal particle light emitter in which an organic ligand is substituted according to an embodiment of the present disclosure.

Referring to FIG. 65, the method for manufacturing a metal halide perovskite nanocrystal particle light emitter substituted with an organic ligand according to the present disclosure includes preparing a solution including a metal halide perovskite nanocrystal particle light emitter (S100), and substituting a second organic ligand for the first organic ligand of the metal halide perovskite nanocrystal particle light emitter in the solution (S200).

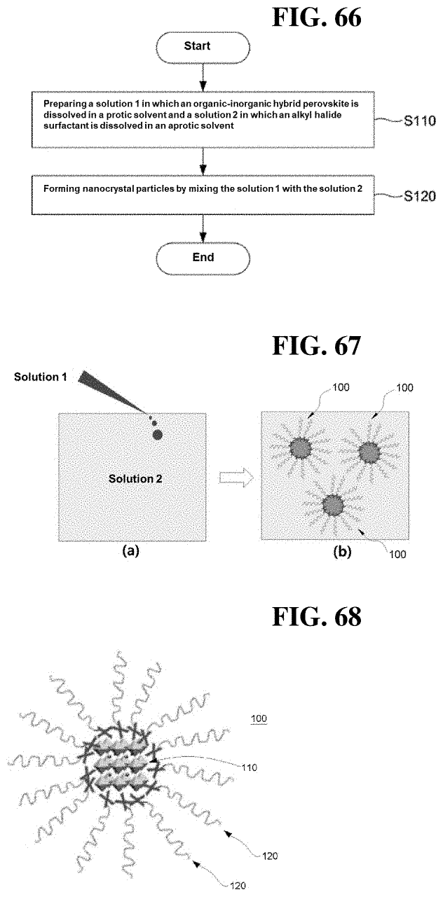

First, a solution containing a metal halide perovskite nanocrystal particle light emitter is prepared (S100). A manufacturing example for this will be described with reference to FIG. 66 to 69.

FIG. 66 is a flow chart showing a method of manufacturing a metal halide perovskite nanocrystal particle light emitter according to an embodiment of the present disclosure.

Referring to FIG. 66, a metal halide perovskite nanocrystal particle light emitter according to the present disclosure may be fabricated through an inverse nano-emulsion method.

First, a first solution in which a metal halide perovskite is dissolved in a protic solvent and a second solution in which an alkyl halide surfactant is dissolved in an aprotic solvent are prepared (S110).

The protic solvent at this time may include dimethylformamide, gamma-butyrolactone or N-methylpyrrolidone, dimethylsulfoxide, but is not limited thereto.

The metal halide perovskite may be a material having a three-dimensional crystal structure, a two-dimensional crystal structure, a one-dimensional crystal structure, or a zero-dimensional crystal structure.

The metal halide perovskite is ABX₃(3D), A₄BX₆(0D), AB₂X₅(2D), A₂BX₄(2D), A2BX₆(0D), A₂B⁺B³⁺X₆(3D), A₃B₂X₉(2D) or it may include a structure of Aₙ₋₁BₙX₃ₙ₊₁ (quasi-2D) (n is an integer between 2 and 6). A is a monovalent cation, B is a metal material, and X may be a halogen element. The quasi-2D structure may be a Ruddlesden-Popper phase or a Dion-Jacobson phase.

Since specific examples of A, B, and X are the same as described above, they are omitted to avoid redundant description.

These metal halide perovskites can be prepared by combining AX and BX₂ in a certain ratio. For example, a first solution in which A₂BX₃ metal halide perovskite is dissolved may be prepared by dissolving AX and BX₂ in a ratio of 2:1 in a protic solvent. In addition, the aprotic solvent at this time may include dichloroethylene, trichloroethylene, chloroform, chlorobenzene, dichlorobenzene, styrene, dimethylformamide, dimethyl sulfoxide, xylene, toluene, cyclohexene or isopropyl alcohol, but is not limited to.

The alkyl halide surfactant may have an alkyl-X structure. The halogen element corresponding to X at this time may include Cl, Br, or I. In addition, the alkyl structure at this time includes an acyclic alkyl having a structure of CₙH₂ₙ₊₁, a primary alcohol having a structure such as CₙH₂ₙ₊₁OH, a secondary alcohol, and a tertiary alcohol, alkylamine having the structure of alkyl-N (ex. Hexadecyl amine, 9-Octadecenylamine 1-Amino-9-octadecene (C₁₉H₃₇N)), p-substituted aniline, phenyl ammonium ammonium) or fluorine ammonium, but is not limited thereto.

These metal halide perovskites can be prepared by combining AX and BX₂ in a certain ratio. For example, a first solution in which A₂BX₃ metal halide perovskite is dissolved may be prepared by dissolving AX and BX₂ in a ratio of 2:1 in a protic solvent. In addition, the aprotic solvent at this time may include dichloroethylene, trichloroethylene, chloroform, chlorobenzene, dichlorobenzene, styrene, dimethylformamide, dimethyl sulfoxide, xylene, toluene, cyclohexene or isopropyl alcohol, but is not limited thereto.

The alkyl halide surfactant may have an alkyl-X structure. The halogen element corresponding to X at this time may include Cl, Br, or I. In addition, the alkyl structure at this time includes an acyclic alkyl having a structure of CₙH₂ₙ₊₁, a primary alcohol having a structure such as CₙH₂ₙ₊₁OH, a secondary alcohol, and a tertiary alcohol, alkylamine having the structure of alkyl-N (ex. Hexadecyl amine, 9-Octadecenylamine 1-Amino-9-octadecene (C₁₉H₃₇N)), p-substituted aniline, phenyl ammonium ammonium) or fluorine ammonium.

Meanwhile, a carboxylic acid (COOH) surfactant may be used instead of the alkyl halide surfactant, and the type of the carboxylic acid surfactant is as described above.

Then, the first solution is mixed with the second solution to form nanocrystal particle (S200).

In the step of forming a metal halide perovskite nanocrystal layer by mixing the first solution with the second solution, it is preferable to mix the first solution dropwise with the second solution. Further, the second solution may be stirred. For example, nanoparticles may be synthesized by slowly adding a first solution in which an organic/inorganic metal halide perovskite (OIP) is dissolved in a second solution in which an alkyl halide surfactant is dissolved, which is being stirred strongly.

In this case, when the first solution is dropped into the second solution and mixed, organic-inorganic metal halide perovskite (OIP) is precipitated from the first solution due to a difference in solubility. The organic-inorganic metal halide perovskite (OIP) precipitated in the second solution is stabilized by an alkyl halide surfactant to form well-dispersed organic-inorganic metal halide perovskite nanocrystals (OIP-NC). Accordingly, as shown in FIG. 68, a metal halide perovskite nanocrystal particle light emitter including an organic-inorganic metal halide perovskite nanocrystal structure and a plurality of alkyl halide organic ligands surrounding the organic-inorganic metal halide perovskite nanocrystal structure may be prepared.

Next, in the solution, the first organic ligand of the metal halide perovskite nanocrystal particle light emitter is substituted with a second organic ligand (S200).

In this case, a second organic ligand having a length shorter than that of the first organic ligand or containing a phenyl group or a fluorine group may be added to the solution to replace the first organic ligand with the second organic ligand. At this time, a substitution reaction may be performed by applying constant heat.

The second organic ligand at this time may include an alkyl halide. For example, the second organic ligand may have a structure of alkyl-X′. The halogen element corresponding to X′ at this time may include Cl, Br, or I. In addition, the alkyl structure at this time includes an acyclic alkyl having a structure of CₙH₂ₙ₊₁, a primary alcohol having a structure such as CₙH₂ₙ₊₁OH, a secondary alcohol, and a tertiary alcohol, alkylamine having the structure of alkyl-N (ex. Hexadecyl amine, 9-Octadecenylamine, 1-Amino-9-octadecene (C₁₉H₃₇N)), p-substituted aniline, phenyl ammonium ammonium) or fluorine ammonium, but is not limited thereto.

In addition, the second organic ligand contains alkyl halide, and the halogen element of the second organic ligand is characterized by its high affinity with the central metal of the above inorganic metal halide perovskite nanocrystal structure than the halogen element of the first organic ligand.

For example, when the first organic ligand is CH₃(CH₂)17NH3Br, the organic ligand substitution can be done by using a short-length alkyl halide having a halogen element having a higher affinity with the central metal of the metal halide perovskite nanocrystal structure than the first organic ligand and adding CH3(CH2)8NH3I with heat. Accordingly, CH3(CH2)8NH3I will become the second organic ligand surrounding the nanocrystal structure, and eventually the length of the organic ligand of the nanocrystalline light emitter can be reduced.

The metal halide perovskite nanocrystalline particle light emitter according to the present disclosure forms a nanocrystalline structure with the alkyl halide (first organic ligand) used as a surfactant surrounds the surface of the metal halide perovskite to stabilize the surface of the metal halide perovskite deposited as described above.

On the other hand, if the length of the alkyl halide surfactant is short, the size of the formed crystalline particles increases, so it may be formed to exceed 900 nm. In this case, due to the thermal ionization and delocalization of charge carriers in the large nanocrystal particle, there may be a fundamental problem in that excitons do not emit light and are separated into free charges and disappear.

That is, the size of the metal halide perovskite crystalline particles to be formed and the length of the alkyl halide surfactant used to form the nanocrystal particle are inversely proportional.

Therefore, by using an alkyl halide having a certain length or more as a surfactant, the size of the crystalline particles of the metal halide perovskite formed can be controlled to be less than a certain size or less. For example, octadecyl-ammonium bromide as an alkyl halide surfactant may be used to form organic-inorganic hybrid metal halide perovskite nanocrystal particles having a size of 900 nm or less.

Therefore, in order to form nanocrystalline particles of a certain size or less, an alkyl halide (first organic ligand) having a certain length or longer is used, and then, by substituting such a first organic ligand with a second ligand having a short length or containing a phenyl group or a fluorine group, energy transfer or charge injection into the nanocrystalline structure is further increased, thereby increasing luminous efficiency. Furthermore, durability-stability can also be increased by the substituted hydrophobic ligand.

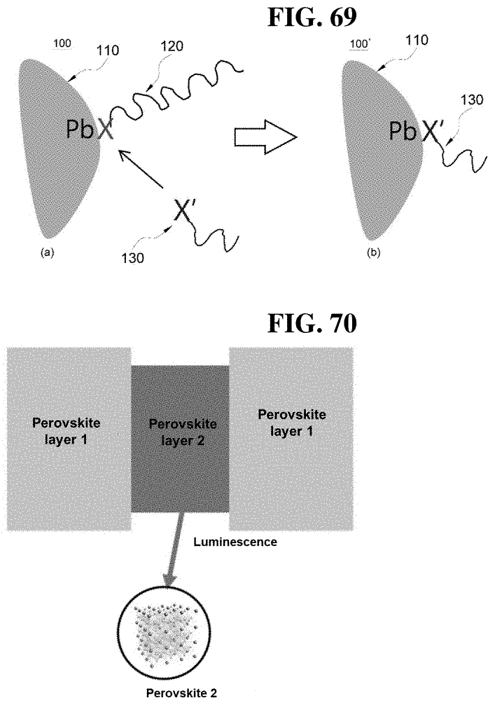

This replacement step (S200) will be described in more detail with reference to FIG. 69.

FIG. 69 is a schematic diagram showing a method of manufacturing a metal halide perovskite nanocrystal particle light emitter in which an organic ligand is substituted according to an embodiment of the present disclosure.

Referring to FIG. 69(a), the perovskite nanocrystal particle light emitter (100) including a metal halide perovskite nanocrystal structure (110) and a first organic ligand (120) surrounding the nanocrystal structure (110) is prepared. The nanocrystal particle light emitter (100) may be prepared in a state contained in a solution (solution state). Meanwhile, as shown, the central metal of the metal halide perovskite nanocrystal structure (110) is Pb.

Then, the second organic ligand (130) is added to the solution containing the nanocrystal particle light emitter (100).

Referring to FIG. 69(b), the first organic ligand (120) is replaced with the second organic ligand (130) by the addition of the second organic ligand (130). This organic ligand substitution may be performed by using a difference in affinity strength between the central metal of the metal halide perovskite nanocrystal structure (110) and the halogen element. For example, the affinity with the central metal is stronger in the order of Cl<Br<I.

Accordingly, when the halogen element (X) of the first organic ligand (120) is Cl, ligand substitution may be performed using the halogen element (X′) of the second organic ligand (130) as Br or I.

Therefore, the organic ligand-substituted metal halide perovskite nanocrystal particle light emitter with improved luminescence efficiency can be formed by replacing the first organic ligand (120) with the second organic ligand (130) having a short length or containing a phenyl group or a fluorine group (100′).

As another example, the above-described organic-inorganic metal halide perovskite nanocrystal particles substituted with the organic ligands or organic ligand-substituted inorganic metal halide perovskite nanocrystal particles can be applied as a photoactive layer in solar cell application. Such a solar cell may include a first electrode, a second electrode, and a photoactive layer including the above-described metal halide perovskite nanocrystal particles, but positioned between the first electrode and the second electrode.

<Metal Halide Perovskite Light Emitting Device Including a Metal Halide Perovskite Light Emitting Layer Having a Tandem Structure>

According to another embodiment of the present disclosure, the emission layer may include a metal halide perovskite having a tandem structure.

In the light-emitting device according to an embodiment of the present disclosure, the light-emitting layer (40) has a tandem structure in which a first light-emitting material layer and a second light-emitting material layer are alternately stacked, and the first light-emitting material layer and the second light-emitting material layer may have different band gaps. The light-emitting layer may be formed by co-depositing a metal halide perovskite of a first light-emitting material layer and a perovskite of a second light-emitting material layer. Until now, the metal halide perovskite light emitting layer used in the metal halide perovskite light emitting device is mainly manufactured through a solution process. However, the solution process has a disadvantage in that the uniformity of the thin film to be formed is low, thickness control is not easy, and materials that can be mixed are limited by the characteristics of the solvent. In the metal halide perovskite light emitting device, the biggest performance impediment factor is the non-uniform thin film. In a thin film device composed of a stacked thin film, the non-uniformity of the thin film is one of the factors that greatly deteriorates the device performance by breaking the charge balance and generating a leakage current. In particular, since the morphology of the thin film varies greatly depending on the conditions for forming the thin film and the surrounding environment, the uniformity of the thin film is very important in the performance of the metal halide perovskite light emitting device. An example of a non-uniform thin film is a general spin coating process that forms CH₃NH₃PbBr₃. If the additional nanocrystal immobilization process is not used, there is a problem that the foil is formed in the form of an isolated crystal due to spontaneous crystallization [Science 2015, 350, 1222]. However, in the case of using the nanocrystal immobilization process, the film quality of the thin film can be greatly influenced by the experimental environment, so even if the same process is used, there is a disadvantage in that the film quality has a large deviation. In addition, since the film quality of the thin film is improved only in the region where the nanocrystal is pinned, there may be limitations in implementing a large-area device. However, there has not been an example of manufacturing the metal halide perovskite light emitting layer by a deposition process. The position of the electron-hole recombination zone in the device, that is, the emission spectrum of the device may be affected by the thickness of the emission layer and may vary depending on the energy level of the material used. Accordingly, in the present disclosure, a thin film was prepared by co-depositing the first light-emitting material layer and the second light-emitting material layer through an evaporation method. By co-depositing the first light-emitting material layer and the second light-emitting material layer, a uniform thin film can be formed, it is easy to control the thickness of the thin film, and the size of the formed metal halide perovskite crystal is reduced. (exciton) or charge carrier (charge carrier) is spatially constrained, the luminescence efficiency can be improved.

FIG. 70 is a cross-sectional view showing a light emitting layer having a tandem structure according to an embodiment of the present disclosure.

Referring to FIG. 70, in the light-emitting device according to an embodiment of the present disclosure, the light-emitting layer (40) has a tandem structure in which a first light-emitting material layer and a second light-emitting material layer are alternately stacked, and the first light-emitting material layer and the second light-emitting material layer may have different band gaps.

In more detail, a band gap of the first light-emitting material layer may be larger than a band gap of the second light-emitting material layer. Specifically, the energy level of the Valance band maximum (VBM) of the first light-emitting material layer may be lower than the energy level of the VBM of the second light-emitting material layer, and the Conduction Band Minimum (CBM) of the first light-emitting material layer may be higher than the energy level of the CBM of the second light-emitting material layer. The energy level of the valence band maximum (VBM) of the emission layer can be lower than the work function of the positive electrode and the HOMO (Highest Occupied Molecular Orbital) energy level of the hole injection layer, and higher than the work function of the cathode and the HOMO energy level of the electron transport layer. The energy level of the conduction band minimum (CBM) of the emission layer may be lower than the work function of the anode and the LUMO (Lowest Unoccupied Molecular Orbital) energy level of the hole injection layer, and higher than the work function of the cathode or the LUMO of the electron transport layer.

That is, in the case of a light emitting layer in which metal halide perovskites having different band gaps are alternately arranged and stacked, the energy transfer behavior may vary according to the energy level of the material. Accordingly, since energy transfer occurs from the first light-emitting material layer having a larger band gap to the second light-emitting material layer having a smaller band gap, light emission may occur only in the second light-emitting material layer. Through this, the electron-hole recombination zone of the light emitting device can be controlled. Therefore, the energy level of the material used to control the location at which light emission occurs may be important.

Measurement of the energy level of the VBM (Valence Band Maximum) of the luminescent material layer containing the metal halide perovskite is performed by UV photoelectron spectroscopy (UPS), which is a method of measuring the ionization potential by irradiating UV on the surface of the thin film and detecting the electrons that come out of the material can be used. Alternatively, cyclic voltammetry (CV), which measures the oxidation potential through voltage sweep after dissolving the substance to be measured in a solvent together with an electrolyte, may be used. In addition, the PYSA (Photoemission Yield Spectrometer in Air) method can be used to measure the ionization potential in the atmosphere using an AC-3 (RKI) device. Also, the energy level of the conduction band minimum (CBM) of the metal halide perovskite can be obtained by measuring IPES (Inverse Photoelectron Spectroscopy) or electrochemical reduction potential. IPES is a method to determine the energy level of CBM by irradiating an electron beam onto a thin film and measuring the light emitted. In addition, in the measurement of the electrochemical reduction potential, a reduction potential may be measured through a voltage sweep after dissolving a substance to be measured in a solvent together with an electrolyte. Alternatively, the energy level of the conduction band minimum (CBM) can be calculated using the energy level of the valence band maximum (VBM) and the singlet energy level obtained by measuring the degree of UV absorption of the target material.

Specifically, the energy level of the Valence Band Maximum (VBM) of the present specification was measured through an AC-3 (RKI) measuring instrument after vacuum deposition of the target material to a thickness of 50 nm or more on the ITO substrate. In addition, the energy level of the conduction band minimum (CBM) is determined by measuring the absorption spectrum (abs.) and the photoluminescence spectrum (PL) of the prepared sample, and then calculating the edge energy of each spectrum, and regarding this as band gap energy, and calculating the energy level of the conduction band minimum (CBM) by subtracting the bandgap difference from the energy level of the valence band maximum (VBM) measured in AC-3.

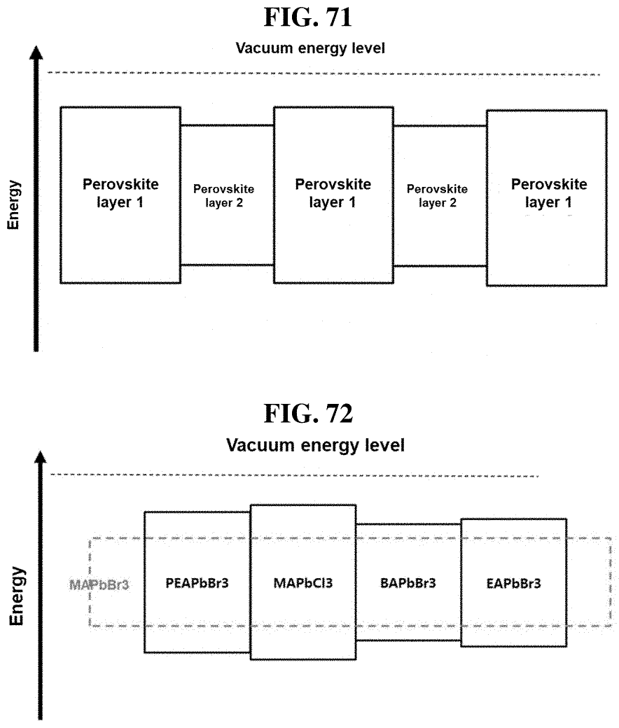

FIG. 71 illustrates energy levels of a light emitting layer having a tandem structure in which first and second light emitting material layers are alternately stacked according to an embodiment of the present disclosure.

In the present disclosure, light emission may occur in the second light-emitting material layer. Accordingly, the metal halide perovskite of the first light-emitting material layer having a larger band gap and the metal halide perovskite of the second light-emitting material layer having a smaller band gap are alternately disposed to have a tandem structure. That is, the energy level of the valence band maximum (VBM) of the first light-emitting material layer may be lower than the energy level of the VBM of the second light-emitting material layer, and the energy level of the conduction band minimum (CBM) of the first light-emitting material layer may be higher than the energy level of the CBM of the second light-emitting material layer.

That is, the energy level of the valence band maximum (VBM) of the first light-emitting material layer may be lower than the energy level of the VBM of the second light-emitting material layer, and the conduction band minimum (CBM) of the first light-emitting material layer may be higher than the energy level of the CBM of the second light-emitting material layer.

FIG. 72 illustrates energy levels of materials used in a light emitting layer having a tandem structure in which first and second light emitting material layers are alternately stacked according to an embodiment of the present disclosure.

Referring to FIG. 72, the energy level of the Valence Band Maximum (VBM) of MAPbBr₃ is (−)5.9, and the energy level of the Conduction Band Minimum (CBM) is (−)3.6. The MAPbBr₃ may be used as a metal halide perovskite included in the second light-emitting material layer. At this time, in the case of PEAPbBr₃, since the energy level of the highest valence band (VBM) is (−)6.4, it is lower than the energy level of the highest valence band (VBM, Valence Band Maximum) of MAPbBr₃, and the lowest electron band (CBM, Conduction Band minimum) is (−)2.5, which is higher than the energy level of the electron band (CBM, Conduction Band Minimum) of MAPbBr₃. Therefore, when MAPbBr₃ is used as a metal halide perovskite included in the second light-emitting material layer, PEAPbBr₃ can be used as a metal halide perovskite included in the first light-emitting material layer. That is, according to the present disclosure, the first light-emitting material layer including PEAPbBr₃ and the second light-emitting material layer including MAPbBr₃ are alternately stacked to manufacture a light-emitting layer having a tandem structure. In addition, when MAPbBr₃ is used as a metal halide perovskite included in the second light-emitting material layer, MAPbCl₃, BAPbBr₃, or EAPbBr₃ may also be used as a metal halide perovskite included in the first light-emitting material layer, but is not limited thereto.

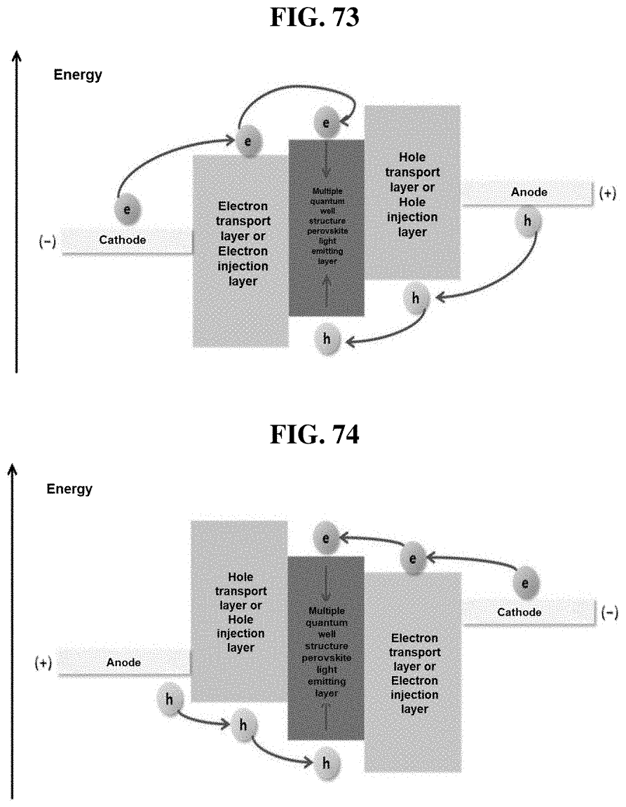

FIG. 73 shows energy levels of constituent layers in a light-emitting device (normal structure) including a light-emitting layer according to an embodiment of the present disclosure, and FIG. 74 shows the energy levels of the constituent layers in a light-emitting device (inverted structure) including a light-emitting layer according to another embodiment of the present disclosure.

FIGS. 73 and 74, in the light emitting device according to an embodiment of the present disclosure, the energy level of the VBM (Valence Band Maximum) of the light emitting layer (40) is the highest occupied molecular orbital (HOMO) of the hole injection layer is lower than the energy level of the electron transport layer, and higher than the energy level of the highest occupied molecular orbital (HOMO) of the electron transport layer. When having such an energy level, when a forward bias is applied to the light emitting device, it becomes easier for holes from the anode (20) to flow into the light emitting layer (40) through the hole injection layer (30). In addition, in the light emitting device according to an embodiment of the present disclosure, it is preferred that the energy level of the electron band minimum (CBM) of the light emitting layer is lower than that of the lower unoccupied molecular orbital (LUMO) of the hole injection layer, and higher than the energy level of LUMO (lowest unoccupied molecular orbital) of the electron transport layer. By having such an energy level, electrons and holes introduced into the light emitting layer (40) are combined to form excitons, and light may be emitted while the excitons transition to the ground state.

<Stacked Tandem Metal Halide Perovskite Light Emitting Diode>

According to another embodiment of the present disclosure, the metal halide perovskite described above may be used in a stacked hybrid light emitting diode.

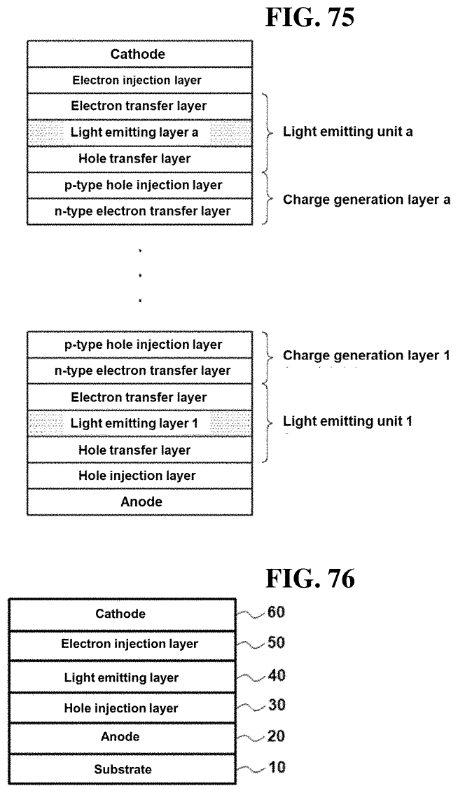

Referring to FIG. 75, a hybrid light emitting diode according to an embodiment includes an anode, first to ath light emitting units, 1 to a-1 charge generation layers, and a cathode (here, a is an integer greater than or equal to 2). Hereinafter, for convenience, a hybrid light-emitting diode including up to the a-th light-emitting unit may be referred to as a-th light-emitting diode.

The anode may include ITO, FTO, graphene, nanowires, and polymer electrodes, but is not limited thereto. The anode may be formed of a polymer electrode or CNT through a solution process, or may include a transparent electrode material such as ITO and IZO through a sputtering process.

The charge generation layer may include an n-type layer for generating and injecting electrons and a p-type layer for generating and injecting holes, and electrons and holes may be injected into adjacent light emitting units without resistance. The n-type layer may be an electron transport layer, and the p-type layer may be a hole injection layer.

The n-type electron transport layer may be an organic material alone or an organic material doped with an n-type dopant in an amount of about 5 to 40%. The electron transfer organic material may include Quinoline derivatives, especially tris(8-hydroxyquinoline)aluminum(Alq₃), bis(2-methyl-8-quinolinolate)-4-(phenylphenolato)aluminum(Balq), bis(10-hydroxybenzo[h]quinolinato)-beryllium (Bebg₂), 2,9-dimethyl-4,7-diphenyl-1,10-phenanthroline (BCP), 4,7-diphenyl-1,10-phenanthroline (Bphen), 2,2′, 2″-(benzene-1,3,5-triyl)-tris(1-phenyl-1H-benzimidazole) (TPBI), 3-(4-biphenyl)-4-(phenyl-5-tert-butylphenyl-1,2,4-triazole (TAZ), 4-(naphthalen-1-yl)-3,5-diphenyl-4H-1,2, 4-triazole (NTAZ), 2,9-bis(naphthalen-2-yl)-4,7-Diphenyl-1,10-phenanthroline (NBphen), Tris(2,4,6-trimethyl-3-(pyridin-3-yl)phenyl)borane (3TPYMB), phenyl-phenyl-dipyrenylphosphine oxide (POPy₂), 3,3′, 5,5′-tetra[(m-pyridyl)-phen-3-yl]biphenyl (BP4mPy), 1,3,5-tri[(3-pyridyl)-phen-3-yl]benzene (TmPyPB), 1,3-bis[3,5-di(pyridin-3-yl)phenyl]benzene (BmPyPhB), bis(10-hydroxybenzo[h]quinolinato)beryllium (Bepq₂), Diphenylbis(4-(pyridin-3-yl)phenyl)silane (DPPS), 1,3,5-tri(p-pyrid-3-Yl-phenyl)benzene (TpPyPB), 1,3-bis[2-(2,2′-bipyridin-6-yl)-1,3,4-oxadiazo-5-yl]benzene (Bpy-OXD), 6,6′-bis[5-(biphenyl-4-yl)-1,3,4-oxadiazo-2-yl]-2,2′-bipyridyl (BP-OXD-Bpy), TSPO1 (diphenylphosphine oxide-4-(triphenylsilyl)phenyl), TPBi(1,3,5-tris(N-phenylbenzimidazol-2-yl)benzene), Tris(8-quinolinorate)aluminum (Alq3), 2,5-diaryl silol derivative (PyPySPyPy), perfluorinated compound (PF-6P), COTs (Octasubstituted cyclooctatetraene), but is not limited thereto.

The n-type dopant may be an alkali metal, alkaline earth metal such as Li, Na, K, Rb, Cs, Be, Mg, Ca, Sr, Ba, or a carbonate-based compound, azide-based compound, a nitride-based compound, a nitrate-based compound, a phosphate-based compound, or a quinolate-based compound. As an example of a compound based on an alkali metal or alkaline earth metal, Li₂CO₃, LiNO₃, RbNO₃, Rb₂CO₃, AgNO₃, Ba(NO₃)₂, Mn(NO₃)₂, Zn(NO₃)₂, CsNO₃, Cs₂CO₃, CsF, CsN₃, FePo₄ and NaN₃, but are not limited thereto.

The thickness of the n-type electron transport layer may be about 5 to 50 nm.

The p-type hole injection layer may be an organic material alone or an organic material doped with a p-type dopant in an amount of about 5 to 40%.

Specifically, the hole transport organic material may be at least one selected from the group consisting of Fullerene(C₆₀), HAT-CN, F₁₆CuPC, CuPC, m-MTDATA [4,4′, 4″-tris(3-methylphenylphenylamino)triphenylamine], NPB [N,N′-Di(1-naphthyl)-N,N′-diphenyl-(1,1′-biphenyl)-4,4′-diamine)], TDATA, 2T-NATA, Pani/DBSA (Polyaniline/Dodecylbenzenesulfonic acid:Polyaniline/Dodecylbenzenesulfonic acid), PEDOT/PSS (Poly(3,4-ethylenedioxythiophene)/Poly(4-styrenesulfonate):Poly(3,4-ethylenedioxythiophene)/poly(4-styrene) Sulfonate)), Pani/CSA (Polyaniline/Camphor sulfonic acid:polyaniline/camphor sulfonic acid) and PANI/PSS (Polyaniline)/Poly(4-styrenesulfonate):polyaniline)/poly(4-styrenesulfonate)), 1,3-bis(carbazol-9-yl)benzene (MCP), 1,3,5-tris(carbazol-9-yl)benzene (TCP), 4,4′,4″-tris(carbazol-9-yl)triphenylamine (TCTA), 4,4′-bis(carbazol-9-yl)biphenyl (CBP), N,N′-Bis(naphthalen-1-yl)-N,N′-bis(phenyl)benzidine (NPB), N,N′-bis(naphthalen-2-yl)-N,N′-bis(phenyl)-benzidine (β-NPB), N,N′-bis(naphthalen-1-yl)-N,N′-bis(phenyl)-2,2′-dimethylbenzidine (α-NPD), di-[4,-(N,N-ditolyl-amino)-phenyl]cyclohexane (TAPC), N,N,N′,N′-tetra-naphthalen-2-yl-benzidine (β-TNB) and N4,N4,N4′,N4′-tetra(biphenyl-4-yl)biphenyl-4,4′-diamine(TPD15), poly(9,9-dioctylfluorene-co-bis-N,N′-(4-butylphenyl)-bis-N,N′-phenyl-1,4-phenylenediamine) (PFB), poly(9,9′-dioctylfluorene-co-N-(4-butylphenyl)diphenylamine) (TFB), poly(9,9′-dioctylfluorene-co-bis-N,N′-(4-butylphenyl)-bis-N,N′-phenylbenzidine) (BFB), poly(9,9-dioctylfluorene-co-bis-N,N′-(4-methoxyphenyl)-bis-N,N′-phenyl-1, and 4-phenylenediamine) (PFMO), but is not limited thereto.

The p-type dopant may be F4-TCNQ, FeCl₃, WO₃, MoO₃, ReO₃, Fe₃O₄, MnO₂, SnO₂, CoO₂, CuPC, metal oxide, or a hole injection organic material having a deep LUMO level.

The thickness of the p-type hole injection layer may be about 5 to 30 nm.

The stacked hybrid light emitting diode according to the embodiment includes at least two or more light emitting units. In FIG. 76, each light emitting unit is illustrated to include a hole transport layer, a light emitting layer, and an electron transport layer, but is not limited thereto.

In addition, a hole blocking layer (not shown) may be located between the emission layer (40) and the electron transport layer (50). In addition, an electron blocking layer (not shown) may be located between the light emitting layer (40) and the hole transport layer. However, the present disclosure is not limited thereto, and the electron transport layer (50) may serve as a hole blocking layer, or the hole transport layer may serve as an electron blocking layer.

The anode (20) may be a conductive metal oxide, a metal, a metal alloy, or a carbon material. Conductive metal oxides include ITO, AZO(Al-doped ZnO), GZO(Ga-doped ZnO), IGZO(In,Ga-doped ZnO), MZO(Mg-doped ZnO), Mo-doped ZnO, Al-doped MgO, Ga-doped MgO, F-doped SnO₂, Nb-doped TiO₂ or CuAlO₂, or a combination thereof. Metals or metal alloys suitable as the anode 20 may be Au and CuI. The carbon material may be graphite, graphene, or carbon nanotubes.

The negative electrode (70) is a conductive film having a lower work function than the positive electrode (20), for example, metals such as aluminum, magnesium, calcium, sodium, potassium, indium, yttrium, lithium, silver, lead, cesium, or it can be formed using a combination of two or more types.

The anode (20) and the cathode (70) may be formed using a sputtering method, a vapor deposition method, or an ion beam deposition method. The hole injection layer (30), the hole transport layer, the light emitting layer (40), the hole blocking layer, the electron transport layer (50), and the electron injection layer (60) can be deposited independent of each other by a vapor deposition method or a coating method such as spraying and spin coating, dipping, printing, doctor blading, or electrophoresis.

The hole injection layer (30) and/or the hole transport layer is a layer having a HOMO level between the work function level of the anode (20) and the HOMO level of the emission layer (40), and it functions to increase the efficiency of hole injection or transport from the anode (20) to the emission layer (40).

The hole injection layer (30) or the hole transport layer may include a material commonly used as a hole transport material, and one layer may include different hole transport material layers. The hole transport material may be, for example, mCP (N,Ndicarbazolyl-3,5-benzene); PEDOT:PSS (poly(3,4-ethylenedioxythiophene):polystyrenesulfonate); NPD (N,N′-di(1-naphthyl)-N,N′-diphenylbenzidine); N,N′-Bis(3-methylphenyl)-N,N′-diphenylbenzidine(TPD); DNTPD (N4,N4′-Bis[4-[bis(3-methylphenyl)amino]phenyl]-N4,N4′-diphenyl-[1,1′-biphenyl]-4,4′-diamine); N,N′-diphenyl-N,N′-dinaphthyl-4,4′-diaminobiphenyl; N,N,N′N′-tetra-p-tolyl-4,4′-diaminobiphenyl; N,N,N′N′-tetraphenyl-4,4′-diaminobiphenyl; Porphyrin compound derivatives such as copper(II)1,10,15,20-tetraphenyl-21H,23H-porphyrin; TAPC (1,1-Bis[4-[N,N′-Di(p-tolyl)Amino]Phenyl]Cyclohexane); N,N,N-tri(p-tolyl)amine, 4,4′, Triarylamine derivatives such as 4′-tris[N-(3-methylphenyl)-N-phenylamino]triphenylamine; Carbazole derivatives such as N-phenylcarbazole and polyvinylcarbazole; Phthalocyanine derivatives such as metal-free phthalocyanine and copper phthalocyanine; Starburst amine derivatives; Enaminestilbene derivatives; Derivatives of aromatic tertiary amines and styrylamine compounds; polysilane. Such a hole transport material may serve as an electron blocking layer.

The hole injection layer (30) may also include a hole injection material. For example, the hole injection layer may include at least one of a metal oxide and a hole injection organic material.

When the hole injection layer (30) includes a metal oxide, the metal oxide may contain one or more metal oxides selected from the group consisting of MoO₃, WO₃, V₂O₅, nickel oxide (NiO), copper oxide (Copper(II) Oxide: CuO), copper aluminum oxide (CAO, CuAlO₂), Zinc Rhodium Oxide (ZRO, ZnRh₂O₄), GaSnO, and metal-sulfide (FeS, ZnS or CuS).

When the hole injection layer (30) contains a hole injection organic material, the hole injection layer (30) may be formed according to a method arbitrarily selected from a variety of known methods such as vacuum deposition method, spin coating method, cast method, Langmuir-Blodgett (LB) method, spray coating method, dip coating method, gravure coating method, reverse offset coating method, screen printing method, slot-die coating method, and nozzle printing method.

The hole injecting organic material may be at least one selected from the group consisting of Fullerene(C₆₀), HAT-CN, F₁₆CuPC, CuPC, m-MTDATA [4,4′, 4″-tris(3-methylphenylphenylamino)triphenylamine], NPB [N,N′-Di(1-naphthyl)-N,N′-diphenyl-(1,1′-biphenyl)-4,4′-diamine)], TDATA, 2T-NATA, Pani/DBSA (Polyaniline/Dodecylbenzenesulfonic acid:Polyaniline/Dodecylbenzenesulfonic acid), PEDOT/PSS (Poly(3,4-ethylenedioxythiophene)/Poly(4-styrenesulfonate):Poly(3,4-ethylenedioxythiophene)/poly(4-styrene) Sulfonate)), Pani/CSA (Polyaniline/Camphor sulfonic acid:polyaniline/camphor sulfonic acid) and PANI/PSS (Polyaniline)/Poly(4-styrenesulfonate):polyaniline)/poly(4-styrenesulfonate)).

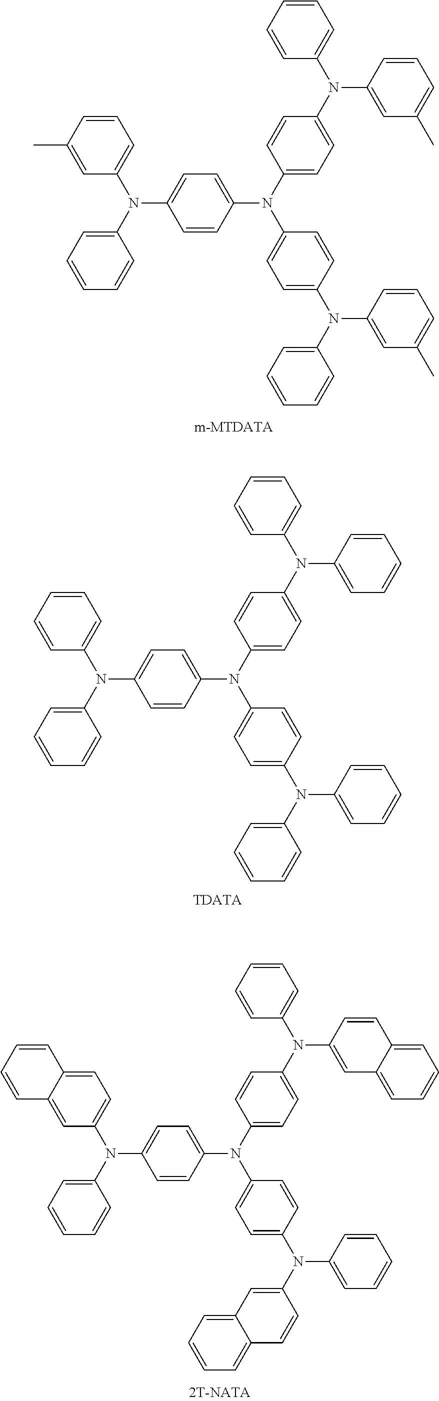

For example, the hole injection layer may be a layer doped with the metal oxide on the hole injection organic material matrix. In this case, the doping concentration is preferably 0.1 wt % to 80 wt % based on the total weight of the hole injection layer.

The hole injection layer may have a thickness of 1 nm to 1000 nm. For example, the thickness of the hole injection layer may include a range in which the lower value of the number is the lower limit value and the higher value is the upper limit value selected from the group of 1 nm, 2 nm, 3 nm, 4 nm, 5 nm, 6 nm, 7 nm, 8 nm, 9 nm, 10 nm, 11 nm, 12 nm, 13 nm, 14 nm, 15 nm, 16 nm, 17 nm, 18 nm, 19 nm, 20 nm, 21 nm, 22 nm, 23 nm, 24 nm, 25 nm, 26 nm, 27 nm, 28 nm, 29 nm, 30 nm, 31 nm, 32 nm, 33 nm, 34 nm, 35 nm, 36 nm, 37 nm, 38 nm, 39 nm, 40 nm, 41 nm, 42 nm, 43 nm, 44 nm, 45 nm, 46 nm, 47 nm, 48 nm, 49 nm, 50 nm, 51 nm, 52 nm, 53 nm, 54 nm, 55 nm, 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 101 nm, 102 nm, 103 nm, 104 nm, 105 nm, 106 nm, 107 nm, 108 nm, 109 nm, 110 nm, 111 nm, 112 nm, 113 nm, 114 nm, 115 nm, 116 nm, 117 nm, 118 nm, 119 nm, 120 nm, 125 nm, 130 nm, 135 nm, 140 nm, 145 nm, 150 nm, 155 nm, 160 nm, 165 nm, 170 nm, 175 nm, 180 nm, 185 nm, 190 nm, 195 nm, 200 nm, 210 nm, 220 nm, 230 nm, 240 nm, 250 nm, 260 nm, 270 nm, 280 nm, 290 nm, 300 nm, 310 nm, 320 nm, 330 nm, 340 nm, 350 nm, 360 nm, 370 nm, 380 nm, 390 nm, 400 nm, 450 nm, 500 nm, 550 nm, 600 nm, 650 nm, 700 nm, 750 nm, 800 nm, 850 nm, 900 nm, 950 nm, 1000 nm. Also, preferably, the thickness of the hole injection layer may be 10 nm to 200 nm. When the thickness of the hole injection layer satisfies the above-described range, the driving voltage is not increased, so that a high-quality organic device can be implemented.

In addition, a hole transport layer may be further formed between the light emitting layer and the hole injection layer.

The hole transport layer may include a known hole transport material. For example, the hole transport material that may be included in the hole transport layer may be at least one selected from the group consisting of 1,3-bis(carbazol-9-yl)benzene (MCP), 1,3,5-tris(carbazol-9-yl)benzene (TCP), 4,4′, 4″-tris(carbazol-9-yl)triphenylamine (TCTA), 4,4′-bis carbazol-9-yl)biphenyl (CBP), N,N′-bis(Naphthalen-1-yl)-N,N′-bis(phenyl)benzidine (NPB), N,N′-bis(naphthalen-2-yl)-N,N′-bis(phenyl)-benzidine (β-NPB), N,N′-bis(naphthalen-1-yl)-N,N′-bis(phenyl)-2,2′-dimethylbenzidine (α-NPD), di-[4,-(N,N-ditolyl-amino)-phenyl]cyclohexane (TAPC), N,N,N′,N′-tetra-naphthalen-2-yl-benzidine (NO-TNB) and N4,N4,N4′,N4′-tetra(biphenyl-4-yl)biphenyl-4,4′-diamine (TPD15), poly(9,9-dioctylfluorene-co-bis-N,N′-(4-butylphenyl)-bis-N,N′-phenyl-1,4-phenylenediamine) (PFB), poly(9,9′-dioctylfluorene-co-N-(4-butylphenyl)diphenylamine)(TFB), poly(9,9′-dioctylfluorene-co-bis-N,N′-(4-butylphenyl)-bis-N,N′-phenylbenzidine)(BFB), poly(9,9-dioctylfluorene-co-bis-N,N′-(4-methoxyphenyl)-bis-N,N′-phenyl-1, and 4-phenylenediamine) (PFMO), but is not limited thereto.

Among the hole transport layers, for example, in the case of TCTA, in addition to the hole transport role, it may play a role of preventing diffusion of excitons from the emission layer.

The hole transport layer may have a thickness of 1 nm to 100 nm. For example, the thickness of the hole transport layer may include a range in which the lower value of the number is the lower limit value and the higher value is the upper limit value selected from the group of 1 nm, 2 nm, 3 nm, 4 nm, 5 nm, 6 nm, 7 nm, 8 nm, 9 nm, 10 nm, 11 nm, 12 nm, 13 nm, 14 nm, 15 nm, 16 nm, 17 nm, 18 nm, 19 nm, 20 nm, 21 nm, 22 nm, 23 nm, 24 nm, 25 nm, 26 nm, 27 nm, 28 nm, 29 nm, 30 nm, 31 nm, 32 nm, 33 nm, 34 nm, 35 nm, 36 nm, 37 nm, 38 nm, 39 nm, 40 nm, 41 nm, 42 nm, 43 nm, 44 nm, 45 nm, 46 nm, 47 nm, 48 nm, 49 nm, 50 nm, 51 nm, 52 nm, 53 nm, 54 nm, 55 nm, 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm. Also, preferably, the thickness of the hole injection layer may be 10 nm to 60 nm. When the thickness of the hole transport layer satisfies the above-described range, light efficiency of the organic light emitting diode may be improved and luminance may be increased.

The electron injection layer (60) and/or the electron transport layer (50) are layers having an LUMO level between the work function level of the cathode (70) and the LUMO level of the emission layer (40), and it functions to increase the efficiency of injection or transport of electrons from the cathode (70) to the emission layer (40).

The electron injection layer (60) may be, for example, LiF, NaCl, CsF, Li₂O, BaO, BaF₂, or Liq (lithium quinolate).

The electron transport organic material may include Quinoline derivatives, especially tris(8-hydroxyquinoline)aluminum(Alq₃), bis(2-methyl-8-quinolinolate)-4-(phenylphenolato)aluminum(Balq), bis(10-hydroxybenzo[h]quinolinato)-beryllium (Bebg₂), 2,9-dimethyl-4,7-diphenyl-1,10-phenanthroline (BCP), 4,7-diphenyl-1,10-phenanthroline (Bphen), 2,2′, 2″-(benzene-1,3,5-triyl)-tris(1-phenyl-1H-benzimidazole) (TPBI), 3-(4-biphenyl)-4-(phenyl-5-tert-butylphenyl-1,2,4-triazole (TAZ), 4-(naphthalen-1-yl)-3,5-diphenyl-4H-1,2, 4-triazole (NTAZ), 2,9-bis(naphthalen-2-yl)-4,7-Diphenyl-1,10-phenanthroline (NBphen), Tris(2,4,6-trimethyl-3-(pyridin-3-yl)phenyl)borane (3TPYMB), phenyl-phenyl-dipyrenylphosphine oxide (POPy2), 3,3′, 5,5′-tetra[(m-pyridyl)-phen-3-yl]biphenyl (BP4mPy), 1,3,5-tri[(3-pyridyl)-phen-3-yl]benzene (TmPyPB), 1,3-bis[3,5-di(pyridin-3-yl)phenyl]benzene (BmPyPhB), bis(10-hydroxybenzo[h]quinolinato)beryllium (Bepq2), Diphenylbis(4-(pyridin-3-yl)phenyl)silane (DPPS), 1,3,5-tri(p-pyrid-3-Yl-phenyl)benzene (TpPyPB), 1,3-bis[2-(2,2′-bipyridin-6-yl)-1,3,4-oxadiazo-5-yl]benzene (Bpy-OXD), 6,6′-bis[5-(biphenyl-4-yl)-1,3,4-oxadiazo-2-yl]-2,2′-bipyridyl (BP-OXD-Bpy), TSPO1 (diphenylphosphine oxide-4-(triphenylsilyl)phenyl), TPBi(1,3,5-tris(N-phenylbenzimidazol-2-yl)benzene), Tris(8-quinolinorate)aluminum (Alq3), 2,5-diaryl silol derivative (PyPySPyPy), perfluorinated compound (PF-6P), COTs (Octasubstituted cyclooctatetraene).

The hole transport layer may have a thickness of 5 nm to 100 nm. For example, the thickness of the hole transport layer may include a range in which the lower value of the number is the lower limit value and the higher value is the upper limit value selected from the group of 5 nm, 6 nm, 7 nm, 8 nm, 9 nm, 10 nm, 11 nm, 12 nm, 13 nm, 14 nm, 15 nm, 16 nm, 17 nm, 18 nm, 19 nm, 20 nm, 21 nm, 22 nm, 23 nm, 24 nm, 25 nm, 26 nm, 27 nm, 28 nm, 29 nm, 30 nm, 31 nm, 32 nm, 33 nm, 34 nm, 35 nm, 36 nm, 37 nm, 38 nm, 39 nm, 40 nm, 41 nm, 42 nm, 43 nm, 44 nm, 45 nm, 46 nm, 47 nm, 48 nm, 49 nm, 50 nm, 51 nm, 52 nm, 53 nm, 54 nm, 55 nm, 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm. Also, preferably, the thickness of the hole injection layer may be 15 nm to 60 nm. When the thickness of the electron transport layer satisfies the above-described range, excellent electron transport characteristics can be obtained without an increase in driving voltage.

The electron injection layer (60) may include a metal oxide. Since the metal oxide has n-type semiconductor properties, it has excellent electron transport capability, and further, it is a material that is not reactive to air or moisture, and may be selected from semiconductor materials having excellent transparency in a visible light region.

The electron injection layer (60) may include, for example, one or more metal oxides selected from aluminum doped zinc oxide (AZO), alkali metal (Li, Na, K, Rb, Cs or Fr) doped AZO, TiOₓ (x is a real number of 1 to 3), indium oxide (In₂O₃), tin oxide (SnO₂), zinc oxide (ZnO), zinc tin oxide (Zinc Tin Oxide), gallium oxide (Ga₂O₃), tungsten oxide (WO₃), aluminum oxide, titanium oxide, vanadium oxide (V₂O₅, vanadium (IV))) oxide (VO₂), V₄O₇, V₅O₉, or V₂O₃), molybdenum oxide (MoO₃ or MoOₓ), copper oxide (CuO), nickel oxide (NiO), copper aluminum oxide (CAO, CuAlO₂), Zinc Rhodium Oxide (ZRO, ZnRh₂O₄), iron oxide, chromium oxide, bismuth oxide, IGZO (indium-Gallium Zinc Oxide), and ZrO₂, but is not limited thereto. As an example, the electron injection layer (60) may be a metal oxide thin film layer, a metal oxide nanoparticle layer, or a layer including metal oxide nanoparticles in the metal oxide thin film.

The electron injection layer (60) may be formed using a wet process or a vapor deposition method.

As an example of a wet process, when the electron injection layer (60) is formed by a solution method (ex. a sol-gel method). The electron injection layer (60) may be formed with heat treatment after applying the mixture for an electron injection layer containing at least one of a sol-gel precursor of a metal oxide and a metal oxide in the form of nanoparticles and a solvent. In this case, the solvent may be removed by heat treatment or the electron injection layer (60) may be crystallized. The method of providing the mixed solution for the electron injection layer on the substrate (10) is a known coating method, for example, a spin coating method, a cast method, a Langmuir-Blodgett (LB) method, a spray coating method, a dip coating method, gravure coating method, a reverse offset coating method, a screen printing method, a slot-die coating method, a nozzle printing method, and a dry transfer printing method may be selected, but the present disclosure is not limited thereto.

The sol-gel precursor of the metal oxide may contain at least one selected from the group consisting a metal salt (eg, metal halide, metal sulfate, metal nitrate, metal perchlorate, metal acetate, metal carbonate, etc.), metal salt hydrate, metal hydroxide, metal alkyl, metal of alkoxide, metal carbide, metal acetylacetonate, metal acid, metal acid salt, metal acid hydrate, metal sulfide, metal acetate, metal alkanoate, metal phthalocyanine, metal nitride, and metal carbonate.

When the metal oxide is ZnO, the ZnO sol-gel precursor may be at least one selected from the group consisting of Zinc sulfate, zinc fluoride, zinc chloride, zinc bromide, zinc iodide, zinc perchlorate, zinc hydroxide (Zn(OH)₂), zinc acetate (Zn(CH₃COO)₂), zinc acetate hydrate (Zn(CH₃(COO)₂nH₂O), Diethyl zinc (Zn(CH₃CH₂)₂), zinc nitrate (Zn(NO₃)₂), zinc nitrate (Zn(NO₃)₂nH₂O), zinc carbonate (Zn(C₀₃)), zinc acetylacetonate (Zn(CH₃COCHCOCH₃))2), and zinc acetylacetonate hydrate (Zn(CH₃COCHCOCH₃)₂nH₂O), but is not limited thereto.

When the metal oxide is indium oxide (In₂O₃), at least one selected from the group consisting of Indium nitrate hydrate (In(NO₃)₃nH₂O), indium acetate (In(CH₃COO)₂), indium acetate hydrate (In(CH₃(COO)₂nH₂O), indium chloride (InCl, InCl₂, InCl₃), indium nitrate (In(NO₃)₃), indium nitrate hydrate (In(NO₃)₃nH₂O), indium acetylacetonate In(CH₃COCHCOCH₃)₂), and indium acetylacetonate hydrate (In(CH₃COCHCOCH₃)₂nH₂O) may be used as the In₂O₃ sol-gel precursor.

When the metal oxide is tin oxide (SnO₂), at least one selected from the group consisting of tin acetate (Sn(CH₃COO)₂), tin acetate hydrate (Sn(CH₃(COO)₂nH₂O), tin chloride (SnCl₂, SnCl₄), tin chloride hydrate (SnClₙnH₂O), tin acetylacetonate (Sn(CH₃COCHCOCH₃)₂), and tin acetylacetonate hydrate (Sn(CH₃COCHCOCH₃)₂nH₂O) may be used as the SnO₂ sol-gel precursor.

When the metal oxide is gallium oxide (Ga₂O₃), at least one selected from the group consisting of gallium nitrate (Ga(NO₃)₃), gallium nitrate hydrate (Ga(NO₃)₃nH₂O), gallium acetylacetonate (Ga(CH₃COCHCOCH₃)₃), Gallium acetylacetonate hydrate (Ga(CH₃COCHCOCH₃)₃H₂O), and gallium chloride (Ga₂Cl₄, GaCl₃) may be used as the Ga₂O₃ sol-gel precursor.

When the metal oxide is tungsten oxide (WO₃), at least one selected from the group consisting of tungsten carbide (WC), tungstic acid powder (H₂WO₄), tungsten chloride (WCl₄, WCl₆), tungsten isopropoxide (W(OCH(CH₃)₂)₆), sodium tungstate (Na₂WO₄), sodium tungstate hydrate (Na₂WO₄nH₂O), ammonium tungstate ((NH₄)₆H₂W₁₂O₄₀), ammonium tungstate hydrate ((NH₄)₆H₂W₁₂O₄₀nH₂O), and tungsten ethoxide (W(OC₂H₅)₆ may be used as the WO₃ sol-gel precursor.

When the metal oxide is aluminum oxide, at least one selected from the group consisting of aluminum chloride (AlCl₃), aluminum nitrate (Al(NO₃)₃), aluminum nitrate hydrate (Al(NO₃)₃nH₂O), and aluminum butoxide (Al(C₂H₅CH(CH₃)O)) may be used as the aluminum oxide sol-gel precursor.

When the metal oxide is titanium oxide, at least one selected from the group consisting of titanium isopropoxide (Ti(OCH(CH₃)₂)₄), titanium chloride (TiCl₄), titanium ethoxide (Ti(OC₂H₅)₄), and titanium butoxide (Ti(OC₄H₉)₄) may be used as the titanium oxide sol-gel precursor.

When the metal oxide is vanadium oxide, at least one selected from the group consisting of vanadium isopropoxide (VO(OC₃H₇)₃), ammonium vanadate (NH₄VO₃), vanadium acetylacetonate (V(CH₃COCHCOCH₃)₃), and vanadium acetylacetonate hydrate (V(CH₃COCHCOCH₃)₃nH₂O) may be used as the vanadium oxide sol-gel precursor.

When the metal oxide is molybdenum oxide, at least one selected from the group consisting of molybdenum isopropoxide (Mo(OC₃H₇)₅), molybdenum chloride isopropoxide (MoCl₃(OC₃H₇)₂), ammonium molybdenate ((NH₄)₂MoO₄), and ammonium molybdenate hydrate ((NH₄)₂MoO₄nH₂O) may be used as the molybdenum oxide sol-gel precursor.

When the metal oxide is copper oxide, at least one selected from the group consisting of copper chloride (CuCl, CuCl₂), copper chloride hydrate (CuCl₂nH₂O), copper acetate (Cu(CO₂CH₃), Cu(CO₂CH₃)₂), Copper acetate hydrate (Cu(CO₂CH₃)₂nH₂O), copper acetylacetonate (Cu(C₅H₇O₂)₂), copper nitrate (Cu(NO₃)₂), copper nitrate hydrate (Cu(NO₃)₂nH₂O), copper bromide (CuBr, CuBr₂)), copper carbonate (CuCO₃Cu(OH)₂), copper sulfide (Cu₂S, CuS), copper phthalocyanine (C₃₂H₁₆N₈Cu), copper trifluoroacetate (Cu(CO₂CF₃)₂), copper isobutyrate (C₈H₁₄CuO₄), copper ethylacetoacetate (C₁₂H₁₈CuO₆), copper 2-ethylhexanoate ([CH₃(CH₂)₃CH(C₂H₀)CO₂]₂Cu), copper fluoride (CuF₂), copper formate hydrate ((HCO₂)₂CuH₂O), copper gluconate (C₁₂H₂₂CuO₁₄), Copper hexafluoroacetylacetonate (Cu(C₅HF₆O₂)₂), copper hexafluoroacetylacetonate hydrate (Cu(C₅HF₆O₂)₂nH₂O), copper methoxide (Cu(OCH₃)₂), copper neodecanoate (C₁₀H₁₉O₂Cu), copper perchlorate (Cu(ClO₄)₂6H₂O), copper sulfate (CuSO₄), copper sulfate hydrate (CuSO₄nH₂O), copper tartrate hydrate ([—CH(OH)CO₂]₂CunH₂O), copper trifluoroacetylacetonate (Cu(C₅H₄F₃O₂)₂), copper trifluoromethanesulfonate ((CF₃SO₃)₂Cu), and tetraamine copper sulfate hydrate (Cu(NH₃)₄SO₄H₂O) may be used as the copper oxide sol-gel precursor.

When the metal oxide is nickel oxide, at least one selected from the group consisting of nickel chloride (NiCl₂), nickel chloride hydrate (NiCl₂nH₂O), nickel acetate hydrate (Ni(OCOCH₃)₂4H₂O), nickel nitrate hydrate (Ni(NO₃)₂6H₂O), nickel acetylacetonate (Ni(C₅H₇O₂)₂), nickel hydroxide (Ni(OH)₂), nickel phthalocyanine (C₃₂H₁₆N₈Ni), and nickel carbonate hydrate (NiCO₃₂Ni(OH)₂nH₂O) may be used as the nickel oxide sol-gel precursor.

When the metal oxide is iron oxide, at least one selected from the group consisting of iron acetate (Fe(CO₂CH₃)₂), iron chloride (FeCl₂, FeCl₃), iron chloride hydrate (FeCl₃nH₂O), iron acetylacetonate (Fe(C₅H₇O₂)₃), iron nitrate hydrate (Fe(NO₃)₃₉H₂O), iron phthalocyanine (C₃₂H₁₆FeN₈), and iron oxalate hydrate (Fe(C₂O₄)nH₂O, and Fe₂(C₂O₄)₃6H₂O) may be used as the sol-gel precursor of iron oxide.

When the metal oxide is chromium oxide, at least one selected from a group consisting of chromium chloride (CrCl₂, CrCl₃), chromium chloride hydrate (CrCl₃nH₂O), chromium carbide (Cr₃C₂), chromium acetylacetonate (Cr(C₅H₇O₂)₃), at least one selected from the group consisting of chromium nitrate hydrate (Cr(NO₃)₃nH₂O), chromium hydroxide (CH₃CO₂)₇Cr₃(OH)₂, and chromium acetate hydrate ([(CH₃CO₂)₂CrH₂O]₂) may be used as the chromium oxide sol-gel precursor.

When the metal oxide is bismuth oxide, at least one selected from a group consisting of bismuth chloride (BiCl₃), bismuth nitrate hydrate (Bi(NO₃)₃nH₂O), bismuth acetic acid ((CH₃CO₂)₃Bi), and bismuth carbonate ((BiO)₂CO₃) may be used as the bismuth oxide sol-gel precursor.

When the metal oxide nanoparticles are contained in the mixed solution for the electron injection layer, the average particle diameter of the metal oxide nanoparticles may be 10 nm to 100 nm.

The solvent may be a polar solvent or a non-polar solvent. For example, examples of the polar solvent include alcohols and ketones, and examples of the nonpolar solvent include aromatic hydrocarbons, alicyclic hydrocarbons, and aliphatic hydrocarbon-based organic solvents. As an example, the solvent is ethanol, dimethylformamide, ethanol, methanol, propanol, butanol, isopropanol. Methyl ethyl ketone, propylene glycol (mono)methyl ether (PGM), isopropyl cellulose (IPC), ethylene carbonate (EC), methyl cellosolve (MC), ethyl cellosolve, 2-methoxy ethanol and ethanol amine It may be one or more selected from among, but is not limited thereto.

For example, when forming the electron injection layer (60) made of ZnO, the mixture for the electron injection layer contains zinc acetate dehydrate as a precursor of ZnO, and 2-methoxy as a solvent It may include a combination of ethanol and ethanol amine, but is not limited thereto.

The heat treatment conditions can vary depending on the type and content of the selected solvent, but it is generally preferably performed within the range of 100° C. to 350° C. and 0.1 hour to 1 hour. When the heat treatment temperature and time satisfy this range, the solvent removal effect is good and the device may not be deformed.

When the electron injection layer (60) is formed using a vapor deposition method, evaporation can be performed by various known methods such as electron beam deposition, thermal evaporation, sputter deposition, atomic layer deposition, chemical vapor deposition. The deposition conditions vary depending on the target compound, the structure of the target layer, and thermal properties, but for example, it is preferred that the deposition temperature range is of 25 to 1500° C., specifically 100 to 500° C., and the vacuum degree range of 10⁻¹⁰ to 10⁻³ torr, and the deposition rate is performed within the range of 0.01 to 100 Å/sec.

The electron injection layer (60) may have a thickness of 5 nm to 100 nm. For example, the thickness of the electron injection layer is a range in which the lower value of the number is the lower limit value and the higher value is the upper limit value selected from the group of 5 nm, 6 nm, 7 nm, 8 nm, 9 nm, 10 nm, 11 nm, 12 nm, 13 nm, 14 nm, 15 nm, 16 nm, 17 nm, 18 nm, 19 nm, 20 nm, 21 nm, 22 nm, 23 nm, 24 nm, 25 nm, 26 nm, 27 nm, 28 nm, 29 nm, 30 nm, 31 nm, 32 nm, 33 nm, 34 nm, 35 nm, 36 nm, 37 nm, 38 nm, 39 nm, 40 nm, 41 nm, 42 nm, 43 nm, 44 nm, 45 nm, 46 nm, 47 nm, 48 nm, 49 nm, 50 nm, 51 nm, 52 nm, 53 nm, 54 nm, 55 nm, 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm. In addition, preferably, the thickness of the electron injection layer may be 15 nm to 60 nm.

The above hole injection layer (30), the vacuum transport layer, the electron injection layer (60), or the electron transport layer (50) may normally be applied to substances used in conventional organic light emitting diodes.

The above hole injection layer (30), hole transport layer, electronic injection layer (60), or electron transport layer (50) may be formed by performing in a randomly selected manner among the various methods including vacuum deposition method, spin coating method, spray method, deep coating method, bar coating method, nozzle printing method, slot-die coating method, gravure printing method, cast method or Langmuir-blodgett(LB) method. At this time, the conditions and coating conditions for thin film formation may vary depending on the purpose compound, the structure and thermal properties of the intended layer.

The substrate (10) serves as a support for the light emitting device, and may be a transparent material. In addition, the substrate (10) may be a flexible material or a hard material, and preferably may be a flexible material.

The material of the substrate (10) is one of glass, sapphire, quartz, silicon, polyethylene terephthalate (PET), polystyrene (PS), polyimide (polyimide, PI), polyvinyl chloride (PVC), polyvinylpyrrolidone (PVP), polyethylene (PE), etc., but is not limited thereto.

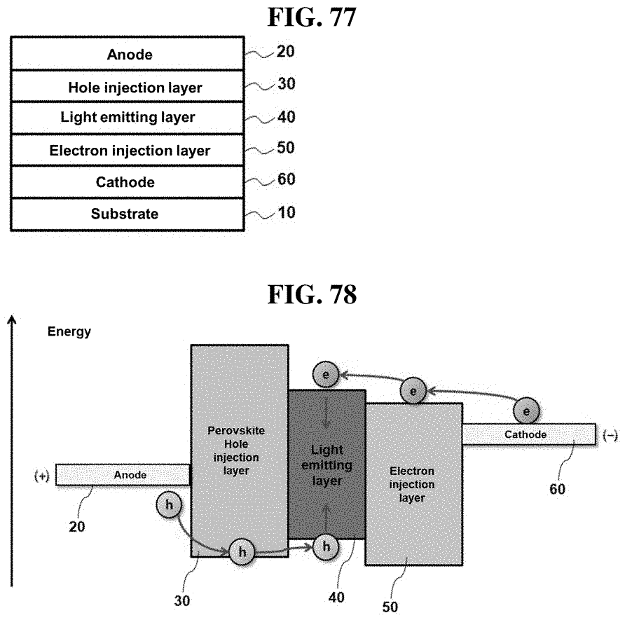

The substrate (10) may be located under the anode (20) or may be located above the cathode (70). In other words, the anode (20) may be formed before the cathode (70) on the substrate, or the cathode (70) may be formed before the anode (20). Accordingly, the light emitting device may have both the normal structure of FIG. 76 and the inverse structure of FIG. 77.

The emission layer (40) is formed between the hole injection layer (30) and the electron injection layer (60), and the holes (h) introduced from the anode (20) and the electrons (e) introduced from the cathode (70) combines to form excitons, and excitons transition to a ground state and light is emitted to cause light emission.

A stacked hybrid light emitting diode according to an embodiment includes at least one organic light emitting layer and at least one metal halide perovskite light emitting layer. Specifically, a first emission layer including a metal halide perovskite emitter and a second emission layer including an organic emitter may be included. Depending on the embodiment, the first emission layer may include a metal halide perovskite emitter, and the second emission layer may include an organic emitter.

An embodiment may include a combination of orange-red and sky-blue emitters or a combination of red, green, and blue emitters. As described above, in the stacked hybrid white light emitting diode according to an exemplary embodiment, light emitters emitting different colors may emit light at the same time to emit white light.

Depending on the embodiment, an organic light emitter and a metal halide perovskite light emitter that emit light of the same wavelength in a visible light region may be included. In this case, the current efficiency of the stacked hybrid high-efficiency light-emitting diode according to an exemplary embodiment may be equal to the sum of the current efficiencies of each light-emitting unit.

As an organic light-emitting diode, a fluorescent low-molecular organic material, a phosphorescent low-molecular organic material, a thermally activated delayed fluorescence (TADF) organic material and a polymer may be used, but the present disclosure is not limited thereto.

The fluorescent organic light-emitter may include at least one of the dopants from the group of 4-(dicyanomethylene)-2-t-butyl-6-(1,1,7,7-tetramethyljulolidyl-9-enyl)-4H-pyran(DCJ TB), (E)-2-(2-(4-(Dimethylamino)styryl)-6-methyl-4H-pyran-4-ylidene)malononitrile(DCM), 5,6-bis(4-(9,9-dimethylacridin-10(9H)-yl)phenyl)pyrazine-2,3-dicarbonitrile (Ac-CNP).

Phosphorescent organic emitters may include at least one of the dopants from the group of Bt2Ir(acac), tris(1-phenylisoquinoline) iridium(III) (Ir(piq)3), Bis(2-(3,5-dimethylphenyl)-4-phenylpyridine)(2,2,6,6-tetramethylheptane-3,5-diketonate)iridium(III)(Ir(dmppy-ph)2tmd), Bis(2-benzo[b]thiophen-2-yl-pyridine)(acetylacetonate)iridium(III)(Ir(btp)2(acac)), Bis[1-(9,9-dimethyl-9H-fluoren-2-yl)-isoquinoline](acetylacetonate)iridium(III)(Ir(fliq)2(acac)), Bis[2-(9,9-dimethyl-9H-fluoren-2-yl)quinoline](acetylacetonate)iridium(III)(Ir(flq)2(acac)), Bis[2-(4-n-hexylphenyl)quinoline](acetylacetonate)iridium(III)(Hex-Ir(phq)2 (acac)), Tris[2-(4-n-hexylphenyl)quinoline)]iridium(III)(Hex-Ir(phq)3, Bis(2-phenylquinoline)(2-(3-methylphenyl)pyridinate)iridium(III)(Ir(phq)2tpy).

Thermally activated delayed fluorescence (TADF) organic light emitter may include at least one of the dopants from the group of Dibenzo{[f,f′]-4,4′, 7,7′-tetraphenyl}diindeno[1,2,3-cd:1′, 2′, 3′-lm]perylene(DBP), 2,3,5,6-Tetrakis[3,6-bis(1,1-dimethylethyl)-9H-carbazol-9-yl]benzonitrile(4CzBN), 7,10-Bis(4-(diphenylamino)phenyl)-2,3-dicyanopyrazino phenanthrene(TPA-DCPP), 2,8-Di-tert-butyl-5,11-bis(4-tert-butylphenyl)-6,12-diphenyltetracene(TBRb), 2-(9-phenyl-9H-carbazol-3-yl)-10, 10-dioxide-9H-thioxanthen-9-one (TXO-PhCz).

Since the metal halide perovskite is the same as described above, a detailed description will be omitted.

The hybrid light-emitting diode according to an embodiment of the present disclosure includes a halide metal halide perovskite light-emitting body having a half-width (FWHM) of about 20 nm or less together with an organic light-emitting body, so that the manufacturing cost can be significantly reduced, and white light of high color purity can be realized by performing solution process up to at least the second light-emitting unit.

<Light-Emitting Device Including a Metal Halide Perovskite Charge Transport Layer>

The light emitting device according to an embodiment of the present disclosure may be characterized in that it includes a metal halide perovskite charge transport layer.

In the present specification, the “charge transport layer” refers to a hole injection layer, a hole transport layer, an electron transport layer, or an electron injection layer adjacent to the light emitting layer that moves holes or electrons from the anode or the cathode to the light emitting layer. In general, the charge transport layer refers to a hole injection layer or an electron injection layer adjacent to the emission layer, but in the case of a light emitting device including a hole transport layer between the emission layer and the hole injection layer, and an electron transport layer between the emission layer and the electron injection layer, A hole transport layer or an electron transport layer adjacent to the light emitting layer is also included in the charge transport layer.

Referring to FIG. 76, a light emitting device including a metal halide perovskite charge transport layer according to the present disclosure includes an anode (20) and a cathode (60), and a light emitting layer (40) located between the two electrodes, and the hole injection layer (30) may be provided between the anode (20) and the light emitting layer (40) to facilitate injection of holes. In addition, an electron injection layer (50) for facilitating injection of electrons may be provided between the light emitting layer (40) and the cathode (60).

In addition, the light emitting device according to the present disclosure may further include a hole transport layer (35) for transporting holes between the hole injection layer (30) and the light emitting layer (40).

In addition, the light emitting device according to the present disclosure may further include an electron transport layer (45) for transporting electrons between the hole injection layer (30) and the light emitting layer (40).

In addition, a hole blocking layer (not shown) may be located between the emission layer (40) and the electron transport layer (45). In addition, an electron blocking layer (not shown) may be located between the light emitting layer (40) and the hole transport layer (35). However, the present disclosure is not limited thereto, and the electron transport layer (45) may function as a hole blocking layer, or the hole transport layer (35) may function as an electron blocking layer.

In addition, a hole transport layer may be further formed between the light emitting layer and the hole injection layer.

The hole injection layer (30), the emission layer (40), the hole transport layer, the electron injection layer (60), or the electron transport layer (50) may be materials used in conventional organic light emitting diodes.

On the other hand, in the light emitting device, a feature of the present disclosure is at least one selected from the group consisting of the hole injection layer (30), the hole transport layer (35), the electron transport layer (45), and the electron injection layer (50). The charge transport layer includes a thin film of metal halide perovskite.

Specifically, the present disclosure provides

anode and cathode,

a light emitting layer located between the anode and the cathode,

At least one first charge transport layer of a hole injection layer and a hole transport layer located between the anode and the light emitting layer, and

A light emitting device comprising at least one second charge transport layer of an electron injection layer and an electron transport layer located between the light emitting layer and the cathode,

It provides a light emitting device, characterized in that the first charge transport layer or the second charge transport layer adjacent to the light emitting layer is a metal halide perovskite thin film.

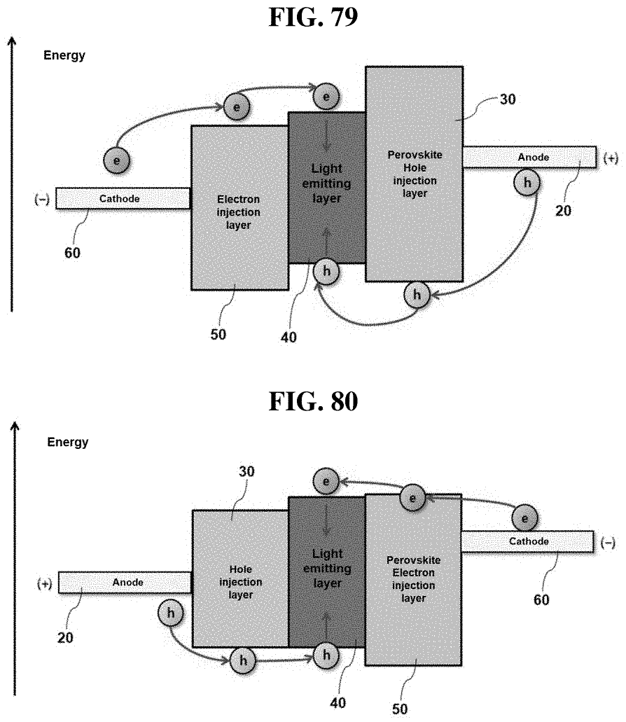

In the light emitting device according to an embodiment of the present disclosure, the first charge transport layer (eg, the hole injection layer (30)) may be a metal halide perovskite thin film (see FIGS. 78 and 79).

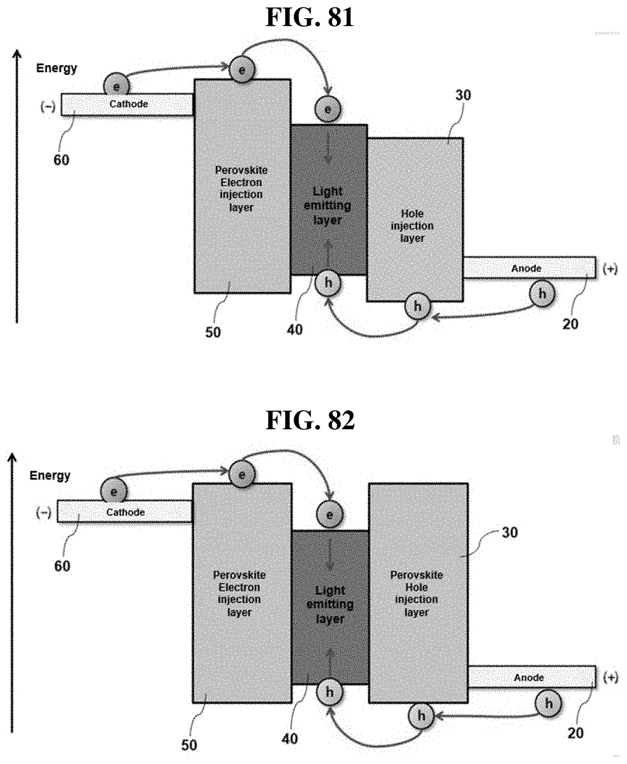

In the light emitting device according to an embodiment of the present disclosure, the second charge transport layer (eg, the electron injection layer (50)) may be a metal halide perovskite thin film (see FIGS. 80 and 81).

In addition, the present disclosure provides

A first charge transport layer of at least one of an anode and a cathode, a light emitting layer located between the anode and the cathode, a hole injection layer and a hole transport layer located between the anode and the light emitting layer, and

A light emitting device comprising at least one second charge transport layer of an electron injection layer and an electron transport layer located between the light emitting layer and the cathode, wherein the first charge transport layer and the second charge transport layer adjacent to the light emitting layer are metal halide perovskite thin film.

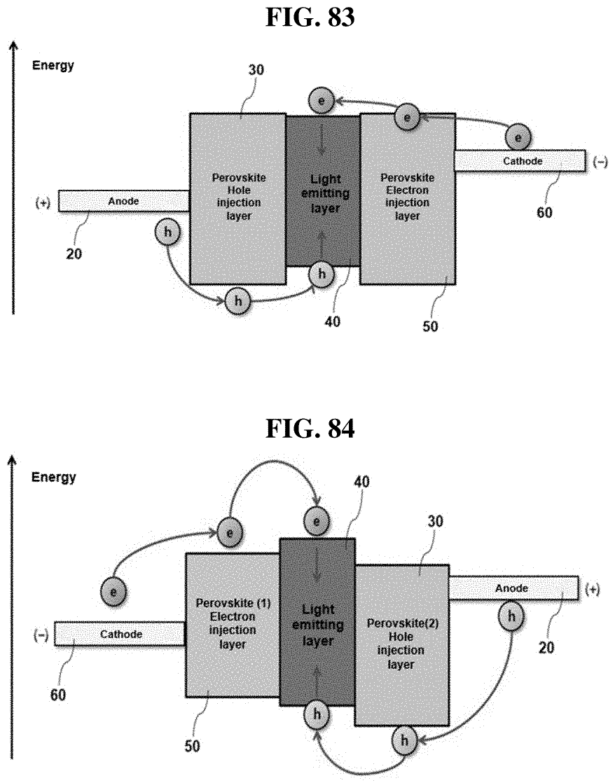

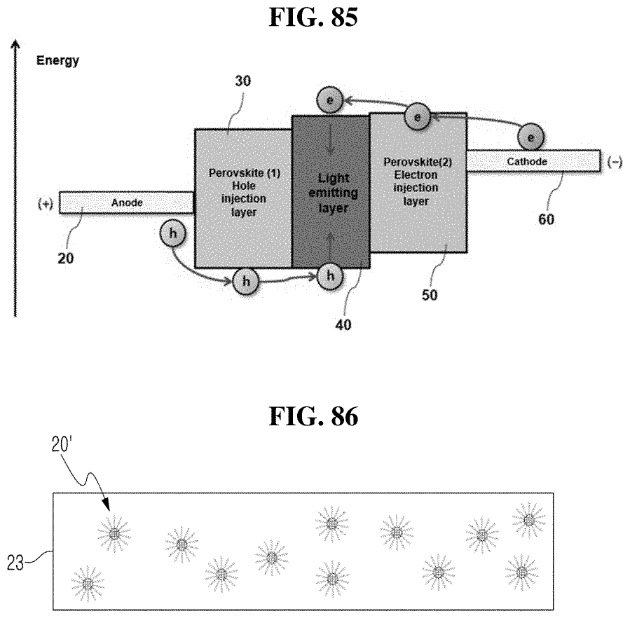

In the light emitting device according to an embodiment of the present disclosure, a first charge transport layer (eg, a hole injection layer (30)) and a second charge transport layer (eg, an electron injection layer (50)) are all metal halide perovskites. It may be a thin film. In this case, the metal halide perovskite thin films constituting the first and second charge transport layers may be the same (see FIGS. 82 and 83) or different (see FIGS. 84 and 85).

Until now, the metal halide perovskite thin film used in the metal halide perovskite light emitting device has been used only as a light emitting layer. However, since the metal halide perovskite has an energy level similar to that of an organic semiconductor material used in a light emitting device and has a much higher charge mobility than an organic semiconductor, it is very promising not only as a light emitting layer but also a charge transport layer.

At this time, since the metal halide perovskite used is the same as described above, a detailed description will be omitted.

<Metal Halide Perovskite Wavelength Converting Body>

According to another embodiment of the present disclosure, the metal halide perovskite described above may be used as a wavelength converting body.

A light emitting diode (LED) is a semiconductor device that converts current into light, and is mainly used as a light source of a display device. These light-emitting diodes are very small compared to conventional light sources, have low power consumption, have a long lifespan, and exhibit very excellent characteristics, such as a fast reaction speed. In addition, since it does not emit harmful electromagnetic waves such as ultraviolet rays and does not use mercury and other discharge gases, it is environmentally friendly. The light emitting device is mainly formed through a combination of a light emitting diode light source using wavelength conversion particles such as phosphors.

This wavelength converting body is different from the fluorescence of general semiconductor materials in that it can be used in a light emitting device in a form combined with an excitation light source. Wavelength conversion particles play a role of converting the wavelength of a light emitting diode light source into a wavelength of low energy. Therefore, it is possible to perform a function of inducing light to emit white light or simultaneously emit light at multiple wavelengths of the monochromatic light-emitting diode by using the wavelength conversion particles. In addition, preferably, wavelength conversion particles having excellent color purity characteristics can be used to effectively improve the low color gamut of an existing light emitting device that is difficult to realize vivid colors.

The wavelength conversion layer (100) is also referred to as a wavelength conversion layer or a color conversion film, and preferably has a flat shape to prevent scattering, and preferably has a surface roughness of 50 nm or less. More preferably, the surface roughness may be 20 nm or less.

The wavelength converting body emits wavelength-converted light when light (incident light) incident from the outside reaches the above-described metal halide perovskite wavelength conversion particles. Therefore, the wavelength converting body according to the present disclosure functions to convert the wavelength of light by means of a metal halide perovskite. Hereinafter, among the incident light, light having a wavelength shorter than the emission wavelength of the aforementioned metal halide perovskite wavelength conversion particles is referred to as excitation light. In addition, a light source emitting the above-described excitation light is referred to as an excitation light source.

The wavelength converting body according to an embodiment of the present disclosure is a wavelength converting body that converts the wavelength of light generated from an excitation light source into a specific wavelength, and it is characterized in that it contains a dispersion medium that disperses the metal halide perovskite and the metal halide perovskite.

In the hybrid wavelength converting body according to an embodiment of the present disclosure, the dispersion medium may be in a liquid state, and the metal halide perovskite may be uniformly dispersed, and if the material is irradiated with ultraviolet rays, it is hardened and thus can serve to fix the metal halide perovskite.

The dispersion medium may be a photopolymerizable polymer formed by a photopolymerization reaction of a photopolymerizable monomer.

Also preferably, the polymer may play a role of protecting the metal halide perovskite from external chemical species such as oxygen or moisture by surrounding the metal halide perovskite. In addition, the metal halide perovskite may play a role in preventing migration of halide ions that may occur in the electric drive.

The photopolymerizable monomer is not particularly limited as long as it contains at least one of a carbon-carbon double bond and a triple bond and can be polymerized by light. In addition, preferably, the photopolymerizable monomer may be a monofunctional or polyfunctional ester of acrylic acid having at least one ethylenic double bond.

The photopolymerizable monomer including at least one of a carbon-carbon double bond and a triple bond is at least one or a combination of them selected from a group of a diacrylate compound, a triacrylate compound, a tetraacrylate compound, and a pentaacrylate. It may be selected from (pentaacrylate) compounds, hexaacrylilate compounds, and combinations thereof.

Specific examples of the photopolymerizable monomer including at least one of the carbon-carbon double bond and triple bond may include ethylene glycol diacrylate, triethylene glycol diacrylate, and diethylene glycol Diethylenehlyc diolacrylate, 1,4-butanediol diacrylate, 1,6-hexanediol diacrylate, neopentyl glycol diacrylate (neopentylglycol diacrylate), pentaerythritol diacrylate, pentaerythritol triacrylate, pentaerythritol tetraacrylate, dipentaerythritol diacrylate, dipentaerythritol diacrylate, dipentaerythritol pentaacrylate, dipentaerythritol hexaacrylate, bisphenol A epoxyacrylate, bisphenol A diacrylate, trimethylolpropane triacrylate, novolac epoxy acrylate, ethyleneglycolmonomethyletheracrylate, trisacryllooxyethyl phosphate, diethylenegl ycol diacrylate, triethylene glycol, triethyleneglycol diacrylate or propyleneglycol diacrylate, but is not limited thereto.

The cured product of the photopolymerizable monomer may be a cured product of a photopolymerizable monomer including at least one of a carbon-carbon double bond and a triple bond and a thiol compound having at least two thiol groups.

In addition, the photopolymerizable monomer may be a photoresist material. The photoresist material may be a silicone or epoxy material.

The photoresist material may be a commercial photoresist. The commercial photoresist materials can be AZ Electronics Materials' AZ 5214E PR, AZ 9260 PR, AZ AD Promoter-K (HMDS), AZ nLOF 2000 Series, AZ LOR-28 PR, AZ 10xT PR, AZ 5206-E, AZ GXR-601, AZ 04629; SU-8 from MICROCHEM, 950 PMMA, 495 PMMA; S1800 from micropossit; DNR-L300, DSAM, DPR, DNR-H200, DPR-G from Dongjin Semichem, or CTPR-502 from Kotem, but is not limited thereto.

A photoinitiator may be used to cure the photopolymerizable monomer, and a photoinitiator may be included in the metal halide perovskite-polymer composite. The kind of the photoinitiator is not particularly limited and may be appropriately selected. For example, usable photoinitiators include triazine-based compounds, acetophenone-based compounds, benzophenone-based compounds, thioxanthone-based compounds, benzoin-based compounds, Oxime compounds, carbazole compounds, diketone compounds, sulfonium borate compounds, diazo compounds, nonimidazolium compounds, or It may be selected from a combination of these, but is not limited thereto.

Examples of the triazine-based compound are 2,4,6-trichloro-s-triazine (2,4,6-trichloro-s-triazine), 2-phenyl-4,6-bis(trichloromethyl)-s-triazine (2-phenyl-4,6-bis(trichloro methyl), 2-(3′, 4′-dimethoxy styryl)-4,6-bis(trichloromethyl)-s-tri Azine (2-3′, 4′-dimethoxy styryl)-4,6-bis(trichloro methyl)-s-triazine), 2-(4′-methoxy naphthyl)-4,6-bis(trichloromethyl))-s-triazine (2-(4′-methoxynaphtyl)-4,6-bis(trichl oromethyl)-s-triazine), 2-(p-methoxy phenyl)-4,6-bis(trichloromethyl)-s-triazine (2-(p-methoxyphenyl)-4,6-bis(trichloro methyl)-s-triazine), 2-(p-tolyl)-4,6-bis(trichloromethyl)-s-Triazine (2-(p-tolyl)-4,6-bis(trichloromethy 1)-s-triazine), 2-biphenyl-4,6-bis(trichloromethyl)-s-triazine (2-biphenyl-4,6,-bis(trichloro methyl), bis(trichloromethyl)-6-styryl-s-triazine (bis(trichloro methyl)-6-styryl-s-triazine), 2-(naphtho-1-yl)-4,6-bis(trichloromethyl)-s-triazine (2-nafto-1-yl)-4,6-bis(trichloromethyl), 2-(4-methoxy naphtho-1-yl)-4,6-bis(trichloromethyl)-s-triazine (2-(4-methoxynafto-1-yl)-4,6-bis(trichloromethyl)-s-triazine, 2,4-Trichloromethyl(piperonyl)-6-triazine (2,4-trichloro methyl(piperonyl)-6-triazine), 2,4-(trichloromethyl(4′-methoxy styryl)-6-Triazine (2,4-(trichloro methyl(4′-methoxy styryl)-6-triazine) Including, but not limited to.

Examples of the acetophenone-based compound are 2,2′-diethoxy acetophenone (2,2-diethoxy acetophenone), 2,2′-dibutoxy acetophenone (2,2,-dibutoxy acetophenone), 2-2-hydroxy-2-methyl propiophenone, pt-butyl trichloro acetophenone, pt-butyl dichloro acetophenone, 4-Chloro acetophenone (4-chloro acetophenone), 2,2′-dichloro-4-phenoxy acetophenone (2,2-dichloro-4-phenoxy acetophenone), 2-methyl-1-(4-(methylthio)Phenyl)-2-morpholino propan-1-one (2-methyl-1-(4-(methylthio)phenyl)-2-mopholino propan-1-one), 2-benzyl-2-dimethyl amino-1-(4-morpholino phenyl)-butan-1-one (2-benzyl-2-dimethyl amino-1-(4-mopholino phenyl)-butan-1-one), and the like, but is not limited thereto.

Examples of the benzophenone-based compound include benzophenone, benzoyl benzoate, methyl benzoyl benzoate(methyl 2-benzoylbenzoate), 4-phenyl benzophenone, hydroxy Benzophenone (hydroxybeonzophenone), acrylate benzophenone(benzophenone acrylate), 4,4′-bis(dimethylamino)benzophenone(4,4-bis(dimethylamino)benzophenone), 4,4′-dichloro benzophenone(4,4-dichlorobenzophenone), 3,3′-dimethyl-2-methoxy benzophenone(3,3-dimethyl-2-methoxy benzophenone), and the like, but is not limited thereto.

Examples of the thioxanthone-based compound include thioxanthone, 2-methyl thioxantone, isopropyl thioxantone, and 2,4-diethyl thioxantone. Santon (2,4-diethyl thioxantone), 2,4-diiospropyl thioxantone (2,4-diiospropyl thioxantone), 2-chloro thioxantone (2-chloro thioxantone), and the like, but is not limited thereto.

Examples of the benzoin-based compound include benzoine, benzoine methyl ether, benzoine ethyl ether, benzoine isopropyl ether, benzoin iso Butyl ether (benzoine isobutyl ether), benzyl dimethyl ketal, and the like, but is not limited thereto.

Examples of the oxime compound are 2-(o-benzoyloxime)-1-[4-(phenylthio)phenyl]-1,2-octanedione (2-(o-benzoyloxime)-1-[4-(phenylthio)phenyl]-1,2,-octandione and 1-(o-acetyloxime)-1-[9-ethyl-6-(2-methylbenzoyl)-9H-carbazol-3-yl]ethane On (1-(o-acetyloxime)-1-[9-ethyl-6-(2-methylbenzoyl)-9H-carbazol-3-yl]ethanone), but is not limited thereto. The photopolymerizable monomer The metal halide perovskite-polymer composite obtained by curing the photopolymerizable monomer is formed in a form in which a polymer surrounds the surface of the metal halide perovskite described above.

The metal halide perovskite-polymer composite may be preferably attached on a substrate or may be an independent film.

In the present specification, the “metal halide perovskite-polymer composite film” includes all films made of the metal halide perovskite-polymer composite.

FIG. 86 is a schematic diagram showing a metal halide perovskite-polymer composite film according to another embodiment of the present disclosure.

In addition, when the metal halide perovskite-polymer composite is manufactured in the form of a film attached to a specific substrate, the metal halide perovskite-polymer composite film may further include a polymeric binder. In this case, a plurality of metal halide perovskites are dispersed in a polymer composed of a cured product of a photopolymerizable monomer including a carbon-carbon unsaturated bond and a polymeric binder. The polymeric binder may serve to improve adhesion between the substrate and the metal halide perovskite-polymer composite.

The substrate (10) serves as a support for a light emitting device, and may be a transparent material. In addition, the substrate (10) may be a flexible material or a hard material, and preferably may be a flexible material.

The material of the substrate (10) is glass, sapphire, quartz, silicon, polyethylene terephthalate (PET), polystyrene (PS), polyimide (polyimide, PI), polyvinyl chloride (PVC), polyvinylpyrrolidone (PVP), polyethylene (PE), or the like, but is not limited thereto.

The polymer binder may be an acrylic polymer binder, a cardo polymer binder, or a polymer of a combination thereof, but is not limited thereto.

The acrylic polymer binder may be a copolymer of a first unsaturated monomer containing a carboxyl group and a second unsaturated monomer copolymerizable therewith.

The first unsaturated monomer may be a carboxylic acid vinyl ester compound such as acrylic acid, maleic acid, methacrylic acid, vinyl acetate, itaconic acid, 3-butenoic acid, fumaric acid, vinyl benzoate, or a combination thereof, but is not limited thereto.

The second unsaturated monomer is an alkenyl aromatic compound, an unsaturated carboxylic acid ester compound, an unsaturated carboxylic acid amino alkyl ester compound, an unsaturated carboxylic acid glycidyl ester compound, a vinyl cyanide compound, a hydroxy alkyl acrylate, or It may be a combination thereof, but is not limited thereto.

Also preferably, the second unsaturated monomer is styrene, α-methylstyrene, vinyltoluene, vinylbenzylmethylether, methyl acrylate, ethyl acrylate, butyl acrylate, benzyl acrylate, cyclohexyl acrylate, phenyl Acrylate, 2-aminoethylacrylate, 2-dimethylaminoethylacrylate, N-phenylmaleimide, N-benzylmaleimide, N-alkylmaleimide, 2-dimethylaminoethylmethacrylate, acrylonitrile, gly Unsaturated amide compounds such as cidyl acrylate and acrylamide; It may be 2-hydroxyethyl acrylate, 2-hydroxy butyl acrylate, or a combination thereof, but is not limited thereto.

The acrylic polymer binder is a methacrylic acid/benzyl methacrylate copolymer, methacrylic acid/benzyl methacrylate/styrene copolymer, methacrylic acid/benzyl methacrylate/2-hydroxyethyl methacrylate copolymer It may be a combination, methacrylic acid/benzyl methacrylate/styrene/2-hydroxyethyl methacrylate copolymer, or a combination thereof, but is not limited thereto.

The weight-average molecular weight of the polymeric binder may be about 1,000 to about 150,000 g/mol. Also, preferably, it may be about 2,000 to about 30,000 g/mol. hen the weight average molecular weight of the polymeric binder is about 2,000 to about 30,000 g/mol, the metal halide perovskite-polymer composite film has excellent physical and chemical properties and has an appropriate viscosity, and has excellent adhesion when fabricating metal halide perovskite-polymer composite film.

The metal halide perovskite-polymer composite film may further include a light diffusing agent. The light diffusing agent may be metal oxide particles, metal particles, and combinations thereof, but is not limited thereto. The light diffusing agent may serve to increase the probability of encountering the metal halide perovskite with the incident light of the composition by increasing the refractive index of the composition.

The light diffusing agent may include inorganic oxide particles such as alumina, silica, zirconia, titania, and zinc oxide, and metal particles such as gold, silver, copper, and platinum, but is not limited thereto. At this time, a dispersant may be added to increase the dispersibility of the light diffusing agent.

The wavelength conversion layer may further include a barrier film. The barrier film may be positioned above and below the wavelength conversion layer to prevent penetration of moisture and oxygen. When a barrier film is attached to the wavelength conversion layer, the barrier film may serve to protect the wavelength conversion layer from external air including moisture and oxygen. In particular, since the metal halide perovskite has poor stability against moisture and oxygen, the stability of the wavelength conversion layer including the barrier film can be greatly improved.

It is advantageous to prevent moisture and oxygen penetration by placing the barrier film on both sides above and below the wavelength conversion layer. Ultimately, it is desirable to secure stability with just one layer without an additional barrier film by putting the function of such a barrier film into the wavelength conversion layer. Such a barrier film may be made of a polymer or ceramic material.

Hereinafter, a method of manufacturing a metal halide perovskite wavelength conversion layer according to another embodiment of the present disclosure will be described.

First, a metal halide perovskite wavelength conversion particle is prepared.

Thereafter, the above-described wavelength conversion particles are dispersed in a dispersion medium.

Wavelength conversion particles are dispersed in the dispersion medium described above. The dispersion medium may be in a liquid state. When the dispersion medium is in a liquid state, when the dispersion medium and the wavelength conversion particles dispersed in the dispersion medium are sealed by a sealing member to be described later, the shape thereof is not restricted, and thus it can be applied to various types of devices. The dispersion medium may be, for example, an epoxy resin or silicone. Since the wavelength conversion particles must receive excitation light and emit wavelength converting light, the dispersion medium is preferably a material that is not discolored or deteriorated by excitation light or the like.

After that, the metal halide perovskite wavelength conversion particles and the dispersion medium are sealed with a sealing member.

FIG. 87 is a cross-sectional view showing a method of sealing a wavelength converting body according to an embodiment of the present disclosure.

Referring to FIG. 87(a), a first sealing member (10a) and a second sealing member (10b) are stacked.

Sealing members may use polymers or silicone that are not corroded by dispersive medium (30) distributed by metal halide perovskite wavelength conversion particles (20). In particular, since the polymer resin can be heated and gradually recovered, it can be used to form a pack-type wavelength converting body with dispersion medium (30) dispersed wavelength converting body by using a heat adhesion process.

Referring to FIG. 87(b), one (1) side of the first sealing member (10a) and the second sealing member (10b) may be heated and bonded using a heat-adhesion process to prevent the aforementioned metal halide perovskite wavelength conversion particles (20) and dispersion media (30) from leaking from the sealing member (10a, 10b). However, if the aforementioned metal halide perovskite wavelength conversion particles (20) and distributed media (30) do not leak, it is possible to use other adhesive processes besides the thermal adhesion process.

Referring to FIG. 87(c), the above metal halide perovskite wavelength converting body (20) is injected with a dispersive medium (30) dispersed between the first sealing member (10a) and the second sealing member (10b) of the other side without the aforementioned first sealing member (10a) and second sealing member (10b).

Referring to FIG. 87(d), the one side (1) of the aforementioned 1st sealing member (10a) and 2nd sealing member (10b) is bonded using a heat-adhesion process to seal the dispersion medium (30) dispersed by metal halide perovskite wavelength change particles (10a, 10b).

Referring to FIG. 87(e), it can be seen that a dispersion medium (30) with a distributed wavelength change material (20) forms a metal halide perovskite wavelength converting body (400) sealed with a sealing member (10). The aforementioned metal halide perovskite wavelength converting body (400) has the advantage of being able to be applied to light emitting devices without the need for a separate ligand refining process, as nano wavelength-converting particles (20) containing metal halide perovskite nanocrystals are sealed. Thus, it can prevent oxidation of wavelength conversion particles during ligand refining, indicating high color purity and luminous effects when applied to light emitting devices. In addition, the process can be simplified.

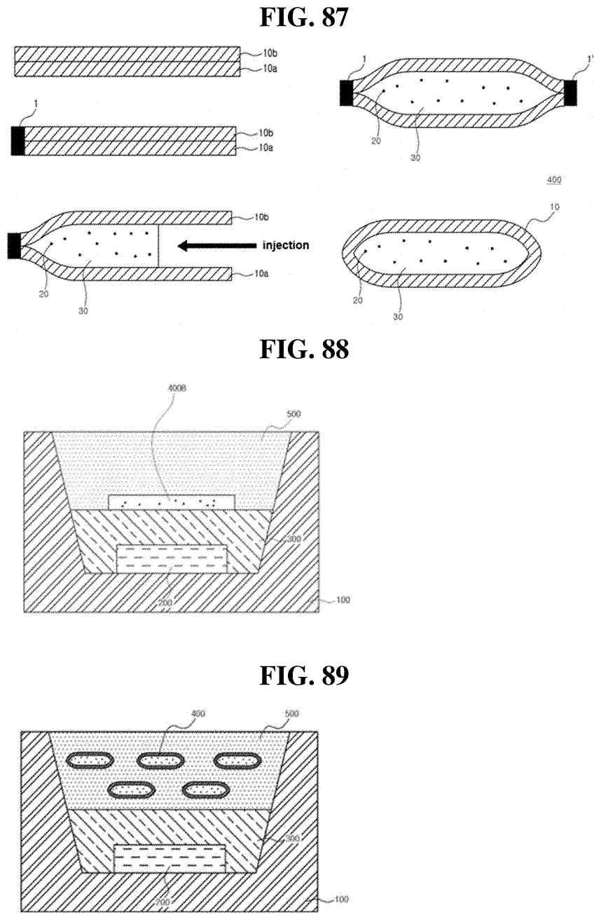

FIG. 88 is a cross-sectional view of a light emitting device including a wavelength conversion layer according to an embodiment of the present disclosure.

Referring to FIG. 88, the light emitting element under one example of this disclosure includes a wavelength conversion layer (400B) that is placed on a base structure (100), a aforementioned base structure (100), including at least one here light source (200) and a aforementioned wavelength conversion particle (20).

The base structure (100) described above may be a package frame or a base substrate. When the base structure (100) is a package frame, the package frame may include the base substrate. The base substrate may be a submount substrate or a light emitting diode wafer. The light-emitting diode wafer is a state before being separated into light-emitting diode chips, and indicates a state in which a light-emitting diode device is formed on the wafer. The base substrate may be a silicon substrate, a metal substrate, a ceramic substrate, or a resin substrate.

The base structure (100) described above may be a package lead frame or a package pre-mold frame. The base structure (100) may include a bonding pad (not shown). Bonding pads may contain Au, Ag, Cr, Ni, Cu, Zn, Ti, Pd, and the like. External connection terminals (not shown) connected to bonding pads may be located on the outer side of the base structure (100). The bonding pads and the external connection terminals may be those provided in the package lead frame.

The excitation light source (200) is located on the base structure (100) described above. It is preferable that the above-described excitation light source (200) emit light having a wavelength shorter than that of the wavelength conversion particles of the wavelength conversion layer 400B. The above-described excitation light source (200) may be any one of a light emitting diode and a laser diode. In addition, when the base structure (100) is a light emitting diode wafer, the step of arranging the excitation light source may be omitted. For example, the excitation light source (200) may use a blue LED, and as the blue LED, a gallium nitride-based LED emitting blue light of 420 nm to 480 nm may be used.

As shown in FIG. 89, the first encapsulation part (300) may be formed by filling the encapsulating material for encapsulating the excitation light source (200) described above. The above-described first encapsulation part (300) may not only serve to encapsulate the above-described excitation light source (200), but may also serve as a protective film. In addition, when the above-described wavelength conversion layer (400B) is positioned on the first encapsulation part (300), a second encapsulation part (500) may be further formed to protect and fix the wavelength conversion layer (400B). The sealing material may include at least one of epoxy, silicone, acrylic polymer, glass, carbonate polymer, and mixtures thereof.

The first encapsulation part (300) can be formed using various methods such as a compression molding method, a transfer molding method, a dotting method, a blade coating method, a screen coating method, dip coating, spin coating, spray, or inkjet printing. However, the first encapsulation part (300) may be omitted.

In the one-time example of this disclosure, the above light-emitting elements are designed specific to the unit cell, but if the base structure is a submount substrate or light-emitting diode wafer, the above submount substrate or light-emitting diode wafer can be cut and processed into each unit cell.

In addition, preferably, the wavelength conversion layer may have stretchable properties.

The stretchable wavelength conversion layer according to the present disclosure is characterized in that it includes the metal halide perovskite described above.

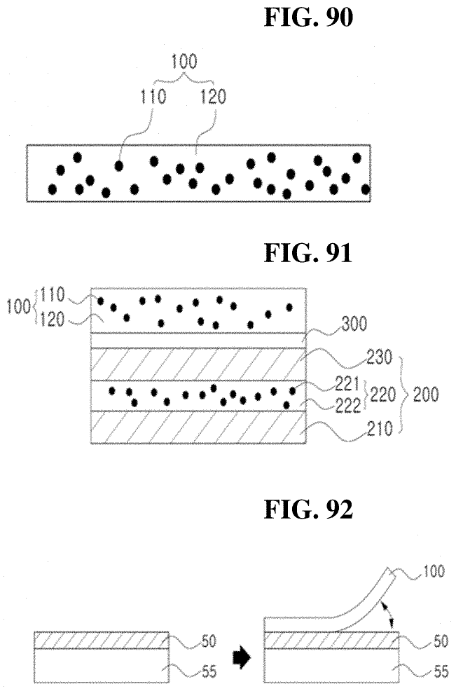

FIG. 90 is a cross-sectional view schematically illustrating a stretchable wavelength conversion layer according to an embodiment of the present disclosure.

Referring to FIG. 90, the stretchable wavelength conversion layer (100) according to this disclosure may contain the color conversion particles (110) and the color conversion particles (110) dispersed stretchable polymers (120).

The color conversion particles (110) of this disclosure may be dispersed within the stretchable polymer (120). The wavelength conversion layer (100) may contain a stretchable polymer (120) to be flexible.

The wavelength conversion layer (100) is also called the wavelength conversion layer or color conversion film, and it is desirable to have a flat shape to prevent scattering, and to have a surface roughness of 50 nm or less. More preferably, the above surface roughness may not be more than 20 nm.

Above mentioned stretchable polymer (120) is polydimethylsiloxane (PDMS), polyurethane (PU), styrene butadiene styrene (SBS), styrene ethylene butylene styrene (SEBS), ecoflex, hydrogel, organic Gel (organogel), PEO (Polyethylene oxide), PS (Polystyrene), PCL (Polycaprolactone), PAN (Polyacrylonitrile), PMMA (Poly(methyl methacrylate)), polyimide (Polyimide), PVDF (Poly(vinylidene fluoride)), PVK (Poly(n-vinylcarbazole)), PVC (Polyvinylchloride), polyethylene terephthalate, polyethylene naphthalene, polycarbonate, polyacrylate, polyether sulfone), polypropylene, polymethylphenylsiloxane, polydiphenylsiloxane, polysiloxane, ORMOCER, a homo copolymer including at least one selected from the group consisting of their respective derivatives and combinations thereof from thereof, an alternating copolymer, a random copolymer, a block copolymer, a multiblock copolymer, or a graft copolymer.

In addition, the polydimethylsiloxane (PDMS), polyurethane (PU), styrene butadiene styrene (SBS), styrene ethylene butylene styrene (SEBS), ecoflex, hydrogel, organic gel, PEO (Polyethylene oxide), PS(Polystyrene), PCL(Polycaprolactone), PAN(Polyacrylonitrile), PMMA(Poly(methyl methacrylate)), Polyimide, PVDF(Poly(vinylidene fluoride)), PVK(Poly(n-vinylcarbazole)), PVC (Polyvinylchloride), polyethylene terephthalate, polyethylene naphthalene, polycarbonate, polyacrylate, polyether sulfone, polypropylene), polymethylphenylsiloxane, polydiphenylsiloxane, polysiloxane, and derivatives of ORMOCER may contain hydrogen bonds. As an example, the hydrogen bond is F—H . . . F, O—H . . . N, O—H . . . O, N—H . . . N, N—H . . . O, OH—H . . . OH₃⁺.

The stretchable polymer (120) may be capable of self-healing by having a self-repairing ability due to scratches and damage.

The stretchability of the stretchable polymer (120) may be 5% or more along the tensile direction. Preferably it may be 10% or more, more preferably 20% or more, more preferably 30% or more, more preferably 50% or more, more preferably 100% or more. Accordingly, the stretchable wavelength conversion layer (100) may be stretched by 5% or more without breaking by the applied strain, and it is preferable that the stretchable wavelength conversion layer (100) is usually stretched by 20% or more.

FIG. 91 is a cross-sectional view of a stretchable light emitting device according to an embodiment of the present disclosure.

Referring to FIG. 91, the stretchable light emitting device includes a stretchable wavelength conversion layer (100), a stretchable light source (200), and a bonding layer (300).

The wavelength conversion layer (100) includes color conversion particles (110) for generating wavelength conversion light by wavelength conversion of excitation light and a stretchable polymer (120) in which the color conversion particles (110) are dispersed. In addition, a bonding layer (300) is formed between the wavelength conversion layer (100) and the stretchable light source (200). The bonding layer (300) may be any material that maintains bonding even when the wavelength conversion layer (100) and the light source 200 are elongated, and minimizes absorption of light formed by the light source.

The configuration and material of the wavelength conversion layer are the same as those described in FIG. 90.

In addition, the stretchable light source (200) includes light-emitting particles (221) and a dispersion polymer (221) in which the light-emitting particles (221) are dispersed. It is preferable that the dispersion polymer (222) also has a stretchability.

The light emitted from the stretchable light source (200) can reach the stretchable wavelength conversion layer (100). Light (hereinafter referred to as excitation light) that reaches the stretchable wavelength conversion layer (100) may transform the wavelength area of the light and emit light (hereinafter referred to as wavelength conversion light) that has a different wavelength area than the incident wavelength area.

Stretchable light source (200) may be inorganic light-emitting diode (LED), organic light-emitting diode (OLED), perovskite light emitting diode (PeLED), light-emitting electrochemical cell (LEEC), alternative-current electroluminescence (ACEL), quantum dot light-emitting diodes (QDLEDs), light-emitting capacitors (LEC), or light-emitting transistors (LET). For example, the stretchable light source (200) may be a stretchable perovskite light-emitting diode (PeLED), and may be a QDLED. In particular, QDLED is provided in a form in which quantum dots are dispersed in a dispersion polymer (222).

When the stretchable light source (200) is stretched by 5%, more preferably 10% or more, electroluminescence, external quantum efficiency, and current efficiency of the device do not change or It can be reduced to less than 50%. Accordingly, the stretchability of the stretchable light source (200) may be 5% or more along the stretching direction.

The stretchable light source (200) includes a lower electrode layer (210), a light emitting layer (220), and an upper electrode layer (230). It is preferable that the lower electrode layer (210) and the upper electrode layer (230) are stretchable materials and have conductivity. For example, it is preferable that the lower electrode layer (210) and the upper electrode layer (230) are ionic hydrogel.

It is desirable for stretchable light sources to emit light with shorter wavelengths than those emitted from color conversion particles in the wavelength conversion layer. For example, if a stretchable light source is a blue stretchable light source that emits blue light (400-490 nm), the color conversion particles within the wavelength conversion layer placed on the light source may absorb the above blue light. The color-conversion particles here may be particles that can be converted to red or green. In other words, the absorbed blue light in the wavelength conversion layer can be emitted by the red conversion particles into the red light, and the green light by the green conversion particles can be released. In addition, if a stretchable light source is UV (less than 400 nm) stretchable light sources, the stretchable color-converting layer may emit blue, green and red light, including both blue, green and red-converting particles.

FIG. 92 is a schematic diagram illustrating a method of manufacturing a stretchable wavelength conversion layer according to an embodiment of the present disclosure.

In the present disclosure, by forming a thin film on the surface of the substrate treated with the self-assembled monomolecular film, the polymer thin film can be easily peeled off the substrate due to the low surface energy of the substrate surface. Therefore, the thickness of the polymer thin film, which is the wavelength conversion layer, can be precisely controlled.

Referring to FIG. 92, a self-assembly monomolecular membrane (50) is prepared. The above self-assembly monomolecular membranes (50) need to have low surface energy. This allows the wavelength conversion layer (100) to be easily dislodged from the substrate (55). It is desirable that the above self-assembly monomolecular membrane (50) consists of Octadecyltrimethoxysilane (OTMS).

When the OTMS surface-treated substrate surface is used, the wavelength conversion layer (100) can be formed thinly (about 70 μm level) by spin coating, and the thin film can be precisely laminated to a desired thickness. It has the advantage of being able to control it. At this time, the thickness of the wavelength conversion layer (100) is preferably greater than 70 μm and less than 1 mm, and more preferably 70 μm or more and 140 μm. More preferably, it may be from 80 μm to 130 μm, and even more preferably from 80 μm to 100 μm.

Furthermore, if the above wavelength conversion layer (100) thin films are formed on Si or glass substrates that are not surface treated with OTMS, the film is not easily removed from the substrates due to the high adhesion of the wavelength conversion layer thin films, it is difficult to stack on a stretchable light source.

In addition, if the above wavelength conversion layer (100) thin film is formed on an OTMS surface-untreated Si or glass substrate, the metal halide perovskite is highly viscous, making it difficult to form a thin wavelength conversion layer less than 100 μm.

A color conversion precursor solution in which metal halide perovskite nanocrystals or quantum dots are dispersed is coated on the self-assembled monolayer (50). It is preferable that the color conversion precursor solution contains SEBS (Styrene Ethylene Butylene Styrene). The SEBS is preferable for the purpose of dispersing metal halide perovskite nanocrystals because there is no additional crosslinking process unlike other stretchable polymers.

After spin coating, the wavelength conversion layer (100) formed of a film may be easily peeled off from the substrate (55).

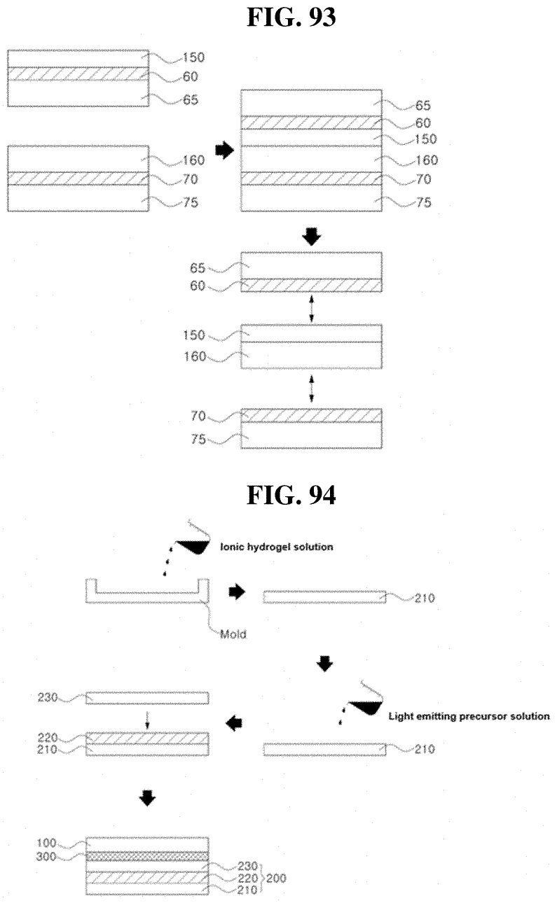

FIG. 93 is another schematic diagram for explaining a method of manufacturing a stretchable wavelength conversion layer according to an embodiment of the present disclosure.

Referring to FIG. 93, the above wavelength conversion layer may be formed by a plurality. For example, the first magnetic particle membrane (60) is formed on the first substrate (65) and the first wavelength conversion layer (150) is formed on the first magnetic particle membrane (60). Apart from the first wavelength conversion layer (150), a second magnetic particle membrane (70) is formed on the second substrate (75) and a second wavelength conversion layer (160) is formed on the second magnetic particle membrane (70).

The first wavelength conversion layer (150) may form green light and may have metal halide perovskite nanoparticles. In addition, the second wavelength conversion layer (160) forms red light and may have quantum dots. The formed first wavelength conversion layer (150) and the second wavelength conversion layer (160) are bonded to each other. Since SEBS is contained in the polymer constituting the wavelength conversion layer, each wavelength conversion layer is easily bonded without the intervention of other bonding agents. Accordingly, the bonded at least two types of wavelength conversion layers are separated from the bonded self-assembled monolayers.

FIG. 94 is a schematic diagram illustrating a method of manufacturing the stretchable light emitting device of FIG. 91 according to an embodiment of the present disclosure.

Referring to FIG. 94, an ionic hydrogel solution is prepared. The ionic hydrogel solution is put into a mold having a specific shape, and when cured, it is formed into an ionic hydrogel. The ionic hydrogel functions as a lower electrode or an upper electrode of FIG. 91. The ionic hydrogel has a network structure in which a water-soluble polymer forms a three-dimensional crosslink by a physical or chemical bond. Therefore, it does not dissolve in an aqueous environment and may contain a significant amount of moisture. In addition, since it has ions inside, it has the feature that current can be transferred through the movement of ions by an applied electric field.

In addition, when the lower electrode (210) formed of the lower first ionic hydrogel is formed, the light emitting layer (220) is formed on the first ionic hydrogel. The light-emitting layer has light-emitting particles evenly dispersed in the dispersible polymer. The light-emitting particles may include quantum dots or metal halide perovskite particles of FIG. 91. In addition, it is preferable that the dispersible polymer constituting the light emitting layer (220) is a material having an elongation force, such as PDMS.

A second ionic hydrogel serving as the upper electrode (230) is formed on the emission layer (220). Through this, the two ionic hydrogels function as positive and negative electrodes with the light emitting layer as the center. Through this, the light-emitting layer may perform a light-emitting operation.

In addition, the wavelength conversion layer (100) illustrated in FIG. 91 may be adhered to the second ionic hydrogel. When bonding between the stretchable light source (200) and the wavelength conversion layer (100) is not easy, it is preferable to use a material having less absorption of light formed in the light emitting layer (220) as the adhesive layer (300) used. The material of the adhesive layer (300) may be variously selected by a person skilled in the art.

In the embodiment of the present disclosure, PDMS is used as a polymer in order to have the stretchability in the light emitting layer (220). Since PDMS is a non-conductor, the light emitting particles of the light emitting layer (220) are excited by an electric field by an AC voltage applied between the two layers of ionic hydrogels, and the excited state and the ground state are repeated by the AC voltage. Through this, the light emission operation is performed.

Further, light formed by performing a light emission operation is incident on the wavelength conversion layer. If the light source emits blue light and the wavelength conversion layer has metal halide perovskite nanocrystals or quantum dots forming red and green light, a light emitting device having a stretchability forms white light. Through this, a white light source can be obtained even with a very thin thickness, and various display environments can be used.

<Hybrid Wavelength Conversion Layer>

The above-described wavelength conversion layer may be a hybrid wavelength conversion layer further including a non-metal halide perovskite-based quantum dot or a non-metal halide perovskite-based phosphor.

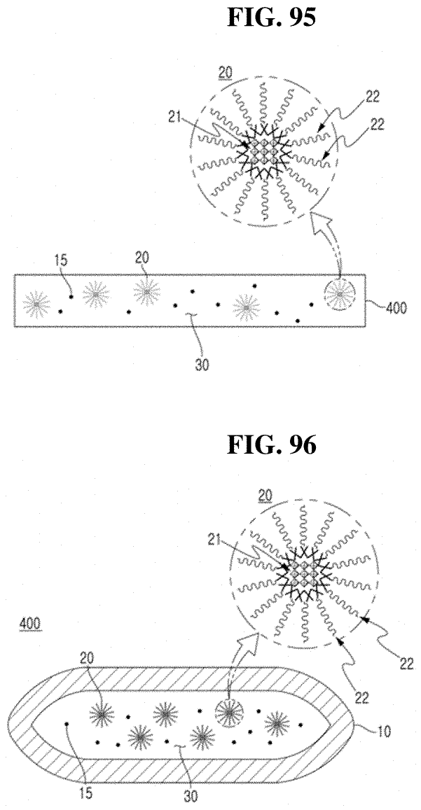

FIG. 95 shows a hybrid wavelength converting body according to an embodiment of the present disclosure.

Referring to FIG. 95, the hybrid wavelength converting body (400) following the one-run example of this disclosure includes metal halide perovskite nanocrystal particles (20), non-metallic halide perovskite quantum dots (15) and dispersive media (30).

When light incident from the outside (incident light) reaches the metal halide perovskite nanocrystal particles, wavelength-converted light is emitted. Therefore, the hybrid wavelength converting body (400) according to the present disclosure functions to convert the wavelength of light by means of metal halide perovskite nanocrystal particles and non-metal halide perovskite quantum dots.

In this case, among the incident light, light having a wavelength shorter than that of the aforementioned metal halide perovskite nanocrystal particles is referred to as excitation light. In addition, a light source that emits the above-described excitation light is referred to as an excitation light source.

The hybrid wavelength converting body according to the present disclosure is a wavelength conversion particle, a metal halide perovskite nanocrystal particle (20) having a lamellar structure in which organic and inorganic planes are alternately stacked, and a non-metal halide perovskite quantum dot It characterized in that it includes (15) at the same time.

In addition, the hybrid wavelength converting body according to the present disclosure may simultaneously include the metal halide perovskite nanocrystal particle (20) and the non-metal halide perovskite phosphor.

In the present disclosure, the non-metal halide perovskite wavelength converting body can be classified into a quantum dot and a phosphor. The quantum dots are semiconductor particle having a size of several nanometers or less, and have a diameter smaller than a Bohr radius, thereby exhibiting a quantum confinement effect. Therefore, the smaller the size of the quantum dot, the greater the bandgap energy, and the emission wavelength can be adjusted according to the size of the quantum dot. On the other hand, since the phosphor has a diameter larger than the Bohr radius, the bandgap energy does not change according to the size of the particle or crystal, and it refers to a material that emits light depending on the crystal structure or molecular structure.

In the hybrid wavelength converting body according to the present disclosure, hereinafter, as an example of a non-metal halide perovskite-based wavelength converting body, it will be described centering on a non-metal halide perovskite-based quantum dot, but is not limited thereto, and non-metal halide perovskite phosphors are also included in the scope of the present disclosure.

Since the metal halide perovskite is the same as described above, a detailed description will be omitted.

The metal halide perovskite nanocrystal may further include a plurality of organic ligands (20) surrounding the halide metal halide perovskite nanocrystal (10). The organic ligands (20) at this time are a material used as a surfactant and may include an alkyl halide. Accordingly, the alkyl halide used as a surfactant to stabilize the surface of the halide metal halide perovskite precipitated as described above becomes an organic ligand surrounding the surface of the halide metal halide perovskite nanocrystal. On the other hand, when the length of the alkyl halide surfactant is short, the size of the formed nanocrystal increases, so it can be formed in excess of 10 μm, and excitons are emitted by thermal ionization and delocalization of the charge carrier in the large nanocrystal. There may be a fundamental problem of being separated by a free charge and extinguishing. Accordingly, the size of the metal halide perovskite nanocrystals formed by using an alkyl halide having a predetermined length or longer as a surfactant can be controlled to a predetermined size or less.

The metal halide perovskite nanocrystal particle (100) according to the present disclosure may include a metal halide perovskite nanocrystal structure (110) that can be dispersed in an organic solvent. The organic solvent at this time may be a polar solvent or a non-polar solvent.

For example, the polar solvent may include acetic acid, acetone, acetonitrile, dimethylformamide, gamma butyrolactone, N-methylpyrrolidone, ethanol or dimethylsulfoxide, and the non-polar solvent may include dichloroethylene, trichloroethylene, chloroform, chlorobenzene, dichlorobenzene, styrene, dimethylformamide, dimethyl sulfoxide, xylene, toluene, cyclohexene, or isopropyl alcohol, but is not limited thereto.

In addition, the shape of the metal halide perovskite nanocrystal may be generally used in this field. The shape of the metal halide perovskite nanocrystal may be a 0-dimensional, 1-dimensional or 2-dimensional. As an example, it may be in the shape of a sphere, an ellipsoid cube, a hollow cube, a pyramid, a cylinder, a cone, an elliptic column, hollow sphere, Janus particle, prism, multipod, polyhedron, nano tube, nano wire, nano fiber, or nanoplatelet.

In addition, the size of the crystalline particles may be 1 nm to 10 μm or less. For example, 1 nm, 2 nm, 3 nm, 4 nm, 5 nm, 6 nm, 7 nm, 7.5 nm, 8 nm, 8.5 nm, 9 nm, 9.5 nm, 10 nm, 10.5 nm, 11 nm, 11.5 nm, 12 nm, 12.5 nm, 13 nm, 13.5 nm, 14 nm, 14.5 nm, 15 nm, 16 nm, 17 nm, 18 nm, 19 nm, 20 nm, 25 nm, 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 150 nm, 200 nm, 250 nm, 300 nm, 350 nm, 400 nm, 450 nm, 500 nm, 600 nm, 700 nm, 800 nm, 900 nm, 1 μm, 2 μm, 5 μm, or 10 μm. The size of the particle can be defined as a region with the lower value as the minimum value and the larger value as the maximum value among the two numbers selected above. It is preferably 8 nm or more and 300 nm or less, and more preferably 10 nm or more and 30 nm or less. On the other hand, the size of the crystalline particles at this time means a size that does not take into account the length of a ligand to be described later, that is, the size of the remaining portions excluding the ligand. If the size of the crystalline particle is greater than 1 μm, there may be a fundamental problem in which excitons are decimated by thermal ionization and delocalization of charge carriers in large crystals. More desirable, the size of the above crystalline particle may also be greater than the Bohr diameter, as noted above. The phenomenon of thermal ionization and non-universification of the charge carrier above may gradually occur when the nanocrystals exceed 100 nm in size. If it is over 300 nm, the phenomenon will appear more, and if it is over 1 μm, it will be governed by the phenomenon because it is a complete bulk area.

For example, when the crystalline particles are spherical, the diameter of the crystalline particles may be 1 nm to 10 μm. Preferably, it may be 1 nm, 3 nm, 5 nm, 6 nm, 7 nm, 8 nm, 9 nm, 10 nm, 11 nm, 12 nm, 13 nm, 14 nm, 15 nm, 16 nm, 17 nm, 18 nm, 19 nm, 20 nm, 25 nm, 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 150 nm, 200 nm, 250 nm, 300 nm, 350 nm, 400 nm, 450 nm, 500 nm, 600 nm, 700 nm, 800 nm, 900 nm, 1 μm, 2 μm, 5 μm or 10 μm.

In addition, the band gap energy of such crystalline particles may be 1 eV to 5 eV. Preferably, the band gap energy of the nanocrystal particle is a range in which the lower value of the number is the lower limit value and the higher value is the upper limit value selected from the group of 1 eV, 1.1 eV, 1.2 eV, 1.3 eV, 1.4 eV, 1.5 eV, 1.6 eV, 1.7 eV, 1.8 eV, 1.81 eV, 1.82 eV, 1.83 eV, 1.84 eV, 1.85 eV, 1.86 eV, 1.87 eV, 1.88 eV, 1.89 eV, 1.9 eV, 1.91 eV, 1.92 eV, 1.93 eV, 1.94 eV, 1.95 eV, 1.96 eV, 1.97 eV, 1.98 eV, 1.99 eV, 2 eV, 2.01 eV, 2.02 eV, 2.03 eV, 2.04 eV, 2.05 eV, 2.06 eV, 2.07 eV, 2.08 eV, 2.09 eV, 2.1 eV, 2.11 eV, 2.12 eV, 2.13 eV, 2.14 eV, 2.15 eV, 2.16 eV, 2.17 eV, 2.18 eV, 2.19 eV, 2.2 eV, 2.21 eV, 2.22 eV, 2.23 eV, 2.24 eV, 2.25 eV, 2.26 eV, 2.27 eV, 2.28 eV, 2.29 eV, 2.3 eV, 2.31 eV, 2.32 eV, 2.33 eV, 2.34 eV, 2.35 eV, 2.36 eV, 2.37 eV, 2.38 eV, 2.39 eV, 2.4 eV, 2.41 eV, 2.42 eV, 2.43 eV, 2.44 eV, 2.45 eV, 2.46 eV, 2.47 eV, 2.48 eV, 2.49 eV, 2.5 eV, 2.51 eV, 2.52 eV, 2.53 eV, 2.54 eV, 2.55 eV, 2.56 eV, 2.57 eV, 2.58 eV, 2.59 eV, 2.6 eV, 2.61 eV, 2.62 eV, 2.63 eV, 2.64 eV, 2.65 eV, 2.66 eV, 2.67 eV, 2.68 eV, 2.69 eV, 2.7 eV, 2.71 eV, 2.72 eV, 2.73 eV, 2.74 eV, 2.75 eV, 2.76 eV, 2.77 eV, 2.78 eV, 2.79 eV, 2.8 eV, 2.9 eV, 3 eV, 3.1 eV, 3.2 eV, 3.3 eV, 3.4 eV, 3.5 eV, 3.6 eV, 3.7 eV, 3.8 eV, 3.9 eV, 4 eV, 4.1 eV, 4.2 eV, 4.3 eV, 4.4 eV, 4.5 eV, 4.6 eV, 4.7 eV, 4.8 eV, 4.9 eV, and 5 eV.

In general, the metal halide perovskite can control the emission wavelength by controlling the halide ion at the X site. However, since the halide ion of the metal halide perovskite has very high mobility, halide ion migration may occur. For this reason, when metal halide perovskite nanoparticle of different halide ion composition are used in a wavelength converting body, the composition of the metal halide perovskite nanoparticle changes due to ion migration, so that the emission wavelength band of the wavelength converting body is easy changed. Therefore, it is very difficult to obtain stable light emission of two or more wavelengths with a wavelength converting body using only metal halide ion metal halide perovskite. In addition, aggregation of the metal halide perovskite nanoparticle may occur due to the high reactivity of the metal halide perovskite, and luminescence efficiency may decrease.

In addition, the existing inorganic quantum dots used as wavelength converting bodys essentially contain cadmium (Cd) for color purity and luminescence performance, and the cadmium is very harmful to the human body, so according to the Restriction of Hazardous Substances Directive (RoHS) standards, after 2022, it can only be used at less than 100 ppm, and in the case of quantum dots that do not use cadmium, the color purity is very low as the half width (FWHM) is 35 nm or more. In addition, semiconductor materials constituting quantum dots are very expensive, and a large amount of quantum dots is required for manufacturing a wavelength converting body due to the low absorbance of the quantum dots, so there is a problem of cost increase.

However, the hybrid wavelength converting body according to the present disclosure replaces a part of the inorganic quantum dot wavelength converting body with metal halide perovskite nanoparticle that do not contain cadmium, thus the cadmium content in the wavelength converting body can be greatly reduced. This is of great commercial importance as it can reduce the harmfulness of wavelength converting bodys and also enable wavelength converting bodys to meet RoHS criteria. In particular, metal halide perovskite nanoparticle have a greater absorbance than the above inorganic quantum dots, so only a smaller amount of luminescence than conventional inorganic quantum dots can be used to obtain equivalent or higher efficiency properties.

In addition, in the hybrid wavelength converting body according to the present disclosure, the non-metal halide perovskite quantum dots do not contain halide ions. Therefore, halide ion migration does not occur between the non-metal halide perovskite quantum dots and the metal halide perovskite nanocrystal particle. Thus, the composition of metal halide perovskite nanocrystal particle within the hybrid wavelength converting body under this disclosure does not change, so the above metal halide perovskite nanocrystal particle can obtain stable luminescence without changing the luminous wavelength.

Therefore, the hybrid wavelength converting body according to the present disclosure is essentially different from the conventional non-metal halide perovskite wavelength converting body or metal halide perovskite wavelength converting body, and is a more advanced wavelength converting body.

The metal halide perovskite nanocrystal particle and the non-metal halide perovskite quantum dots may convert light generated from an excitation light source into different wavelengths. Specifically, with respect to blue light generated from an excitation light source, the metal halide perovskite nanocrystal particle may emit green light, and the non-metal halide perovskite quantum dots may emit red light.

The green light is in a range in which the lower value of the number is the lower limit value and the higher value is the upper limit value selected from the group of 500 nm, 501 nm, 502 nm, 503 nm, 504 nm, 505 nm, 506 nm, 507 nm, 508 nm, 509 nm, 510 nm, 511 nm, 512 nm, 513 nm, 514 nm, 515 nm, 516 nm, 517 nm, 518 nm, 519 nm, 520 nm, 521 nm, 522 nm, 523 nm, 524 nm, 525 nm, 526 nm, 527 nm, 528 nm, 529 nm, 530 nm, 531 nm, 532 nm, 533 nm, 534 nm, 535 nm, 536 nm, 537 nm, 538 nm, 539 nm, 540 nm, 541 nm, 542 nm, 543 nm, 544 nm, 545 nm, 546 nm, 547 nm, 548 nm, 549 nm, 550 nm, 560 nm, 570 nm, 580 nm. The red light is in a range in which the lower value of the number is the lower limit value and the higher value is the upper limit value selected from the group of 590 nm, 600 nm, 601 nm, 602 nm, 603 nm, 604 nm, 605 nm, 606 nm, 607 nm, 608 nm, 609 nm, 610 nm, 611 nm, 612 nm, 613 nm, 614 nm, 615 nm, 616 nm, 617 nm, 618 nm, 619 nm, 620 nm, 621 nm, 622 nm, 623 nm, 624 nm, 625 nm, 626 nm, 627 nm, 628 nm, 629 nm, 630 nm, 631 nm, 632 nm, 633 nm, 634 nm, 635 nm, 636 nm, 637 nm, 638 nm, 639 nm, 640 nm, 641 nm, 642 nm, 643 nm, 644 nm, 645 nm, 646 nm, 647 nm, 648 nm, 649 nm, 650 nm, 660 nm, 670 nm, 680 nm, 690 nm, 700 nm.

When a metal halide perovskite nanocrystal emits green light and a nonmetal halide perovskite quantum dot emits red light, the excitation light is effectively converted into green light due to the high absorbance of the metal halide perovskite nanocrystal. It is possible to induce energy transfer from metal halide perovskite nanocrystals to non-metal halide perovskite quantum dots. Therefore, it is possible to secure efficiency characteristics equal to or higher than that of conventional non-metal halide perovskite quantum dot wavelength converting bodys with a smaller amount of luminous material, and stable light emission by effectively reducing the self-energy transfer of metal halide perovskite nanoparticle.

In the hybrid wavelength converting body according to the present disclosure, since the metal halide perovskite nanocrystal particle and the non-metal halide perovskite quantum dots are heterogeneous wavelength conversion particle, a large difference in absorption and light emission characteristics exist. In addition, the metal halide perovskite has very high absorbance. Therefore, it is difficult to match the mixing ratio when compared to the conventional quantum dot wavelength converting body. Accordingly, in the hybrid wavelength converting body according to the present disclosure, it is important to adjust the mixing ratio of the metal halide perovskite nanocrystal particle and the non-metal halide perovskite quantum dots so that the luminance of green light and red light is at the same level.

In this case, based on the weight ratio of metal halide perovskite nanocrystal particle to the sum of the weights of the quantum dots, the mixing ratio of the metal halide perovskite nanocrystal particle and the quantum dots may be from 20 wt % to 80 wt %. For example, the weight ratio of the metal halide perovskite nanocrystal particle to the sum of the weights of the metal halide perovskite nanocrystal particle and the quantum dots is a range in which the lower value of the number is the lower limit value and the higher value is the upper limit value selected from the group of 20 wt %, 25 wt %, 30 wt %, 35 wt %, 40 wt %, 45 wt %, 50 wt %, 51 wt %, 52 wt %, 53 wt %, 54 wt %, 55 wt %, 56 wt %, 57 wt %, 58 wt %, 59 wt %, 60 wt %, 61 wt %, 62 wt %, 63 wt %, 64 wt %, 65 wt %, 66 wt %, 70 wt %, 75 wt %, 80 wt %. In addition, preferably, the weight ratio of the metal halide perovskite nanocrystal particle to the sum of the weights of the metal halide perovskite nanocrystal particle and the quantum dots may be 50 wt % to 66 wt %, outside the above range, if the mixing ratio of the metal halide perovskite nanocrystal particle is large, the aggregation of the metal halide perovskite may occur, so stable wavelength conversion cannot be performed, and self-energy transition between the metal halide perovskite nanocrystal particle (self-absorption) occurs, thus there is a problem that the luminescence efficiency is greatly reduced or the luminous wavelength is changed.

The non-metal halide perovskite quantum dots (15) are Si-based nanocrystals, II-IV group compound semiconductor nanocrystals, III-V group compound semiconductor nanocrystals, IV-VI group compound semiconductor nanocrystals, boron quantum dots, carbon quantum dots, metal quantum dots, or it may include at least one of and mixtures thereof.

The II-IV-based compound semiconductor nanocrystals are CdS, CdSe, CdTe, ZnS, ZnSe, ZnTe, HgS, HgSe, HgTe, CdSeS, CdSeTe, CdTe, ZnSeS, ZnSeTe, ZnSTe, HgSe S, HgSeTe, CdZnSe, CdZnTe, CdHgS, CdHgSe, CdHgTe, HgZnS, HgZnSe, HgZnTe, CdZn SeS, CdZnSeTe, CdZnSTe, CdHgSeS, CdHgSeS, CdHgSeTe, CdHgSe and may be any one selected from the group consisting of but is not limited thereto.

The III-V group compound semiconductor nanocrystals are GaN, GaP, GaAs, AlN, AlP, AlAs, InN, InP, InAs, GaNP, GaNAs, GaPAs, AlNP, AlNAs, AlPAs, InNP, InNAs, InPAs, GaAlNP, GaAlNAs, GaAlPAs, GaInNP, GaInNAs, GaInPAs, InAlNP, InAlNAs, InAlPAs, and may be any one selected from the group consisting of, but is not limited thereto.

The IV-VI compound semiconductor nanocrystal may be SbTe, but is not limited thereto.

The carbon quantum dots may be graphene quantum dots, carbon quantum dots, C3N4 alternating quantum dots, and polymer quantum dots, but are not limited thereto.

The metal quantum dots may be Au, Ag, Al, Cu, Li, Cu, Pd, Pt, and alloys thereof, but are not limited thereto.

For hybrid wavelength converting body under this disclosure, the above dispersive medium may be liquid, uniformly distributing the above metal halide perovskite nanoparticle and the above nonmetallic halide perovskite quantum dots, and immobilizing them when cured with ultraviolet irradiation to be hardened. These distributed media can be at least one of the epoxy resins, silicon, and their mixtures, but are not limited to them.

FIG. 96 is a schematic diagram showing a hybrid wavelength converting body according to another embodiment of the present disclosure.

Referring to FIG. 96, the hybrid wavelength converting body (400) under other examples of this disclosure may contain metal halide perovskite nanocrystal particle (20), non-metallic halide perovskite quantum dots (15), distributed media (30), and additional sealing members (10) sealing the above distributed media.

In addition, hybrid wavelength converting bodys in other examples of this disclosure may include metal halide perovskite nanocrystal particle, non-metallic halide perovskite fluorescence, and sealing members that seal the dispersion medium.

These sealing members (10) may be consist of materials of a type that are not corroded by dispersive media distributed by metal halide perovskite nanocrystal particle and non-metallic halide perovskite quantum dots or non-metallic halide perovskite phosphor, which is, preferably, be at least one of epoxy resin, acrylic polymer, glass, carbonate polymer, silicon, and their mixture, but not limited thereof. For example, the polymer resin can be heated and gradually recovered, so it can be used as a sealing material to form a pack with metal halide perovskite nanocrystal particles and non-metal halide perovskite quantum dots dispersed inside. The manufacturing method of hybrid wavelength converting bodys (400) using these sealing members shall be described in detail in the following <Manufacturing methods of hybrid wavelength converting bodys>.

Hereinafter, a method of manufacturing a hybrid wavelength converting body according to the present disclosure will be described.

First, metal halide perovskite nanocrystal particles and non-metal halide perovskite quantum dots are prepared as wavelength conversion particle.

Since the description of the metal halide perovskite nanocrystal particle and the non-metal halide perovskite quantum dots is the same as described above, it will be omitted to avoid redundant description.

At times, non-metallic halide perovskite quantum dots can be used commonly used in the industry, used in the market, or manufactured by methods commonly used in the industry.

The metal halide perovskite nanocrystal particle may be prepared according to the following method, but is not limited thereto.

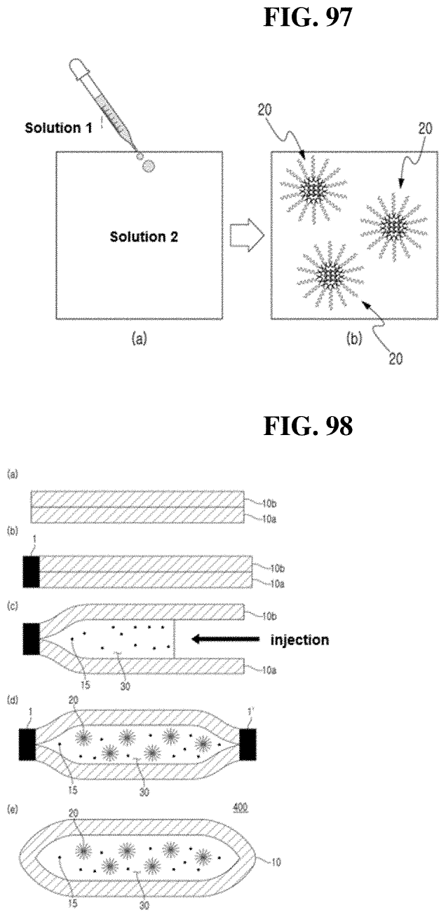

FIG. 97 is a schematic diagram showing a method of manufacturing a metal halide perovskite nanocrystal particle used as a wavelength conversion particle in a hybrid wavelength converting body according to an embodiment of the present disclosure.

Referring to FIG. 97, the metal halide perovskite nanocrystal particle may be prepared through the aforementioned inverse nano-emulsion method or ligand-assisted reprecipitation method, but is not limited thereto. Since the inverse nano-emulsion method and the ligand-assisted reprecipitation method are as described above, detailed descriptions are omitted.

The metal halide perovskite nanocrystal particle described above can be dispersed in all organic solvents. Accordingly, since the size, emission wavelength spectrum, ligand, and constituent elements can be easily adjusted, it can be applied to various electronic devices.

Meanwhile, the size of the crystalline particles of the metal halide perovskite can be controlled by controlling the length or shape factor of the alkyl halide surfactant. For example, shape factor control can be sized through linear, tapered, or inverted triangular surfactants.

Furthermore, the form of metal halide perovskite nanocrystals may be commonly used in the field. The form of metal halide perovskite nanocrystals can be zero-dimensional, one-dimensional or two-dimensional. Examples include spherical (sphere), ellipsoid cube, hollow cube, pyramid, cylinder, cone, elliptic column, hollow sphere, Janus particle, prism, multipod, polyhedron, nano tube, nano wire, nano fiber, or nanoplatelet.

In addition, the size of the crystalline particles may be 1 nm to 10 μm or less. For example, 1 nm, 2 nm, 3 nm, 4 nm, 5 nm, 6 nm, 7 nm, 7.5 nm, 8 nm, 8.5 nm, 9 nm, 9.5 nm, 10 nm, 10.5 nm, 11 nm, 11.5 nm, 12 nm, 12.5 nm, 13 nm, 13.5 nm, 14 nm, 14.5 nm, 15 nm, 16 nm, 17 nm, 18 nm, 19 nm, 20 nm, 25 nm, 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 150 nm, 200 nm, 250 nm, 300 nm, 350 nm, 400 nm, 450 nm, 500 nm, 600 nm, 700 nm, 800 nm, 900 nm, 1 μm, 2 μm, 5 μm, or 10 μm. The size of the particle can be defined as a region with the lower value as the minimum value and the larger value as the maximum value among the two numbers selected above. It is preferably 8 nm or more and 300 nm or less, and more preferably 10 nm or more and 30 nm or less. On the other hand, the size of the crystalline particles at this time means a size that does not take into account the length of a ligand to be described later, that is, the size of the remaining portions excluding the ligand. When the size of the crystalline particles is 1 μm or more, there is a fundamental problem that excitons do not emit light due to thermal ionization and delocalization of charge carriers in a large crystal, but are separated into free charges and disappeared. In addition, more preferably, as described above, the size of the crystalline particles may be greater than or equal to a Bohr diameter. The thermal ionization and delocalization of the charge carrier may gradually appear when the size of the nanocrystal exceeds 100 nm. If it is more than 300 nm, the phenomenon will appear more, and if it is more than 1 μm, it is completely bulky and is subject to the above phenomenon.

For example, when the crystalline particles are spherical, the diameter of the crystalline particles may be 1 nm to 10 μm. Preferably 1 nm, 3 nm, 5 nm, 6 nm, 7 nm, 8 nm, 9 nm, 10 nm, 11 nm, 12 nm, 13 nm, 14 nm, 15 nm, 16 nm, 17 nm, 18 nm, 19 nm, 20 nm, 25 nm, 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 150 nm, 200 nm, 250 nm, 300 nm, 350 nm, 400 nm, 450 nm, 500 nm, 600 nm, 700 nm, 800 nm, 900 nm, 1 μm, 2 μm, 5 μm or 10 may be μm.

In addition, the band gap energy of the nanocrystal particle may be 1 eV to 5 eV. Preferably, the band gap energy of the nanocrystal particle is a range in which the lower value of the number is the lower limit value and the higher value is the upper limit value selected from the group of 1 eV, 1.1 eV, 1.2 eV, 1.3 eV, 1.4 eV, 1.5 eV, 1.6 eV, 1.7 eV, 1.8 eV, 1.81 eV, 1.82 eV, 1.83 eV, 1.84 eV, 1.85 eV, 1.86 eV, 1.87 eV, 1.88 eV, 1.89 eV, 1.9 eV, 1.91 eV, 1.92 eV, 1.93 eV, 1.94 eV, 1.95 eV, 1.96 eV, 1.97 eV, 1.98 eV, 1.99 eV, 2 eV, 2.01 eV, 2.02 eV, 2.03 eV, 2.04 eV, 2.05 eV, 2.06 eV, 2.07 eV, 2.08 eV, 2.09 eV, 2.1 eV, 2.11 eV, 2.12 eV, 2.13 eV, 2.14 eV, 2.15 eV, 2.16 eV, 2.17 eV, 2.18 eV, 2.19 eV, 2.2 eV, 2.21 eV, 2.22 eV, 2.23 eV, 2.24 eV, 2.25 eV, 2.26 eV, 2.27 eV, 2.28 eV, 2.29 eV, 2.3 eV, 2.31 eV, 2.32 eV, 2.33 eV, 2.34 eV, 2.35 eV, 2.36 eV, 2.37 eV, 2.38 eV, 2.39 eV, 2.4 eV, 2.41 eV, 2.42 eV, 2.43 eV, 2.44 eV, 2.45 eV, 2.46 eV, 2.47 eV, 2.48 eV, 2.49 eV, 2.5 eV, 2.51 eV, 2.52 eV, 2.53 eV, 2.54 eV, 2.55 eV, 2.56 eV, 2.57 eV, 2.58 eV, 2.59 eV, 2.6 eV, 2.61 eV, 2.62 eV, 2.63 eV, 2.64 eV, 2.65 eV, 2.66 eV, 2.67 eV, 2.68 eV, 2.69 eV, 2.7 eV, 2.71 eV, 2.72 eV, 2.73 eV, 2.74 eV, 2.75 eV, 2.76 eV, 2.77 eV, 2.78 eV, 2.79 eV, 2.8 eV, 2.9 eV, 3 eV, 3.1 eV, 3.2 eV, 3.3 eV, 3.4 eV, 3.5 eV, 3.6 eV, 3.7 eV, 3.8 eV, 3.9 eV, 4 eV, 4.1 eV, 4.2 eV, 4.3 eV, 4.4 eV, 4.5 eV, 4.6 eV, 4.7 eV, 4.8 eV, 4.9 eV, 5 eV.

Hereinafter, a method of manufacturing a hybrid wavelength converting body according to an embodiment of the present disclosure will be described in detail.

(a) Dispersion Medium Curing Method

In the method of manufacturing a hybrid wavelength converting body according to the present disclosure, the dispersion medium curing method comprises: preparing a first dispersion solution by dispersing metal halide perovskite nanocrystal particle and nonmetal halide perovskite quantum dots or nonmetal halide perovskite phosphors as wavelength conversion particle in a dispersion solvent; Preparing a second dispersion solution by dispersing a dispersion medium in the first dispersion solution; And coating the second dispersion solution on a substrate and irradiating ultraviolet rays to polymerize and cure the dispersion medium to form a hybrid wavelength converting body.

First, in the step of preparing the first dispersion solution, metal halide perovskite nanocrystal particle and nonmetal halide perovskite quantum dots or nonmetal halide perovskite phosphors are dispersed together as wavelength conversion particle in the dispersion solvent to form a colloidal solution.

In this case, the above dispersion solvent is a material having a property that does not affect the performance of metal halide perovskite nanocrystal particle, nonmetal halide perovskite quantum dots, and nonmetal halide perovskite phosphors as wavelength conversion particle. The dispersion solvent may be selected from methanol, ethanol, tert-butanol, xylene, toluene, hexane, octane, cyclohexane, dichloroethylene, chloroform, and chlorobenzene, but is not limited thereto.

In the hybrid wavelength converting body, since the metal halide perovskite nanocrystal particle and the non-metal halide perovskite quantum dots are heterogeneous wavelength conversion particle, they exhibit a large difference in absorption and light emission depending on the wavelength. In addition, the metal halide perovskite has a very large absorbance. Therefore, it is difficult to match the mixing ratio compared to the conventional quantum dot wavelength converting body. At this time, the mixing ratio of the metal halide perovskite nanocrystal particle and the non-metal halide perovskite quantum dots is preferably 1:1 to 2:1 by weight, outside the above range, large mixing ratios of metal halide perovskite nanocrystal particle can cause metal halide perovskite aggregation, resulting in no stable wavelength conversion, and significant self-absorption among metal halide perovskite nanocrystal particle.

Next, a second dispersion solution is prepared by mixing a dispersion medium with the first dispersion solution. The dispersion medium may be in a liquid state, and uniformly disperse the metal halide perovskite nanoparticle and the non-metal halide perovskite-based quantum dots or non-ferrosite-based phosphors, and serves to immobilize the metal halide perovskite nanoparticle and the non-metal halide perovskite-based quantum dots or non-metal halide perovskite-based phosphors when irradiated with ultraviolet rays. The dispersion medium may be at least one of epoxy resin, silicone, and mixtures thereof, but is not limited thereto.

Next, a second dispersion solution is coated on the substrate. While coating the second dispersion solution, the dispersion solvent is removed to form a wavelength converting body in which metal halide perovskite nanocrystal particle and non-metal halide perovskite quantum dots or non-metal halide perovskite phosphors are uniformly mixed in a dispersion medium.

At this time, the coating method be selected from a variety of methods of a spin coating method, a spray method, a dip coating method, a bar coating method, a nozzle printing method, a slot-die coating method, a gravure printing method, a screen printing method, a brush painting method or a roll coating method, etc.

Next, the dispersion medium is polymerized and cured. The polymerization and curing may be performed by irradiating ultraviolet rays, and the ultraviolet rays used may be those having a wavelength of, for example, 350 to 400 nm, but are not limited thereto.

As the dispersion medium is polymerized and cured, a fixed wavelength in a state in which metal halide perovskite nanocrystal particle and non-metal halide perovskite quantum dots or non-metal halide perovskite phosphors are uniformly mixed in the dispersion medium A transformant is produced.

Thereafter, if necessary, the step of additionally removing the substrate may be further included.

(b) Synthesis of In-Situ Metal Halide Perovskite Nanocrystal Particle

In the method of manufacturing a hybrid wavelength converting body according to the present disclosure, the in-situ metal halide perovskite nanocrystal particle synthesis method comprises: preparing a metal halide perovskite precursor solution by dissolving a metal halide perovskite precursor in a solvent; Preparing a third dispersion solution by mixing a non-metal halide perovskite-based quantum dot and a dispersion medium with the metal halide perovskite precursor solution; And forming a hybrid wavelength converting body by coating and crystallizing the third dispersion solution on a substrate, and polymerizing and curing the dispersion medium by irradiating ultraviolet rays.

First, in the step of preparing a metal halide perovskite precursor solution, the metal halide perovskite precursor may be dissolved in a solvent.

In this case, the solvent may dissolve a metal halide perovskite precursor material, and may be a material having a property that does not affect the performance of a non-metal halide perovskite quantum dot. The solvent may be selected from dimethylformamide, dimethylsulfoxide, acetonitrile, gamma butyrolactone, methylpyrrolidone, and isopropyl alcohol, but is not limited thereto.

In the step of preparing the third dispersion solution, a colloidal solution is formed by dispersing a non-metal halide perovskite-based quantum dot and a dispersion medium together in the metal halide perovskite precursor solution. The third dispersion solution prepared is thus a colloidal solution in which a metal halide perovskite precursor is dissolved, and quantum dots and polymers are dispersed.

After that, a third dispersion solution is coated on the substrate. While coating the third dispersion solution, the dispersion solvent is removed to crystallize the metal halide perovskite precursor material on the substrate, so that a mixed wavelength converting body is formed in which metal halide perovskite nanocrystal particle and non-metal halide perovskite quantum dots are uniformly mixed in the dispersion medium is prepared.

At this time, the coating method can be selected from a variety of methods of a spin coating method, a spray method, a dip coating method, a bar coating method, a nozzle printing method, a slot-die coating method, a gravure printing method, a screen printing method, a brush painting method or a roll coating method, etc.

Next, the dispersion medium is polymerized and cured. The polymerization and curing may be performed by irradiating ultraviolet rays, and the ultraviolet rays used may be those having a wavelength of, for example, 350 to 400 nm, but are not limited thereto.

As the dispersion medium is polymerized and cured, a wavelength converting body in which metal halide perovskite nanocrystal particle and non-metal halide perovskite quantum dots are uniformly mixed in the dispersion medium is prepared.

In this case, the step of coating the third dispersion solution on the substrate and the step of irradiating ultraviolet rays may be performed in a different order or may be performed simultaneously.

Thereafter, if necessary, the step of additionally removing the substrate may be further included.

(c) Dispersion Medium Sealing Method

In the method of manufacturing a hybrid wavelength converting body according to the present disclosure, the dispersion medium sealing method comprises: stacking a first sealing member and a second sealing member; Adhering one side of the first sealing member and the second sealing member; Injecting dispersive medium with dispersion of metal halide perovskite nanocrystal particle and nonmetallic halide perovskite quantum dots as wavelength-converting particle between the first and second sealing members of other parts where the first and second sealing members are not attached; And sealing the dispersion medium in which metal halide perovskite nanocrystal particle and non-metal halide perovskite quantum dots are dispersed as wavelength conversion particle by bonding the other side of the first sealing member and the second sealing member with a sealing member.

FIG. 98 is a cross-sectional view showing a method of manufacturing a hybrid wavelength converting body using a sealing method according to an embodiment of the present disclosure.

Hereinafter, a method of manufacturing a hybrid wavelength converting body using the sealing method will be described in detail with reference to FIG. 98.

Referring to FIG. 98(a), a first sealing member (10a) and a second sealing member (10b) are stacked.

The sealing member is a polymer resin or silicone that is not corroded by the dispersion medium (30) in which the metal halide perovskite nanocrystal particle (20) and the non-metal halide perovskite quantum dots (15) are dispersed as wavelength conversion particle. In particular, since the polymer resin can be heated and gradually recovered, it can be used to form a pack-type wave converter with dispersion medium (30) dispersed wavelength conversion particle (15, 20) by using a heat-adhesion process.

Referring to FIG. 98(b), one side (1) of the first sealing member (10a) and the second sealing member (10b) may be heated and adhered via heat bonding process so that the above-described wavelength conversion particles (15, 20) and the dispersion medium (30) do not leak out of the sealing member (10a, 10b).

Referring to FIG. 98(c), inject the dispersive medium (30) with the above wavelength conversion particle (15, 20) dispersed between the first sealing member (10a) and the second sealing member (10b) of the other side where the aforementioned first sealing member (10a) and second sealing member (10b) are not bonded.

Referring to FIG. 98(d), the dispersion medium (30) in which the wavelength conversion particle (15, 20) are dispersed is sealed with sealing members (10a) and (10b) by using a heat bonding process to adhere the other side portion 1 of the first sealing member (10a) and the second sealing member (10b).

Referring to FIG. 98(e), it can be seen that the hybrid wavelength converting body (400) in which the dispersion medium (30) in which the wavelength conversion particle (15, 20) are dispersed is sealed with a sealing member (10).

Hybrid wavelength converting bodys (400) manufactured in the above method have the advantage of being able to apply to light emitting devices without the need for a separate ligand refining process, as they are sealed by dispersing metal halide perovskite nanocrystal particle (20) and non-metallic halide perovskite quantum dots (15). Therefore, it can prevent oxidation of wavelength converting particles during ligand refining, which results in high color purity and luminous effects when applied to luminescence devices. In addition, the process can be simplified.

In addition, the above hybrid wavelength converting body (400) can significantly reduce the cadmium content by replacing some of the conventional quantum dot wavelength converting bodys with metal halide perovskite nanoparticles that do not contain cadmium. In particular, since metal halide perovskite nanoparticle have a greater absorbance than quantum dots, only a smaller amount of luminescence than conventional quantum dots can be used to obtain more than equivalent efficiency properties.

In addition, the present disclosure provides a light emitting device including the hybrid wavelength converting body.

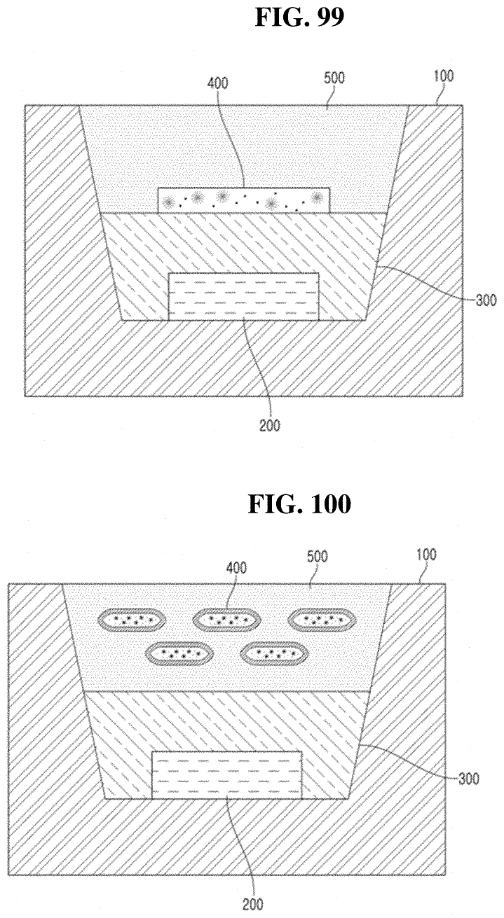

FIG. 99 and FIG. 100 are cross-sectional views of a light emitting device according to an embodiment of the present disclosure.

Referring to FIGS. 99 and 100, the light emitting element under one example of this disclosure includes the aforementioned hybrid wavelength converting body (400) placed on the base structure (100) and the aforementioned base structure (100), on at least one here light source (200) and the aforementioned light source (200).

The base structure (100) described above may be a package frame or a base substrate. When the base structure (100) is a package frame, the package frame may include the base substrate. The base substrate may be a submount substrate or a light emitting diode wafer. The light-emitting diode wafer is a state before being separated into light-emitting diode chips, and indicates a state in which a light-emitting diode device is formed on the wafer. The base substrate may be a silicon substrate, a metal substrate, a ceramic substrate, or a resin substrate.

The base structure (100) described above may be a package lead frame or a package pre-mold frame. The base structure (100) may include a bonding pad (not shown). Bonding pads may contain Au, Ag, Cr, Ni, Cu, Zn, Ti, Pd, and the like. External connection terminals (not shown) connected to bonding pads may be located on the outer side of the base structure (100). The bonding pads and the external connection terminals may be those provided in the package lead frame.

Place the excitation light source (200) on the aforementioned base structure (100). The aforementioned light source (200) is preferable to emit light that has a shorter wavelength than the luminous wavelength of the wavelength converting body (400) of the hybrid wavelength converting body (metallic halide perovskite nanocrystal particle, nonmetallic halide perovskite quantum dot) according to this disclosure. The aforementioned light source (200) may be either a light emitting diode or a laser diode. In addition, if the base structure (100) is a light emitting diode wafer, the stage of placing the light source here may be omitted. For example, here in the light source (200) can use blue LEDs, which can use gallium nitride LEDs that emit blue light from 420 nm to 480 nm.

As in FIGS. 99 and 100, the first encapsulation part (300) may be formed by the filling of the encapsulation material encapsulating the aforementioned light source (200). The aforementioned first encapsulation part (300) may serve as a protective shield as well as to cover the aforementioned light source (200). In addition, if the aforementioned wavelength converting body (400) is located on the first encapsulation part (300), the second encapsulation part (500) may be formed to protect and secure it. The encapsulation material may contain epoxy, silicon, acrylic polymer, glass, carbonate polymer, and at least one of these compounds.

The first encapsulation part (300) can be formed by using various methods such as a compression molding method, a transfer molding method, a dotting method, a blade coating method, a screen coating method, dip coating, spin coating, spray, or inkjet printing. However, the first encapsulation part (300) may be omitted.

Since the detailed description of the hybrid wavelength converting body (400) is the same as described above, it will be omitted to avoid redundant description.

As shown in FIGS. 99 and 100, the second encapsulation part (500) may be formed by filling the encapsulating material for encapsulating the wavelength converting body (400) on the wavelength converting body (400) described above. The second encapsulation part (500) may use the same material as the first encapsulation part (300) described above, and may be formed through the same manufacturing method.

In addition, the light emitting device under this disclosure may support the grooves containing the floor surface where the light source is to be installed and the side where the reflection part is formed, and may include more support for the electrodes connected to the light source above.

The above-described light emitting device can be applied to lighting, backlight units, as well as light emitting devices.

In the embodiment of this disclosure, the above light emitting device is designed specific to the unit cell, but if the base structure is a submount substrate or light emitting diode wafer, the above submount substrate or light emitting diode wafer may be cut and processed into each unit cell.

On the other hand, in the manufacture of a metal halide perovskite wavelength converting body, the metal halide perovskite agglomerates with each other in the dispersion medium, so that the metal halide perovskite crystals are not uniformly dispersed. Self-absorption may occur, resulting in a decrease in luminescence efficiency, and a luminous wavelength band may be changed. Therefore, it is very important that the metal halide perovskite is uniformly dispersed in the dispersion medium.

When the metal halide perovskite emitter further includes a plurality of organic ligands surrounding the metal halide perovskite nanocrystal, in general, the organic ligands have hydrophobic properties, thus the type of dispersion medium that can be prepared is limited.

Meanwhile, the metal halide perovskite has very low stability against oxygen and moisture. Therefore, it is preferable to use a dispersion medium having a low transmittance for oxygen and moisture in order to prepare a stable metal halide perovskite wavelength converting body. However, since such a dispersion medium having a low transmittance to oxygen and moisture is generally not compatible with a hydrophobic material, it may be difficult to obtain a uniform dispersion when mixed with the metal halide perovskite. In order to solve this problem, a method of mixing a metal halide perovskite and a dispersion medium at a high temperature may be used, but luminescence efficiency may decrease due to the heat-sensitive property of the metal halide perovskite.

To solve this problem, if the metal halide perovskite luminescence contains more than one organic ligand surrounding the metal halide perovskite nanocrystals, preferably the above wavelength converting body may be a particle-dispersed form of metal halide perovskite by the metal halide perovskite.

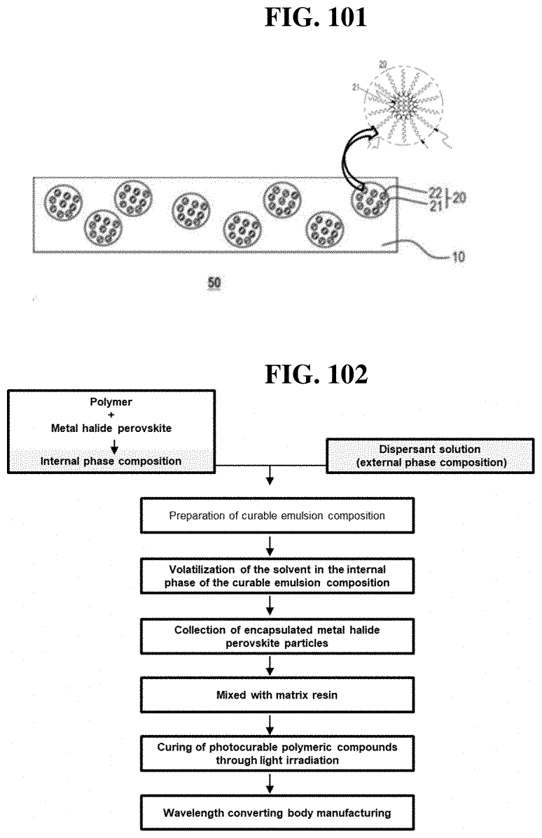

FIG. 101 is a cross-sectional view of an encapsulated metal halide perovskite wavelength conversion layer film according to an embodiment of the present disclosure.

Referring to FIG. 101, the metal halide perovskite may have a structure encapsulated by the first dispersion medium, and the encapsulated metal halide perovskite may have a structure dispersed in the second dispersion medium.

In addition, preferably, the first dispersion medium may be characterized by uniformly dispersing the metal halide perovskite due to good compatibility with the organic ligand.

In addition, preferably, the dispersion medium may be a polymer. Preferably, the polymer may be characterized in that it has a polar group in at least one of a backbone or a side chain. The polar group may be adsorbed on the surface of the metal halide perovskite to increase the dispersibility of the metal halide perovskite.

In the case of having a polar group in the main chain of the polymer, the main chain of the polymer may be characterized in that it includes polyester, ethyl cellulose, polyvinylpyridine, and combinations thereof, but is not limited thereto.

In the case of having a polar group in the side chain of the polymer, the polar group may be characterized in that it contains an oxygen component, preferably the polar group —OH, —COOH, —COH, —CO—, —O— And a combination thereof, but is not limited thereto.

In addition, it is preferable that the polymer has a number average molecular weight of about 300 g/mol to 100,000 g/mol. If the number average molecular weight of the polymer is out of the above range and is less than 300 g/mol, the distance between the quantum dots in the quantum dot-polymer bead is insufficient, so that the luminescence efficiency may decrease, and if it exceeds 100,000 g/mol, the bead size becomes excessively large. Defects may occur in the film forming process. The polymer may be a thermosetting resin or a wax-based compound.

Specifically, the thermosetting resin may be selected from a combination of them from a group of a silicone resin, epoxy resin, petroleum resin, phenol resin, urea resin, melamine resin, unsaturated polyester resin, amino resin, butyl rubber, isobutylene rubber, acrylic rubber, urethane rubber, but is not limited thereto.

The silicone resin may be a liquid siloxane polymer. The siloxane polymer is a dimethyl silicone oil, methylphenyl silicone oil, diphenyl silicone oil, polysiloxane, a diphenyl siloxane copolymer, methyl Hydrogen silicone oil, methyl hydroxyl silicone oil, fluoro silicone oil, polyoxyether copolymer, amino-modified silicone oil silicone oil), epoxy-modified silicone oil, carboxyl-modified silicone oil, carbonyl-modified silicone oil, methacryl-modified silicone oil modified silicone oil), mercapto-modified silicone oil, polyether-modified silicone oil, methylstyryl silicone oil, alkyl-modified silicone oil modified silicone oil) or fluoro-modified silicone oil, but is not limited thereto.

The epoxy resin may be bisphenol A, bisphenol F, bisphenol AD, bisphenol S, hydrogenated bisphenol A, and combinations thereof, but are not limited thereto.

The thermosetting resin may additionally be used as a catalyst or a curing agent according to a thermosetting mechanism. In addition, preferably, the catalyst may be a platinum catalyst, and the curing agent may be an organic peroxide or an amine having a liquid aromatic ring at room temperature.

Also preferably, the organic peroxide is 2,4-dichlorobenzoyl peroxide, benzoyl peroxide, dicumyl peroxide, di-tertiary-It may be butyl perbenzoate(methyl-tert-butylperbenzoate) and 2,5-bis(tert-butylperoxy)benzoate (2,5-bis(tert-butylperoxy)benzoate), but is not limited thereto.

The amine having a liquid aromatic ring at the above or at room temperature is at least one or a combination of them selected from a group of diethyltoluenediamine, 1-methyl-3,5-diethyl-2,4-diaminobenzene (1-methyl-3,5-diethyl-2,4-diaminobenzene), 1-methyl-3,5-diethyl-2,6-diaminobenzene (1-methyl-3,5-diethyl-2,6-diaminobenzene), 1,3,5-Triethyl-2,6-diaminobenzene (1,3,5-triehyl-2,6-diaminobenzene), 3,3-diethyl-4,4-diaminodiphenylmethane (3,3-diethyl-4,4-diaminodimethylphenylmethane), 3,3,5,5-tetramethyl-4,4′-diaminodiphenylmethane (3,3,5,5-tetramethyl-4,4-diaminodiphenylmethane), but is not limited thereto.

The wax-based compound may be in a solid state at room temperature, but may have a melting point of 40° C. to 150° C., and may be a resin having a molecular weight of 100 to 100,000. In addition, it may be preferably petroleum wax, animal natural wax, vegetable natural wax, or synthetic wax, but is not limited thereto.

The second dispersion medium serves to disperse the encapsulated metal halide perovskite, and preferably may be a material having low oxygen and moisture permeability.

In addition, preferably, the above secondary distributed medium may be characterized by photorespiratory polymerization compounds.

For example, the second dispersion medium may be an acrylic resin.

The photocurable polymerization compound may be a photopolymerizable monomer, a photopolymerizable oligomer, and a combination thereof. The photopolymerizable monomer and the photopolymerizable oligomer are not particularly limited as long as they contain at least one of a carbon-carbon double bond and a triple bond and are polymerizable by light.

In particular, when the second dispersion medium is an acrylic resin, the photopolymerizable monomer and the photopolymerizable oligomer may be an acrylic monomer and an acrylic oligomer, respectively.

The acrylic oligomer may be an epoxy acrylic resin. The epoxy acrylic resin may be a resin in which an epoxide group of the epoxy resin is substituted with an acrylate group. Like the epoxy resin, the epoxy acrylate resin may have a low moisture permeability and air permeability due to its main chain properties.

Also preferably, the epoxy acrylate resin is bisphenol-A glycerolate diacrylate, bisphenol-A ethoxylate diacrylate, bisphenol-A glycerolate dimethacrylate (bisphenol A glycerolate dimethacrylate), bisphenol-A ethoxylate dimethacrylate (bisphenol A ethoxylate dimethacrylate), and may be a combination thereof, but is not limited thereto.

The acrylic monomer may be an unsaturated group-containing acrylic monomer, an amino group-containing acrylic monomer, an epoxy group-containing acrylic monomer, a carboxylic acid group-containing acrylic monomer, and combinations thereof, but is not limited thereto.

The unsaturated group-containing acrylic monomer is at least one or a combination of them selected from a group of methyl acrylate, methyl methacrylate, ethyl acrylate, ethyl methacrylate, n-propyl acrylate (n-propylacrylate), n-propyl methacrylate, i-propylacrylate, i-propyl methacrylate, n-butylacrylate, n-butyl methacrylate, i-butylacrylate, i-butyl methacrylate, sec-butyl acrylate, sec-Butyl methacrylate (sec-butyl methacrylate), t-butyl acrylate (t-butylacrylate), t-butyl methacrylate (t-butyl methacrylate), 2-hydroxyethyl acrylate (2-hydroxyethyl acrylate), 2-hydroxyethyl methacrylate, 2-hydroxypropyl acrylate, 2-hydroxypropyl methacrylate, 3-hydroxypropyl acrylic 3-hydroxypropyl acrylate, 3-hydroxypropyl methacrylate, 2-hydroxybutyl acrylate, 2-hydroxybutyl methacrylate), 3-hydroxybutyl acrylate, 3-hydroxybutyl methacrylate, 4-hydroxybutyl acrylate (4-hydroxybutyl acrylate), 4-hydroxybutyl methacrylate, allyl acrylate, allyl methacrylate, benzyl acrylate, benzyl methacrylate methacrylate), cyclohexyl acrylate, cyclohexyl methacrylate, phenyl acrylate, phenyl methacrylate, 2-methoxyehtyl acrylate), 2-methoxyethyl methacrylate, 2-phenoxyethyl acrylate, 2-phenoxyethyl methacrylate, methoxydiethylene methoxydiethyneglycol acrylate, methoxydiethyleneglycol methacrylate, methoxytriethyleneglycol acrylate, methoxytriethyleneglycol methacrylate, met hoxypropylene glycol acrylic Methoxy propyleneglycol acrylate, methoxypropyleneglycol methacrylate, methoxydipropyleneglycol acrylate, methoxydipropyleneglycol methacrylate, isobornyl acrylate), isobornyl methacrylate, dicyclopentaacrylate, dicyclopentamethacrylate, 2-hydroxy-3-phenoxypropyl acrylate, 2-hydroxy 2-hydroxy-3-phenoxypropyl methacrylate, glycerol monoacrylate, glycerol monomethacrylate, but are not limited thereto.

The amino group-containing acrylic monomer is at least one or a combination of them selected from a group of 2-aminoethyl acrylate, 2-aminoethyl methacrylate, 2-dimethylaminoethyl acrylate, 2-dimethylaminoethyl methacrylate, 2-aminopropyl acrylate, 2-aminopropyl methacrylate, 2-dimethylaminopropyl acrylate, 2-dimethylaminopropyl methacrylate, 3-aminopropyl acrylate, 3-aminopropyl methacrylate, 3-dimethylaminopropyl acrylate, 3-dimethylaminopropyl methacrylate, but is not limited thereto.

The epoxy group-containing acrylic monomer is at least one or a combination of them selected from a group of glycidyl acrylate, glycidyl methacrylate, glycidyloxyethyl acrylate, glycidyloxyethyl methacrylate, glycidyloxypropyl acrylate, glycidyloxypropyl methacrylate, glycidyloxybutyl acrylate, glycidyloxybutyl methacrylate, but is not limited thereto.

The carboxylic acid group-containing acrylic monomer is at least one or a combination of them selected from a group of acrylic acid, methacrylic acid, acrylo oxyacetic acid, methacrylo oxyacetic acid, acryloyl oxypropionic acid, methacryloyl oxypropionic acid, acrylo oxybutric acid, methacrylo oxybutric acid, but is not limited thereto.

In addition, the photopolymerizable monomer may be a photoresist material. The photoresist material may be a silicone or epoxy material.

The photoresist material may be a commercial photoresist. The commercial photoresist materials may be AZ 5214E PR, AZ 9260 PR from, AZ AD Promoter-K (HMDS), AZ nLOF 2000 Series, AZ LOR-28 PR, AZ 10xT PR, AZ 5206-E, AZ GXR-601, AZ 04629 from AZ Electronics Materials; SU-8 from MICROCHEM, 950 PMMA, 495 PMMA; S1800 from micropossit; DNR-L300, DSAM, DPR, DNR-H200, DPR-G from Dongjin Semichem; or CTPR-502 from Kotem, but is not limited thereto.

In addition, preferably, the second dispersion medium may further include a photoinitiator.

The kind of the photoinitiator is not particularly limited and may be appropriately selected. Preferably, the photoinitiator is at least one or a combination of them selected from a group of a triazine-based compound, acetophenone-based compound, benzophenone-based compound, thioxanthone-based compound, benzoin-based compound, oxime (oxime) compounds, carbazole compounds, diketone compounds, sulfonium borate compounds, diazo compounds, nonimidazolium compounds, but is not limited thereto.

Examples of the triazine-based compound are 2,4,6-trichloro-s-triazine, 2-phenyl-4,6-bis(trichloromethyl)-s-triazine, 2-(3′, 4′-dimethoxy styryl)-4,6-bis(trichloromethyl)-s-triazine, 2-(4′-methoxy naphthyl)-4,6-bis(trichloromethyl))-s-triazine, 2-(p-methoxyphenyl)-4,6-bis(trichloro methyl)-s-triazine, 2-(p-tolyl)-4,6-bis(trichloromethyl)-s-triazine, 2-biphenyl-4,6-bis(trichloromethyl)-s-triazine, bis(trichloromethyl)-6-styryl-s-triazine (bis(trichloro methyl)-6-styryl-s-triazine), 2-(naphtho-1-yl)-4,6-bis(trichloromethyl)-s-triazine, 2-(4-methoxy naphtho-1-yl)-4,6-bis(trichloromethyl)-s-triazine, 2,4-Trichloromethyl(piperonyl)-6-triazine, 2,4-(trichloromethyl(4′-methoxy styryl)-6-Triazine, but is not limited thereto.

Examples of the acetophenone-based compound are 2,2-diethoxy acetophenone, 2,2,-dibutoxy acetophenone, 2-2-hydroxy-2-methyl propiophenone, pt-butyl trichloro acetophenone, pt-butyl dichloro acetophenone, 4-Chloro acetophenone, 2,2-dichloro-4-phenoxy acetophenone, 2-methyl-1-(4-(methylthio)phenyl)-2-mopholino propan-1-one, 2-benzyl-2-dimethylamino-1-(4-mopholinophenyl)-butan-1-one, but is not limited thereto.

Examples of the benzophenone-based compound include benzophenone, benzoyl benzoate, methyl 2-benzoylbenzoate, 4-phenyl benzophenone, hydroxy Benzophenone, benzophenone acrylate, 4,4-bis(dimethylamino)benzophenone, 4,4-dichlorobenzophenone, 3,3-dimethyl-2-methoxy benzophenone, and the like, but is not limited thereto.

Examples of the thioxanthone-based compound include thioxanthone, 2-methyl thioxantone, isopropyl thioxantone, and 2,4-diethyl thioxantone, 2,4-diiospropyl thioxantone, 2-chloro thioxantone, but is not limited thereto.

Examples of the benzoin-based compound are benzoine, benzoine methyl ether, benzoine ethyl ether, benzoine isopropyl ether, benzoine isobutyl ether, benzyl dimethyl ketal, but is not limited thereto.

Example of the oxime compound are 2-(o-benzoyloxime)-1-[4-(phenylthio)phenyl]-1,2-octanedione and 1-(o-acetyloxime)-1-[9-ethyl-6-(2-methylbenzoyl)-9H-carbazol-3-yl]ethanone, but is not limited thereto.

Meanwhile, the second dispersion medium may further include a crosslinking agent for crosslinking.

Preferably, the crosslinking agent is at least one or a combination of them selected from a group of ethylene glycol (di(metha) acrylate, polyethyleneglycol di(metha) acrylate, trimethylolpropane di(metha)acrylate, trimethylolpropane tri(metha)acrylate, pentaerythritol tri(metha)acrylate, pentaerythritol tetra(metha)acrylate)Acrylate, 2-trisacrylo oxymethylethylpthalic acid, propylene glycol di(metha)acrylate, polypropyleneglycol di(metha)acrylate, dipentaerythritol penta(metha)acrylate, and dipentaerythritol hexa(metha)acrylate), but is not limited thereto.

In addition, when the metal halide perovskite-polymer composite is manufactured in the form of a film attached to a specific substrate, the second dispersion medium may further include a polymer binder. The polymeric binder may serve to improve adhesion between the substrate and the metal halide perovskite-polymer composite.

The substrate (10) serves as a support for a light emitting device, and may be a transparent material. In addition, the substrate (10) may be a flexible material or a hard material, and preferably may be a flexible material.

The material of the substrate (10) is glass, sapphire, quartz, silicon, polyethylene terephthalate (PET), polystyrene (PS), polyimide (polyimide, PI), polyvinyl chloride (PVC), polyvinylpyrrolidone (PVP), polyethylene (PE), etc., but is not limited thereto.

The polymer binder may be an acrylic polymer binder, a cardo polymer binder, or a polymer of a combination thereof, but is not limited thereto.

The acrylic polymer binder may be a copolymer of a first unsaturated monomer containing a carboxyl group and a second unsaturated monomer copolymerizable therewith. The first unsaturated monomer may be a carboxylic acid vinyl ester compound such as acrylic acid, maleic acid, methacrylic acid, vinyl acetate, itaconic acid, 3-butenoic acid, fumaric acid, vinyl benzoate, or a combination thereof, but is not limited thereto.

The second unsaturated monomer is at least one or a combination of them selected from a group of an alkenyl aromatic compound, an unsaturated carboxylic acid ester compound, an unsaturated carboxylic acid amino alkyl ester compound, an unsaturated carboxylic acid glycidyl ester compound, a vinyl cyanide compound, a hydroxy alkyl acrylate, but is not limited thereto.

Also preferably, the second unsaturated monomer is at least one or a combination of them selected from a group of styrene, α-methylstyrene, vinyltoluene, vinylbenzylmethylether, methylacrylate, ethylacrylate, butylacry late, benzyl acrylate, cyclohexylacrylate, phenyl Acrylate, 2-aminoethylacrylate, 2-dimethylaminoethylacrylate, N-phenylmaleimide, N-benzylmaleimide, N-alkylmaleimide, 2-dimethylaminoethylmethacrylate, acrylonitrile, unsaturated amide compounds such as glycidyl acrylate and acrylamide;2-hydroxy ethyl acrylate, 2-hydroxy butyl acrylate, but is not limited thereto.

The acrylic polymer binder is at least one or a combination of them selected from a group of a methacrylic acid/benzyl methacrylate copolymer, methacrylic acid/benzyl methacrylate/styrene copolymer, methacrylic acid/benzyl methacrylate/2-hydroxyethyl methacrylate copolymer, methacrylic acid/benzyl methacrylate/styrene/2-hydroxyethyl methacrylate copolymer, but is not limited thereto.

The metal halide perovskite-polymer composite film may further include a light diffusing agent. The light diffusing agent may be metal oxide particles, metal particles, and combinations thereof, but is not limited thereto. The light diffusing agent may serve to increase the probability of encountering the metal halide perovskite with the incident light of the composition by increasing the refractive index of the composition.

The light diffusing agent may include inorganic oxide particles such as alumina, silica, zirconia, titania, and zinc oxide, and metal particles such as gold, silver, copper, and platinum, but is not limited thereto. At this time, a dispersant may be added to increase the dispersibility of the light diffusing agent.

Hereinafter, a method of manufacturing a metal halide perovskite wavelength converting body having a structure in which particles capsulated metal halide perovskite are dispersed in the matrix resin will be described.

The first dispersion material and the second dispersion material may be cured sequentially.

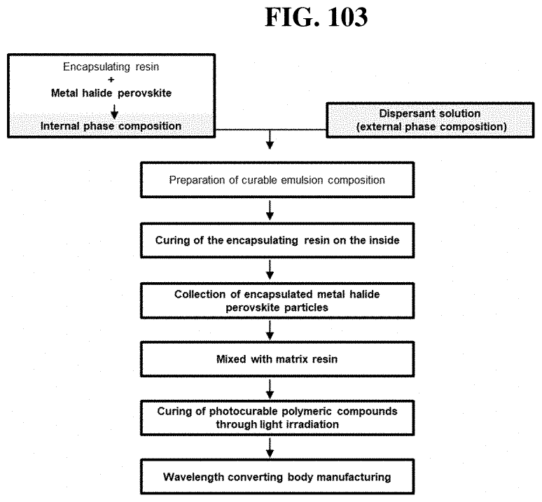

FIGS. 102 and 103 are schematic diagrams showing a method of manufacturing a metal halide perovskite wavelength converting body having a structure in which encapsulated particles are dispersed according to an embodiment of the present disclosure.

Referring to FIGS. 102 and 103, a method of manufacturing a metal halide perovskite wavelength converting body is characterized with having a structure in which encapsulated particles are dispersed according to an embodiment of the present disclosure uses a curable emulsion composition. As described above, the emulsion refers to a solution in which a liquid droplet is uniformly dispersed in different types of droplets that are not miscible (immiscible).

In the present specification, the fine droplets discontinuously present in the curable emulsion composition are defined as ‘inner phase’, and the composition continuously present in the emulsion composition in addition to the inner phase is defined as ‘outer phase’.

Referring to FIGS. 102 and 103, first, a solution capable of forming an inner phase is prepared.

Referring to FIG. 103, the above internal phase is for the manufacture of metal halide perovskites encapsulated by the first distributed medium and may be characterized by the inclusion of metal halide perovskites and polymers.

The metal halide perovskite may be a material having a three-dimensional crystal structure, a two-dimensional crystal structure, a one-dimensional crystal structure, or a zero-dimensional crystal structure.

The metal halide perovskite is ABX₃(3D), A₄BX₆(0D), AB₂X₅(2D), A₂BX₄(2D), A₂BX₆(0D), A₂B⁺B³⁺X₆(3D), A₃B₂X₉(2D) or Aₙ₋₁BₙX₃a+1 (quasi-2D) (n is an integer between 2 and 6) may be included. A is a monovalent cation, B is a metal material, and X may be a halogen element. The quasi-2D structure may be a Ruddlesden-Popper phase or a Dion-Jacobson phase.

The monovalent cation may be a monovalent organic cation or an alkali metal. For example, the monovalent organic cation is organic ammonium (RNH₃⁺), organic amidinium derivative (RC(═NR₂)NR₂)⁺, organic guanidinium derivative (R₂NC(═NR₂)NR₂)⁺, organic diammonium (CₓH₂ₓ₋ₙ₊₄)(NH₃)ₙ⁺, ((CₓH₂ₓ₊₁)ₙNH₃)(CH₃NH₃)ₙ⁺, (RNH₃)₂⁺, (CₙH₂ₙ₊₁NH₃)²⁺, (CF₃NH₃)⁺, (CF₃NH₃)ₙ⁺, ((CₓF₂ₓ₊₁)ₙNH₃)₂(CF₃NH₃)ₙ⁺, ((CₓF₂ₓ₊₁)ₙNH₃)₂⁺ or (CₙF₂ₙ₊₁NH₃)₂⁺ (x, n is an integer of 1 or more, R=hydrocarbon derivative, alkyl, Alkyl fluoride derivatives, H, F, Cl, Br, I), and combinations thereof, but are not limited thereto. The alkali metal may be Li⁺, Na⁺, K⁺, Rb⁺, Cs⁺, Fr⁺, and combinations thereof, but is not limited thereto.

In addition, preferably, the organic cation is at least one or a combination of them selected from a group of acetamidinium, azaspironanium, benzene diammonium, benzylammonium, butanediammonium, iso-butylammonium, n-butylammonium, t-butylammonium, cyclohexylammonium, cyclohexylmethylammonium, diazobicyclooctanedinium, diethylammonium, N,N-diethylpropane diammonium, dimethylammonium, N,N-dimethylethane diammonium, dimethylpropane diammonium, dodecylammonium, ethanediammonium, ethylammoniuium, 4-fluoro-benzylammonium, 4-fluoro-phenylethylammonium, 4-fluoro-phenylammonium, formamidinium, guanidinium, hexanediammonium, hexylammonium, imidazolium, 2-methoxyethylammonium, 4-methoxy-phenlylethylammonium, 4-methoxy-phenylammonium, methylammonium, morpholinium, octylammonium, pentylammonium, pipeazinediium, piperidinium, propanediammonium, iso-propylammonium, di-iso-propylammonium, n-propylammonium, pyridinium, 2-pyrrolid-lium-1-yethylammonium, pyrrolidinium, quinclidin-1-ium, 4-trifluoromethyl-benzylammonium, 4-trifluoromethyl ammonium but is not limited thereto.

B may be a divalent transition metal, a rare earth metal, an alkaline earth metal, a monovalent metal, a combination of a trivalent metal, an organic substance (a monovalent, divalent, or trivalent cation), and a combination thereof. Also, preferably, the divalent transition metal, rare earth metal, and alkaline earth metal are Be²⁺, Mg²⁺, Ca²⁺, Sr²⁺, Ba²⁺, Ra²⁺, Mn²⁺, Fe²⁺, Co²⁺, Ni²⁺, Cu²⁺, Zn²⁺, Ru²⁺, Pd²⁺, Cd²⁺, Pt²⁺, Hg²⁺, Ge²⁺, Sn²⁺, Pb²⁺, Se²⁺, Te²⁺, Po²⁺, Bi²⁺, Eu²⁺, No²⁺, and combinations thereof, but are not limited thereto. The monovalent metal may be Li⁺, Na⁺, K⁺, Rb⁺, Cs⁺, Fr⁺, Ag⁺, Hg⁺, Ti⁺, and combinations thereof, and the trivalent metal is Cr³⁺, Fe³⁺, Co³⁺, Ru³⁺, Rh³⁺, Eu³⁺, Au³⁺, Al³⁺, Ga³⁺, In³⁺, Ti³⁺, As³⁺, Sb³⁺, Bi³⁺, La³⁺, Ce³⁺, Pr³⁺, Nd³⁺, Pm³⁺, Sm³⁺, Eu³⁺, Gd³⁺, Tb³⁺, Dy³⁺, Ho³⁺, Er³⁺, Tm³⁺, Yb³⁺, Lu³⁺, Ac³⁺, Lu³⁺, Bk³⁺, Cf³⁺, Es³⁺, Fm³⁺, Md³⁺, Lr³⁺, and combinations thereof.

In addition, X may be F⁻, Cl⁻, Br⁻, I⁻, At⁻, and combinations thereof.

The metal halide may be in the form of nano crystalline particles.

The metal halide perovskite nanocrystal may further include a plurality of organic ligands (20) surrounding the halide metal halide perovskite nanocrystal (10). The organic ligands (20) at this time are a material used as a surfactant, and may include an alkyl halide, an amine ligand, and a carboxylic acid or a phosphonic acid. Detailed descriptions of the alkyl halide, amine ligand, carboxylic acid, and phosphonic acid are as described in <Metal Halide perovskite nanocrystal particles.

In addition, the form of the metal halide perovskite nanocrystal may be a form generally used in the field. The shape of the metal halide perovskite nanocrystal may be a 0-dimensional, 1-dimensional or 2-dimensional shape. As an example, it may be in the form of a sphere, an ellipsoid cube, a hollow cube, a pyramid, a cylinder, a cone, an elliptic column, Hollow sphere, Janus particle, prism, multipod, polyhedron, nano tube, nano wire, nano fiber, or nanoplatelet.

Preferably, the polymer may be characterized in that it has a polar group in at least one of a backbone or a side chain. The polar group may be adsorbed on the surface of the metal halide perovskite to increase the dispersibility of the metal halide perovskite.

In the case of having a polar group in the main chain of the polymer, the main chain of the polymer may be characterized in that it includes polyester, ethyl cellulose, polyvinylpridine, and combinations thereof, but is not limited thereto.

In the case of having a polar group in the side chain of the polymer, the polar group may be characterized in that it contains an oxygen component, preferably the polar group —OH, —COOH, —COH, —CO—, —O— and combinations thereof, but is not limited thereto.

In addition, it is preferable that the polymer has a number average molecular weight of about 300 g/mol to 100,000 g/mol. If the number average molecular weight of the polymer is out of the above range and is less than 300 g/mol, the distance between the quantum dots in the quantum dot-polymer bead is insufficient, so that the luminescence efficiency may decrease, and if it exceeds 100,000 g/mol, the bead size becomes excessively large. Defects may occur in the film forming process.

Referring to FIG. 103, the above internal phase is for the manufacture of particles encapsulated by the first distributed medium, metal halide perovskite and encapsulated resin, which can be characterized by the inclusion of metal halide perovskite and encapsulated resin.

The encapsulated resin encapsulates multiple metal halide perovskites to form a uniform dispersion within the matrix resin, and is not limited to materials that can uniformly distribute metal halides with hydrophilic properties.

Meanwhile, the encapsulating resin may be a thermosetting resin or a wax-based compound.

Preferably, the thermosetting resin may be a liquid resin that exists as a liquid at room temperature. In addition, preferably, the thermosetting resin may include a thermosetting resin that is cured by heat or a phase-mixed curable resin that is cured by heat, but is not limited thereto.

In addition, preferably, the thermosetting resin may be characterized in that thermal curing occurs or is accelerated at a temperature of 100° C. When the temperature at which thermal curing occurs or is accelerated exceeds 100° C., the metal halide perovskite crystal structure vulnerable to heat may be decomposed.

Specifically, the thermosetting resin is at least one or a combination of them selected from a group of a silicone resin, epoxy resin, petroleum resin, phenol resin, urea resin, melamine resin, unsaturated polyester resin, amino resin, butyl rubber, isobutylene rubber, acrylic rubber, urethane rubber, but is not limited thereto.

The silicone resin may be a liquid siloxane polymer. The siloxane polymer may be a dimethyl silicone oil, methylphenyl silicone oil, diphenyl silicone oil, polysiloxane, a diphenyl siloxane copolymer, methyl Hydrogen silicone oil, methyl hydroxyl silicone oil, fluoro silicone oil, polyoxyether copolymer, amino-modified silicone oil silicone oil), epoxy-modified silicone oil, carboxyl-modified silicone oil, carbonyl-modified silicone oil, methacryl-modified silicone oil modified silicone oil), mercapto-modified silicone oil, polyether-modified silicone oil, methylstyryl silicone oil, alkyl-modified silicone oil modified silicone oil) or fluoro-modified silicone oil, but is not limited thereto.

The epoxy resin may be bisphenol A, bisphenol F, bisphenol AD, bisphenol S, hydrogenated bisphenol A, and combinations thereof, but are not limited thereto.

The thermosetting resin may additionally be used as a catalyst or a curing agent according to a thermosetting mechanism. In addition, preferably, the catalyst may be a platinum catalyst, and the curing agent may be an organic peroxide or an amine having a liquid aromatic ring at room temperature.

The amine having a liquid aromatic ring at the above or at room temperature is diethyltoluenediamine, 1-methyl-3,5-diethyl-2,4-diaminobenzene, 1-methyl-3,5-diethyl-2,6-diaminobenzene, 1,3,5-triehyl-2,6-diaminobenzene, 3,3-diethyl-4,4-diaminodimethylphenylmethane, 3,3,5,5-tetramethyl-4,4-diaminodiphenylmethane and it may be a combination of them, but is not limited thereto.

The wax-based compound may be in a solid state at room temperature, but may have a melting point of 40° C. to 150° C., and may be a resin having a molecular weight of 100 to 100,000. In addition, it may be preferably petroleum wax, animal natural wax, vegetable natural wax, or synthetic wax, but is not limited thereto.

The inner phase may include a metal halide perovskite and a solvent capable of dispersing the encapsulating resin. The solvent is preferably a non-polar solvent, but is not limited thereto. For example, the non-polar solvent is dichloroethylene, trichloroethylene, chloroform, chlorobenzene, dichlorobenzene, styrene, dimethylformamide (dimethylformamide), dimethylsulfoxide, xylene, toluene, cyclohexane, or isopropylalcohol, but is not limited thereto.

After the inner phase solution is formed, the inner phase solution is mixed with a dispersant solution to form a curable emulsion composition. The solvent of the dispersant solution is not limited as long as it can form an emulsion with the inner phase solution, and may preferably be a polar solvent. Specifically, the polar solvent is acetic acid, acetone, acetonitrile, dimethylformamide, gamma butyrolactone, N-methylpyrrolidone, ethanol, or dimethylsulfoxide, but is not limited thereto. The dispersant solution forms an external phase in the curable emulsion composition formed by mixing.

If the above encapsulation resin is a wax-based compound, it may be converted to liquid phase by applying heat above the melting point of the wax-based compound in order to apply it to the hardened emulsion composition. The melting point of the waxy compound may vary depending on the type of waxy compound, but preferably the melting point of the waxy compound below 100° C. If the melting point exceeds 100° C. to apply to hardened emulsion compound, the heat-vulnerable metal halide perovskite crystal structure may be decomposed.

The curable emulsion composition may be performed while stirring with a magnetic stirrer, and preferably, the stirring speed may be 500 rpm or more. When the stirring speed is out of the above range and is less than 500 rpm, droplets of the inner phase solution may be aggregated to separate the inner phase solution and the dispersant solution from each other.

When the curable emulsion composition is formed by the above method, the metal halide perovskite can be encapsulated in various ways depending on the composition of the inner phase. The obtained encapsulated particles may be recovered after removing the solvent, and further include a subsequent encapsulation process using a matrix resin and an encapsulating resin.

Referring to FIG. 102, when the inner phase includes metal halide perovskite and polymer, the solvent of the inner phase may be volatilized. Volatilizing the solvent in the inner phase may be performed by a method of decompressing the curable emulsion composition. When the solvent in the inner phase is removed by the above process, the metal halide perovskite may be encapsulated by a polymer contained in the inner phase. At this time, the first dispersion medium becomes the polymer.

Referring to FIG. 103, metal halide perovskite can be encapsulated in a variety of ways depending on the type of encapsulated resin, if the above internal phase includes metal halide perovskite and encapsulated resin. At this time, the first dispersion medium above may be formed by curing the above encapsulated resin.

In particular, if the encapsulated resin is thermosetting resin, the encapsulated particle may be manufactured by heat curing the encapsulated resin inside the hardened emulsion composition by applying heat to the hardened emulsion composition. The temperature at the time of thermal hardening and the type of thermosetting resin may be selected, preferably not more than 100° C. If the temperature at the time of thermal hardening exceeds 100° C., the metal halide perovskite crystal structure may be decomposed.

When the encapsulating resin is a wax-based compound, the metal halide perovskite may be encapsulated by removing heat applied to form a curable emulsion composition.

Referring to FIG. 103, after forming the encapsulated metal halide perovskite particles by the above process, the solvent present in the droplet may be removed and the metal halide perovskite particles can be collected.

Thereafter, the obtained encapsulated metal halide perovskite particles are mixed with a matrix resin to prepare a metal halide perovskite particle-matrix resin mixture. Preferably, the matrix may be a material having low oxygen and moisture permeability. In addition, preferably, the matrix resin may be a photocurable polymerization compound.

For example, the photocurable polymerization compound may be an acrylic resin.

The photocurable polymerization compound may be a photopolymerizable monomer, a photopolymerizable oligomer, and a combination thereof. The photopolymerizable monomer and the photopolymerizable oligomer are not particularly limited as long as they contain at least one of a carbon-carbon double bond and a triple bond are included and polymerizable by light.

In particular, when the photocurable polymerization compound is an acrylic resin, the photopolymerizable monomer and the photopolymerizable oligomer may be an acrylic monomer and an acrylic oligomer, respectively.

The acrylic oligomer may be an epoxy acrylic resin. The epoxy acrylic resin may be a resin in which an epoxide group of the epoxy resin is substituted with an acrylate group. Like the epoxy resin, the epoxy acrylate resin may have low moisture permeability and air permeability due to its main chain properties.

Also preferably, the epoxy acrylate resin is bisphenol-A glycerolate diacrylate, bisphenol-A ethoxylate diacrylate, bisphenol A glycerolate dimethacrylate, bisphenol A ethoxylate dimethacrylate, and may be a combination thereof, but is not limited thereto.

The acrylic monomer may be an unsaturated group-containing acrylic monomer, an amino group-containing acrylic monomer, an epoxy group-containing acrylic monomer, a carboxylic acid group-containing acrylic monomer, and combinations thereof, but is not limited thereto.

The unsaturated group-containing acrylic monomer may be methylacrylate, methyl methacrylate, ethylacrylate, ethyl methacrylate, n-propylacrylate, n-propyl methacrylate, i-propylacrylate, i-propyl methacrylate, n-butylacrylate, n-Butyl methacrylate, i-butylacrylate, i-butyl methacrylate, sec-butylacrylate, sec-butyl methacrylate, t-butylacrylate, t-butyl methacrylate, 2-hydroxyethyl acrylate, 2-hydroxy 2-hydroxyethyl methacrylate, 2-hydroxypropyl acrylate, 2-hydroxypropyl methacrylate, 3-hydroxypropyl acrylate, 3-hydroxypropyl methacrylate, 2-hydroxybutyl acrylate, 2-hydroxybutyl methacrylate, 3-hydroxybutyl acrylate, 3-hydroxybutyl methacrylate, 4-hydroxybutyl acrylate, 4-hydroxybutyl methacrylate, allyl acrylate, allyl methacrylate, benzyl acrylate, benzyl methacrylate, cyclohexyl acrylate, cyclohexyl methacrylate, phenyl acrylate, phenyl methacrylate, 2-methoxyehtyl acrylate, 2-methoxyethyl methacrylate, 2-phenoxyethyl acrylate, 2-phenoxyethyl methacrylate, methoxydiethylene methoxydiethyleneglyco acrylate, methoxydiethyleneglycol methacrylate, methoxytriethyleneglycol acrylate, methoxytriethyleneglycol methacrylate, methoxy propyleneglycol acrylate, methoxypropyleneglycol acrylate, methoxypropyleneglycol methacrylate, methoxydipropyleneglycol acrylate, methoxydipropyleneglycol methacrylate, isoboronyl acrylate, isoboronyl methacrylate, dicyclopenta acrylate, dicyclopenta methacrylate, 2-hydroxy-3-phenoxypropyl acrylate, 2-hydroxy-3-phenoxypropyl methacrylate, glycerol monoacrylate, glycerol monomethacrylate, or a combination thereof, but is not limited thereto.

The amino group-containing acrylic monomer may be 2-aminoethyl acrylate, 2-aminoethyl methacrylate, 2-dimethylaminoethyl acrylate, and 2-dimethyl 2-dimethylaminoethyl methacrylate, 2-aminopropyl acrylate, 2-aminopropyl methacrylate, 2-dimethylaminopropyl acrylate, 2-dimethylaminopropyl methacrylate, 3-aminopropyl acrylate, 3-aminopropyl methacrylate, 3-dimethylaminopropyl acrylate, 3-dimethylaminopropyl methacrylate, or combination thereof, but is not limited thereto.

The epoxy group-containing acrylic monomer may be glycidyl acrylate, glycidyl methacrylate, glycidyloxyethyl acrylate, glycidyloxyethyl methacrylate, glycidyloxypropyl acrylate, glycidyloxypropyl methacrylate, glycidyloxybutyl acrylate, glycidyloxybutyl methacrylate), or combinations thereof, but is not limited thereto.

The carboxylic acid group-containing acrylic monomers may be acrylic acid, methacrylic acid, acrylo oxyacetic acid, methacrylo oxyacetic acid, and acryloyl oxypropionic acid, methacryloyl oxypropionic acid, acrylo oxybutric acid, methacrylo oxybutric acid, ort combinations thereof, but are limited thereto.

In addition, the photopolymerizable monomer may be a photoresist material. The photoresist material may be a silicone or epoxy material.

The photoresist material may be a commercial photoresist. The commercial photoresist materials may be AZ 5214E PR, AZ 9260 PR, AZ AD Promoter-K (HMDS), AZ nLOF 2000 Series, AZ LOR-28 PR, AZ 10xT PR, AZ 5206-E, AZ GXR-601, AZ 04629 from AZ Electronics Materials; SU-8 from MICROCHEM, 950 PMMA, 495 PMMA; S1800 from micropossit; DNR-L300, DSAM, DPR, DNR-H200, DPR-G from Dongjin Semichem; CTPR-502 from Kotem, but is not limited thereto.

The mixture may further include a photoinitiator for photocuring according to the type of the photocurable polymer compound.

Examples of the benzophenone-based compound include benzophenone, benzoyl benzoate, methyl 2-benzoylbenzoate, 4-phenyl benzophenone, hydroxybeonzophenone, acrylated benzophenone acrylate, 4,4-bis(dimethylamino)benzophenone, 4,4-dichlorobenzophenone, 3,3-dimethyl-2-methoxy benzophenone, and the like, but are not limited thereto.

Examples of the thioxanthone-based compound include thioxantone, 2-methyl thioxantone, isopropyl thioxantone, and 2,4-diethyl thioxantone, 2,4-diiospropyl thioxantone, 2-chloro thioxantone, and the like, but is not limited thereto.

Examples of the benzoin-based compound include benzoine, benzoine methyl ether, benzoine ethyl ether, benzoine isopropyl ether, benzoine isobutyl ether, benzyl dimethyl ketal, and the like, but is not limited thereto.

Examples of the oxime compound are 2-(o-benzoyloxime)-1-[4-(phenylthio)phenyl]-1,2-octanedione (2-(o-benzoyloxime)-1-[4-(phenylthio)phenyl]-1,2,-octandione and 1-(o-acetyloxime)-1-[9-ethyl-6-(2-methylbenzoyl)-9H-carbazol-3-yl]ethanone (1-(o-acetyloxime)-1-[9-ethyl-6-(2-methylbenzoyl)-9H-carbazol-3-yl]ethanone).

Meanwhile, the mixture may further include a crosslinking agent for crosslinking.

Preferably, the crosslinking agent is ethylene glycol di(meth)acrylate (di(metha) acrylate), polyethylene glycol di(meth)acrylate (polyethyleneglycol di(metha) acrylate), trimethylolpropane di(meth)acrylate (trimethylolpropane)di(metha)acrylate), trimethylolpropane tri(metha)acrylate, pentaerythritol tri(metha)acrylate, pentaerythritol tetra(meth)acrylate (pentaerythritol tetra(metha)acrylate), 2-trisacrylo oxymethylethylpthalic acid, propylene glycol di(metha)acrylate, polypropylene glycol di(metha)acrylate)Acrylate (polypropyleneglycol di(metha)acrylate), dipentaerythritol penta(metha)acrylate and dipentaerythritol hexa(metha)acrylate), and a combination thereof, but is not limited thereto.

After preparing the mixture, the mixed solution may be cured to obtain a wavelength converting body having a structure in which a metal halide perovskite encapsulated in a second dispersion medium formed by curing the matrix resin is dispersed.

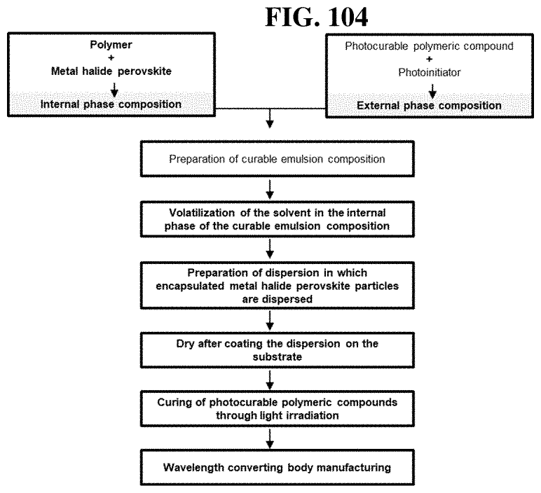

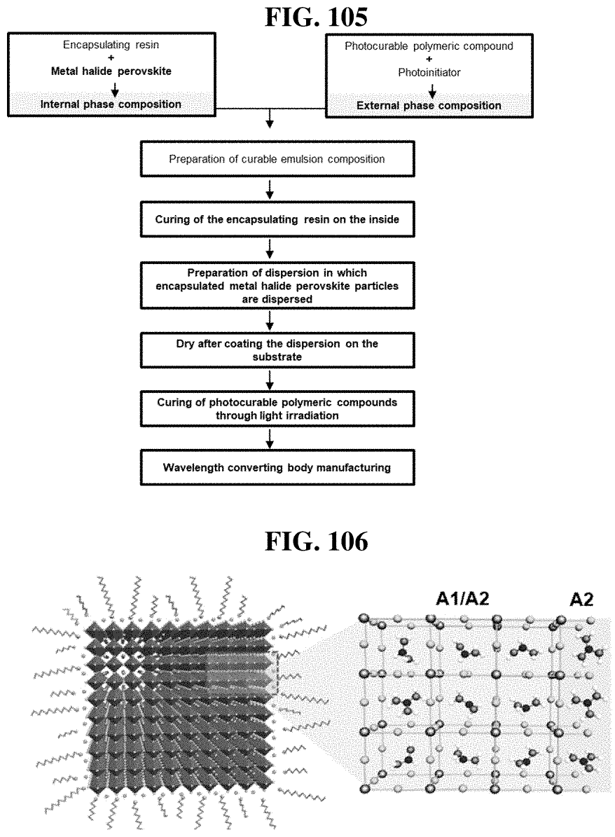

FIGS. 104 and 105 are schematic diagrams showing a method of manufacturing a metal halide perovskite wavelength converting body having a structure in which encapsulated particles are dispersed according to another embodiment of the present disclosure.

Referring to FIGS. 104 and 105, the manufacturing method of metal halide perovskite wavelength converting body with encapsulated particles distributed according to the example of this disclosure is characterized by the use of hardened emulsion composites, without the need for a separate encapsulated metal halide perovskite collection.

Since the description of the metal halide perovskite is the same as described above, a detailed description will be omitted.

The metal halide may be in the form of nano crystalline particles.

The metal halide perovskite nanocrystal may further include a plurality of organic ligands (20) surrounding the halide metal halide perovskite nanocrystal (10). The organic ligands (20) at this time are a material used as a surfactant, and may include an alkyl halide, an amine ligand, and a carboxylic acid or a phosphonic acid.

At this time, examples of the available alkyl halide, amine ligand, carboxylic acid, or phosphonic acid are the same as described above, and thus are omitted to avoid redundant description.

In addition, the form of the metal halide perovskite nanocrystal may be a form generally used in the art. The shape of the metal halide perovskite nanocrystal may be a 0-dimensional, 1-dimensional, or 2-dimensional shape. As an example, a sphere, an ellipsoid, a hollow cube, a pyramid, a cylinder, a cone, an elliptic column, Hollow sphere, Janus particle, prism, multipod, polyhedron, nano tube, nano wire, nano fiber It may be in the form of (nano fiber) or nanoplatelet.

Referring to FIGS. 104 and 105, first manufacture a solution that can form an internal phase.

Referring to FIG. 104, the above internal phase is intended to manufacture particles encapsulated by the first distributed medium, metal halide perovskite, and may be characterized by the inclusion of metal halide perovskite and polymers.

Preferably, the polymer may be characterized in that it has a polar group in at least one of a backbone or a side chain. The polar group may be adsorbed on the surface of the metal halide perovskite to increase the dispersibility of the metal halide perovskite.

In the case of having a polar group in the main chain of the polymer, the main chain of the polymer may be characterized in that it includes polyester, ethyl cellulose, polyvinylpridine, and combinations thereof, but is not limited thereto.

When having a polar group in the side chain of the polymer, the polar group may be characterized in that it contains an oxygen component, preferably the polar group —OH, —COOH, —COH, —CO—, —O— and combination thereof, but is not limited thereto.

Furthermore, it is recommended that the above polymers have a horizontal molecular weight of between 300 g/mol and 100,000 g/mol. If the horizontal molecular weight of the polymer is less than 300 g/mol outside the above range, the difference of quantum dots within the quantum dot-high molecular bead may be insufficient, and if the bead size exceeds 100,000 g/mol, it may become too large and cause defects in the milling process.

Referring to degree 105, the above internal phase is intended to manufacture particles encapsulated by the first distributed medium, metal halide perovskite, and may be characterized by metal halide perovskite and encapsulated resin.

The encapsulated resin encapsulates multiple metal halide perovskites to form a uniform dispersion within the matrix resin, and is not limited to materials that have hydrophilic properties that can uniformly distribute metal halides.

Meanwhile, the above encapsulated resin may be thermosetting resin, or waxy compound.

Preferably, the thermosetting resin above may be a liquid resin present as a liquid at room temperature. In addition, preferably, the above thermosetting resin may contain, but is not limited to, thermosetting resin, which is cured by heat, or is precipitated by heat.

In addition, preferably, the above thermosetting resin may be characterized by heat hardening or facilitation at 100° C. Metal halide perovskite crystal structures vulnerable to heat can be dismantled if the temperature at which heat hardening occurs or is promoted exceeds 100° C. outside the above range.

Specifically, the thermosetting resin may be selected from, but not limited to, silicone resin, epoxy resin, petroleum resin, phenolic resin, element resin, melamine resin, unsaturated polyester resin, amino resin, butyl rubber, isobutylene rubber, acrylic rubber, urethane rubber and their combination.

The silicone resin may be a liquid siloxane polymer. The siloxane polymer is dimethyl silicone oil, methylphenyl silicone oil, diphenyl silicone oil, polysiloxane, a diphenyl siloxane copolymer, methyl Hydrogen silicone oil, methyl hydroxyl silicone oil, fluoro silicone oil, polyoxyether copolymer, amino-modified silicone oil silicone oil), epoxy-modified silicone oil, carboxyl-modified silicone oil, carbonyl-modified silicone oil, methacryl-modified silicone oil, mercapto-modified silicone oil, polyether-modified silicone oil, methylstyryl silicone oil, alkyl-modified silicone oil or fluoro-modified silicone oil, but is not limited thereto.

The epoxy resin may be bisphenol A, bisphenol F, bisphenol AD, bisphenol S, hydrogenated bisphenol A, and combinations thereof, but is not limited thereto.

The thermosetting resin may additionally be used as a catalyst or a curing agent according to a thermosetting mechanism. In addition, preferably, the catalyst may be a platinum catalyst, and the curing agent may be an organic peroxide or an amine having a liquid aromatic ring at room temperature.

In addition, preferably, the organic peroxide is 2,4-dichlorobenzoyl peroxide, benzoyl peroxide, dicumyl peroxide, methyl-tert-butylperbenzoate and 2,5-bis(tert-butylperoxy)benzoate, but is not limited thereto.

The amine having a liquid aromatic ring at the above or at room temperature is diethyltoluenediamine, 1-methyl-3,5-diethyl-2,4-diaminobenzene, 1-methyl-3,5-diethyl-2,6-diaminobenzene, 1,3,5-triehyl-2,6-diaminobenzene, 3,3-diethyl-4,4-diaminodimethylphenylmethane, 3,3,5,5-tetramethyl-4,4-diaminodiphenylmethane and combinations thereof, but is not limited thereto.

The wax-based compound may be solid at room temperature, but may have a melting point of 40° C. to 150° C., and may be a resin having a molecular weight of 100 to 100,000. In addition, it may be preferably petroleum wax, animal natural wax, vegetable natural wax, or synthetic wax, but is not limited thereto.

The inner phase may include a metal halide perovskite and a solvent capable of dispersing the encapsulating resin. The solvent is preferably a non-polar solvent, but is not limited thereto. For example, the non-polar solvent is dichloroethylene, trichloroethylene, chloroform, chlorobenzene, dichlorobenzene, styrene, dimethylformamide, dimethylsulfoxide, xylene, toluene, cyclohexane, or isopropylalcohol, but is not limited thereto.

After forming the inner phase solution, the inner phase solution is mixed with a dispersant solution to form a curable emulsion composition. The solvent of the dispersant solution is not limited as long as it can form an emulsion with the inner phase solution, and may preferably be a polar solvent. Specifically, the polar solvent is acetic acid, acetone, acetonitrile, dimethylformamide, gamma butyrolactone, N-methylpyrrolidone, ethanol, or dimethylsulfoxide, but is not limited thereto. The above dispersant solution forms an external phase in the hardened emulsion composition formed by mixing.

When the encapsulating resin is a wax-based compound, in order to apply it to a curable emulsion composition, it may be converted into a liquid state by applying heat above the melting point of the wax-based compound. The melting point of the wax-based compound may vary depending on the type of the wax-based compound, but it is preferable to select a melting point of the wax-based compound having a melting point of 100° C. or less, and if the melting point exceeds 100° C., in the process of applying heat above the melting point of the wax-based compound for application to the curable emulsion composition, the metal halide perovskite crystal structure which is vulnerable to heat may be decomposed.

The curable emulsion composition may be performed while stirring with a magnetic stirrer, and preferably the stirring speed may be 500 rpm or more. When the stirring speed is out of the above range and is less than 500 rpm, droplets of the inner phase solution may be aggregated to separate the inner phase solution and the dispersant solution from each other.

Referring to FIGS. 104 and 105, the external phase includes a photocurable compound. For example, the photocurable polymerization compound may be an acrylic resin.

The photocurable polymerization compound may be a photopolymerizable monomer, a photopolymerizable oligomer, and a combination thereof. The photopolymerizable monomer and the photopolymerizable oligomer are not particularly limited as long as they contain at least one of a carbon-carbon double bond and a triple bond and are polymerizable by light.

In particular, when the photocurable polymerization compound is an acrylic resin, the photopolymerizable monomer and the photopolymerizable oligomer may be an acrylic monomer and an acrylic oligomer, respectively.

The acrylic oligomer may be an epoxy acrylic resin. The epoxy acrylic resin may be a resin in which an epoxide group of the epoxy resin is substituted with an acrylate group. Like the epoxy resin, the epoxy acrylate resin may have low moisture permeability and air permeability due to its main chain properties.

Also preferably, the epoxy acrylate resin is bisphenol-A glycerolate diacrylate, bisphenol-A ethoxylate diacrylate, bisphenol-A glycerolate dimethacrylate, bisphenol A ethoxylate dimethacrylate, and may be a combination thereof, but is not limited thereto.

The acrylic monomer may be an unsaturated group-containing acrylic monomer, an amino group-containing acrylic monomer, an epoxy group-containing acrylic monomer, a carboxylic acid group-containing acrylic monomer, and combinations thereof, but is not limited thereto.

The unsaturated group-containing acrylic monomer is methylacrylate, methyl methacrylate, ethylacrylate, ethyl methacrylate, n-propylacrylate, n-propyl methacrylate, i-propylacrylate, i-propyl methacrylate, n-butylacrylate, n-Butyl methacrylate, i-butylacrylate, i-butyl methacrylate, sec-butylacrylate, sec-butyl methacrylate, t-butylacrylate, t-butyl methacrylate, 2-hydroxyethyl acrylate, 2-hydroxy 2-hydroxyethyl methacrylate, 2-hydroxypropyl acrylate, 2-hydroxypropyl methacrylate, 3-hydroxypropyl acrylate, 3-hydroxypropyl methacrylate, 2-hydroxybutyl acrylate, 2-hydroxybutyl methacrylate, 3-Hydroxybutyl acrylate, 3-hydroxybutyl methacrylate, 4-hydroxybutyl acrylate, 4-hydroxybutyl methacrylate, allyl acrylate, allyl methacrylate, benzyl acrylate, benzyl methacrylate, cyclohexyl acrylate, cyclohexyl methacrylate, phenyl acrylate, phenyl methacrylate, 2-methoxyehtyl acrylate, 2-methoxyethyl methacrylate, 2-phenoxyethyl acrylate, 2-phenoxyethyl methacrylate, methoxydiethylene Methoxydiethyneglycol acrylate, methoxydiethyleneglycol methacrylate, methoxytriethyleneglycol acrylate, methoxytriethyleneglycol methacrylate, methoxypropylene glycol acrylic Methoxy propyleneglycol acrylate, methoxypropyleneglycol methacrylate, methoxydipropyleneglycol acrylate, methoxydipropyleneglycol methacrylate, isoboronyl acrylate, isoboronyl methacrylate, dicyclopenta acrylate, dicyclopenta methacrylate, 2-hydroxy-3-phenoxypropyl acrylate, 2-hydroxy-3-phenoxypropyl methacrylate, glycerol monoacrylate, glycerol monomethacrylate, and may be a combination thereof, but is not limited thereto.

The amino group-containing acrylic monomer is 2-aminoethyl acrylate, 2-aminoethyl methacrylate, 2-dimethylaminoethyl acrylate, and 2-dimethyl 2-dimethylaminoethyl methacrylate, 2-aminopropyl acrylate, 2-aminopropyl methacrylate, 2-dimethylaminopropyl acrylate, 2-dimethylaminopropyl methacrylate, 3-aminopropyl acrylate, 3-aminopropyl methacrylate, 3-dimethylaminopropyl acrylate, 3-dimethylaminopropyl methacrylate, and a combination thereof, but is not limited thereto.

The epoxy group-containing acrylic monomer is glycidyl acrylate, glycidyl methacrylate, glycidyloxyethyl acrylate, glycidyloxyethyl methacrylate, glycidyloxypropyl acrylate, glycidyloxypropyl methacrylate, glycidyloxybutyl acrylate, glycidyloxybutyl methacrylate and a combination thereof, but is not limited thereto.

The carboxylic acid group-containing acrylic monomers include acrylic acid, methacrylic acid, acrylo oxyacetic acid, methacrylo oxyacetic acid, and acryloyloxypropionic acid (acryloyl oxypropionic acid), methacryloyl oxypropionic acid, acrylo oxybutric acid, methacrylo oxybutric acid, and combinations thereof, but are limited thereto. no.

In addition, the photopolymerizable monomer may be a photoresist material. The photoresist material may be a silicone or epoxy material.

The photoresist material may be a commercial photoresist. The commercial photoresist materials are AZ Electronics Materials' AZ 5214E PR, AZ 9260 PR, AZ AD Promoter-K (HMDS), AZ nLOF 2000 Series, AZ LOR-28 PR, AZ 10xT PR, AZ 5206-E, AZ GXR-601, AZ 04629; SU-8 from MICROCHEM, 950 PMMA, 495 PMMA; S1800 from micropossit; DNR-L300, DSAM, DPR, DNR-H200, DPR-G from Dongjin Semichem; Kotem's CTPR-502, but is not limited thereto.

The mixture may further include a photoinitiator for photocuring according to the type of the photocurable polymer compound. The photoinitiator may be a benzophenone-based compound, a thioxanthone-based compound, a benzoin-based compound, or an oxime-based compound, but is not limited thereto.

Examples of the benzophenone-based compound include benzophenone, benzoyl benzoate, methyl 2-benzoylbenzoate, 4-phenyl benzophenone, hydroxybeonzophenone, acrylated benzophenone acrylate, 4,4-bis(dimethylamino)benzophenone, 4,4-dichlorobenzophenone, 3,3-dimethyl-2-methoxy benzophenone, and the like, but is not limited thereto.

Examples of the thioxanthone-based compound include thioxantone, 2-methyl thioxantone, isopropyl thioxantone, and 2,4-diethyl thioxantone, 2,4-diiospropyl thioxantone, 2-chloro thioxantone, and the like, but are not limited thereto.

Examples of the benzoin-based compound include benzoine, benzoine methyl ether, benzoine ethyl ether, benzoine isopropyl ether, benzoine isobutyl ether, benzyl dimethyl ketal, and the like, but are not limited thereto.

Examples of the oxime compound are 2-(o-benzoyloxime)-1-[4-(phenylthio)phenyl]-1,2-octanedione(2-(o-benzoyloxime)-1-[4-(phenylthio)phenyl]-1,2,-octandione and 1-(o-acetyloxime)-1-[9-ethyl-6-(2-methylbenzoyl)-9H-carbazol-3-yl]ethanone(1-(o-acetyloxime)-1-[9-ethyl-6-(2-methylbenzoyl)-9H-carbazol-3-yl]ethanone), but is not limited thereto.

Meanwhile, the mixture may further include a crosslinking agent for crosslinking.

Preferably, the crosslinking agent is ethylene glycol di(metha) acrylate, polyethyleneglycol di(metha) acrylate, trimethylolpropane)di(metha)acrylate, trimethylolpropane tri(metha)acrylate, pentaerythritol tri(metha)acrylate, pentaerythritol tetra(metha)acrylate, 2-trisacrylo oxymethylethylpthalic acid, propylene glycol di(metha)acrylate, polypropyleneglycol di(metha)acrylate, dipentaerythritol penta(metha)acrylate and dipentaerythritol hexa(metha)acrylate), and combinations thereof, but is not limited thereto.

Referring to FIG. 104, when the inner phase is characterized in that it contains a metal halide perovskite and a polymer, the solvent of the inner phase may be volatilized. Volatilizing the solvent in the inner phase may be performed by a method of decompressing the curable emulsion composition. When the solvent on the inner phase is removed by the above process, the metal halide perovskite may be encapsulated by a polymer contained in the inner phase.

Referring to FIG. 105, when the inner phase includes a metal halide perovskite and an encapsulating resin, the metal halide perovskite may be encapsulated in various ways depending on the type of the encapsulating resin.

In particular, when the encapsulation resin is a thermosetting resin, it may be converted to liquid phase by applying heat above the melting point of the wax-based compound in order to apply it to the hardened emulsion composition. The melting point of the waxy compound may vary depending on the type of waxy compound, but preferably the melting point of the waxy compound below 100° C. If the melting point exceeds 100° C. to apply to hardened emulsion compounds, the heat-vulnerable metal halide perovskite crystal structure may be decomposed.

If the above encapsulation resin is a wax-based compound, it may be converted to liquid phase by applying heat above the melting point of the wax-based compound in order to apply it to the hardened emulsion composition. The melting point of a waxy compound may vary depending on the type of waxy compound, but preferably the melting point of the waxy compound below 100° C. If the melting point exceeds 100° C., in the process of applying heat beyond the melting point of the above wax-based compound for application to the hardened emulsion composition, the metal halide perovskite crystal structure vulnerable to heat may be decomposed.

When the encapsulating resin is a wax-based compound, the metal halide perovskite may be encapsulated by removing heat applied to form a curable emulsion composition.

A dispersion in which encapsulated metal halide perovskite particles are dispersed is prepared by the encapsulation process.

Referring to FIGS. 104 and 105, the curable emulsion composition is then coated on a substrate and dried to form a coating film.

The material of the substrate (10) is glass, sapphire, quartz, silicon, polyethylene terephthalate (PET), polystyrene (PS), polyimide (PI), polyvinyl chloride (PVC), polyvinylpyrrolidone (PVP), polyethylene (PE), or the like, but is not limited thereto.

The method provided on the substrate (10) can be selected from known coating method, for example, a spin coating method, a cast method, a Langmuir-Blodgett (LB) method, a spray coating method, a dip coating method, a gravure coating method, a reverse offset method, a screen printing method, a slot-die coating method, a nozzle printing method, and a dry transfer printing method, but the present disclosure is not limited thereto. Drying may be performed by a known drying method generally known in the art, for example, a hot air heating method, or an induction heating method, but is not limited thereto.

The coating film formed by the coating and drying has a structure in which the metal halide perovskite encapsulated in the photocurable polymer compound in the external phase composition is uniformly dispersed. Subsequently, light is irradiated to the coating film to cure the photocurable polymer compound to prepare a wavelength converting body having a structure in which the metal halide perovskite encapsulated in the second dispersion medium is uniformly dispersed. At this time, the second dispersion medium is characterized in that it is prepared by curing the photocurable polymer compound.

<Perovskite Nanoparticles with Suppressed Defects Through the Addition of Medium-Sized Organic Cations>

Hereinafter, a perovskite nanoparticle in which defect generation is suppressed through the addition of medium-sized organic cation, which is the core of the present disclosure, is provided.

In general, the crystal structure of perovskite forms a BX₆ octahedron between a B metal substance and a halogen element, and a cation A is located between the formed BX₆ octahedron to form a crystal structure. Therefore, the size of the A cation is limited by the size of the BX₆ octahedron. At this time, the combination of A, B, and X that can make the perovskite crystal can be determined simply by calculating the tolerance factor (t). The tolerance factor is defined by the following equation.

$$t = \frac{{R}_{A} + {R}_{X}}{\sqrt{2} ( {R}_{B} + {R}_{X} )}$$

$$( {R}_{A} , {R}_{B} , {R}_{X} are the ionic radii of A , B and X , respectively )$$

In order for the perovskite to have a three-dimensional crystal structure, it is preferable that the tolerance factor has a value of about 0.8 or more and about 1.0 or less. Since the tolerance factor also depends on the central metal B and the halide anion X, a reference point for this disclosure should be set. To determine the boundary point of the tolerance factor, the reference element of central metal B can be set to Pb and the reference element of the X can be set to Bromide, and FAPbBr₃ can be taken as the reference crystal. This is because FAPbBr₃ is a single cation and has a tolerance factor close to 1 (approximately 1.01), the crystal itself is stable, and the luminescence efficiency is high. Therefore, in order to prevent confusion in the present disclosure, a table is presented directly. Therefore, if this criterion (B=Pb and X=Br criterion) has a tolerance factor of 1.01 or more, considering the radius of A, A cannot be included in the space between the BX₆ octahedron and the crystal is distorted. For example, when B is Pb²⁺ and X is Br⁻, the cation at site A may be Rb⁺, Cs⁺, methylammonium, or formamidinium.

However, when the metal halide perovskite light emitter is formed only with the combination of the A site cation that satisfies the above-described tolerance factor condition of 0.8 or more and 1.01 or less, the crystal structure becomes unstable due to the small size of the A site particle. Since the bonding force of the metal halide perovskite is weakened, there are many defects that may inevitably reduce the luminescence efficiency and stability of the metal halide perovskite light emitter. At this time, when included in the perovskite crystal alone, if medium-sized organic cations having a tolerance factor greater than 1.01 and smaller than 3 are included in the crystal, defects of the perovskite light emitter can be effectively controlled.

Accordingly, in the present disclosure, the first monovalent cation (A₁) capable of making a tolerance factor of 1.01 or less when included in the A-site of a perovskite crystal alone, and a tolerance factor of 1.01 or more and less than 3 are produced. In a state in which the second monovalent organic cation (A₂) is mixed; It provides a colloidal perovskite light emitting particle, characterized in that the second monovalent organic cation (A₂) is simultaneously included both in the inside and the surface of the perovskite crystal.

A₂ organic cations having a tolerance factor greater than 1.01 are relatively difficult to be included inside metal halide perovskite crystals because they have a size larger than the space between BX₆ octahedra. Therefore, a small amount of A₂ organic cation can be accommodated to form a metal halide perovskite crystal, but when a larger amount of A₂ organic cation is added than the amount capable of forming a crystal, an excessive amount of A₂ organic cation is not included in a perovskite crystal and is located on the surface of perovskite nanoparticles or at the grain boundaries of perovskites.

Since it is located on the surface of nanoparticles and plays a role of suppressing defects while enclosing particles like a shell, the high luminescence efficiency can be maintained without gradual decrease. In addition, the size of the particles is reduced by the heavy ions surrounding the surface like a shell, and the confinement of excitons or charges is better, thereby increasing the radiative recombination. Furthermore, if it has a symmetrical structure such as guanidinium among medium-sized cations, light emission can be more efficiently and stably emitted.

When only one type of cation is used purely, the tolerance factor (t) of the metal halide perovskite material is determined by the related literature [Nature Photonics, 2015, 11, 582; Chemical Science, 2016, 7, 4548; Chemical Science, 2015, 6, 3430; Science, 2016, 354, 206; Journal of Materials Chemistry A, 2017, 5, 18561. For example:

When the tolerance factors are calculated for the representative A, B, and X site ions constituting the perovskite, the following table is shown.

| | | X site | | | |
|---|---|---|---|---|---|
| B site = Be | | Floride | Chloride | Bromide | Iodide |
| A site | Ammonium | 1.119 | 1.023 | 1.003 | 0.977 |
| | Hydroxylammonium | 1.404 | 1.242 | 1.209 | 1.163 |
| | Methylammonium | 1.408 | 1.245 | 1.212 | 1.166 |
| | Hydrazinium | 1.408 | 1.245 | 1.212 | 1.166 |
| | Azetidinium | 1.543 | 1.349 | 1.309 | 1.254 |
| | Formamidinium | 1.555 | 1.358 | 1.317 | 1.262 |
| | Imidaozolium | 1.575 | 1.374 | 1.332 | 1.275 |
| | Dimethylammonium | 1.632 | 1 417 | 1.373 | 1.313 |
| | Pyrrolinium | 1.632 | 1.417 | 1.373 | 1.313 |
| | Ethylammonium | 1.64 | 1.424 | 1.379 | 1.318 |
| | Guanidinium | 1.657 | 1.436 | 1.391 | 1.329 |
| | Tetramethylammonium | 1.714 | 1.48 | 1.432 | 1.366 |
| | Thiazolium | 1.828 | 1.568 | 1.514 | 1.441 |
| | Tropylium | 1.881 | 1.608 | 1.552 | 1.476 |
| | | X site | | | |
| B site = Mg | | Floride | Chloride | Bromide | Iodide |
| A site | Ammonium | 0.968 | 0.914 | 0.902 | 0.886 |
| | Hydroxylammonium | 1.215 | 1.11 | 1.087 | 1.056 |
| | Methylammonium | 1.218 | 1.112 | 1.09 | 1.058 |
| | Hydrazinium | 1.218 | 1.112 | 1.09 | 1.058 |
| | Azetidinium | 1.335 | 1.205 | 1.177 | 1.138 |
| | Formamidinium | 1.345 | 1.213 | 1.185 | 1.145 |
| | Imidaozolium | 1.363 | 1.227 | 1.198 | 1.158 |
| | Dimethylammonium | 1.412 | 1.266 | 1.235 | 1.191 |
| | Pyrrolinium | 1.412 | 1.266 | 1.235 | 1.191 |
| | Ethylammonium | 1.42 | 1.272 | 1.24 | 1.196 |
| | Guanidinium | 1.434 | 1.283 | 1.251 | 1.206 |
| | Tetramethylammonium | 1.483 | 1.322 | 1.288 | 1.24 |
| | Thiazolium | 1.582 | 1.4 | 1.361 | 1.308 |
| | Tropylium | 1.628 | 1.437 | 1.396 | 1.339 |
| | | X site | | | |
| B site = Pb | | Floride | Chloride | Bromide | Iodide |
| A site | Ammonium | 0.784 | 0.771 | 0.768 | 0.763 |
| | Hydroxylammonium | 0.984 | 0.936 | 0.925 | 0.909 |
| | Methylammonium | 0.987 | 0.938 | 0.927 | 0.912 |
| | Hydrazinium | 0.987 | 0.938 | 0.927 | 0.912 |
| | Azetidinium | 1.081 | 1.016 | 1.001 | 0.98 |
| | Formamidinium | 1.09 | 1.023 | 1.008 | 0.987 |
| | Imidaozolium | 1.104 | 1.035 | 1.019 | 0.997 |
| | Dimethylammonium | 1.144 | 1.068 | 1.051 | 1.026 |
| | Pyrrolinium | 1.144 | 1.068 | 1.051 | 1.026 |
| | Ethylammonium | 1.15 | 1.072 | 1.055 | 1.03 |
| | Guanidinium | 1.161 | 1.087 | 1.064 | 1.039 |
| | Tetramethylammonium | 1.201 | 1.115 | 1.095 | 1.068 |
| | Thiazolium | 1.281 | 1.181 | 1.158 | 1.126 |
| | Tropylium | 1.319 | 1.212 | 1.187 | 1.153 |
| | | X site | | | |
| B site = Sn | | Floride | Chloride | Bromide | Iodide |
| A site | Ammonium | 0.797 | 0.781 | 0.778 | 0.773 |
| | Hydroxylammonium | 1 | 0.948 | 0.937 | 0.92 |
| | Methylammonium | 1.003 | 0.951 | 0.939 | 0.922 |
| | Hydrazinium | 1.003 | 0.951 | 0.989 | 0.922 |
| | Azetidinium | 1.099 | 1.03 | 1.014 | 0.992 |
| | Formamidinium | 1.108 | 1.037 | 1.021 | 0.998 |
| | Imidaozolium | 1.122 | 1.049 | 1.032 | 1.009 |
| | Dimethylammonium | 1.163 | 1.082 | 1.064 | 1.038 |
| | Pyrrolinium | 1.163 | 1.082 | 1.064 | 1.038 |
| | Ethylammonium | 1.169 | 1.087 | 1.069 | 1.043 |
| | Guadnidinium | 1.18 | 1.096 | 1.078 | 1.051 |
| | Tetramethylammonium | 1.221 | 1.13 | 1.11 | 1.081 |
| | Thiazolium | 1.302 | 1.197 | 1.173 | 1.14 |
| | Tropylium | 1.34 | 1.228 | 1.203 | 1.167 |
| | | X site | | | |
| B site = Eu | | Floride | Chloride | Bromide | Iodide |
| A site | Ammonium | 0.791 | 0.776 | 0.773 | 0.768 |
| | Hydroxylammonium | 0.992 | 0.942 | 0.931 | 0 915 |
| | Methylammonium | 0.995 | 0.944 | 0.933 | 0.917 |
| | Hydrazinium | 0.995 | 0.944 | 0.933 | 0.917 |
| | Azetidinium | 1.09 | 1.023 | 1.008 | 0.986 |
| | Formamidinium | 1.099 | 1.03 | 1.014 | 0.992 |
| | Imidaozolium | 1.113 | 1.042 | 1.026 | 1.003 |
| | Dimethylammonium | 1.154 | 1.075 | 1.057 | 1.032 |
| | Pyrrolinium | 1.154 | 1.075 | 1.057 | 1.032 |
| | Ethylammonium | 1.159 | 1.08 | 1.062 | 1 037 |
| | Guadnidinium | 1.171 | 1.089 | 1.071 | 1.045 |
| | Tetramethylammonium | 1.211 | 1.122 | 1.102 | 1.074 |
| | Thiazolium | 1.292 | 1.189 | 1.166 | 1.133 |
| | Tropylium | 1.329 | 1.22 | 1.195 | 1.16 |

The A₂ organic cation may be ethylammonium, guanidinium, tert-butylammonium, diethylammonium, dimethylammonium, ethane-1.2.-diammonium, imidazolium, n-propylammonium, iso-propylammonium, pyrrolidinium, or combinations thereof, but is not limited thereto.

At this time, the amount of the A₂ organic cation that may be included in the crystal may vary depending on the type of A₁ cation and A₂ organic cation that are added to the perovskite crystal. When the A₂ cation are included in the crystal, the crystal is unstable in terms of enthalpy due to steric hinderance due to the large size of A₂, but the crystal may be stabilized due to an increase in entropy due to mixing. Therefore, the amount of the A₂ organic cation that can be included in the crystal can be determined by extracting the range that the generated energy by summing the enthalpy energy change and the entropy energy change is negative with respect to the proportion of the A2 precursor. The enthalpy energy change and the entropy energy change can be obtained by DFT calculation. In particular, when the A₁ organic cation is formamidinium, the A₂ organic cation is guanidinium, the B-site cation is Pb²⁺, and the X-site anion is Br⁻. When the proportion of the mixture changes to 0%, 12.5%, 25%, 50%, 75%, 100%, the enthalpy energy increases to 0 meV, 7.7 meV, 17.5 meV, 44.5 meV, 72.7 meV, and 82.5 meV, and, the entropy energy changes to 0 meV, −10 meV, −14.7 meV, −17.9 meV, −14.7 meV, and 0 meV, respectively.

The A₂ organic cation contained in the crystal can stabilize the perovskite crystal due to the entropy effect and suppress the generation of defects in the crystal, and an excessive amount of A₂ organic cation that is not contained inside the perovskite crystal can passivate defects generated on the surface of the perovskite nanocrystal particles by forming a structure surrounding the perovskite nanocrystal particles. (see FIG. 114).

It may be characterized in that the ratio of the A₂ organic cation to the mixture of the A1 cation and A₂ organic cation among the monovalent cation of the A site (i.e. A site cation precursor ratio) is more than the ratio that can be maximally included in the perovskite crystal, and is less than or equal to a ratio when the surface of the metal halide perovskite nanoparticles is completely enclosed, for example, 5% or more and 60% or less. In addition, preferably the ratio may includes a range where the lower value among two numbers selected from 5%, 6%, 7%, 8%, 9%, 10%, 11%, 12%, 13%, 14%, 15%, 16%, 17%, 18%, 19%, 20%, 22%, 24%, 26%, 28%, 30%, 32%, 34%, 36%, 38%, 40%, 45%, 50%, 55%, and 60% is a lower limit value and a higher value of the two numbers is an upper limit value.

If the ratio of the A₂ organic cations is less than 5% outside the above range, all of the mixed A₂ organic cations are contained inside the perovskite crystal, so that the defects formed on the surface of the perovskite nanoparticles cannot be effectively controlled, and if it exceeds 60%, the size of the perovskite nanoparticles is greatly reduced because an excess of A₂ organic cations contained in the perovskite nanoparticles are more than the amount that can completely cover the surface of the perovskite nanoparticles. As the surface-to-volume ratio increases, the quantum efficiency decreases, and the color purity decreases due to light emission depending on the quantum confinement effect.

Preferably, the A₂ organic cation may be guanidinium. When the A₂ ion is guanidinium, the number of hydrogen bonds that can be formed in the crystal increases, and thus the inside of the perovskite crystal may be additionally stabilized.

The A₂ organic cation is ethylammonium, guanidinium, tert-butylammonium, diethylammonium, dimethylammonium, ethane-1.2.-diammonium, imidazolium, n-propylammonium, iso-propylammonium, pyrrolidinium, and combinations thereof, but is not limited thereto.

At this time, the amount of the A₂ organic cation that may be included in the crystal may vary depending on the type of A₁ cation and A₂ organic cation that are added to the perovskite crystal. When the A₂ cation are included in the crystal, the crystal is unstable in terms of enthalpy due to steric hinderance due to the large size of A₂, but the crystal may be stabilized due to an increase in entropy due to mixing. Therefore, the amount of the A₂ organic cation that can be included in the crystal can be determined by extracting a range that the generated energy summing the enthalpy energy change and the entropy energy change is negative with respect to the cation precursor ratio (i.e. the ratio of A2 precursor to the total A site cation precursors). The enthalpy energy change and the entropy energy change can be obtained by DFT calculation.

The A₂ organic cation contained in the crystal can stabilize the perovskite crystal due to the entropy effect and suppress the generation of defects in the crystal, and an excessive amount of A₂ organic cation that is not contained inside the perovskite crystal can passivate defects generated on the surface of the perovskite nanocrystal particles by forming a structure surrounding the perovskite nanocrystal particles. (see FIG. 114).

It may be characterized in that the ratio of the A₂ organic cation to the mixture of the A₁ cation and A₂ organic cation among the monovalent cation of the A site (i.e. A site cation ratio) is more than the ratio that can be maximally included in the perovskite crystal, and is less than or equal to a ratio when the surface of the metal halide perovskite nanoparticles is completely enclosed, for example, 5% or more and 60% or less.

In addition, preferably the ratio may include a range between two numbers selected from 5%, 6%, 7%, 8%, 9%, 10%, 11%, 12%, 13%, 14%, 15%, 16%, 17%, 18%, 19%, 20%, 22%, 24%, 26%, 28%, 30%, 32%, 34%, 36%, 38%, 40%, 45%, 50%, 55%, and 60%, in which a lower value of the two numbers is a lower limit value and a higher value of the two numbers is an upper limit value.

If the ratio of the A₂ organic cations is less than 5% outside the above range, all of the mixed A₂ organic cations are contained inside the perovskite crystal, so that the defects formed on the surface of the perovskite nanoparticles cannot be effectively controlled, and if it exceeds 60%, the size of the perovskite nanoparticles is greatly reduced because an excess of A₂ organic cations contained in the perovskite nanoparticles are more than the amount that can completely cover the surface of the perovskite nanoparticles. As the surface-to-volume ratio increases, the quantum efficiency decreases, and the color purity decreases due to light emission by the quantum confinement effect.

Preferably, the A₂ organic cation may be guanidinium. When the A₂ ion is guanidinium, the number of hydrogen bonds that can be formed in the crystal increases, and thus the inside of the perovskite crystal may be additionally stabilized.

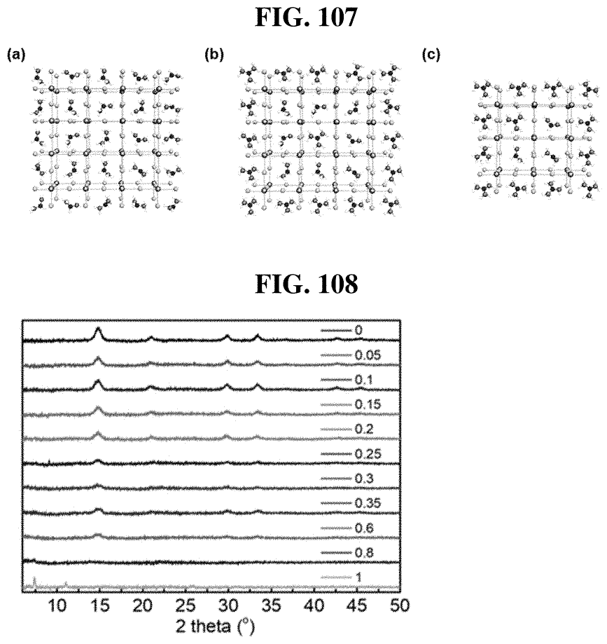

In an embodiment of the present disclosure, the A₁ cation may be formamidinium (FA), B may be Pb, X may be Br, and the A₂ organic cation may be guanidinium. Referring to the schematic diagram of FIG. 106, as A₂ is added to the A₁BX₃ perovskite nanocrystal particles (FIG. 107(a)), when the appropriate amount (more than the ratio that can be maximally contained in the crystal and less than the ratio when the surface of metal halide perovskite nanocrystal particles is completely enclosed) added, only a part of A₂ ions are contained in the crystal and the remaining A₂ ions form a surrounding structure (FIG. 107(b)) and A₂ exceeds an appropriate amount (When added in excess than the amount capable of completely covering the surface of the perovskite nanoparticles), the size of the perovskite nanocrystals decreases (FIG. 107(c)).

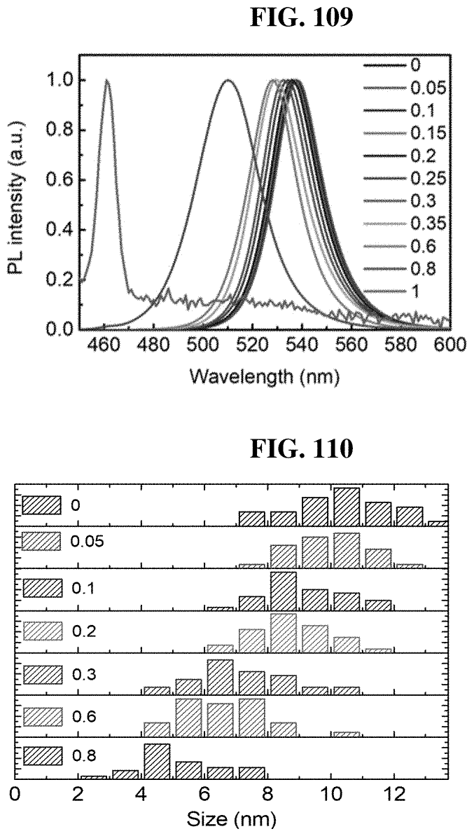

Accordingly, referring to FIGS. 108 and 109, in the above embodiment, when the mixing ratio of A₂ is 5% or less, all of A₂ is contained inside the perovskite crystal to expand the crystal to make the steady-state photoluminescence wavelength red-shifted. On the other hand, when 5% or more of A₂ is added, the crystal lattice does not change and the steady-state photoluminescence wavelength is blue-shifted. The blue-shift may be due to a decrease in the size of perovskite nanoparticles (FIG. 110).

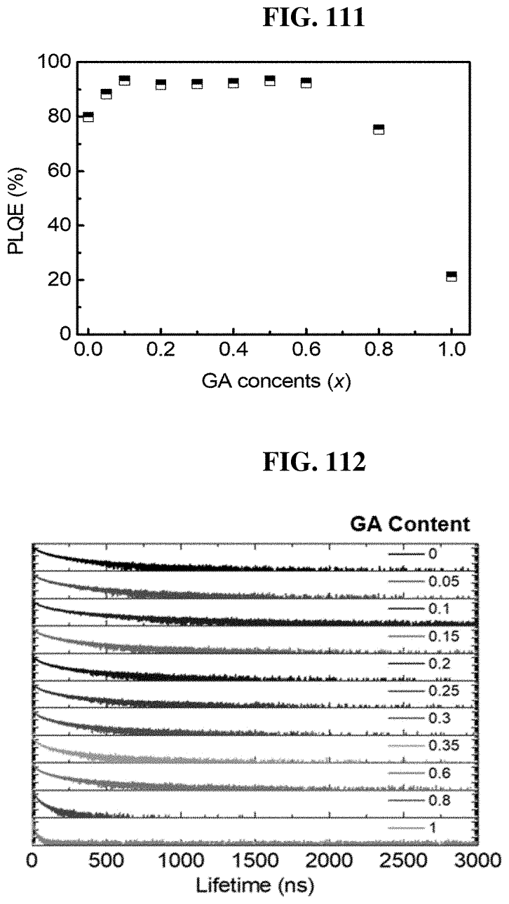

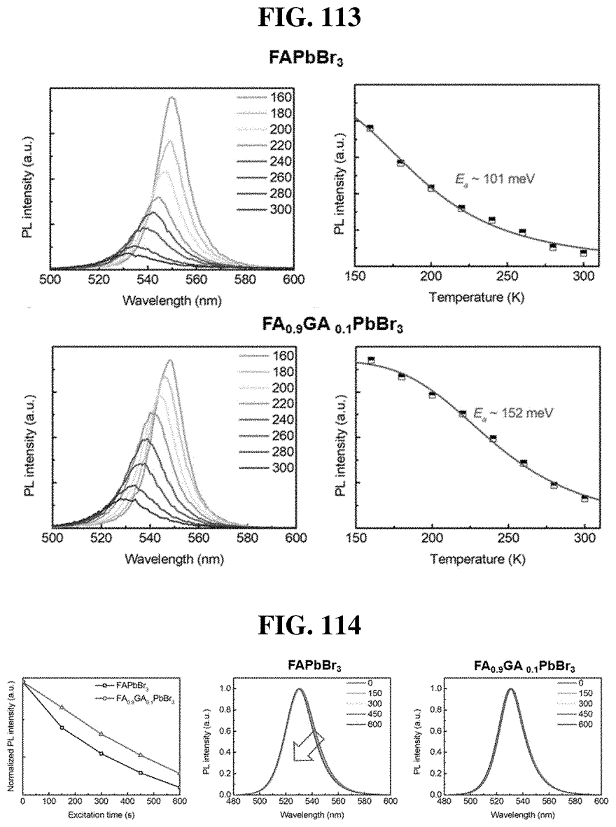

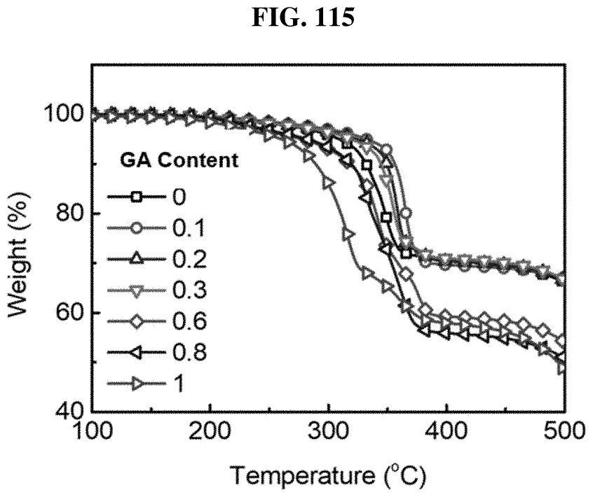

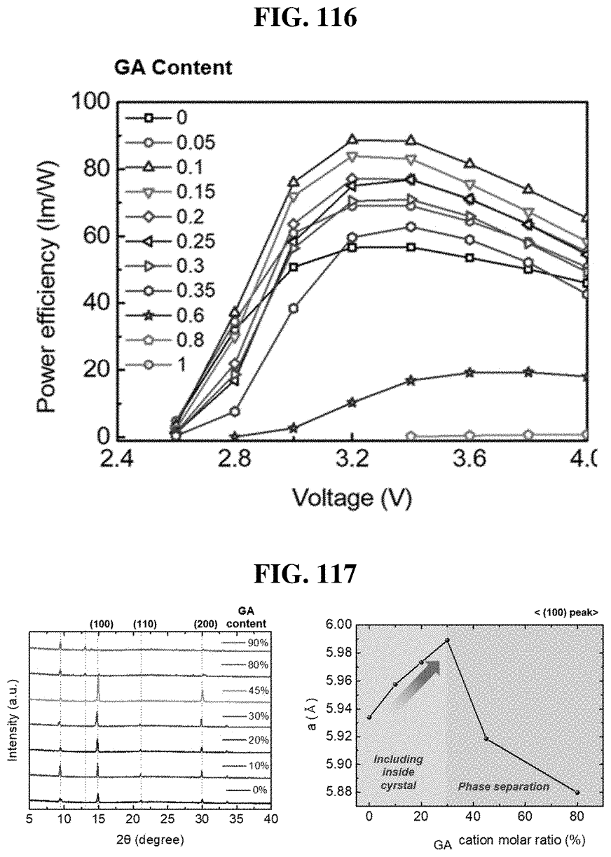

In the above embodiment, as a result of measuring the photoluminescence properties before and after adding A₂ organic cation with ratio a 5% or more and 60% or less, which corresponds to ratio range where the ratio of the A₂ organic cation to the mixture of the A₁ cation and A₂ organic cation among the monovalent cations at the A site is greater than or equal to the ratio that can be maximally included in the perovskite crystal, and the metal halide perovskite is less than the ratio when completely surrounding the surface of perovskite nanoparticles, it was confirmed that after adding A₂ organic cations, photoluminescence quantum efficiency (PLQY, photoluminescence quantum yield) (FIG. 111) increased, the photoluminescence lifetime (PL lifetime) was extended (FIG. 112), and the exciton binding energy determined by temperature dependent photoluminescence increased (FIG. 113), and the stability against thermal decomposition upon UV irradiation was improved (FIG. 114), the stability against thermal decomposition was improved (FIG. 115), and the luminous efficiency when manufacturing a light emitting diode was improved (FIG. 116).

Thus, in the perovskite material according to the present disclosure, the medium-sized monovalent organic cations (A₂) contained in the perovskite crystal stabilize the perovskite crystal due to the entropy effect and suppress defects in the crystal. A₂ cations that are not included in the perovskite crystal form a structure surrounding the perovskite nanocrystal particles, and passivates defects that are formed on the surface of the perovskite nanocrystal particles. As a result, photoluminescence quantum efficiency, photoluminescence lifetime, and stability are improved, and thus, it can be effectively used in a light-emitting layer or a wavelength conversion layer of a light-emitting device.

An example of a light-emitting device including a perovskite material in which defect generation is controlled through the addition of a medium-sized organic cation according to the present disclosure is the same as the description of the light-emitting device described above, and a detailed description is omitted to avoid redundant description.

<Perovskite with Suppressed Defects Through 4 Types of Mixed Cationic Structure>

Hereinafter, there is provided a perovskite light emitter in which the generation of defects is controlled using the four kinds of mixed cationic structure, which is the core of the present disclosure.

In general, the crystal structure of perovskite is that the B metal substance and the halogen element form a BX₆ octahedron, and a cation A is located between the formed BX₆ octahedron to form a crystal structure. Therefore, the size of the A cation is limited by the size of the BX₆ octahedron. At this time, the combination of A, B, and X that can make the perovskite crystal can be determined simply by calculating the tolerance factor (t). The tolerance factor is defined by the following equation.

$$t = \frac{{R}_{A} + {R}_{X}}{\sqrt{2} ( {R}_{B} + {R}_{X} )}$$

$$( {R}_{A} , {R}_{B} , {R}_{X} are the ionic radii of A , B and X , respectively )$$

In order for the perovskite to have a three-dimensional crystal structure, it is preferable that the tolerance factor has a value of 0.8 or more and 1.1 or less. When the tolerance factor exceeds the above range and has a value of 1.01 or more, the radius of A is not included in the interspace between the BX₆ octahedra and the crystal is distorted. For example, when B is Pb²⁺ and X is Br⁻, the cation at site A may be Rb⁺, Cs⁺, methylammonium, or formamidinium.

However, when the metal halide perovskite light emitter is formed only with the combination of the A site cation that satisfies the above-described tolerance factor condition of 0.8 or more and 1.01 or less, the crystal structure becomes unstable due to the small size of the A site particle. Since the bonding force of the metal halide perovskite is weakened, there are many defects that may inevitably reduce the luminescence efficiency and stability of the metal halide perovskite light emitter. At this time, when included in the perovskite crystal alone, if medium-sized organic cations having a tolerance factor greater than 1.01 and smaller than 3 are included in the crystal, defects of the perovskite light emitter can be effectively controlled.

The tolerance factor (t) of the APbX₃ (A=medium cation, X=I, Br, Cl) structure obtained using only the medium cation added here is 1.1, 1.15, 1.2, 1.25, 1.3, 1.35, 1.4, 1.45, 1.5, It can be 1.55, 1.6, 1.65, 1.7, 1.75, 1.8, 1.85, 1.9, 1.95, 2.0, 2.1, 2.2, 2.3, 2.4, 2.5, 2.6, 2.7, 2.8, 2.9, 3.0. The range can be determined by using the smaller value of the two numbers selected from the above numbers as the lower limit value and the larger value as the upper limit value. The most preferred range when adding a medium-sized cation (e.g. guanidinium) to FAPbBr₃ is in the range of 1.6-2.1.

In the present disclosure, a material includes at least one type of cation having a large t value such as guanidinium is unconditionally, and includes the cations of 2, 3, 4, 5, 6, and 7 types.

Examples of embodiments of the present disclosure include a halide perovskite polycrystalline thin film containing the mixed cation and a device using the same.

Examples of embodiments of the present disclosure include halide perovskite nanocrystal particles containing the mixed cations and a device using the same.

According to the present disclosure, when formed as nanoparticles, it was possible to achieve high efficiency even with two cations including heavy ions, and when formed as a polycrystalline thin film, it was possible to implement high efficiency with more than four cations.

In addition, when organic cations with an above tolerance factor greater than 1.01 and less than 3 are included in the crystal in excess of a certain level or more, the tolerance factor of the mixed cationic perovskite crystal can become larger than 1, resulting in deterioration of the crystal stability and the luminescence properties. At this time, if some of the A-site cations that satisfy the tolerance factor condition of 0.8 or more and less than 1.01 are replaced with cations providing a lower tolerance factor, the tolerance factor of the entire mixed cation perovskite crystal decreases. At time, it is possible to maximize the defect suppression effect by including the medium-sized cations in the crystal at a higher ratio that provide a tolerance factor of 1.01 or more and less than 3.

The content of the medium-sized organic cation can range from 5% to 60%. For example, it may be 5%, 6%, 7%, 8%, 9%, 10%, 11%, 12%, 13%, 14%, 15%, 20%, 30%, 40%, 50%, and 60%. In this proportion, the photoluminescence efficiency is best. Looking at the optimum point of the external quantum efficiency from the viewpoint of the electroluminescent device, it may be preferably 8% to 20% or less, and more preferably 8% to 15% or less.

In this disclosure, a perovskite polycrystalline thin film characterized by the simultaneous inclusion of cations inside and on the surface of perovskite crystals, in which the cation is comprised of the first monovalent cation (A₁, A₃, A₄) that can make a tolerance factor of less than 1 and the second monovalent cation (A₂) that can make a tolerance factor of more than 1.01 and less than 3.

A₂ organic cations having a tolerance factor greater than 1.01 are relatively difficult to be included in metal halide perovskite crystals because they have a size larger than the interspace between BX6 octahedra. Therefore, a small amount of A₂ organic cations can form a metal halide perovskite crystal, but when a larger amount of A₂ organic cations are added than the amount capable of forming a crystal, an excess of A₂ organic cations will not be used to form a perovskite crystal but instead are included in the perovskite grain boundaries or are located on the surface of the perovskite polycrystalline thin film.

The A₂ organic cation is ethylammonium, guanidinium, tert-butylammonium, diethylammonium, dimethylammonium, ethane-1.2.-diammonium, imidazolium, n-propylammonium, iso-propylammonium, pyrrolidinium, and combinations thereof, but is not limited thereto.

At this time, the amount of A₂ organic cations that may be included in the crystal may vary depending on the types of A₁, A₃, and A₄ cations constituting the perovskite crystal and the type of A₂ organic cations added. When the A₂ particles are included in the crystal, the crystal is unstable in terms of enthalpy due to steric hinderance caused by the large size of A₂, but the crystal may be stabilized due to an increase in entropy by mixing. Therefore, the amount of the A₂ organic cation that can be included in the crystal can be determined by extracting a range that the generated energy summing the enthalpy energy change and the entropy energy change is negative with respect to the precursor ratio of the cation. The enthalpy energy change and the entropy energy change can be obtained by DFT calculation.

The A₂ organic cation contained inside the crystal can stabilize the perovskite crystal and suppress the generation of defects in the crystal due to the entropy effect.

Among the monovalent cations at the A site, the ratio of the A₂ organic cation to the mixture of the A₁, A₃, A₄ cation and A₂ organic cation is the ratio that can be included in the perovskite crystal. Hereinafter, for example, it may be characterized in that it is 5% or more and 60% or less.

In addition, preferably, the ratio is 5%, 6%, 7%, 8%, 9%, 10%, 11%, 12%, 13%, 14%, 15%, 16%, 17%, 18%, 19%, 20%, 22%, 24%, 26%, 28%, 30%, 32%, 34%, 36%, 38%, 40%, 45%, 50%, 55%, and 60% and include a range in which a lower value among of two numbers selected from the above is a lower limit value and a higher value is an upper limit value.

When the ratio of A₂ organic cations is less than 5% outside the above range, all of the mixed A₂ organic cations are contained inside the perovskite crystal, so that defects formed on the surface of the perovskite crystal cannot be effectively suppressed, and in the case of exceeding 30%, an excessive amount of A₂ organic cations more than the amount that can completely cover the surface of the perovskite crystal will cause phase separation of the perovskite crystal, and the formation of perovskite crystal other than three-dimensional perovskite crystal, which results in poor luminescence efficiency and electrical conduction characteristics. Preferably, the A₂ organic cation may be guanidinium. When the A₂ ion is guanidinium, the number of hydrogen bonds that can be formed inside the crystal increases, so that the inside of the perovskite crystal can be additionally stabilized.

In addition, preferably, the ratio is 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, and 60%. It may include a range in which the lower value of the two numbers out of the above ratios is the lower limit value and the higher value has the upper limit value. Preferably the ratio includes a range from 8% to 30%. In the case of a polycrystalline thin film, when quadruple ions containing guanidinium are used, the most optimal device luminous efficiency can be obtained in a range near 10% of the guanidinium ratio, that is, 8%-20%. Even when all including particles and polycrystals are considered, the most optimal device luminous efficiency can be obtained in a range around 10%, that is, in a range of 8%-15%.

If the proportion of A₂ organic cations is less than 5% outside the above range, both mixed A₂ organic cations are contained inside the perovskite crystal and the defects formed on the perovskite crystal surface cannot be effectively controlled, and if the proportion exceeds 30%, the remaining A₂ organic cations can cause phase separation of perovskite crystals and thus form perovskite crystals other than three-dimensional perovskite crystals, which can cause lower luminescence efficiency and electrical conductivity than those in three-dimensional perovskite crystals.

The ratio of the A₁, A₃ and A₄ cations of the monovalent cations of the above A site to the all A cation mixture can be taken as the rest of proportion excluding the proportion of the above A₂ organic cations, and the above disclosure can be characterized by the combination that can still allow the tolerance factor to be 1.01 or less even after the tolerance factors increases by including A₂ organic cation in the perovskite crystal.

Preferably, A₁ and A₃ are formamidinium (FA) and cesium (Cs), respectively, and A₄ is methylammonium (MA). The ratio of A₃ is 1%, 2%, 3%, 4%, 5%, 6%, 7%, 8%, 9%, 10%, 11%, 12%, 13%, 14%, 15%, 16%, 17%, 18%, 19%, and 20%. The lower value of the two numbers selected from the above is the lower limit and the higher value has the upper limit. The ratio of A₄ is 5%, 6%, 7%, 8%, 9%, 9.1%, 9.2%, 9.3%, 9.4%, 9.5%, 9.6%, 9.7%, 9.8%, 9.9%, 10%, 10.1%, 10.2%, 10.3%, 10.4%, 10.5%, 10.6%, 10.7%, 10.8%, 10.9%, 11%, 12%, 13%, 14%, and 15%, and may include a range in which the lower value of the two numbers selected from the above is the lower limit value and the higher value have the upper limit value. The ratio of A₁ may fall into the remaining range excluding A₃ and A₄ above.

In an embodiment of the present disclosure, the combination of A₁, A₃, and A₄ cation is composed of formamidinium (FA), methylammonium (MA), cesium (Cs), B is taken as Pb, and X is taken as Br. Accordingly, the A₂ organic cation can be used as guanidinium.

In an embodiment of the present disclosure, the combination of A₁, A₃, and A₄ cation is composed of formamidinium (FA), methylammonium (MA), cesium (Cs), B is taken as Pb, and X is taken as Br. Accordingly, the A₂ organic cation can be used as guanidinium.

Referring to FIG. 117, according to the mixing ratio of adding A₂ to the A₁BX₃ perovskite polycrystalline thin film (FIG. 117(a)), A₂ ions are contained in the crystal up to about 25%, thereby increasing the lattice constant of the crystal. (FIG. 117(b)), when an A₂ organic cation in excess of an appropriate amount is added, a new crystal is formed and the crystal structure is changed to show a different pattern.

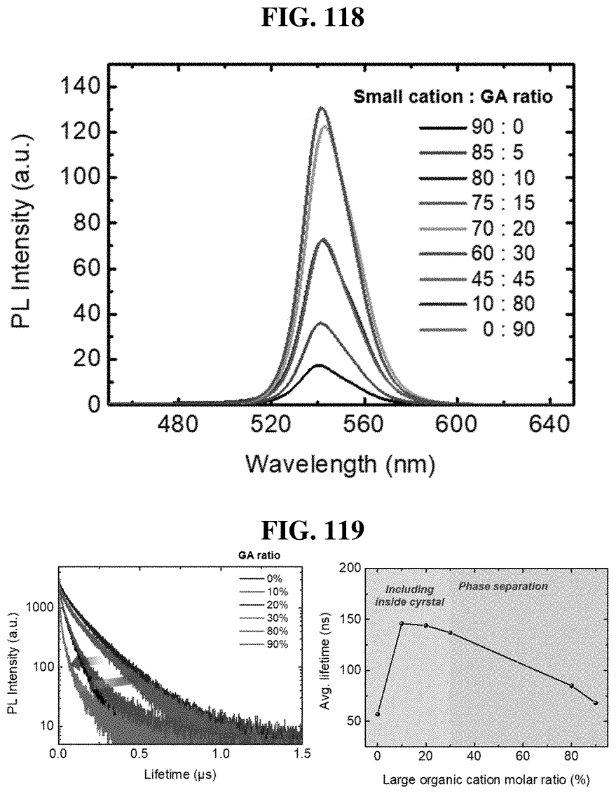

In the above embodiment, the ratio of the A₂ organic cation in the mixture of the A₁, A₃, A₄ cation and A₂ organic cation among the monovalent (monovalent) cations at the A site is the ratio that can be contained in the perovskite crystal at the maximum. As a result of measuring the photoluminescence properties before and after adding 5% or more and 90% or less of the A₂ organic cation, until the A₂ organic cation was added at a ratio of about 30% or less, the steady-state photoluminescence gradually increased (see FIG. 118), and the photoluminescence (PL) lifetime became longer (see FIGS. 118 and 119).

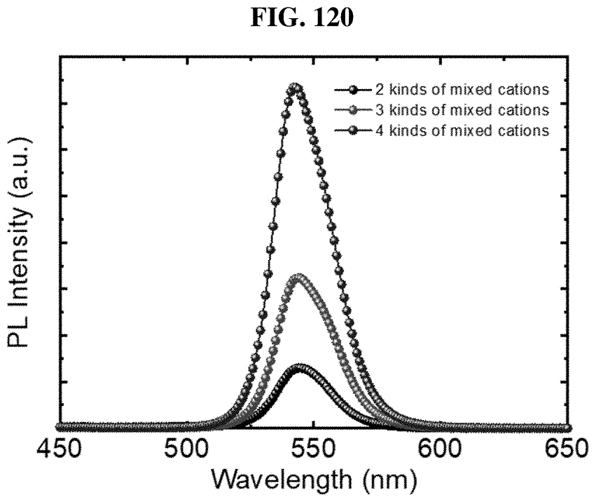

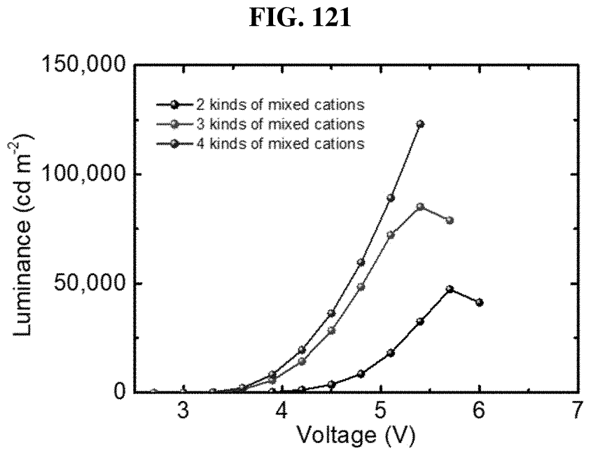

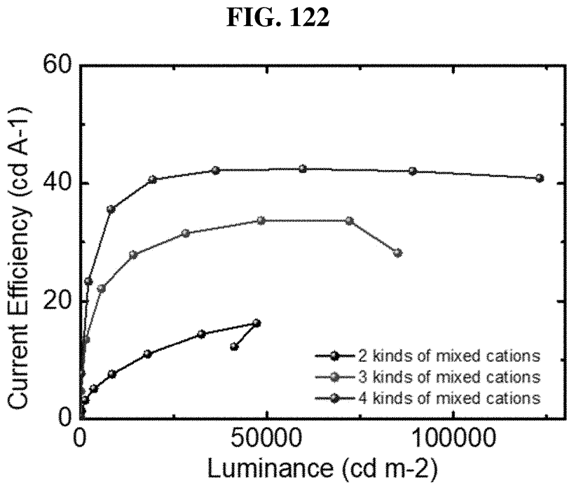

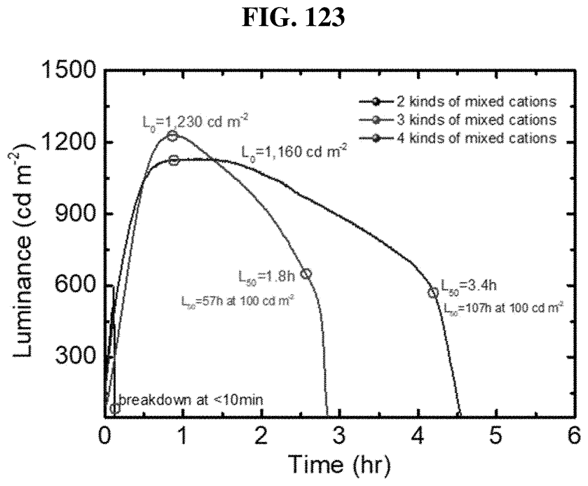

In the above embodiment, the two types of mixed cationic structures of A₁ and A₂ are the basic mixing structures that can form a three-dimensional perovskite crystals. The three types of mixed cationic structures of A₁, A₂, and A₄ have additionally a percentage of A₄ cations that can be contained within the perovskite crystal at the maximum rate. A₁, A₂, A₃ and A₄ mixed cation structures are a combination that A₃ cation with a tolerance factor of 1.01 or less is mixed in a ratio that allows an optimal crystal stabilization. Measurement of the luminescence characteristics of perovskite polycrystalline thin films of these two, three and four mixed cationic structures shows improved rectified light emission intensity in order of two<three<four mixed cationic structures (see FIG. 120), maximum luminance of perovskite light emitting devices in the same order (see FIG. 121), improved current efficiency and reduced roll-off in the same order (see FIG. 122), and also the improved operational lifetime of the light-emitting diode in the same order (see FIG. 123).

Thus, medium-sized monovalent cation (A₂) contained within perovskite crystals can stabilize perovskite crystals and inhibit the production of defects within the crystals due to its entropy effect, and the excess A₂ cations not contained within perovskite crystals form a structure surrounding perovskite nanocrystal particles to passivate the surface of perovskite nanocrystal particles, thereby improving photoluminescence quantum efficiency, photoluminescence lifetime and stability, thus it can be useful for the light emitting layer or wavelength conversion layer of the light emitting element.

An example of a light-emitting device including a perovskite material in which defect generation is controlled through the addition of a medium-sized organic cation according to the present disclosure is the same as the description of the light-emitting device described above, and a detailed description will be omitted to avoid redundant description.

### MODE FOR CARRYING OUT THE DISCLOSURE

Hereinafter, the present disclosure will be described in detail by examples and experimental examples. However, the following examples and experimental examples are merely illustrative of the present disclosure, and the contents of the present disclosure are not limited by the following examples and experimental examples.

### <Example 1> Preparation of Green Light-Emitting Metal Halide Perovskite Nanoparticle Solution and Film Containing Guanidinium Cations

A precursor solution was prepared by dissolving metal halide perovskite in a polar solvent. The polar solvent at this time was dimethylformamide, and metal halide perovskite precursors used formamidinium bromide (FABr) and guanidinium bromide (GABr) and PbBr₂. The ratio of used (FABr+GABr) to PbBr₂ is 2:1, and the mixing ratio of used FABr and GABr can be adjusted to adjust the composition of GABr in metal halide perovskite nanoparticles.

The mixing ratio of GABr to the total amount of FABr and GABr can be adjusted to 5%, 6%, 7%, 8%, 9%, 10%, 11%, 12%, 13%, 14%, 15%, 16%, 17%, 18%, 19%, 20%, 22%, 24%, 26%, 28%, 30%, 32%, 34%, 36%, 38%, 40%, 45%, 50%, 55%, 60%, 80%, 100%.

Thereafter, an anti-solvent solution containing a ligand was prepared. As a solvent of the anti-solvent solution, toluene and 1-butanol were used. The anti-solvent solution in which toluene and 1-butanol were mixed in a ratio of 5:2 was used. As the ligand, oleic acid and octyl amine were used.

Thereafter, metal halide perovskite nanoparticle crystallization was induced by dropping the metal halide perovskite precursor solution into the anti-solvent solution. As the metal halide perovskite precursor solution was mixed with the anti-solvent solution, the solubility decreased rapidly, and thus metal halide perovskite crystals surrounded by the ligand were precipitated. At this time, the composition of the precipitated metal halide perovskite crystal has FA₁₋ₓGAₓPbBr₃, and x value can be 0.05, 0.06, 0.07, 0.08, 0.09, 0.1, 0.11, 0.12, 0.13, 0.14, 0.15, 0.16, 0.17, 0.18, 0.19, 0.20, 0.22, 0.24, 0.26, 0.28, 0.3, 0.32, 0.34, 0.36, 0.38, 0.40, 0.45, 0.5, 0.55, 0.6, 0.8, 1.0 according to the mixing ratio of FABr and GABr in the precursor solution.

After spreading the prepared metal halide perovskite nanoparticle solution on a glass substrate, spin coating was performed while rotating the glass substrate at a speed of 500 rpm to prepare a perovskite film.

### Embodiment 2-10

A perovskite nanoparticle solution and film were prepared in the same method as in Embodiment 1 except for using ethylammonium bromide, tert-butylammonium bromide, diethylammonium bromide, dimethylammonium bromide, ethane-1,2-diammonium bromide, imidazolium bromide, n-propylammonium bromide, iso-propylammonium bromide, and pyrrolidinium bromide instead of guanidinium bromide.

### Embodiment 11-30

A perovskite nanoparticle solution and film were prepared in the same method as in Embodiment 1-10 except for using methylammonium bromide and Cesium bromide instead of formamidinium bromide.

### <Embodiment 31> Fabrication of Red Light-Emitting Metal Halide Perovskite Nanoparticle Solution and Film

A precursor solution was prepared by dissolving a metal halide perovskite in a polar solvent. At this time, as the polar solvent, dimethylformamide was used, and as a metal halide perovskite precursor, formamidinium iodide (FAI), guanidinium iodide (GAI), PbI₂ was used. At this time, the ratio of (FAI+GAI) and PbI₂ is 2:1, and by adjusting the mixing ratio of FAI and GAI, the composition of GAI contained in metal halide perovskite nanoparticles can be adjusted.

The mixing ratio of GAI to the total amount of FAI and GAI is 5%, 6%, 7%, 8%, 9%, 10%, 11%, 12%, 13%, 14%, 15%, 16%, 17%, 18%, 19%, 20%, 22%, 24%, 26%, 28%, 30%, 32%, 34%, 36%, 38%, 40%, 45%, 50%, 55%, 60%, 80%, 100%

Thereafter, an anti-solvent solution containing a ligand was prepared. As a solvent of the anti-solvent solution, toluene and 1-butanol were used. The anti-solvent solution in which toluene and 1-butanol were mixed in a ratio of 5:2 was used. As the ligand, oleic acid and octyl amine were used.

Thereafter, metal halide perovskite nanoparticle crystallization was induced by dropping the metal halide perovskite precursor solution into the anti-solvent solution. As the metal halide perovskite precursor solution was mixed with the anti-solvent solution, the solubility decreased rapidly, and thus metal halide perovskite crystals surrounded by the ligand were precipitated. At this time, the composition of the precipitated metal halide perovskite crystal has FA₁₋ₓGAₓPbI₃, and x value can be 0.05, 0.06, 0.07, 0.08, 0.09, 0.1, 0.11, 0.12, 0.13, 0.14, 0.15, 0.16, 0.17, 0.18, 0.19, 0.20, 0.22, 0.24, 0.26, 0.28, 0.3, 0.32, 0.34, 0.36, 0.38, 0.40, 0.45, 0.5, 0.55, 0.6, 0.8, 1.0 according to the mixing ratio of FAI and GAI in the precursor solution.

After spreading the prepared metal halide perovskite nanoparticle solution on a glass substrate, spin coating was performed while rotating the glass substrate at a speed of 500 rpm to prepare a perovskite film.

### Embodiment 32-40

A perovskite nanoparticle solution and film were prepared in the same method as in Embodiment 31 except for using ethylammonium iodide, tert-butylammonium iodide, diethylammonium iodide, dimethylammonium iodide, ethane-1,2-diammonium iodide, imidazolium iodide, n-propylammonium iodide, iso-propylammonium iodide, and pyrrolidinium iodide instead of guanidinium iodide.

### Embodiment 41-60

A perovskite nanoparticle solution and film were prepared in the same method as in Embodiment 31-40 except for using methylammonium iodide and cesium iodide instead of formamidinium iodide.

### <Embodiment 61> Fabrication of Blue Light-Emitting Metal Halide Perovskite Nanoparticle Solution and Film

A precursor solution was prepared by dissolving a metal halide perovskite in a polar solvent. At this time, as the polar solvent, dimethylformamide was used and, as a metal halide perovskite precursor, formamidinium chloride (FACl), guanidinium chloride (GACl), PbCl₂ was used. At this time, the ratio of (FACl+GACl) and PbCl₂ is 2:1, and by adjusting the mixing ratio of FACl and GACl, the composition of GACl contained in metal halide perovskite nanoparticles can be adjusted.

The mixing ratio of GACl to the total amount of FACl and GACl is 5%, 6%, 7%, 8%, 9%, 10%, 11%, 12%, 13%, 14%, 15%, 16%, 17%, 18%, 19%, 20%, 22%, 24%, 26%, 28%, 30%, 32%, 34%, 36%, 38%, 40%, 45%, 50%, 55%, 60%, 80%, 100%.

Thereafter, an anti-solvent solution containing a ligand was prepared. As a solvent of the anti-solvent solution, toluene and 1-butanol were used. The anti-solvent solution in which toluene and 1-butanol were mixed in a ratio of 5:2 was used. As the ligand, oleic acid and octyl amine were used.

Thereafter, metal halide perovskite nanoparticle crystallization was induced by dropping the metal halide perovskite precursor solution into the anti-solvent solution. As the metal halide perovskite precursor solution was mixed with the anti-solvent solution, the solubility decreased rapidly, and thus metal halide perovskite crystals surrounded by the ligand were precipitated. At this time, the composition of the precipitated metal halide perovskite crystal has FA₁₋ₓGAₓPbCl₃, and x value can be 0.05, 0.06, 0.07, 0.08, 0.09, 0.1, 0.11, 0.12, 0.13, 0.14, 0.15, 0.16, 0.17, 0.18, 0.19, 0.20, 0.22, 0.24, 0.26, 0.28, 0.3, 0.32, 0.34, 0.36, 0.38, 0.40, 0.45, 0.5, 0.55, 0.6, 0.8, 1.0 according to the mixing ratio of FACl and GACl in the precursor solution.

After spreading the prepared metal halide perovskite nanoparticle solution on a glass substrate, spin coating was performed while rotating the glass substrate at a speed of 500 rpm to prepare a perovskite film.

### Embodiment 62-70

A perovskite nanoparticle solution and film were prepared in the same method as in Embodiment 61 except for using ethylammonium chloride, tert-butylammonium chloride, diethylammonium chloride, dimethylammonium chloride, ethane-1,2-diammonium chloride, imidazolium chloride, n-propylammonium chloride, iso-propylammonium chloride, pyrrolidinium chloride instead of guanidinium chloride/

### Embodiment 71-90

A perovskite nanoparticle solution and film were prepared in the same method as in Embodiment 61-70 except for using methylammonium chloride and cesium chloride instead of Formamidinum chloride.

### <Comparative Example 1> Preparation of Metal Halide Perovskite Nanoparticle Solution without Guanidinium Cations

The mixing ratio of GACl to the total amount of FACl and GACl is 0%, that is, the perovskite nanoparticle solution was prepared in the same method as in Embodiment 1, while GACl is not used.

### <Experimental Example 1> Changes in the Structural Properties of Metal Halide Perovskite Nanoparticle Solution Containing Guanidinium Cations

In the preparation of perovskite nanoparticles according to the present disclosure, to investigate the effect of GA addition on the luminescence properties of perovskite films, X-ray diffraction structure analysis (XRD) was measured for the perovskite films prepared in Embodiment 1 and Comparative Example 1 and the results are shown in FIG. 108, and photoluminescence characteristics were measured using a spectrofluorometer and the results are shown in FIG. 109. In addition, the size of the perovskite nanoparticles prepared in Embodiment 1 and Comparative Example 1 was measured through scanning electron microscope analysis (TEM), and the results are shown in FIG. 110.

As shown in FIGS. 108 and 109, when the mixing ratio of guanidinium is 5% or less, all GA is included in the perovskite crystal to expand the crystal and the steady-state photoluminescence wavelength is red-shifted. On the other hand, when 5% or more of GA was added, the crystal lattice did not change and the steady-state photoluminescence wavelength was blue-shifted

In addition, according to FIG. 110, it can be seen that the growth of crystals is suppressed and the size of the nanoparticles decreases according to the addition of GA.

### <Experimental Example 2> Changes in Steady-State Photoluminescence Characteristics of Metal Halide Perovskite Nanoparticle Solution Containing Guanidinium Cation

In the metal halide perovskite light emitting particles according to an embodiment of the present disclosure, the photoluminescence quantum efficiency (PLQY) of the perovskite nanoparticle solutions prepared in Embodiment 1 and Comparative Example 1 was measured, and the results are shown in FIG. 111 and Table 4 below.

TABLE 4

| GA mixing ratio (%) | PLQY (%) |
|---|---|
| 0 | 6.67 |
| 5 | 76.38 |
| 6 | 75.39 |
| 7 | 78.19 |
| 8 | 75.23 |
| 9 | 81.55 |
| 10 | 83.05 |
| 11 | 82.71 |
| 12 | 82.94 |
| 13 | 81.59 |
| 14 | 82.54 |
| 15 | 82.02 |
| 16 | 82.83 |
| 17 | 82.07 |
| 18 | 82.33 |
| 19 | 82.55 |
| 20 | 82.71 |
| 22 | 84.45 |
| 24 | 82.00 |
| 26 | 85.62 |
| 28 | 83.18 |
| 30 | 84.87 |
| 32 | 81.91 |
| 34 | 82.90 |
| 36 | 84.39 |
| 38 | 83.41 |
| 40 | 82.74 |
| 45 | 79.11 |
| 50 | 80.80 |
| 55 | 77.49 |
| 60 | 84.80 |
| 80 | 75.34 |
| 100 | 20.22 |

As shown in FIG. 111 and Table 4 below, it can be seen that the photoluminescence quantum efficiency (PLQY) of the perovskite nanoparticles increases as the GA contained in the crystal is added by 5% or more, and it was confirmed that the highest photoluminescence quantum efficiency can be obtained at the GA mixing ratio of 10% to 30%.

### <Experimental Example 3> Changes in Steady-State Photoluminescence Characteristics of Red Light-Emitting Metal Halide Perovskite Nanoparticle Solution Containing Guanidinium Cation

In the metal halide perovskite light emitting particles according to an embodiment of the present disclosure, the photoluminescence quantum efficiency (PLQY) of the perovskite nanoparticle solutions prepared in Embodiment 31 was measured, and the results are shown Table 5 below.

TABLE 5

| GA mixing ratio (%) | PLQY (%) |
|---|---|
| 0 | 62.38 |
| 5 | 63.53 |
| 6 | 68.31 |
| 7 | 74.69 |
| 8 | 70.71 |
| 9 | 74.81 |
| 10 | 80.06 |
| 11 | 76.53 |
| 12 | 74.77 |
| 13 | 74.54 |
| 14 | 77.81 |
| 15 | 77.06 |
| 16 | 77.02 |
| 17 | 77.43 |
| 13 | 77.01 |
| 19 | 77.94 |
| 20 | 74.85 |
| 22 | 78.31 |
| 24 | 75.51 |
| 26 | 79.22 |
| 28 | 76.87 |
| 30 | 80.55 |
| 32 | 78.15 |
| 34 | 77.75 |
| 36 | 79.77 |
| 38 | 79.03 |
| 40 | 79.57 |
| 45 | 73.94 |
| 50 | 77.70 |
| 55 | 70.42 |
| 60 | 79.41 |
| 80 | 68.18 |
| 100 | 20.75 |

As shown in Table 5, it can be seen that the photoluminescence quantum efficiency (PLQY) of the perovskite nanoparticles increases as the GA contained in the crystal is added by 5% or more, and it was confirmed that the highest photoluminescence quantum efficiency can be obtained at the GA mixing ratio of 10% to 40%

### <Experimental Example 4> Changes in Steady-State Photoluminescence Characteristics of Blue Light-Emitting Metal Halide Perovskite Nanoparticle Solution Containing Guanidinium Cation

In the metal halide perovskite light emitting particles according to an embodiment of the present disclosure, the photoluminescence quantum efficiency (PLQY) of the perovskite nanoparticle solution prepared in Embodiment 61 is shown in Table 6 below. [Table 6]

| GA mixing ratio (%) | PLQY (%) |
|---|---|
| 0 | 57.09 |
| 5 | 60.90 |
| 6 | 64.75 |
| 7 | 67.67 |
| 8 | 66.73 |
| 9 | 73.49 |
| 10 | 71.43 |
| 11 | 76.49 |
| 12 | 79.72 |
| 13 | 78.53 |
| 14 | 77.64 |
| 15 | 80.83 |
| 16 | 79.00 |
| 17 | 77.88 |
| 18 | 76.76 |
| 19 | 77.82 |
| 20 | 79.57 |
| 22 | 77.23 |
| 24 | 76.60 |
| 26 | 76.81 |
| 28 | 73.24 |
| 30 | 70.72 |
| 32 | 63.51 |
| 34 | 62.12 |
| 36 | 65.13 |
| 38 | 64.14 |
| 40 | 60.88 |
| 45 | 57.09 |
| 50 | 50.33 |
| 55 | 45.99 |
| 60 | 43.65 |
| 80 | 33.30 |
| 100 | 12.29 |

As shown in Table 6, it can be seen that the photoluminescence quantum efficiency (PLQY) of the perovskite nanoparticles increases as the GA contained in the crystal is added by 5% or more, and it was confirmed that the highest photoluminescence quantum efficiency can be obtained at the GA mixing ratio of 10% to 40%

### <Experimental Example 5> Changes in Luminescence Lifetime Characteristics of Metal Halide Perovskite Nanoparticle Solutions Containing Guanidinium Cation

In the metal halide perovskite light emitting particles according to an embodiment of the present disclosure, the time-resolved photoluminescence of the perovskite nanoparticle solutions prepared in Embodiment 1 and Comparative Example 1 was measured, and the results are shown in FIG. 112 and Table 7 below.

TABLE 7

| GA mixing ratio (%) | Average lifetime (ns) |
|---|---|
| 0 | 261 |
| 5 | 273 |
| 6 | 276 |
| 7 | 271 |
| 8 | 268 |
| 9 | 273 |
| 10 | 312 |
| 11 | 345 |
| 12 | 361 |
| 13 | 392 |
| 14 | 408 |
| 15 | 417 |
| 16 | 44 |
| 17 | 503 |
| 18 | 541 |
| 19 | 598 |
| 20 | 657 |
| 22 | 646 |
| 24 | 638 |
| 26 | 635 |
| 28 | 555 |
| 30 | 524 |
| 32 | 477 |
| 34 | 347 |
| 36 | 218 |
| 38 | 146 |
| 40 | 131 |
| 45 | 111 |
| 50 | 80 |
| 55 | 71 |
| 60 | 65 |
| 80 | 52 |
| 100 | 21 |

As shown in Table 7, it can be seen that the photoluminescence quantum efficiency (PLQY) of the perovskite nanoparticles increases as the GA contained in the crystal is added by 5% or more, and it was confirmed that the highest photoluminescence quantum efficiency can be obtained at the GA mixing ratio of 10% to 40%

### <Experimental Example 6> Changes in Exciton Binding Energy of Metal Halide Perovskite Nanoparticle Solution Containing Guanidinium Cations

In the perovskite nanoparticle solutions prepared in Embodiment 1 and Comparative Example 1, FIG. 113 is a result of temperature-dependent steady state photoluminescence of FA_{0.9}GA_{0.1}PbBr₃ and FAPbBr₃ films. The measurement temperature was performed in the range of 160 K or more and 300 K or less. In the above measurement results, exciton binding energy was calculated by calculating the photoluminescence intensity at each temperature by the following equation.

I(T)=I₀*exp(−E_{b}/kT)(I(T): photoluminescence intensity at temperature T, I₀: constant, E_{b}: exciton binding energy, k: gas constant, T: absolute temperature))

As shown in FIG. 113, as GA was added to the perovskite crystal, the calculated exciton binding energy increased from 101 meV to 152 meV.

### <Experimental Example 7> Changes in Stability of Metal Halide Perovskite Nanoparticle Thin Films Containing Guanidinium Cations

In the perovskite nanoparticle solutions prepared in Embodiment 1 and Comparative Example 1, FIG. 114 is a result of steady state photoluminescence according to UV irradiation time of FA_{0.9}GA_{0.1}PbBr₃ and FAPbBr₃ films. As shown in FIG. 114, it is confirmed that the perovskite light-emitting particles to which 10% of GA was added showed higher photostability, and in particular, as GA was added, the binding energy of the crystal increased, and the spectrum did not change and remained stable.

In the metal halide perovskite light emitting particles according to an embodiment of the present disclosure, FIG. 115 is a thermogravimetric analysis result of the perovskite nanoparticle thin films prepared in Embodiment 1 and Comparative Example 1. The perovskite nanoparticle thin film was weighed in a temperature range of 100° C. to 500° C. Compared to the perovskite nanoparticles to which GA was not added, it was confirmed that the addition ratio of GA showed higher stability against heat in the range of 10% to 30%.

### <Preparation Example 1> Light-Emitting Diode Using Metal Halide Perovskite Nanoparticles Containing Guanidinium Cations

First, after preparing an ITO substrate (a glass substrate coated with an ITO anode), PEDOT:PSS (Heraeus, AI4083) was spin-coated, and heat treatment was performed for 30 minutes to form a hole injection layer having a thickness of 50 nm.

Next, the metal halide perovskite solution prepared in Embodiment 1 to 90 or Comparative Example was coated at 500 rpm for 60 seconds to form a perovskite light emitting layer.

Perovskite light-emitting diode was fabricated by depositing 50 nm-thick 1,3,5-Tris(1-phenyl-1H-benzimidazol-2-yl)benzene (TPBI) at a high vacuum of 2×10-7 Torr or less as an electron transport layer, depositing 1 nm-thick LiF thereon to form an electron injection layer and depositing 100 nm-thick aluminum thereon to form a negative electrode.

The luminous efficiency of the light emitting diode according to the content of guanidinium using metal halide perovskite nanoparticles according to an embodiment of the present disclosure is shown in FIG. 116 and Table 8 below.

TABLE 8

| GA mixing | Current Efficiency | GA mixing | Current Efficiency |
|---|---|---|---|
| ratio (%) | (Cd/A)) | ratio (%) | (Cd/A) |
| 0 | 61.38 | 22 | 83.11 |
| 5 | 74.74 | 24 | 83.21 |
| 6 | 81.25 | 26 | 82.67 |
| 7 | 79.25 | 28 | 79.05 |
| 8 | 83.42 | 30 | 76.77 |
| 9 | 83.64 | 32 | 74.21 |
| 10 | 95.70 | 34 | 74.44 |
| 11 | 95.65 | 36 | 68.01 |
| 12 | 94.82 | 38 | 64.72 |
| 13 | 94.54 | 40 | 58.91 |
| 14 | 94.64 | 45 | 49.21 |
| 15 | 89.89 | 50 | 36.24 |
| 16 | 91.54 | 55 | 29.26 |
| 17 | 89.51 | 60 | 21.98 |
| 18 | 86.14 | 80 | 0.54 |
| 19 | 83.79 | 100 | 0.01 |
| 20 | 83.34 | | |

As shown in Table 8, it was confirmed that the luminous efficiency of the perovskite light emitting diode was improved in the range of 5% to 35% of the guanidinium content, and the highest luminous efficiency was achieved at the guanidinium content of 10%. The decrease in the luminous efficiency of the light emitting diode at the guanidinium content over 10% is due to the decrease in the size of the nanoparticles due to the addition of GA (FIG. 110).

### <Embodiment 91> Preparation of Green Light-Emitting Perovskite Film Having Three Mixed Cationic Structures Using Guanidinium Cations

A solution was prepared by dissolving an organic-inorganic hybrid perovskite in a polar solvent. As the polar solvent, dimethylsulfoxide is used, and as the hybrid perovskite precursor, (FA1-xGAx)0.87Cs0.13PbBr₃ was used by mixing three cations of formamidinium Bromide (FABr), guanidinium bromide (GABr), and cesium bromide (CsBr). The ratio of (FABr+GABr+CsBr) and PbBr₂ used at this time is 1.1:1, and the mixing ratio of FABr and GABr can be adjusted to 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 32%, 34%, 36%, 38%, 40%, 45%, 50%, 55%, 60%, 80%, 100%, the mixture solution with the concentration of (FA₁₋ₓGAₓ)_{0.87}Cs_{0.13}PbBr₃ of 1.2M relative to the total precursor solution was used. The composition of the perovskite crystal formed at this time is (FA₁₋ₓGAₓ)_{0.87}Cs_{0.13}PbBr₃, and according to the mixing ratio of FABr and GABr in the precursor solution, x value can be 0.05, 0.06, 0.07, 0.08, 0.09, 0.1, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.3, 0.32, 0.34, 0.36, 0.38, 0.40, 0.45, 0.5, 0.55, 0.6, 0.8, and 1.0.

After spreading the solution on a glass substrate, spin coating was performed while rotating the glass substrate at a speed of 6000 rpm to prepare a perovskite film, followed by heat treatment at 70° C. for 10 minutes.

### Embodiment 92-100

A perovskite film was prepared in the same method as in Embodiment 91 except for using Ethylammonium bromide, Tert-butylammonium bromide, Diethylammonium bromide, Dimethylammonium bromide, Ethane-1.2.-diammonium bromide, Imidazolium bromide, n-propylammonium bromide, iso-propylammonium bromide, and Pyrrolidinium bromide instead of Guanidinium bromide.

### <Embodiment 101> Preparation of a Red Light-Emitting Perovskite Film Having Three Kinds of Mixed Cationic Structure Using Guanidinium Cations

A solution was prepared by dissolving an organic-inorganic hybrid perovskite in a polar solvent. At this time, dimethylsulfoxide is used as the polar solvent, and as a precursor of the organic-inorganic hybrid perovskite, and (FA₁₋ₓGAₓ)_{0.87}Cs_{0.13}PbI₃ in which three kinds of cations of formamidinium iodide (FAI), guanidinium iodide (GAI) and cesium iodide (CsI) were mixed was used. The ratio of (FAI+GAI+CsI) and PbI₂ used at this time is 1.1:1, and the mixing ratio of FAI and GAI can be adjusted to 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 32%, 34%, 36%, 38%, 40%, 45%, 50%, 55%, 60%, 80%, 100%, and a mixed solution was used so that the concentration of (FA₁₋ₓGAₓ)_{0.87}Cs_{0.13}PbI₃ compared to the total precursor solution was 1.2M. The composition of the perovskite crystal formed at this time is (FA₁₋ₓGAₓ)_{0.87}Cs_{0.13}PbI₃, and may have a value of x=0.05, 0.06, 0.07, 0.08, 0.09, 0.1, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.3, 0.32, 0.34, 0.36, 0.38, 0.40, 0.45, 0.5, 0.55, 0.6, 0.8, 1.0 depending on the mixing ratio of FAI and GAI in the precursor solution.

After the solution was applied on a glass substrate, spin coating was performed while rotating the glass substrate at a speed of 6000 rpm to prepare a perovskite film, which was heat-treated at 70° C. for 10 minutes.

### Embodiment 102-110

A perovskite nanoparticle solution and film were prepared in the same method as in embodiment 101, except that ethylammonium iodide, tert-butylammonium iodide, diethylammonium iodide, dimethylammonium iodide, ethane-1,2,-diammonium iodide, imidazolium iodide, n-propylammonium iodide, iso-propylammonium iodide and pyrrolidinium iodide were used instead of guanidinium iodide.

### <Embodiment 111> Preparation of a Blue Light-Emitting Perovskite Film Having Three Mixed Cationic Structures Using Guanidinium Cations

A solution was prepared by dissolving an organic-inorganic hybrid perovskite in a polar solvent. At this time, dimethylsulfoxide is used as the polar solvent, and as a precursor of the organic-inorganic hybrid perovskite, (FA₁₋ₓGAₓ)_{0.87}Cs_{0.13}PbCl₃ in which three kinds of cations of formamidinium chloride (FACl), guanidinium chloride (GACl) and cesium chloride (CsCl) were mixed was used. The ratio of (FACl+GACl+CsCl) and PbCl₂ used at this time is 1.1:1, and the mixing ratio of FACl and GACl can be adjusted to 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 32%, 34%, 36%, 38%, 40%, 45%, 50%, 55%, 60%, 80%, 100%, and a mixed solution was used so that the concentration of (FA₁₋ₓGAₓ)_{0.87}Cs_{0.13}PbCl₃ compared to the total precursor solution was 1.2M. The composition of the perovskite crystal formed at this time is (FA₁₋ₓGAₓ)_{0.87}Cs_{0.13}PbCl₃, and may have a value of x=0.05, 0.06, 0.07, 0.08, 0.09, 0.1, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.3, 0.32, 0.34, 0.36, 0.38, 0.40, 0.45, 0.5, 0.55, 0.6, 0.8, 1.0 depending on the mixing ratio of FACl and GACl in the precursor solution.

After the solution was applied on a glass substrate, spin coating was performed while rotating the glass substrate at a speed of 6000 rpm to prepare a perovskite film, which was heat-treated at 70° C. for 10 minutes.

### Embodiment 112-120

A perovskite nanoparticle solution and film were prepared in the same method as in embodiment 111, except that ethylammonium chloride, tert-butylammonium chloride, diethylammonium chloride, dimethylammonium chloride, ethane-1,2,-diammonium chloride, imidazolium chloride, n-propylammonium chloride, iso-propylammonium chloride and pyrrolidinium chloride were used instead of guanidinium chloride.

### <Experimental Example 8> Changes in Structural Properties of Perovskite Film According to Guanidinium Cation Substitution

In the fabrication of the perovskite film according to the present disclosure, in order to investigate the change of the perovskite structure according to guanidinium cation substitution, the crystal structure of the perovskite film prepared in Embodiment 91 was analyzed using X-ray diffraction, and the results are shown in FIG. 117.

As shown in FIG. 117(a), if the ratio of guanidinium is 0<=x<0.3, it can be seen that the X-ray diffraction spectrum of the perovskite film represents the peak of the cubic structure, such as the diffraction spectrum if x=0. It can be seen that this is the result of when the mixing ratio of the formamidinium cation and the guanidinium cation is x<0.3, the guanidinium cation enters into the crystal to form a crystal structure and does not break the cubic structure. On the other hand, when 0.3<x, it can be seen that a new peak at the position of 2θ=13° is formed due to the formation of a new crystal structure and phase separation. In addition, as shown in FIG. 117(b), considering that the distance between faces increases only when 0<x<0.3 when calculating the interplanar distance of the crystal structure corresponding to the (100) peak of the cubic crystal, it can be seen that up to a ratio of x<0.3, guanidinium cations having a large ionic radius enter into the crystal, and only the size increases while maintaining the cubic crystal structure.

### <Experimental Example 9> Changes in Photoluminescence Properties of Perovskite Films According to Guanidinium Cation Substitution

In the fabrication of the perovskite film according to the present disclosure, in order to investigate the effect on the photoluminescence properties of the perovskite film according to guanidinium cation substitution, photoluminescence properties of the perovskite film prepared in Embodiment 91 were measured using a spectrofluorometer, and the results are shown in FIG. 118.

As shown in FIG. 118, the conventional perovskite film having a composition in which the formamidinium cation is not substituted with the guanidinium cation has a photoluminescence intensity of about less than 20 a.u. near a wavelength of 540 nm, on the other hand, the perovskite film having a mixed cation structure by guanidinium cation substitution according to the present disclosure increases the photoluminescence intensity to about 130 a.u. as the amount of substituted guanidinium cations increases to x=0.3 in the same wavelength range, thereby increasing light emitting characteristics by more than six times compared to a conventional perovskite film using formamidinium cations.

As described above, the mixed cationic structure perovskite film by guanidinium cation substitution according to the present disclosure exhibits remarkably increased light-emitting properties compared to the conventional perovskite film, and thus can be useful as a light-emitting layer of a light-emitting device.

### <Experimental Example 10> Changes in Charge Lifetime Characteristics of Perovskite Films According to Guanidinium Cation Substitution

In the fabrication of the perovskite film according to the present disclosure, in order to examine the effect on the charge lifetime characteristics of the perovskite film according to guanidinium cation substitution, the charge lifetime of perovskite film prepared in Embodiment 91 was measured, and the results are shown in FIG. 119.

FIG. 119 is a graph showing charge lifetime characteristics of a perovskite film according to guanidinium cation substitution according to an embodiment of the present disclosure.

As shown in FIG. 119, while the conventional perovskite film using formamidinium cation has a charge lifetime of 50 ns, perovskite films with mixed cationic structure according to guanidinium cation replacement according to the present disclosure have been shown to have a lifetime of approximately 150 ns in the substitution range of 0<x<0.3, which is approximately three times more than conventional perovskite films.

In addition, as shown in FIG. 119(b), it can be seen that when the excessive guanidinium cation is substituted with 0.3<x, the charge lifetime is rather reduced. This is a result of the formation of a new crystal structure other than a preferred cubic system by the above-described phase separation, and it can be seen that the substitution ratio of the guanidinium cation plays a major role in the light emission characteristics.

As described above, the perovskite film having a mixed cation structure according to guanidinium cation substitution according to the present disclosure exhibits significantly increased lifetime characteristics compared to the conventional perovskite film, so it can be useful as a light emitting layer of a light emitting device.

### <Embodiment 121> Fabrication of Green Light-Emitting Perovskite Film Having 4 Mixed-Cation Structure

A solution was prepared by dissolving an organic-inorganic hybrid perovskite in a polar solvent. At this time, dimethylsulfoxide is used as the polar solvent, and as a precursor of the organic-inorganic hybrid perovskite, (FAₓGA_{y}MA_{z})_{0.87}Cs_{0.13}PbBr₃ in which four kinds of cations of formamidinium bromide (FABr), guanidinium bromide (GABr), cesium bromide (CsBr), and methylammonium bromide (MABr) were mixed was used. The ratio of (FABr+GABr+CsBr+MABr) and PbBr₂ used at this time is 1.1:1, the x, y, z are 0 or more and 1 or less, the mixing ratio of MABr in the 4 kinds of cation mixture can be adjusted to 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, and a mixed solution was used so that the concentration of (FAₓGA_{y}MA_{z})_{0.87}Cs_{0.13}PbBr₃ compared to the total precursor solution was 1.2M.

After the solution was applied on a glass substrate, spin coating was performed while rotating the glass substrate at a speed of 6000 rpm to prepare a perovskite film, which was heat-treated at 70° C. for 10 minutes.

### Embodiment 122-130

A perovskite nanoparticle solution and film were prepared in the same method as in embodiment 121, except that ethylammonium bromide, tert-butylammonium bromide, diethylammonium bromide, dimethylammonium bromide, ethane-1,2,-Diammonium bromide, imidazolium bromide, n-propylammonium bromide, iso-propylammonium bromide and pyrrolidinium bromide were used instead of guanidinium bromide.

### Embodiment 131-170

A perovskite nanoparticle solution and film were prepared in the same method as in embodiment 31-40, except that sodium bromide, ammonium bromide, rubidium bromide, and potassium bromide were used instead of methylammonium bromide.

### <Embodiment 171> Fabrication of Red Light-Emitting Perovskite Film Having 4 Mixed-Cation Structure

A solution was prepared by dissolving an organic-inorganic hybrid perovskite in a polar solvent. At this time, dimethylsulfoxide is used as the polar solvent, and as a precursor of the organic-inorganic hybrid perovskite, (FAₓGA_{y}MA_{z})_{0.87}Cs_{0.13}PbI₃ in which four kinds of cations of formamidinium iodide (FAI), guanidinium iodide (GAI), cesium iodide (CsI), and methylammonium iodide (MAI) were mixed was used. The ratio of (FAI+GAI+CsI+MAI) and PbI₂ used at this time is 1.1:1, the x, y, z are 0 or more and 1 or less, the mixing ratio of MAI in the 4 kinds of cation mixture can be adjusted to 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, and a mixed solution was used so that the concentration of (FAₓGA_{y}MA_{z})_{0.87}Cs_{0.13}PbI₃ compared to the total precursor solution was 1.2M.

After the solution was applied on a glass substrate, spin coating was performed while rotating the glass substrate at a speed of 6000 rpm to prepare a perovskite film, which was heat-treated at 70° C. for 10 minutes.

### Embodiment 172-180

A perovskite nanoparticle solution and film were prepared in the same method as in embodiment 131, except that ethylammonium iodide, tert-butylammonium iodide, diethylammonium iodide, dimethylammonium iodide, ethane-1,2,-Diammonium iodide, imidazolium iodide, n-propylammonium iodide, iso-propylammonium iodide and pyrrolidinium iodide were used instead of guanidinium iodide.

### Embodiment 181-220

A perovskite nanoparticle solution and film were prepared in the same method as in embodiment 171-180, except that sodium iodide, ammonium iodide, rubidium iodide, and potassium iodide were used instead of methylammonium iodide.

### <Embodiment 221> Fabrication of Blue Light-Emitting Perovskite Film Having 4 Mixed-Cation Structure

A solution was prepared by dissolving an organic-inorganic hybrid perovskite in a polar solvent. At this time, dimethylsulfoxide is used as the polar solvent, and as a precursor of the organic-inorganic hybrid perovskite, (FAₓGA_{y}MA_{z})_{0.87}Cs_{0.13}PbCl₃, in which four kinds of cations of formamidinium chloride (FACl), guanidinium chloride (GACl), cesium chloride (CsCl), and methylammonium chloride (MACl) were mixed, was used. The ratio of (FACl+GACl+CsCl+MACl) and PbCl₂ used at this time is 1.1:1, the x, y, z are 0 or more and 1 or less, the mixing ratio of MACI in the 4 kinds of cation mixture can be adjusted to 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, and a mixed solution was used so that the concentration of (FAₓGA_{y}MA_{z})_{0.87}Cs_{0.13}PbCl₃ compared to the total precursor solution was 1.2M.

After the solution was applied on a glass substrate, spin coating was performed while rotating the glass substrate at a speed of 6000 rpm to prepare a perovskite film, which was heat-treated at 70° C. for 10 minutes.

### Embodiment 222-230

Perovskite nanoparticle solution and film were prepared in the same method as in embodiment 221, except that ethylammonium chloride, tert-butylammonium chloride, diethylammonium chloride, dimethylammonium chloride, ethane-1,2,-Diammonium chloride, imidazolium chloride, n-propylammonium chloride, iso-propylammonium chloride and pyrrolidinium chloride were used instead of guanidinium chloride.

### Embodiment 231-270

A perovskite nanoparticle solution and film were prepared in the same method as in embodiment 221-230, except that sodium chloride, ammonium chloride, rubidium chloride, and potassium chloride were used instead of methylammonium chloride.

### <Experimental Example 11> Changes in Photoluminescence Properties of Perovskite Films According to the Formation of 4 Mixed-Cation Structure

In the fabrication of the perovskite film according to the present disclosure, in order to investigate the effect on the photoluminescence properties of the perovskite film according to formation of 4 mixed-cation structure, photoluminescence properties of the perovskite film prepared in Embodiment 121 were measured using a spectrofluorometer, and the results are shown in FIG. 120.

As shown in FIG. 120, When comparing a conventional 2 mixed-cation structure using formamidinium and cesium cations, a 3 mixed-cation structure substituting guanidinium cation thereto, and a 4 mixed-cation structure additionally substituted with methylammonium cation, all of them exhibited photoluminescence properties at a wavelength of 540 nm. However, it was found that the photoluminescence intensity of the 4 mixed-cation structures was improved by about 7 times when compared to the 2 mixed-cation structures.

As described above, the perovskite film by formation of 4 mixed-cation structure according to the present disclosure exhibits remarkably increased light-emitting properties compared to the conventional perovskite film, and thus can be useful as a light-emitting layer of a light-emitting device.

### <Production Example 2> Fabrication of Light Emitting Diodes

First, an FTO substrate (a glass substrate coated with an FTO anode) was prepared, and then a conductive material PEDOT:PSS (AI4083 from Heraeus) was spin-coated on the FTO anode, followed by heat treatment at 150° C. for 30 minutes to form a hole injection layer with thickness of 50 nm.

Next, the 2, 3, 4 mixed-cation structure perovskite bulk polycrystalline precursor solution prepared in Embodiment 121 was applied on the hole injection layer and spin-coated while rotating at a speed of 6000 rpm. The prepared thin film was heat-treated at 70° C. for 10 minutes to form a perovskite light emitting layer having 2, 3, and 4 mixed-cation structures.

Next, to fabricate a perovskite light emitting diode, electron transport layer was formed by depositing 1,3,5-Tris(1-phenyl-1H-benzimidazol-2-yl)benzene (TPBI) with thickness of 50 nm under high vacuum less than 1×10⁻⁷ torr, LiF with thickness of 1 nm was deposited on the electron transport layer to form an electron injection layer, and aluminum with thickness of 100 nm was deposited to form a cathode.

### <Experimental Example 12> Measurement of the Efficiency of the Light Emitting Diodes

In the perovskite light emitting diode according to the present disclosure, luminance and current efficiency were measured for the perovskite light emitting diode prepared in Production Example 2, and the results are shown in FIGS. 121 and 122, respectively.

As shown in FIG. 121, the 2 mixed-cation structure-based perovskite light emitting diode showed the highest luminance of up to 50,000 cd m⁻², the 3 mixed-cation structure-based perovskite light emitting diode showed the highest luminance of up to 90,000 cd m⁻², while the 4 mixed-cation structure-based perovskite light emitting diode showed excellent luminance of more than 120,000 cd m⁻².

In addition, as shown in FIG. 122, compared to the current efficiency of the perovskite light-emitting diode based on the 2 mixed-cation structure and the 3 mixed-cation structure, the current efficiency of the perovskite light emitting diode based on the 4 mixed-cation structure was 40 cd A⁻¹ or more, and it can be seen that it is the best current efficiency among them. Furthermore, when the luminance of 50,000 cd m⁻² and 90,000 cd m⁻² is reached, perovskite light emitting diodes based on a 2 or 3 mixed-cation structure are damaged and cannot be driven anymore or the current efficiency is greatly reduced. On the other hand, the perovskite light emitting diode based on the 4 mixed-cation structure showed excellent driving stability with little current efficiency roll-off (reduction of maximum current efficiency) even at high luminance of 100,000 cd m⁻² or more.

It is considered that this is because in the perovskite light emitting layer having a mixed cation structure according to the present disclosure, the tolerance factor is adjusted through the cation mixing, the crystal structure is stabilized, and the defect density is reduced, thereby preventing the loss of charge in the perovskite crystal.

The device efficiency according to the GA mixing ratio of the (FA₁₋ₓGAₓ)_{0.87}Cs_{0.13}PbBr₃ 3 mixed-cation structure, and the device efficiency according to the mixing ratio of MA within the perovskite light-emitting layer of the (FA_{1-y}GA_{0.2}MA_{y})_{0.87}Cs_{0.13}PbBr₃ 4 mixed-cation structure are arranged in more detail in tables 9 and 10 respectively.

TABLE 9
(FA₁₋ₓGAₓ)_{0.87}Cs_{0.13}PbBr₃

| GA mixing | Maximum | Maximum current | Maximum |
|---|---|---|---|
| ratio (%) | luminance (cd m⁻²) | density (cd A⁻¹) | PLQY (%) |
| 0 | 42,254 | 16.28 | 3.27 |
| 5 | 57,415 | 20.78 | 4.20 |
| 6 | 61,725 | 22.13 | 4.75 |
| 7 | 62,555 | 24.21 | 4.71 |
| 8 | 69,389 | 23.81 | 4.89 |
| 9 | 67,254 | 24.60 | 5.25 |
| 10 | 72,231 | 26.29 | 5.23 |
| 11 | 68,268 | 26.50 | 5.57 |
| 12 | 74,160 | 28.87 | 5.73 |
| 13 | 76,539 | 28.32 | 6.05 |
| 14 | 80,127 | 29.13 | 6.06 |
| 16 | 83,654 | 30.01 | 6.51 |
| 16 | 82,656 | 30.78 | 6.44 |
| 17 | 79,960 | 31.51 | 6.61 |
| 18 | 89,842 | 32.14 | 6.68 |
| 19 | 92,290 | 34.73 | 6.90 |
| 20 | 85,117 | 33.60 | 6.99 |
| 21 | 81,201 | 32.00 | 6.67 |
| 22 | 83,574 | 30.24 | 6.55 |
| 23 | 74,534 | 28.01 | 6.19 |
| 24 | 73,814 | 26.26 | 5.42 |
| 25 | 61,900 | 24.45 | 5.08 |
| 26 | 60,373 | 23.12 | 5.07 |
| 27 | 51,509 | 21.13 | 4.32 |
| 26 | 54,212 | 20.57 | 3.96 |
| 29 | 49,651 | 17.10 | 3.53 |
| 30 | 36,683 | 14.95 | 3.01 |
| 45 | 14,423 | 9.62 | 1.93 |
| 50 | 4,662 | 4.68 | 0.89 |
| 55 | 4,413 | 2.79 | 0.50 |
| 60 | 3,763 | 2.46 | 0.52 |
| 80 | 589 | 1.89 | 0.46 |
| 100 | 369 | 1.12 | 0.17 |

TABLE 10
(FA₁₋ₓGA_{0.2}MA_{y})_{0.87}Cs_{0.13}PbBr₃

| GA mixing | Maximum | Maximum current | Maximum |
|---|---|---|---|
| ratio (%) | luminance (cd m⁻²) | density (cd A⁻¹) | PLQY (%) |
| 0 | 85,117 | 33.60 | 6.99 |
| 5 | 112,238.24 | 39.08 | 8.04 |
| 6 | 115,434 | 39.50 | 8.25 |
| 7 | 113,763 | 40.90 | 8.46 |
| 8 | 119,110 | 42.96 | 8.67 |
| 9 | 128,981 | 42.22 | 8.88 |
| 10 | 123,135 | 42.45 | 9.09 |
| 11 | 128,252.55 | 42.63 | 8.98 |
| 12 | 119,183.82 | 42.88 | 9.11 |
| 13 | 119,301.93 | 41.73 | 8.63 |
| 14 | 114,107.48 | 40.40 | 8.85 |
| 15 | 113,745.28 | 40.06 | 8.56 |
| 16 | 104,803.50 | 39.54 | 8.29 |
| 17 | 107,455.17 | 38.99 | 8.32 |
| 18 | 104,641.98 | 37.74 | 7.88 |
| 19 | 100,264.62 | 37.83 | 7.74 |
| 20 | 92,247.70 | 36.92 | 7.84 |
| 21 | 88,276.89 | 34.46 | 7.54 |
| 22 | 84,018.55 | 35.36 | 7.13 |
| 23 | 83,660.82 | 33.02 | 7.41 |
| 24 | 75,909.52 | 33.30 | 7.00 |
| 25 | 76,354.15 | 31.54 | 6.90 |
| 26 | 76,096.64 | 32.74 | 6.69 |
| 27 | 74,758.27 | 31.99 | 6.52 |
| 28 | 71,306.81 | 30.99 | 6.35 |
| 29 | 66,796.07 | 30.04 | 6.21 |
| 30 | 55,127 | 27.82 | 5.79 |

### <Experimental Example 13> Measurement of the Operational Lifetime of a Light Emitting Diode

In the perovskite light-emitting diode according to the disclosure, the operational lifetime of the perovskite light-emitting diode prepared in Fabrication Example 2 was measured, and the results are shown in FIG. 123.

As shown in FIG. 123, the operational lifetime of the conventional perovskite light emitting diode based on the 2 cation-mixed structure does not exceed about 10 minutes, but operational lifetime (L₅₀: time until the luminance decreases by 50% compared to the driving start point) of perovskite light emitting diodes including perovskite light emitting layers having 3 or 4 mixed-cation structures according to the present disclosure was improved to 1.8 hours at 1,230 cd m⁻² and 3.4 hours at 1,160 cd m⁻², respectively. In addition, this is an excellent operational lifetime of 57 hours at 100 cd m⁻² and 107 hours at 100 cd m⁻² when calculated according to the accelerated lifetime measurement method of the light emitting diode, so it can be seen that the operational lifetime is significantly improved from less than 10 minutes at 100 cd m⁻² in the 2 cation-mixed structure-based perovskite light-emitting diode. It is considered that this is because the movement of the Br⁻ anion, which is most easily moved along the electric field applied during the operation of the perovskite light emitting diode, is prevented by the stabilized crystal structure and low defect density.

L50 according to the mixing ratio of MA within the perovskite light-emitting layer of the (FA₁₋ₓGAₓ)_{0.87}Cs_{0.13}PbBr₃ 3 cation-mixed perovskite light-emitting layer and the GA mixing ratio of the (FA_{1-y}GA_{0.2}MA_{y})_{0.87}Cs_{0.13}PbBr₃ 4 cation-mixed perovskite light-emitting was shown in tables 11 and table 12, respectively.

TABLE 11

| GA mixing ratio (%) | L₅₀ at 100nit (hr ) |
|---|---|
| 0 | 0.17 |
| 5 | 35.99 |
| 6 | 39.16 |
| 7 | 44.48 |
| 8 | 45.07 |
| 9 | 47.38 |
| 10 | 47.25 |
| 11 | 51.23 |
| 12 | 48.99 |
| 13 | 55.20 |
| 14 | 52.94 |
| 15 | 53.81 |
| 16 | 55.48 |
| 17 | 57.57 |
| 18 | 58.54 |
| 19 | 57.51 |
| 20 | 57.43 |
| 22 | 59.60 |
| 24 | 57.99 |
| 26 | 56.41 |
| 28 | 54.80 |
| 30 | 49.70 |
| 32 | 50.53 |
| 34 | 48.08 |
| 36 | 41.10 |
| 38 | 42.04 |
| 40 | 34.79 |
| 45 | 10.97 |
| 50 | 2.86 |
| 55 | 0.03 |
| 60 | 0.06 |
| 80 | 0.06 |
| 100 | 0.02 |

TABLE 12

| GA mixing ratio (%) | L₅₀ at 100nit (hr) |
|---|---|
| 0 | 57.01 |
| 5 | 82.91 |
| 6 | 92.04 |
| 7 | 93.99 |
| 8 | 99.56 |
| 9 | 106.46 |
| 10 | 107.23 |
| 11 | 102.87 |
| 12 | 99.25 |
| 13 | 91.45 |
| 14 | 87.08 |
| 15 | 80.73 |
| 16 | 62.06 |
| 17 | 55.93 |
| 18 | 50.86 |
| 19 | 43.46 |
| 20 | 37.15 |
| 21 | 21.20 |
| 22 | 18.66 |
| 23 | 14.90 |
| 24 | 12.41 |
| 25 | 9.07 |
| 26 | 7.16 |
| 27 | 4.80 |
| 28 | 3.26 |
| 29 | 1.75 |
| 30 | 1.00 |

As described above, since the light emitting device including the perovskite film having the 4 cation-mixed structure according to the present disclosure as a light emitting layer exhibits improved current efficiency and operational lifetime compared to the light emitting device including the conventional perovskite film, it can useful in place of the conventional light emitting device.

On the other hand, the embodiments of the present disclosure disclosed in the specification and figures are only presented as specific examples to aid understanding, and are not intended to limit the scope of the present disclosure. In addition to the embodiments disclosed herein, it is obvious to those of ordinary knowledge in the field that other modified examples based on the technical idea of the present disclosure can be implemented

1. A Perovskite light-emitting material comprising perovskite crystal having structure of ABX₃ (3D), A₄BX₆ (0D), AB₂X₅ (2D), A₂BX₄ (2D), A₂BX₆ (0D), A₂B⁺B³⁺X₆ (3D), A₃B₂X₉ (2D) or Aₙ₋₁BₙX₃ₙ₊₁ (quasi-2D) (n is an integer between 2 and 6), where A is a monovalent cation, B is a metal material, and X is a halogen element,
wherein when tolerance factor (t) is defined as
$$t = \frac{{R}_{A} + {R}_{X}}{\sqrt{2} ( {R}_{B} + {R}_{X} )}$$
(R_{A}, R_{B} and R_{X} are respectively the ionic radius of A, B, X), structure having a first monovalent cation (A1) capable of making the tolerance factor as 1.01 or less at the site A of the perovskite structure, and structure having a second monovalent cation (A2) capable of making the tolerance factor of equal to or greater than 1.01 and less than 3 are mixed in the perovskite crystal,
wherein the second monovalent cation is simultaneously included both in the A site of inner structure of the perovskite crystal and outer surface of the perovskite crystal.

2. The perovskite light-emitting material of claim 1, wherein the perovskite light-emitting material is a colloidal nanoparticle dispersed in a solvent or polycrystalline bulk thin film.

3. The perovskite light-emitting material of claim 1, wherein the first monovalent cations is at least one selected from the group of methylammonium, formamidinium, Cs and Rb capable of making the tolerance factor as 1.01 or less, or combination thereof,
wherein the second monovalent cations is at least one selected from the group of ethylammonium, guanidinium, tert-butylammonium, diethylammonium, dimethylammonium, ethane-1,2,-diammonium, imidazolium, n-propylammonium, iso-propylammonium and pyrrolidinium capable of making the tolerance factor equal to or greater than 1.01 and less than 3.

4. The perovskite light-emitting material of claim 1, wherein a ratio of the second monovalent cation among total monovalent cations of the A site is 5% to 60% relative to the total amount of the mixture of the first monovalent cation and the second monovalent cation.

5. The perovskite light-emitting material of claim 2, wherein the perovskite light-emitting material is a nanoparticle that has crystal size equal to or greater than the exciton Bohr diameter and less than 30 nm.

6. The perovskite light-emitting material of claim 1, wherein the perovskite light-emitting material emits light in the range of 200 nm to 1500 nm.

7. The perovskite light-emitting material of claim 1,
wherein the first monovalent cation is a combination of methylammonium, formamidinium, and cesium,
wherein the second monovalent cation is guanidinium that is contained in an amount of 5% to 60% relative to the total amount of mixture of methylammonium, formamidinium, cesium and guanidinium.

8. The perovskite light-emitting material of claim 2,
wherein the perovskite light-emitting material is a nanoparticle that converts the wavelength of light generated from an excitation light source to a specific wavelength.

9. The perovskite light-emitting material of claim 2,
wherein the perovskite light-emitting material is a nanoparticle that further includes multiple organic ligands surrounding the nanocrystals in the nanoparticle and is dispersed in an organic solvent.

10. A Perovskite light-emitting material comprising perovskite crystal having structure of ABX₃ (3D), A₄BX₆ (0D), AB₂X₅ (2D), A₂BX₄ (2D), A₂BX₆ (0D), A₂B⁺B³⁺X₆ (3D), A₃B₂X₉ (2D) or Aₙ₋₁BₙX₃ₙ₊₁ (quasi-2D) (n is an integer between 2 and 6), where A is a monovalent cation, B is a metal material, and X is a halogen element,
wherein when tolerance factor (t) is defined as
$$t = \frac{{R}_{A} + {R}_{X}}{\sqrt{2} ( {R}_{B} + {R}_{X} )}$$
(R_{A}, R_{B} and R_{X} are respectively the ionic radius of A, B, X), a structure having a first monovalent cation (A1) capable of making the tolerance factor as 1.01 or less at the site A of the perovskite structure, and a structure having a second monovalent cation (A2) capable of making the tolerance factor equal to or greater than 1.01 and less than 3 are mixed in the perovskite crystal,
wherein the first monovalent cations and the second monovalent cation are uniformly positioned at the A site of inner structure of the perovskite crystal.

11. The perovskite light-emitting material of claim 10,
wherein the perovskite light-emitting material is a colloidal nanoparticle dispersed in a solvent or polycrystalline bulk thin film.

12. The perovskite light-emitting material of claim 10,
wherein the first monovalent cations is at least one selected from the group of methylammonium, formamidinium, Cs and Rb capable of making the tolerance factor as 1.01 or less, or combination thereof,
wherein the second monovalent cations is at least one selected from the group of ethylammonium, guanidinium, tert-butylammonium, diethylammonium, dimethylammonium,
ethane-1,2,-diammonium, imidazolium, n-propylammonium, iso-propylammonium and pyrrolidinium capable of making the tolerance factor equal to or greater than 1.01 and less than 3.

13. The perovskite light-emitting material of claim 10,
wherein the ratio of the second monovalent cation among total monovalent cations of the A site is 5% to 60% relative to the total amount of the mixture of the first monovalent cation and the second monovalent cation.

14. The perovskite light-emitting material of claim 11,
wherein the perovskite light-emitting material is a nanoparticle that has crystal size equal to or greater than the exciton Bohr diameter and less than 30 nm.

15. The perovskite light-emitting material of claim 10,
wherein the perovskite light-emitting material emits light in the range of 300 nm to 1500 nm.

16. The perovskite light-emitting material of claim 10,
wherein the first monovalent cation is a combination that includes all of methylammonium, formamidinium, and cesium,
wherein the second monovalent cation is guanidinium that is contained in an amount of 5% to 60% relative to the total amount of mixture of methylammonium, formamidinium, cesium and guanidinium.

17. The perovskite light-emitting material of claim 10,
wherein the first monovalent cation is a combination of methylammonium, formamidinium, and cesium,
wherein the second monovalent cation is guanidinium
wherein the methylammonium among the first monovalent cations is contained in an amount of 1% to 20% relative to the total amount of mixture of methylammonium, formamidinium, cesium and guanidinium.

18. A Perovskite wavelength converting body comprising:
the perovskite light-emitting material of claim 8 converting wavelength of light generated from an excitation light source to a specific wavelength; and
a dispersion medium for dispersing the perovskite light-emitting material.

19. A Perovskite light-emitting device comprising:
substrate;
a first electrode on the substrate;
a light-emitting layer positioned on the first electrode; and
a second electrode positioned on the light-emitting layer,
wherein the light-emitting layer is the perovskite light-emitting material of claim 1.

20. A Perovskite light-emitting device comprising:
base structure;
an excitation light source emitting a predetermined wavelength while disposed on the base structure; and
the perovskite wavelength converting body of claim 18 disposed in the optical path of the excitation light source.
